SEQUENCE LISTING (11 sequences)

SEQ ID NO: 1 - DNA, 13 bp, Artificial Sequence
  First 13 bases of Template 1
aaacttgatgctc

SEQ ID NO: 2 - DNA, 13 bp, Artificial Sequence
  First 13 bases of Template 2
cccaaacttgatg

SEQ ID NO: 3 - DNA, 79 bp, Artificial Sequence
  Template 1
cagaggaagtatcctgtcgtttagcatacccaaattgatgctcctgacctcagctgcacgtaagtgcagctgaggtcag

SEQ ID NO: 4 - DNA, 80 bp, Artificial Sequence
  Template 2
tcacagaggaagtatcctgtcgtttagcatacccaaacttgatgctgacctcagctgcacgtaagtgcagctgaggtcag

SEQ ID NO: 5 - DNA, 17 bp, Artificial Sequence
  Primer E8TT
gataggactcatcacca

SEQ ID NO: 6 - DNA, 114 bp, Artificial Sequence
  Synthetic DNA template
acggagaacgaagagaaaaggataggactcatcaccattagatgaccctgccttgccttgtcgaactccacgcacaaacacggacaggaccctctctggccgcgtgtctccttc

SEQ ID NO: 7 - DNA, 92 bp, Artificial Sequence
  Extended Template 1
cagaggaagtatcctgtcgtttagcatacccaaattgatgctcctgacctcagctgcacgtaagtgcagctgaggtcaggagcatcaagttt

SEQ ID NO: 8 - DNA, 93 bp, Artificial Sequence
  Extended Template 2
tcacagaggaagtatcctgtcgtttagcatacccaaacttgatgctgacctcagctgcacgtaagtgcagctgaggtcagcatcaagtttggg

SEQ ID NO: 9 - DNA, 18 bp, Artificial Sequence
  Extended primer
gataggactcatcaccat

SEQ ID NO: 10 - DNA, 28 bp, Artificial Sequence
  Template
ctatcctgagtagtggtaatctactggg

SEQ ID NO: 11 - DNA, 85 bp, Artificial Sequence
  Extended template
ttcgcacacgcgtttgaccgaagcttgcactgacctcagctgcacgtaagtgcagctgaggtcagtgcaagcttcggtcaaacgc

(19) United States
(12) Patent Application Publication
(10) Pub. No.: US 2022/0372061 A1
(43) Pub. Date: Nov. 24, 2022

(54) NOVEL NUCLEOTIDE ANALOGUES AND METHODS FOR USE

(71) Applicant: The Trustees of Columbia University in the City of New York, New York, NY (US)

(72) Inventors: Jingyue Ju, Englewood Cliffs, NJ (US); Shiv Kumar, Belle Mead, NJ (US); James Russo, New York, NY (US); Xiaoxu Li, New York, NY (US); Xin Chen, Hunan (CN); Minchen Chien, Tenafly, NJ (US); Steffen Jockusch, New York, NY (US); Xuanting Wang, Braintree, MA (US); Irina Morozova, Bronx, NY (US); Chuanjuan Tao, Fort Lee, NJ (US); Sergey Kalachikov, Bronx, NY (US); Shundi Shi, Ozone Park, NY (US)

(73) Assignee: The Trustees of Columbia University in the City of New York, New York, NY (US)

(21) Appl. No.: 17/622,214

(22) PCT Filed: Jul. 9, 2020
(86) PCT No.: PCT/US2020/041455

§ 371 Date: Dec. 22, 2021

Related U.S. Application Data
(60) Provisional application No. 62/872,164, filed on Jul. 9, 2019.

(51) Int. Cl. C07H 19/10; C07H 19/20; C12Q 1/6874; C12Q 1/6816

(52) U.S. Cl. CPC C07H 19/10; C07H 19/20; C12Q 1/6874; C12Q 1/6816

(57) ABSTRACT

The invention provides various orthogonal nucleotide analogues and methods for using combinations of said various orthogonal nucleotide analogues for sequencing by synthesis.

This application claims priority of U.S. Provisional Application No. 62/872,164, filed Jul. 9, 2019, the contents of which are hereby incorporated by reference.

Throughout this application, various publications and patents are referenced. Full citations for these references may be found at the end of the specification immediately preceding the claims. The disclosures of these publications and patents in their entirety are hereby incorporated by reference into this application to more fully describe the state of the art to which this invention pertains.

This application incorporates-by-reference nucleotide and/or amino acid sequences which are present in the file named “200824_90884-A-PCT_Sequence_Listing_BI.txt,” which is 2.81 kilobytes in size, and which was created Aug. 19, 2020 in the IBM-PC machine format, having an operating system compatibility with MS-Windows, which is contained in the text file filed Aug. 24, 2020 as part of this application.

### BACKGROUND OF THE INVENTION

DNA sequencing is a fundamental tool in biological and medical research, and is especially important for the paradigm of personalized medicine. Various new DNA sequencing methods have been investigated with the aim of eventually realizing the goal of the $1,000 genome; the dominant method is sequencing by synthesis (SBS), an approach that determines DNA sequences during the polymerase reaction (Hyman 1988; Ronaghi et al. 1998; Ju et al. 2003; Li 2003; Braslavsky et al. 2003; Ruparel et al. 2005; Margulies et al. 2005; Ju et al. 2006; Wu et al. 2007; Guo et al. 2008; Bentley et al. 2008; Harris et al. 2008; Eid et al. 2009; Rothberg et al. 2011).

### SUMMARY

The invention provides a nucleotide analogue having structure:

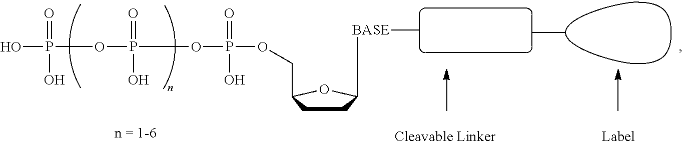

wherein:
   BASE comprises adenine, guanine, cytosine, thymine, uracil, hypoxanthine or analog thereof;
   Cleavable Linker comprises DTM, Azo, 2-Nitrobenzyl, Allyl, Azidomethyl, or TCO Derivative, and is attached to the base via 5 position of pyrimidines (C, U) or 7 position of deazapurines (A, G, I); and
   Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye.

The invention provides a nucleotide analogue having the structure:

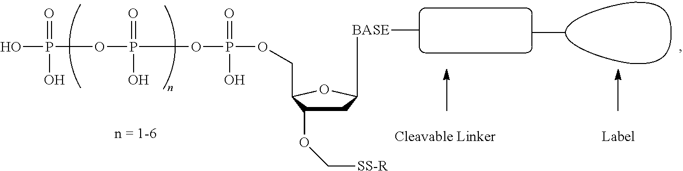

wherein:
   BASE comprises adenine, guanine, cytosine, thymine, uracil, hypoxanthine or analog thereof;
   R comprises methyl, ethyl, propyl, t-butyl, aryl, alkyl aryl;
   Cleavable Linker comprises DTM, Azo, 2-Nitrobenzyl, Allyl, Azidomethyl or TCO Derivative; and
   Label comprises a fluorescent dye, a pH responsive fluorescent dye, a cluster of a fluorescent dye, a cluster of a pH responsive fluorescent dye, an anchor for attachment of a fluorescent dye, a cluster of an anchor for attachment of fluorescent dyes, or an anchor and dye.

The invention provides a nucleotide analogue having structure:

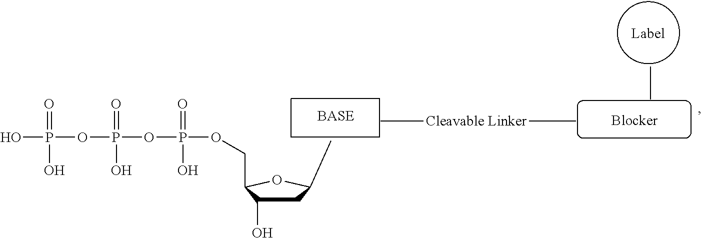

wherein:
   BASE comprises adenine, guanine, cytosine, thymine, uracil, hypoxanthine or analog thereof;
   Cleavable Linker comprises DTM, Azo, 2-Nitrobenzyl, Allyl, Azidomethyl, or TCO Derivative, or more than one of these cleavable linkers, including the special case where one cleavable linker is present between the base and the blocker and a second different cleavable linker is present between the blocker and the label;
   Blocker is a nucleotide or oligonucleotide comprising 2-50 monomer units of abasic sugars or modified nucleosides or a combination thereof; and blocker is connected to the 5-position of pyrimidines (C, U) and 7-position of deazapurines (A, G, I) via a cleavable linker;
   wherein a Blocker is a moiety that, after incorporation, prevents further incorporation of additional nucleotides or nucleotide analogues into a primer strand; and
   Label comprises a fluorescent dye, a pH responsive fluorescent dye, a cluster of a fluorescent dye, a cluster of a pH responsive fluorescent dye, an anchor for attachment of a fluorescent dye, a cluster of an anchor for attachment of fluorescent dyes, or an anchor and dye, wherein the label is attached to the blocker.

The invention provides a nucleotide analogue having structure:

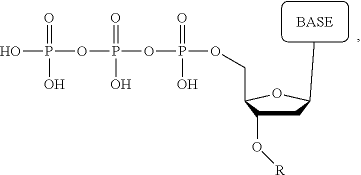

wherein:
   BASE comprises adenine, guanine, cytosine, uracil, thymine, hypoxanthine or analogue thereof; and
   R is a cleavable chemical group comprising alkyl DTM, Azo, 2-Nitrobenzyl, Allyl and Azidomethyl Derivatives.

The invention provides a nucleotide analogue having structure:

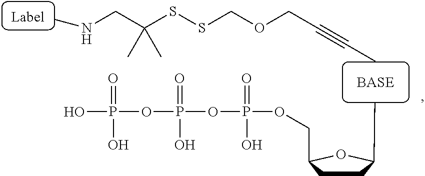

wherein:
   BASE comprises adenine, guanine, cytosine, thymine, uracil, hypoxanthine or analog thereof; and
   Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye.

The invention provides a nucleotide analogue having structure:

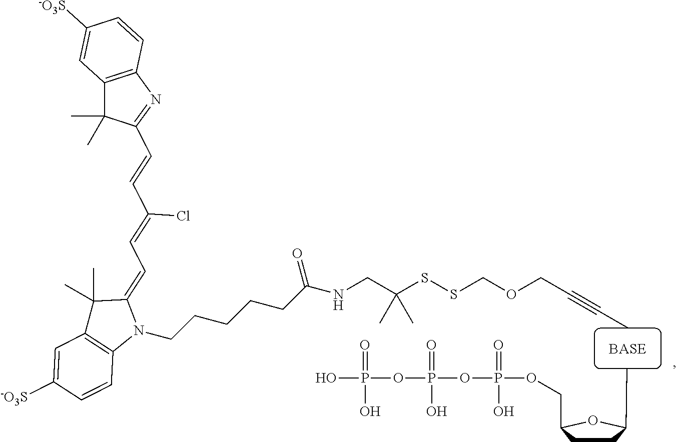

wherein BASE comprises adenine, guanine, cytosine, thymine, uracil, hypoxanthine or analog thereof.

The invention provides a nucleotide analogue having structure:

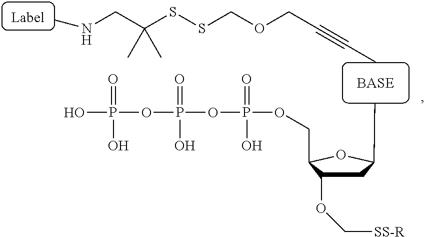

wherein:
   BASE comprises adenine, guanine, cytosine, thymine, uracil, hypoxanthine or analog thereof;
   Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye; and
   R comprises methyl, ethyl, propyl, t-butyl, aryl, alkyl aryl.

The invention provides a nucleotide analogue having structure:

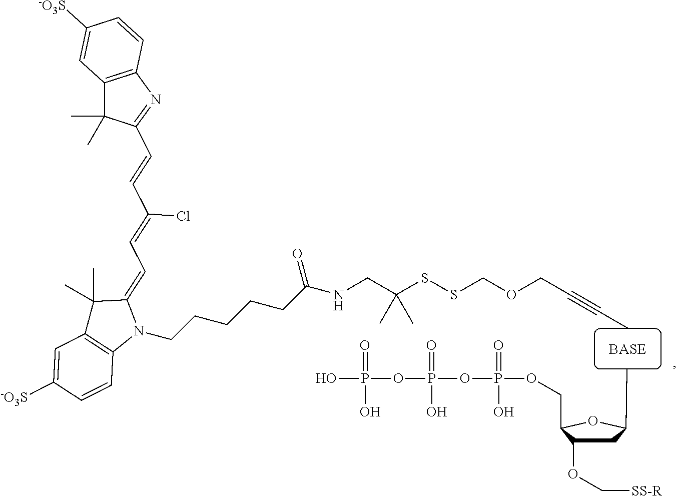

wherein:
   BASE comprises adenine, guanine, cytosine, thymine, uracil, hypoxanthine or analog thereof; and
   R comprises methyl, ethyl, propyl, t-butyl, aryl, alkyl aryl.

The invention provides a nucleotide analogue having structure:

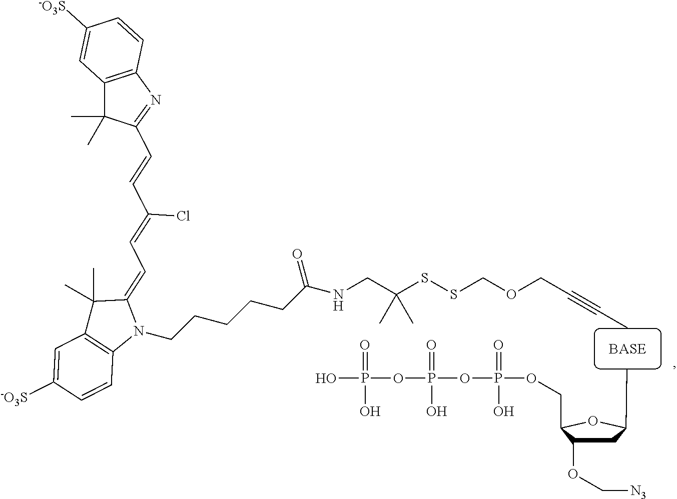

wherein BASE comprises adenine, guanine, cytosine, thymine, uracil, hypoxanthine or analog thereof

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) (A) a fluorescently labeled dideoxynucleotide analogue comprising a base and a fluorescent label linked to the base via a cleavable linker and (B) an anchor labeled dideoxynucleotide analogue comprising a base and an anchor attached to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) an anchor labeled nucleotide analogue comprising a base and an anchor attached to the base via a cleavable linker and a blocking group at the 3′-OH position, wherein said blocking group prevents incorporation of a subsequent nucleotide analogue into the extended primer strand,
      wherein the cleavable linkers and 3′-OH group are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) an anchor labeled nucleotide analogue comprising a base, a blocking group linked to the base via a cleavable linker, and an anchor linked to the base distal to the blocking group, wherein said blocking group prevents or greatly reduces incorporation of a subsequent nucleotide analogue into the extended primer strand,
      wherein the cleavable linkers are cleavable by an identical cleaving agent;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the anchor of the provided anchor labeled nucleotide analogue of step (b), wherein said anchor binding group comprises a fluorescent label identical to the fluorescent label of the fluorescently labeled nucleotide analogue of step (b);
f) identifying any fluorescence signal due to the binding of the anchor binding group to the anchor of any incorporated anchor labeled nucleotide analogue of step (b);
g) repeating steps (b)-(f) with two different labeled nucleotide analogues that are different from the two different labeled nucleotide analogues from the previous iteration of step (b);
h) cleaving the cleavable linkers from the incorporated nucleotide analogue, thereby removing any label, anchor, or blocking group from the incorporated nucleotide analogue of step (b);
i) cleaving the 3′-O blocking group from any incorporated nucleotide analogue from step (c); and
j) iteratively repeating steps (b) to (i) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) fluorescently labeled dideoxynucleotide analogue comprising a base and a fluorescent label linked to the base via a cleavable linker, (B) pH-responsive fluorescently labeled dideoxynucleotide analogue comprising a base and a pH-responsive fluorescent label linked to the base via a cleavable linker, (C) two different anchor labeled dideoxynucleotide analogues, wherein each analogue comprises a different anchor attached to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a pH-responsive fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (C) two different anchor labeled nucleotide analogues comprising a base and an anchor attached to the base via a cleavable linker and a blocking group at the 3′-OH position, wherein said blocking group prevents incorporation of a subsequent nucleotide analogue into the extended primer strand, wherein each analogue comprises a different anchor attached to the base via a cleavable linker,
      wherein the cleavable linkers and 3′-O blocking groups are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand, and (C) two different anchor labeled nucleotide analogues comprising a base, a blocking group linked to the base via a cleavable linker, and an anchor linked to the base distal to the blocking group, wherein said blocking group prevents or greatly reduces incorporation of a subsequent nucleotide analogue into the extended primer strand,
      wherein each analogue comprises a different anchor attached to the base via a cleavable linker,
      wherein the cleavable linkers are cleavable by an identical cleaving agent;
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label has same or similar absorption and emission profile as the fluorescent label on the fluorescently labeled nucleotide analogue and identifying any fluorescence signal due to incorporation of a labeled nucleotide analogue from step (b);
e) contacting the incorporated nucleotide analogue from step (b) with (A) an anchor binding group that binds to the anchor of only one of the anchor labeled nucleotide analogues of step (b), wherein the anchor binding group comprises the same fluorescent label as the fluorescently labeled nucleotide analogue of step (b), and (B) an anchor binding group that binds only to the anchor of the remaining anchor labeled nucleotide analogue, wherein the anchor binding group comprises the same pH-responsive fluorescent label as the pH-responsive fluorescently labeled nucleotide analogue of step (b);
f) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of an anchor labeled nucleotide analogue from step (b);
g) washing the incorporated nucleotide analogue from step (b) at a pH at which the pH-responsive fluorescent label no longer has the same or similar absorption and emission profile as the fluorescent label on the fluorescently labeled nucleotide analogue and identifying any fluorescence signal due to incorporation of a labeled nucleotide analogue from step (b);
h) cleaving the cleavable linker from the incorporated nucleotide analogue, thereby removing any label, anchor, or blocking group from the incorporated nucleotide analogue of step (b);
i) cleaving the 3′-O blocking group from any incorporated nucleotide analogue from step (c); and
j) iteratively repeating steps (b) to (i) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) (A) a fluorescently labeled dideoxynucleotide analogue comprising a base and a fluorescent label linked to the base via a cleavable linker and (B) pH-responsive fluorescently labeled dideoxynucleotide analogue comprising a base and a pH-responsive fluorescent label linked to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a pH-responsive fluorescently labeled nucleotide analogue comprising a base and a pH-responsive fluorescent label linked to the base via a cleavable linker and a blocking group at the 3′-OH position, wherein said blocking group prevents incorporation of a subsequent nucleotide analogue into the extended primer strand,
      wherein the cleavable linkers and the 3′-O blocking group are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand,
      wherein the cleavable linkers are cleavable by an identical cleaving agent;
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label has same or similar absorption and emission profile as the fluorescent label on the fluorescently labeled nucleotide analogue and identifying any fluorescence signal due to incorporation of a labeled nucleotide analogue from step (b);
e) repeating steps (b)-(d) with two different labeled nucleotide analogues that are different from the two different labeled nucleotide analogues from the previous iteration of step (b);
f) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of a labeled nucleotide analogue from step (b),
   wherein steps (e) and (f) may be performed in the reverse order;
g) cleaving the cleavable linkers from the incorporated nucleotide analogue, thereby removing any label or blocking group from the incorporated nucleotide analogue of step (b);
h) cleaving the 3′-O blocking group from any incorporated nucleotide analogue from step (c); and
i) iteratively repeating steps (b) to (h) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via a carbamyl TCO linker, (C) an anchor labeled dideoxynucleotide analogue comprising a base and an anchor attached to the base via the first cleavable linker, and (D) an anchor labeled dideoxynucleotide analogue comprising a base and an anchor attached to the base via a carbamyl TCO linker;
   (ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via a carbamyl TCO linker, and a blocking group at the 3′-OH position, wherein said blocking group prevents incorporation of a subsequent nucleotide analogue into the extended primer strand, (C) an anchor labeled nucleotide analogue comprising a base and an anchor attached to the base via the first cleavable linker, and a blocking group at the 3′-OH position, wherein said blocking group prevents incorporation of a subsequent nucleotide analogue into the extended primer strand, and (D) an anchor labeled nucleotide analogue comprising a base and an anchor attached to the base via a carbamyl TCO linker, and a blocking group at the 3′-OH position, wherein said blocking group prevents incorporation of a subsequent nucleotide analogue into the extended primer strand, wherein the 3′-O blocking group and the first cleavable linker are cleavable by the same cleaving agent; or
   (iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a carbamyl TCO linker, and a fluorescent label linked to the base distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a first cleavable linker, and an anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a carbamyl TCO linker, and an anchor linked to the base distal to the blocking group, wherein said blocking group prevents or greatly reduces incorporation of a subsequent nucleotide analogue into the extended primer strand,
      wherein the fluorescent labels on each analogue are the same,
      wherein the anchors on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the anchor of the provided anchor labeled nucleotide analogues of step (b), wherein said anchor binding group comprises a fluorescent label identical to the fluorescent label of the fluorescently labeled nucleotide analogues of step (b);
f) identifying any fluorescence signal due to the binding of the anchor binding group to the anchor of any incorporated anchor labeled nucleotide analogue of step (b);
g) contacting the incorporated nucleotide analogue with a tetrazine derivative to click to the TCO moiety of the carbamyl TCO linker to release any label or anchor linked by a carbamyl TCO linker and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a cleavable linker, (B) a fluorescently labeled dideoxynucleotide analogue comprising a base and a fluorescent label and a first anchor linked to the base via a cleavable linker, (C) an anchor labeled dideoxynucleotide analogue comprising a base and the first anchor and a second anchor attached to the base via a cleavable linker, and (D) an anchor labeled dideoxynucleotide analogue comprising a base and the second anchor attached to the base via a cleavable linker, wherein the cleavable linkers are cleavable by an identical cleavage agent;
   (ii) (A) a fluorescently labeled nucleotide analogue comprising a base and a fluorescent label linked to the base via a cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label and a first anchor linked to the base via a cleavable linker, and a blocking group at the 3′-OH position, wherein said blocking group prevents incorporation of a subsequent nucleotide analogue into the extended primer strand, (C) an anchor labeled nucleotide analogue comprising a base and the first anchor and a second anchor attached to the base via the cleavable linker, and a blocking group at the 3′-OH position, wherein said blocking group prevents incorporation of a subsequent nucleotide analogue into the extended primer strand, and (D) an anchor labeled nucleotide analogue comprising a base and the second anchor attached to the base via a cleavable linker, and a blocking group at the 3′-OH position, wherein said blocking group prevents incorporation of a subsequent nucleotide analogue into the extended primer strand, wherein the cleavable linkers and the 3′-O blocking group are cleavable by an identical cleavage agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a cleavable linker, and a fluorescent label and a first anchor linked to the base distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a cleavable linker, and the first anchor and a second anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a cleavable linker, and the second anchor linked to the base distal to the blocking group, wherein said blocking group prevents or greatly reduces incorporation of a subsequent nucleotide analogue into the extended primer strand, wherein the cleavable linkers are cleavable by an identical cleavage agent,
   wherein the fluorescent labels on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the second anchor of the nucleotide analogues of step (b), wherein said anchor binding group comprises a fluorescent label identical to fluorescent label of the fluorescently labeled nucleotide analogues of step (b);
f) identifying any fluorescence signal due to the binding of the anchor binding group to the anchor of any incorporated nucleotide analogue of step (b);
g) contacting the incorporated nucleotide analogue with a second anchor binding group that binds to the first anchor of the nucleotide analogues of step (b) and comprises a moiety that quenches the fluorescent signal of any fluorescent label attached to the nucleotide analogue to which the anchor binding group attaches, and identifying any fluorescence signal due to incorporation of a fluorescently labeled nucleotide analogue;
h) contacting the incorporated nucleotide analogue with a cleaving agent that cleaves the cleavable linker and any 3′-O blocking group; and
i) iteratively repeating steps (b) to (h) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via the first cleavable linker and a carbamyl TCO linker attached distal to the first cleavable linker, (C) an anchor labeled dideoxynucleotide analogue comprising a base and an anchor linked to the base via the first cleavable linker and a carbamyl TCO linker attached distal to the first cleavable linker, and (D) an anchor labeled dideoxynucleotide analogue comprising a base and an anchor attached to the base via the first cleavable linker;
   (ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, (C) an anchor labeled nucleotide analogue comprising a base and an anchor attached to the base via the first cleavable linker, and a blocking group at the 3′-OH position, wherein said blocking group prevents incorporation of a subsequent nucleotide analogue into the extended primer strand, and (D) an anchor labeled nucleotide analogue comprising a base and an anchor linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, wherein the 3′-OH blocking group and the first cleavable linker are cleavable by the same agent; or
   (iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via the first cleavable linker, and a fluorescent label linked to the base via a carbamyl TCO linker distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a first cleavable linker, and an anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a the first cleavable linker, and an anchor linked to the base via a carbamyl TCO linker distal to the blocking group, wherein said blocking group prevents or greatly reduces incorporation of a subsequent nucleotide analogue into the extended primer strand,
      wherein the fluorescent labels on each analogue are the same,
      wherein the anchors on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue in step (b);
e) contacting the incorporated nucleotide analogue from step (b) with an anchor binding group that binds to the anchor of the provided anchor labeled nucleotide analogues of step (b), wherein said anchor binding group comprises a fluorescent label identical to the fluorescent label of the fluorescently labeled nucleotide analogues of step (b);
f) identifying any fluorescence signal due to the binding of the anchor binding group to the anchor of any incorporated anchor labeled nucleotide analogue of step (b);
g) contacting the incorporated nucleotide analogue with a tetrazine derivative to click to the TCO moiety of the carbamyl TCO linker to release any label or anchor linked by a carbamyl TCO linker and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via the first cleavable linker and a carbamyl TCO linker attached distal to the first cleavable linker, (C) a pH-responsive fluorescently labeled dideoxynucleotide analogue comprising a base and a pH-responsive fluorescent label linked to the base via the first cleavable linker and a carbamyl TCO linker attached distal to the first cleavable linker, and (D) a pH-responsive fluorescently labeled dideoxynucleotide analogue comprising a base and a pH-responsive fluorescent label attached to the base via the first cleavable linker;
   (ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, (C) a pH-responsive fluorescently labeled nucleotide analogue comprising a base and a pH-responsive fluorescent label attached to the base via the first cleavable linker, and a blocking group at the 3′-OH position, wherein said blocking group prevents incorporation of a subsequent nucleotide analogue into the extended primer strand, and (D) a pH-responsive fluorescently labeled nucleotide analogue comprising a base and a pH-responsive fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, wherein the 3′-OH blocking group and the first cleavable linker are cleavable by the same agent; or
   (iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via the first cleavable linker, and a fluorescent label linked to the base via a carbamyl TCO linker distal to the blocking group, wherein said blocking group prevents or greatly reduces incorporation of a subsequent nucleotide analogue into the extended primer strand, (C) a pH-responsive fluorescently labeled nucleotide analogue nucleotide analogue comprising a base, a blocking group linked to the base via a first cleavable linker, and a pH-responsive fluorescent label linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a the first cleavable linker, and a pH-responsive fluorescent label linked to the base via a carbamyl TCO linker distal to the blocking group, wherein said blocking group prevents or greatly reduces incorporation of a subsequent nucleotide analogue into the extended primer strand,
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label does not have the same or similar absorption and emission profile as the fluorescent label on the fluorescently labeled nucleotide analogues and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b), wherein steps (d) and (e) may be performed in the reverse order;
f) contacting the incorporated nucleotide analogue with tetrazine to click the TCO moiety of the carbamyl TCO linker to release any label linked by a carbamyl TCO linker;
g) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced, thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a cleavable linker, (B) a fluorescently labeled dideoxynucleotide analogue comprising a base and a fluorescent label and an anchor linked to the base via a cleavable linker, (C) a pH-responsive fluorescently labeled dideoxynucleotide analogue comprising a base and a pH-responsive fluorescent label linked to the base via a cleavable linker, and (D) a pH-responsive fluorescently labeled dideoxynucleotide analogue comprising a base and pH-responsive fluorescent label and anchor attached to the base via a cleavable linker, wherein the cleavable linkers are cleavable by an identical cleaving agent;
   (ii) (A) a fluorescently labeled nucleotide analogue comprising a base and a fluorescent label linked to the base via a cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label and an anchor linked to the base via a cleavable linker, and a blocking group at the 3′-OH position, wherein said blocking group prevents incorporation of a subsequent nucleotide analogue into the extended primer strand, (C) a pH-responsive fluorescently labeled nucleotide analogue comprising a base and pH-responsive fluorescent label linked to the base via a cleavable linker, and a blocking group at the 3′-OH position, wherein said blocking group prevents incorporation of a subsequent nucleotide analogue into the extended primer strand, and (D) a pH-responsive fluorescently labeled nucleotide analogue comprising a base and a pH-responsive fluorescent label and anchor attached to the base via a cleavable linker, and a blocking group at the 3′-OH position, wherein said blocking group prevents incorporation of a subsequent nucleotide analogue into the extended primer strand, wherein the cleavable linkers and the 3′-O blocking group are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, (C) a pH-responsive fluorescently labeled nucleotide analogue comprising a base, a blocking group linked to the base via a cleavable linker, a pH-responsive fluorescent label linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a cleavable linker, and a pH-responsive fluorescent label and an anchor linked to the base distal to the blocking group, wherein said blocking group prevents or greatly reduces incorporation of a subsequent nucleotide analogue into the extended primer strand, wherein the cleavable linkers are cleavable by an identical cleavage agent,
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label does not have the same or similar absorption and emission profile as the fluorescent label on the fluorescently labeled nucleotide analogues and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b), wherein steps (d) and (e) may be performed in the reverse order;
f) contacting the incorporated nucleotide analogue from step (b) with an anchor binding group that binds to the anchor of the nucleotide analogues of step (b), wherein said anchor binding group comprises a moiety that quenches the fluorescent label of the fluorescently labeled nucleotide analogues of step (b);
g) washing away any unbound anchor binding group comprising a quenching moiety at a pH at which the pH-responsive fluorescent label has same or similar absorption and emission profile as the fluorescent label on the fluorescently labeled nucleotide analogues and identifying any fluorescence signal due to incorporation of a labeled nucleotide analogue from step (b);
h) contacting the incorporated nucleotide analogue with a cleaving agent that cleaves the cleavable linker and any 3′-O blocking group; and
i) iteratively repeating steps (b) to (h) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) (A) a fluorescently labeled dideoxynucleotide analogue comprising a base and a fluorescent label linked to the base via a cleavable linker and (B) a fluorescently labeled dideoxynucleotide analogue comprising a base and a fluorescent label and anchor linked to the base via a cleavable linker, wherein the cleavable linkers are cleavable by the identical cleavage agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a fluorescently labeled nucleotide analogue comprising a base and a fluorescent label and anchor attached to the base via a cleavable linker and a blocking group at the 3′-OH position, wherein said blocking group prevents incorporation of a subsequent nucleotide analogue into the extended primer strand, wherein the cleavable linkers and 3′-O blocking group are cleavable by the identical cleavage agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, wherein the cleavable linkers are cleavable by the identical cleavage agent,
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) repeating steps (b)-(d) with two different labeled nucleotide analogues that are different from the two different labeled nucleotide analogues from the previous iteration of step (b);
f) contacting the incorporated nucleotide analogue from step (b) with an anchor binding group that binds to the anchor of the nucleotide analogues of step (b), wherein said anchor binding group comprises a moiety that quenches the fluorescent label of the fluorescently labeled nucleotide analogues of step (b);
g) identifying any fluorescence signal due to incorporation of a fluorescently labeled nucleotide analogue in step (b);
h) cleaving the cleavable linkers from the incorporated nucleotide analogue, thereby removing any label, anchor, or blocking group from the incorporated nucleotide analogue of step (b);
i) cleaving the 3′-O blocking group from any incorporated nucleotide analogue from step (c); and
j) iteratively repeating steps (b) to (i) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are:
   (A) fluorescently labeled dideoxynucleotide analogue comprising a base and a fluorescent label linked to the base via a cleavable linker, (B) fluorescently labeled dideoxynucleotide analogue comprising a base and a fluorescent label linked to the base via an uncleavable linker, (C) an anchor labeled dideoxynucleotide analogue comprising a base and an anchor attached to the base via a cleavable linker, and (D) an anchor labeled dideoxynucleotide analogue comprising a base and an anchor attached to the base via an uncleavable linker;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue in step (b);
e) contacting the incorporated nucleotide analogue from step (b) with an anchor binding group that binds to the anchor of the anchor labeled nucleotide analogues of step (b);
f) identifying any fluorescence signal due to incorporation of a fluorescently labeled nucleotide analogue in step (b);
g) contacting the incorporated nucleotide analogue of step (b) with an agent that cleaves the cleavable linker of the nucleotide analogues of step (b) and cleaves the 3′-O blocking group of the nucleotide analogues of step (c);
h) identifying any fluorescence signal due to incorporation of a fluorescently labeled nucleotide analogue in step (b);
i) photobleaching the incorporated nucleotide analogue of step (b) to thereby photobleach any remaining fluorescent label; and
j) iteratively repeating steps (b) to (i) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) a fluorescently labeled dideoxynucleotide analogue comprising a base and a fluorescent label linked to the base via a cleavable linker and (B) a fluorescently labeled dideoxynucleotide analogue comprising a base and a different fluorescent label linked to the base via a cleavable linker, wherein the cleavable linkers are cleavable by an identical cleavage agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a fluorescently labeled nucleotide analogue comprising a base and a different fluorescent label attached to the base via a cleavable linker and a blocking group at the 3′-OH position, wherein the cleavable linkers and 3′-O blocking groups are cleavable by the identical cleavage agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) a fluorescently labeled nucleotide analogue comprising a base, a blocking group linked to the base via a cleavable linker, and a different fluorescent label linked to the base distal to the blocking group, wherein said blocking group prevents or greatly reduces incorporation of a subsequent nucleotide analogue into the extended primer strand, wherein the cleavable linkers are cleavable by an identical cleavage agent;
c) contacting the nucleic acid templates with unlabeled nucleotide analogues (A, C, T, G) without any base modifications and comprising a 3′-O blocking group, wherein said 3′-O blocking groups are cleavable by an identical cleavage agent to the cleavable linkers and and/or blocking groups of the two labeled nucleotide analogues of step (b), and extending any unextended primers with said unlabeled nucleotide analogues, wherein step (c) occurs before, simultaneously, or after step (b);
d) identifying any fluorescence signal due to incorporation of a fluorescently labeled nucleotide analogue;
e) repeating steps (b)-(d) with two different labeled nucleotide analogues that are different from the two different labeled nucleotide analogues from the previous iteration of step (b), but with only two unlabeled nucleotides comprising a 3′-O blocking group different from the two labeled nucleotide analogues added in this step;
f) cleaving the cleavable linkers from the incorporated nucleotide analogue, thereby removing any label or blocking group from the incorporated nucleotide analogue of step (b) and (c);
g) identifying any fluorescence signal due to incorporation of a fluorescently labeled nucleotide analogue in step (b); and
h) iteratively repeating steps (b) to (g) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a cleavable linker, (B) a pH-responsive fluorescently labeled dideoxynucleotide analogue comprising a base and a pH-responsive fluorescent label linked to the base via a cleavable linker, (C) a fluorescently labeled dideoxynucleotide analogue comprising a base and a fluorescent label and anchor linked to the base via a cleavable linker, and (D) a pH-responsive fluorescently labeled dideoxynucleotide analogue comprising a base and a pH-responsive fluorescent label and identical anchor linked to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, (B) a pH-responsive fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, (C) a fluorescently labeled nucleotide analogue comprising a base and a fluorescent label and anchor linked to the base via a cleavable linker and a blocking group at the 3′-OH position, wherein said blocking group prevents incorporation of a subsequent nucleotide analogue into the extended primer strand, and (D) a pH-responsive fluorescently labeled nucleotide analogue comprising a base and a fluorescent label and identical anchor linked to the base via a cleavable linker and a blocking group at the 3′-OH position, wherein said blocking group prevents incorporation of a subsequent nucleotide analogue into the extended primer strand,
      wherein the cleavable linkers and 3′-O blocking groups are cleavable by an identical cleaving agent; or
   (iii) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand, (C) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a cleavable linker, and a pH-responsive fluorescent label and identical anchor linked to the base distal to the blocking group, wherein said blocking group prevents or greatly reduces incorporation of a subsequent nucleotide analogue into the extended primer strand,
      wherein the cleavable linkers are cleavable by an identical cleaving agent;
c) extending unextended primers with a nucleotide analogue without any base modifications and comprising a 3′-O blocking group, wherein step (c) occurs before, simultaneously or after step (b);
d) washing away any unincorporated nucleotide analogues at a pH at which the pH-responsive fluorescent label does not have the same or similar absorption and emission profile as the fluorescent label on the fluorescently labeled nucleotide analogue and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of an anchor labeled nucleotide analogue from step (b);
f) contacting the incorporated nucleotide analogue with an anchor binding group that binds to the anchor of the nucleotide analogues of step (b) and comprises a moiety that quenches the fluorescent signal of any fluorescent label attached to the nucleotide analogue to which the anchor binding group attaches, and identifying any fluorescence signal due to incorporation of a fluorescently labeled nucleotide analogue;
g) contacting the incorporated nucleotide analogue with a cleaving agent that cleaves the cleavable linker and any 3′-O blocking group; and
h) iteratively repeating steps (b) to (g) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) two fluorescently labeled nucleotide analogues comprising a base and a fluorescent label serving as an energy transfer donor linked to the base via a cleavable linker, an anchor for attachment of an energy transfer acceptor label, and a blocking group at the 3′-OH position, wherein said blocking group prevents incorporation of a subsequent nucleotide analogue into the extended primer strand,
      wherein the cleavable linkers and 3′-O blocking groups are cleavable by an identical cleaving agent;
      wherein each of the nucleotide analogues has a different anchor; or
   (ii) two fluorescently labeled nucleotide analogues comprising a base, a blocking group linked to the base via a cleavable linker, and a fluorescent energy transfer donor label and an anchor for attachment of energy transfer acceptor label linked to the base linker distal to the blocking group, wherein said blocking group prevents or greatly reduces incorporation of a subsequent nucleotide analogue into the extended primer strand,
      wherein the cleavable linkers are cleavable by an identical cleaving agent, and
      wherein each of the nucleotide analogues has a different anchor;
c) washing away any unincorporated nucleotide analogues and contacting the incorporated nucleotide analogue with two anchor binding groups that bind specifically to each of the anchors of the nucleotide analogues of step (b) and comprises a moiety that serves as an energy transfer acceptor,
   wherein said energy transfer acceptor on one of the anchor binding groups is a pH-unresponsive label and said energy transfer acceptor on the other anchor binding groups is a pH-responsive label;
d) washing away any free labels at a pH at which the pH-responsive fluorescent energy transfer acceptor dye label has the same or similar absorption and emission profile as the pH-unresponsive fluorescent energy transfer acceptor label;
e) exposing the incorporated nucleotides to a wavelength that can excite the energy transfer donor dye, and identifying any fluorescence signal due to energy transfer and emission of the energy transfer acceptor dyes attached to the nucleotide analogues due to the labeling reaction performed in step (c);
f) repeating steps (b) to (e) with two different labeled nucleotide analogues than the two different labeled nucleotide analogues in (b), but otherwise having all other properties described in (b);
g) changing the buffer to a pH at which the pH-responsive fluorescent label does not have the same or similar absorption and emission profile as the pH-unresponsive fluorescent label on the fluorescently labeled nucleotide analogue and identifying any fluorescence signal due to incorporation of an anchor labeled nucleotide analogue from steps (b) or (f), wherein the order of steps (e) and (g) may be reversed;
h) contacting the incorporated nucleotide analogue with a cleaving agent that cleaves the cleavable linker and the 3′-O blocking group; and
i) iteratively repeating steps (b) to (h) for each residue of the nucleic acid to be sequenced,
   thereby obtaining the sequence of the nucleic acid.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, G, T)) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) a fluorescently labeled nucleotide analogue comprising a base and a fluorescent label serving as an energy transfer donor and an anchor (anchor 1) for attachment of a pH unresponsive energy transfer acceptor label, linked to the base via a first cleavable linker (cleavable linker 1), and a blocking group at the 3′-OH position, wherein said blocking group prevents incorporation of a subsequent nucleotide analogue into the extended primer strand, (B) a fluorescently labeled nucleotide analogue comprising a base and both a fluorescent label serving as an energy transfer donor and a second anchor (anchor 2) for attachment of a pH-responsive energy transfer acceptor label linked to the base via the same cleavable linker (cleavable linker 1), and a blocking group at the 3′-OH position, wherein said blocking group prevents incorporation of a subsequent nucleotide analogue into the extended primer strand, (C) a fluorescently labeled nucleotide analogue comprising a base and both a fluorescent label serving as an energy transfer donor and the first anchor (anchor 1) for attachment of a pH-unresponsive energy transfer acceptor label linked to the base via a second cleavable linker (cleavable linker 2), and a blocking group at the 3′-OH position, wherein said blocking group prevents incorporation of a subsequent nucleotide analogue into the extended primer strand, and (D) a fluorescently labeled nucleotide analogue comprising a base and both a fluorescent label serving as an energy transfer donor linked to the base via the second cleavable linker (cleavable linker 2), the second anchor (anchor 2) for attachment of a pH-responsive energy transfer acceptor label, and a blocking group at the 3′-OH position, wherein said blocking group prevents incorporation of a subsequent nucleotide analogue into the extended primer strand,
      wherein the first cleavable linker and 3′-O blocking groups are cleavable by an identical cleaving agent, and the second cleavable linker is cleavable by a different cleaving agent; or
   (ii) (A) a fluorescently labeled nucleotide analogue comprising a base, a blocking group linked to the base via a first cleavable linker (cleavable linker 1), and a fluorescent energy transfer donor label and an anchor (anchor 1) for attachment of a pH-unresponsive energy transfer acceptor label linked to the base linker distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a first cleavable linker (cleavable linker 1), and a fluorescent energy transfer donor label and a second anchor (anchor 2) for attachment of a pH-responsive energy transfer acceptor label linked to the base linker distal to the blocking group, wherein said blocking group prevents or greatly reduces incorporation of a subsequent nucleotide analogue into the extended primer strand, (C) a fluorescently labeled nucleotide analogue comprising a base, a blocking group linked to the base via a second cleavable linker (cleavable linker 2), and a fluorescent energy transfer donor label and the first anchor (anchor 1) for attachment of a pH-unresponsive energy transfer acceptor label linked to the base linker distal to the blocking group, wherein said blocking group prevents or greatly reduces incorporation of a subsequent nucleotide analogue into the extended primer strand, and (D) a fluorescently labeled nucleotide analogue comprising a base, a blocking group linked to the base via a second cleavable linker (cleavable linker 2), and a fluorescent energy transfer donor label and the second anchor (anchor 2) for attachment of a pH-responsive energy transfer acceptor label linked to the base linker distal to the blocking group, wherein said blocking group prevents or greatly reduces incorporation of a subsequent nucleotide analogue into the extended primer strand,
      wherein each cleavable linker is cleavable by a different cleaving agent;
c) washing away any unincorporated nucleotide analogues and contacting the incorporated nucleotide analogue with two anchor binding groups that bind specifically to each of the anchors of the nucleotide analogues of step (b) and comprises a moiety that serves as an energy transfer acceptor,
   wherein said energy transfer acceptor on one of the anchor binding groups is a pH-unresponsive label and said energy transfer acceptor on the other anchor binding groups is a pH-responsive label;
d) washing away any free labels at a pH at which the pH-responsive fluorescent energy transfer acceptor dye label has the same or similar absorption and emission profile as the pH-unresponsive fluorescent energy transfer acceptor label;
e) exposing the incorporated nucleotides to a wavelength that can excite the energy transfer donor dye, and identifying any fluorescence signal due to energy transfer and emission of the energy transfer acceptor dyes attached to the nucleotide analogues incorporated in step (b) due to the labeling reaction performed in step (c);
f) changing the buffer to a pH at which the pH-responsive fluorescent label does not have the same or similar absorption and emission profile as the pH-unresponsive fluorescent label on the fluorescently labeled nucleotide analogue and identifying any fluorescence signal due to incorporation of an anchor labeled nucleotide analogue from step (b) due to the labeling reaction performed in step (c), wherein steps (d) and (f) may be reversed;
g) contacting the incorporated nucleotide analogue with a cleaving agent that cleaves the second cleavable linker;
h) washing away the cleaving agent and released labels at a pH at which the pH-responsive fluorescent energy transfer acceptor dye label has the same or similar absorption and emission profile as the pH-unresponsive fluorescent energy transfer acceptor label;
i) repeating step (e);
j) contacting the incorporated nucleotide analogue with a cleaving agent that cleaves the first cleavable linker and the 3′-O blocking group; and
k) iteratively repeating steps (b) to (j) for each residue of the nucleic acid to be sequenced,
   thereby obtaining the sequence of the nucleic acid.

### BRIEF DESCRIPTION OF THE DRAWINGS

FIG. 1: Generalized set of dye and anchor labeled cleavable ddNTP analogues and labeling reagent for single color SBS: Two of the dideoxynucleotide analogues have an anchor (e.g., biotin) and two have a dye (e.g., Cy5). The labeling molecule consists of a molecule able to bind specifically to the anchor (streptavidin) and the same dye. A requirement of this hybrid SBS method is a separate set of four unlabeled reversible terminators (e.g., 3′-O-azidomethyl dNTPs).

FIG. 2: Simplified presentation of scheme for single color SBS using cleavable nucleotide analogues such as those presented in FIG. 3. Two of the ddNTPs have Cy5 and the other two have Biotin attached via an SS Linker. The rectangles represent areas on a substrate containing numerous copies of attached primer-loop-template molecules (or other template-bound primer arrangements) in which the next base in the template strand, from left to right is C, G, T or A. After incubation with the four unlabeled nucleotide reversible terminators (NRTs, e.g., 3′-O-azidomethyl dNTPs) to extend the majority of the primers and two of the ddNTP analogues, ddT with Cy5 and ddA with biotin, using Therminator IX polymerase, imaging will reveal a positive signal in the rectangular area on the right (representing extension of the primer strand with T) and a background signal in the remaining areas. After labeling with Streptavidin-Cy5, imaging will reveal a new positive signal in the third area, indicating incorporation of A. Next, incubation with the remaining ddNTP analogues, ddC with Biotin and ddG with Cy5 (along with excess of A and T NRTs) is performed and imaging will reveal a new positive signal in the area on the left, indicating incorporation of G. Another labeling with Streptavidin-Cy5 will result in the appearance of signal in the remaining rectangular area, indicating incorporation of C. Finally, treatment with THP cleaves the SS linker and removes the azidomethyl group on any primers extended with NRTs in preparation for the next sequencing cycle. The 1, 2, 3 and 4 numeral codes at the left represent the cumulative signals at each of the four indicated imaging steps, a positive signal indicated by a 1 and a background signal indicated by a 0. Incorporation of each of the four possible nucleotide analogues will be revealed by a unique digital code (0111 for A, 0001 for C, 0011 for G and 1111 for T considering all four of these imaging steps; 011 for A, 000 for C, 001 for G and 111 for T considering just the first three of these imaging steps).

FIG. 3: Example ddNTP analogues used for FIG. 4.

FIGS. 4A-4B: Single Color Sequencing by Synthesis Using Set of ddNTP Analogues, Two with Biotin and Two with Cy5, with SS Linker only. Use of ddNTP-Cleavable Linker-Dyes (ddGTP-7-SS-Cy5, ddTTP-5-SS-Cy5), ddNTP-Cleavable Linker-Anchors (ddCTP-5-SS-Biotin, ddATP-7-SS-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. Step 1, Addition of Therminator IX DNA polymerase, two of the ddNTP analogues (ddTTP-5-SS-Cy5, ddATP-7-SS-Biotin and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide reversible terminator analogue to the majority of growing DNA strands (>90%) to terminate DNA synthesis, and extension of a small subset of the primers with the ddA or ddT analogues (complementary to T or A in template strand). Step 2, after washing away the unincorporated nucleotide analogues, imaging for Cy5 fluorescence will reveal those primers extended with ddTTP-5-SS-Cy5. Step 3, addition of streptavidin-Cy5 to label any incorporated ddATP-7-SS-Biotin analogues. Step 4, after washing away unused labeling reagents, a second imaging step will reveal incorporation by ddA. Step 5, subsequent extension with Therminator IX DNA polymerase and the remaining ddNTP analogues (ddGTP-SS-Cy5, ddCTP-5-SS-Biotin) along with 3′-O-azidomethyl-dATP and 3′-O-azidomethyl dTTP, to ensure incorporation fidelity will extend most of the remaining immobilized primed DNA templates, in particular those opposite G and C in the template strand. After this step, the growing DNA strands are terminated with one of the four dye labeled dideoxynucleotide analogues (A, C, G, T) or the same one of the four 3′-blocked reversible terminator nucleotide analogues (A, C, G, T) without dye. Step 6, after washing away the unincorporated nucleotides, a third imaging step is performed. A positive signal will indicate incorporation of ddG. At this point, an optional chase step with the four 3′-O-azidomethyl dNTPs may be performed to ensure that nearly every primer has been extended either with one of the ddNTP or NRT analogues. Step 7, labeling with streptavidin-Cy5 is again carried out to attach Cy5 to any incorporated ddC-5-SS-Biotin analogues. Step 8, after washing away the unused labeling reagents, a fourth round of imaging is performed. Gain of Cy5 signal indicates incorporation of ddC. Step 9, cleavage of SS linker by adding THP to the elongated DNA strands results in removal of all the dyes on the ddNTP analogues and also restores the 3′-OH group on any growing strands extended with a 3′-O-azidomethyl-dNTPs. After washing away the cleaved dyes, an optional final round of imaging is performed. The DNA products are ready for the next cycle of the DNA sequencing reaction. Structures of nucleotides used in this scheme are presented in FIG. 3. In the imaging cartoons at each step, black indicates a positive Cy5 signal and white a background signal. The encoding in the summary cartoon at the end indicates the template sequence, not the incorporated nucleotides.

FIG. 5: Generalized set of dye and anchor labeled cleavable dNTP-Blocker (Virtual Terminator) analogues and labeling reagent for single color SBS. Two of the virtual terminator analogues have an anchor (e.g., biotin) and two have a dye (e.g., Cy5). The labeling molecule consists of a molecule able to bind specifically to the anchor (streptavidin) and the same dye. A chase is carried out using four unlabeled reversible terminators (e.g., 3′-O-azidomethyl dNTPs).

FIG. 6: Simplified presentation of scheme for single color SBS using cleavable nucleotide analogues such as those presented in FIG. 5. Two of the dNTP-Blocker virtual terminators have Cy5 and the other two have Biotin attached via an SS Linker. The rectangles represent areas on a substrate containing numerous copies of attached primer-loop-template molecules (or other template-bound primer arrangements) in which the next base in the template, from left to right, is C, G, T or A. After incubation with the four unlabeled nucleotide reversible terminators (NRTs, e.g., 3′-O-azidomethyl dNTPs) to extend the majority of the primers and two of the virtual terminator analogues, dT with Cy5 and dA with biotin, using Therminator IX polymerase, imaging will reveal a positive signal in the rectangular area on the right (representing extension of the primer strand with T) and a background signal in the remaining areas. After labeling with Streptavidin-Cy5, imaging will reveal a new positive signal in the third area, indicating incorporation of A. Next, incubation with the remaining virtual terminator analogues, dC with Biotin and dG with Cy5 (along with excess of A and T NRTs) is performed and imaging will reveal a new positive signal in the area on the left, indicating incorporation of G. Another labeling with Streptavidin-Cy5 will result in the appearance of signal in the remaining rectangular area, indicating incorporation of C. Finally, treatment with THP cleaves the SS linker and removes the azidomethyl group on any primers extended with NRTs in preparation for the next sequencing cycle. The 1, 2, 3 and 4 numeral codes at the left represent the cumulative signals at each of the four indicated imaging steps, a positive signal indicated by a 1 and a background signal indicated by a 0. Incorporation of each of the four possible nucleotide analogues will be revealed by a unique digital code (0111 for A, 0001 for C, 0011 for G and 1111 for T considering all four of these imaging steps; 011 for A, 000 for C, 001 for G and 111 for T considering just the first three of these imaging steps).

FIG. 7: Example of Virtual Terminator analogues and labeling molecule used for FIG. 8.

FIGS. 8A-8B: Single Color Sequencing by Synthesis Using Set of dNTP-Blocker (Virtual Terminator) Analogues, Two with Biotin and Two with Cy5, with SS Linker Only. Use of dNTP-Cleavable Linker-Blocker-Dyes (dGTP-7-SS-Blocker-Cy5, dTTP-5-SS-Blocker-Cy5), dNTP-Cleavable Linker-Blocker-Anchors (dCTP-5-SS-Blocker-Biotin, dATP-7-SS-Blocker-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. Step 1, Addition of Therminator IX DNA polymerase, two of the dNTP-Blocker (virtual terminator) analogues (dTTP-5-SS-Blocker-Cy5, dATP-7-SS-Blocker-Biotin and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide reversible terminator analogue to the majority of growing DNA strands (>90%) to terminate DNA synthesis, and extension of a small subset of the primers with the dA or dT analogues (complementary to T or A in template strand). Step 2, after washing away the unincorporated nucleotide analogues, imaging for Cy5 fluorescence will reveal those primers extended with dTTP-5-SS-Blocker-Cy5. Step 3, addition of streptavidin-Cy5 to label any incorporated dATP-7-SS-Blocker-Biotin analogues. Step 4, after washing away unused labeling reagents, a second imaging step will reveal incorporation by dA. Step 5, subsequent extension with Therminator IX DNA polymerase and the remaining dNTP-Blocker (virtual terminator) analogues (dGTP-SS-Blocker-Cy5, dCTP-5-SS-Blocker-Biotin) along with 3′-O-azidomethyl-dATP and 3′-O-azidomethyl dTTP to ensure incorporation fidelity will extend most of the remaining immobilized primed DNA templates, in particular those opposite G and C in the template strand. After this step, the growing DNA strands are terminated with one of the four labeled virtual terminator nucleotide analogues (A, C, G, T) or the same one of the four 3′-blocked reversible terminator nucleotide analogues (A, C, G, T) without dye. Step 6, after washing away the unincorporated nucleotides, a third imaging step is performed. A positive signal will indicate incorporation of dG. At this point, an optional chase step with the four 3′-O-azidomethyl dNTPs may be performed to ensure that nearly every primer has been extended either with one of the dNTP virtual terminator or NRT analogues. Step 7, labeling with streptavidin-Cy5 is again carried out to attach Cy5 to any incorporated dC-5-SS-Blocker-Biotin analogues. Step 8, after washing away the unused labeling reagents, a fourth round of imaging is performed. Gain of Cy5 signal indicates incorporation of dC. Step 9, cleavage of SS linker by adding THP to the elongated DNA strands results in removal of all the dyes on the dNTP virtual terminator nucleotide analogues and also restores the 3′-OH group on any growing strands extended with a 3′-O-azidomethyl-dNTPs. After washing away the cleaved dyes, an optional final round of imaging is performed. The DNA products are ready for the next cycle of the DNA sequencing reaction. Structures of nucleotides used in this scheme are presented in FIG. 7. In the imaging cartoons at each step, black indicates a positive Cy5 signal and white a background signal. The encoding in the summary cartoon at the end indicates the template sequence, not the incorporated nucleotides.

FIG. 9: Generalized set of dye and anchor labeled cleavable 3′-blocked reversible terminator analogues and labeling reagent for single color SBS: Two of the reversible terminator analogues have an anchor (e.g., biotin) and two have a dye (e.g., Cy5). The labeling molecule consists of a molecule able to bind specifically to the anchor (streptavidin) and the same dye. A chase is carried out using four unlabeled reversible terminators (e.g., 3′-O-azidomethyl dNTPs).

FIG. 10: Simplified presentation of scheme for single color SBS using cleavable nucleotide analogues such as those presented in FIG. 9. Two of the 3′-blocked nucleotide reversible terminators have Cy5 and the other two have Biotin attached via an SS Linker. The rectangles represent areas on a substrate containing numerous copies of attached primer-loop-template molecules (or other template-bound primer arrangements) in which the next base in the template, from left to right is C, G, T or A. After incubation with the four unlabeled nucleotide reversible terminators (NRTs, e.g., 3′-O-azidomethyl dNTPs) to extend the majority of the primers and two of the labeled reversible terminator analogues, dT with Cy5 and dA with biotin, using Therminator IX polymerase, imaging will reveal a positive signal in the rectangular area on the right (representing extension of the primer strand with T) and a background signal in the remaining areas. After labeling with Streptavidin-Cy5, imaging will reveal a new positive signal in the third area, indicating incorporation of A. Next, incubation with the remaining reversible terminator analogues, dC with Biotin and dG with Cy5 (along with excess of A and T unlabeled NRTs) is performed and imaging will reveal a new positive signal in the area on the left, indicating incorporation of G. Another labeling with Streptavidin-Cy5 will result in the appearance of signal in the remaining rectangular area, indicating incorporation of C. Finally, treatment with THP cleaves the SS linker and removes the azidomethyl group on any primers extended with NRTs in preparation for the next sequencing cycle. The 1, 2, 3 and 4 numeral codes at the left represent the cumulative signals at each of the four indicated imaging steps, a positive signal indicated by a 1 and a background signal indicated by a 0. Incorporation of each of the four possible nucleotide analogues will be revealed by a unique digital code (0111 for A, 0001 for C, 0011 for G and 1111 for T considering all four of these imaging steps; 011 for A, 000 for C, 001 for G and 111 for T considering just the first three of these imaging steps).

FIG. 11: Example labeled reversible terminator analogues and labeling molecule used for FIG. 12.

FIG. 12A-12B: Single Color Sequencing by Synthesis Using Set of 3′-O-Blocked Nucleotide Reversible Terminator Analogues, Two with Biotin and Two with Cy5, with SS Linker Only. Use of 3′-O-SS-dNTP-Cleavable Linker-Dyes (3′-O-SS-dGTP-7-SS-Cy5, 3′-O-SS-dTTP-5-SS-Cy5), 3′-O-SS-dNTP-Cleavable Linker-Anchors (3′-O-SS-dCTP-5-SS-Biotin, 3′-O-SS-dATP-7-SS-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. Step 1, Addition of Therminator IX DNA polymerase, two of the 3′-O-SS-dNTP-Dye or -Anchor analogues (3′-O-SS-dTTP-5-SS-Cy5, 3′-O-SS-dATP-7-SS-Biotin) and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary unlabeled nucleotide reversible terminator analogue to the majority of growing DNA strands (>90%) to terminate DNA synthesis, and extension of a small subset of the primers with the labeled dA or dT analogues (complementary to T or A in template strand). Step 2, after washing away the unincorporated nucleotide analogues, imaging for Cy5 fluorescence will reveal those primers extended with 3′-O-SS-dTTP-5-SS-Cy5. Step 3, addition of streptavidin-Cy5 to label any incorporated 3′-O-SS-dATP-7-SS-Biotin analogues. Step 4, after washing away unused labeling reagents, a second imaging step will reveal incorporation by dA. Step 5, subsequent extension with Therminator IX DNA polymerase and the remaining 3′-O-SS-dNTP-Dye or -Anchor analogues (3′-O-SS-dGTP-SS-Cy5, 3′-O-SS-dCTP-5-SS-Biotin) along with 3′-O-azidomethyl-dATP and 3′-O-azidomethyl dTTP, to ensure incorporation fidelity, will extend most of the remaining immobilized primed DNA templates, in particular those opposite G and C in the template strand. After this step, the growing DNA strands are terminated with one of the four labeled revesible terminator nucleotide analogues (A, C, G, T) or the same one of the four 3′-blocked reversible terminator nucleotide analogues (A, C, G, T) without label. Step 6, after washing away the unincorporated nucleotides, a third imaging step is performed. A positive signal will indicate incorporation of dG. An optional chase step with the four 3′-O-azidomethyl dNTPs may be performed to ensure that nearly every primer has been extended either with one of the labeled dNTP reversible terminator or unlabeled NRT analogues. Step 7, labeling with streptavidin-Cy5 is again carried out to attach Cy5 to any incorporated 3′-O-SS-dC-5-SS-Biotin analogues. Step 8, after washing away the unused labeling reagents, a fourth round of imaging is performed. Gain of Cy5 signal indicates incorporation of dC. Step 9, cleavage of SS linker by adding THP to the elongated DNA strands results in removal of all the dyes on the labeled reversible terminator nucleotide analogues and also restores the 3′-OH group on any growing strands. After washing away the cleaved dyes, an optional final round of imaging is performed. The DNA products are ready for the next cycle of the DNA sequencing reaction. Structures of nucleotides used in this scheme are presented in FIG. 11. In the imaging cartoons at each step, black indicates a positive Cy5 signal and white a background signal. The encoding in the summary cartoon at the end indicates the template sequence, not the incorporated nucleotides.

FIG. 13: Generalized set of dye and anchor labeled cleavable ddNTP analogues and labeling reagents for single color SBS with two spectrally equivalent dyes, Cy5 and HCyC-646, where the latter dye is pH-responsive: One of the dideoxynucleotide analogues has a biotin anchor, one has a Tetrazine anchor, one has Cy5 dye and one has HCyC-646. The labeling molecules consist of a molecule able to bind specifically to one of the anchors (streptavidin for biotin or TCO for tetrazine) and the same two dyes. A requirement of this hybrid SBS method is a separate set of four unlabeled reversible terminators (e.g., 3′-O-azidomethyl dNTPs).

FIG. 14: Simplified presentation of scheme for single color SBS using cleavable nucleotide analogues such as those presented in FIG. 15. One of the ddNTPs has Cy5, one has HCyC-646, one has Biotin and one has Tetrazine attached to the base via an SS Linker. The rectangles represent areas on a substrate containing numerous copies of attached primer-loop-template molecules (or other template-bound primer arrangements) in which the next base in the template strand, from left to right is C, G, T or A. After incubation with the four unlabeled nucleotide reversible terminators (NRTs, e.g., 3′-O-azidomethyl dNTPs) to extend the majority of the primers using Therminator IX polymerase, a further extension is performed with Thermo Sequenase and the four ddNTP analogues: ddA attached to Biotin, ddT attached to Cy5, ddC attached to Tetrazine, and ddG attached to HCyC-646. Finally, a wash is carried out at pH 5 to ensure that the HCyC-646 will emit a signal (at the same or similar emission wavelength as Cy5). Imaging will reveal a positive signal in the rectangular areas at the left and right (representing extension of the primer strand with either G or T) and a background signal in the remaining areas. After simultaneous labeling with Streptavidin-Cy5 and TCO-HCyC-646 which will bind to biotin and tetrazine respectively, followed by another wash at pH 5, imaging will reveal new positive signals in the two central rectangular areas, indicating incorporation of either A or C. Next, a wash at pH 8.5-9 will eliminate the signal from the HCyC-646 dye, which is now present on the C and G ddNTP analogues. An optional chase with the four 3′-O-azidomethyl dNTPs is carried out to ensure essentially all primers have been extended. Finally, treatment with THP cleaves the SS linkers, removing the dyes on incorporated ddNTPs, and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (011 for A, 010 for C, 110 for G and 111 for T considering all three of these imaging steps: 01 for A, 00 for C, 10 for G and 11 for T considering just the first and third of these imaging steps).

FIG. 15: Example ddNTP analogues and labeled binding molecules used for FIG. 16.

FIG. 16A-16B: Single Color Sequencing by Synthesis Using a Set of ddNTP Analogues, One with Cy5, One with HCyC-646, One with Biotin and One with Tetrazine, all with SS Linkers. Use of ddNTP-Cleavable Linker-Dyes (ddGTP-7-SS-HCyC-646, ddTTP-5-SS-Cy5), ddNTP-Cleavable Linker-Anchors (ddCTP-5-SS-Tetrazine, ddATP-7-SS-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dyes (Streptavidin-Cy5, TCO-HCyC-646) to perform 1-color DNA SBS. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddGTP-7-SS-HCyC-646, ddTTP-5-SS-Cy5, ddCTP-5-SS-Tetrazine, ddATP-7-SS-Biotin) to the immobilized primed DNA template enables incorporation of ddNTPs on most of the remaining template-loop-primers. Step 3, after washing away the unincorporated nucleotide analogues at pH 5, fluorescence imaging will reveal those primers extended with either ddTTP-5-SS-Cy5 or ddGTP-SS-HCyC-646. Step 4, addition of streptavidin-Cy5 and TCO-HCyC-646 to label any incorporated ddATP-7-SS-Biotin or ddCTP-5-SS-Tetrazine analogues. Step 5, after washing away unused labeling reagents at pH 5, a second imaging step will reveal incorporation by either ddA or ddC. Step 6, after an additional washing step, this time at pH 9, imaging is performed for the third time. Because the ability of HCyC-646 to fluoresce is pH responsive, occurring below pH 6, it will not exhibit fluorescence at this step. Thus, loss of fluorescence first demonstrated in Step 3 will indicate extension of the ddG analogue, while remaining fluorescence will indicate that the ddT analogue was incorporated. Similarly, loss of fluorescence first visualized in Step 5 will indicate ddC analogue extension, and remaining fluorescence would be indicative of ddA analogue incorporation. At this point, an optional chase step with the four 3′-O-azidomethyl dNTPs may be performed to ensure that nearly every primer has been extended either with one of the ddNTP or NRT analogues. Step 8, cleavage of SS linkers by adding THP to the elongated DNA strands results in removal of all the dyes on the ddNTP analogues and also restores the 3′-OH group on any growing strands extended with a 3′-O-azidomethyl-dNTPs. After washing away the cleaved dyes, an optional final round of imaging is performed. The DNA products are ready for the next cycle of the DNA sequencing reaction. Structures of nucleotides used in this scheme are presented in FIG. 15. In the imaging cartoons at each step, black indicates a positive fluorescent signal and white a background signal. The encoding in the summary cartoon at the end indicates the template sequence, not the incorporated nucleotides.

FIG. 17: Generalized set of dye and anchor labeled cleavable dNTP-Blocker (Virtual Terminator) analogues and labeling reagents for single color SBS with two spectrally equivalent dyes, Cy5 and HCyC-646, where the latter dye is pH-responsive: One of the virtual terminator analogues has a biotin anchor, one has a tetrazine anchor, one has Cy5 dye and one has HCyC-646. The labeling molecules consist of a molecule able to bind specifically to one of the anchors (streptavidin for biotin or TCO for tetrazine) and the same two dyes. Chase molecules are a separate set of four unlabeled reversible terminators (e.g., 3′-O-azidomethyl dNTPs).

FIG. 18: Simplified presentation of scheme for single color SBS using cleavable nucleotide analogues such as those presented in FIG. 17. One of the dNTP-Blocker (virtual terminator) nucleotides has Cy5, one has HCyC-646, one has Biotin and one has Tetrazine attached to the base via an SS Linker. The rectangles represent areas on a substrate containing numerous copies of attached primer-loop-template molecules (or other template-bound primer arrangements) in which the next base in the template strand, from left to right is C, G, T or A. Extension is performed with Thermo Sequenase and the four virtual terminator analogues (containing a blocker group between the base and the label): dA attached to Biotin, dT attached to Cy5, dC attached to Tetrazine, and dG attached to HCyC-646. A chase is then performed with the four unlabeled nucleotide reversible terminators (NRTs, e.g., 3′-O-azidomethyl dNTPs) to extend the remaining primers using Therminator IX polymerase. Finally, a wash is carried out at pH 5 to ensure that the HCyC-646 will emit a signal (at the same or similar emission wavelength as Cy5). Imaging will reveal a positive signal in the rectangular areas at the left and right (representing extension of the primer strand with either G or T) and a background signal in the remaining areas. After simultaneous labeling with Streptavidin-Cy5 and TCO-HCyC-646 which will bind to biotin and tetrazine respectively, followed by another wash at pH 5, imaging will reveal new positive signals in the two central rectangular areas, indicating incorporation of either A or C. Next, a wash at pH 8.5-9 will eliminate the fluorescence signal from the HCyC-646 dye, which is now present on the C and G ddNTP analogues. An optional chase with the four 3′-O-azidomethyl dNTPs is carried out to ensure essentially all primers have been extended. Finally, treatment with THP cleaves the SS linkers, removing the dyes on incorporated virtual terminators, and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (011 for A, 010 for C, 110 for G and 111 for T considering all three of these imaging steps; 01 for A, 00 for C, 10 for G and 11 for T considering just the first and third of these imaging steps).

FIG. 19: Example 3′-blocker (Virtual Terminator) analogues and labeling molecules used for FIG. 20.

FIGS. 20A-20B: Single Color Sequencing by Synthesis Using a Set of dNTP-Blocker (Virtual Terminator) Analogues, One with Cy5, One with HCyC-646, One with Biotin and One with Tetrazine, all with SS Linkers. Use of dNTP-Cleavable Linker-Blocker-Dyes (dGTP-7-SS-Blocker-HCyC-646, dTTP-5-SS-Blocker-Cy5), dNTP-Cleavable Linker-Blocker-Anchors (dCTP-5-SS-Blocker-Tetrazine, dATP-7-SS-Blocker-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dyes (Streptavidin-Cy5, TCO-HCyC-646) to perform 1-color DNA SBS. Step 1, Addition of Thermo Sequenase and the four dNTP-Blocker virtual terminator analogues (dGTP-7-SS-Blocker-HCyC-646, dTTP-5-SS-Blocker-Cy5, dCTP-5-SS-Blocker-Tetrazine, dATP-7-SS-Blocker-Biotin) to the immobilized primed DNA template enables incorporation of these virtual terminators. Step 2, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to any primer strands not extended with the virtual terminators. Step 3, after washing away the unincorporated nucleotide analogues at pH 5, fluorescence imaging will reveal those primers extended with either dTTP-5-SS-Blocker-Cy5 or dGTP-SS-Blocker-HCyC-646. Step 4, addition of streptavidin-Cy5 and TCO-HCyC-646 to label any incorporated dATP-7-SS-Blocker-Biotin or dCTP-5-SS-Blocker-Tetrazine analogues. Step 5, after washing away unused labeling reagents at pH 5, a second imaging step will reveal incorporation by either dA or dC. Step 6, after an additional washing step, this time at pH 9, imaging is performed for the third time. Because the ability of HCyC-646 to fluoresce is pH responsive, occurring below pH 6, it will not exhibit fluorescence at this step. Thus, loss of fluorescence demonstrated in Step 3 will indicate extension of the dG analogue, while remaining fluorescence will indicate that dT analogue was incorporated. Similarly, loss of fluorescence first visualized in Step 5 will indicate dC analogue extension, and remaining fluorescence would be indicative of dA analogue incorporation. At this point, an optional chase step with the four 3′-O-azidomethyl dNTPs may be performed to ensure that nearly every primer has been extended either with one of the virtual terminator or NRT analogues. Step 7, cleavage of SS linkers by adding THP to the elongated DNA strands results in removal of all the dyes on the virtual terminator analogues and also restores the 3′-OH group on any growing strands extended with a 3′-O-azidomethyl-dNTPs. After washing away the cleaved dyes, an optional final round of imaging is performed. The DNA products are ready for the next cycle of the DNA sequencing reaction. Structures of nucleotides used in this scheme are presented in FIG. 19. In the imaging cartoons at each step, black indicates a positive fluorescent signal and white a background signal. The encoding in the summary cartoon at the end indicates the template sequence, not the incorporated nucleotides.

FIG. 21: Generalized set of dye and anchor labeled 3′-blocked reversible terminator analogues and labeling reagents for single color SBS with two spectrally equivalent dyes, Cy5 and HCyC-646, where the latter dye is pH-responsive: One of the 3′-blocked reversible terminator analogues has a biotin anchor, one has a tetrazine anchor, one has Cy5 dye and one has HCyC-646. The labeling molecules consist of a molecule able to bind specifically to one of the anchors (streptavidin for biotin or TCO for tetrazine) and the same two dyes. Chase molecules are a separate set of four unlabeled reversible terminators (e.g., 3′-O-azidomethyl dNTPs).

FIG. 22: One of the 3′-blocked nucleotide reversible terminators has Cy5, one has HCyC-646, one has Biotin and one has Tetrazine attached to the base via an SS Linker. The rectangles represent areas on a substrate containing numerous copies of attached primer-loop-template molecules (or other template-bound primer arrangements) in which the next base in the template strand, from left to right is C, G, T or A. Extension is performed with Therminator IX and the four 3′-blocked labeled nucleotide reversible terminator analogues: dA attached to Biotin, dT attached to Cy5, dC attached to Tetrazine, and dG attached to HCyC-646. A chase is then performed with the four unlabeled nucleotide reversible terminators (NRTs, e.g., 3′-O-azidomethyl dNTPs) to extend the remaining primers using Therminator IX polymerase. Finally, a wash is carried out at pH 5 to ensure that the HCyC-646 will emit a signal (at the same or similar emission wavelength as Cy5). Imaging will reveal a positive signal in the rectangular areas at the left and right (representing extension of the primer strand with either G or T) and a background signal in the remaining areas. After simultaneous labeling with Streptavidin-Cy5 and TCO-HCyC-646 which will bind to biotin and tetrazine respectively, followed by another wash at pH 5, imaging will reveal new positive signals in the two central rectangular areas, indicating incorporation of either A or C. Next, a wash at pH 8.5-9 will eliminate the fluorescence signal from the HCyC-646 dye, which is now present on the C and G reversible terminator analogues. An optional chase with the four 3′-O-azidomethyl dNTPs is carried out to ensure essentially all primers have been extended. Finally, treatment with THP cleaves the SS linkers, removing the dyes on incorporated nucleotide reversible terminators, and removes the blocking groups on primers extended with either labeled or unlabeled NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (011 for A, 010 for C, 110 for G and 111 for T considering all three of these imaging steps; 01 for A, 00 for C, 10 for G and 11 for T considering just the first and third of these imaging steps).

FIG. 23: Example labeled reversible terminator analogues and labeling molecules used for FIG. 24-25.

FIGS. 24-25: Single Color Sequencing by Synthesis Using a Set of Reversibly 3′-Blocked Nucleotide Terminator Analogues, One with Cy5, One with HCyC-646, One with Biotin and One with Tetrazine, all with SS Linkers. Use of 3′-O-SS-dNTP-Cleavable Linker-Dyes (3′-O-SS-dGTP-7-SS-HCyC-646, 3′-O-SS-dTTP-5-SS-Cy5), 3′-O-SS-dNTP-Cleavable Linker-Anchors (3′-O-SS-dCTP-5-SS-Tetrazine, 3′-O-SS-dATP-7-SS-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dyes (Streptavidin-Cy5, TCO-HCyC-646) to perform 1-color DNA SBS. Step 1, Addition of Therminator IX and the four 3′-O-SS-dNTP reversible terminator analogues (3′-O-SS-dGTP-7-SS-HCyC-646, 3′-O-SS-dTTP-5-SS-Cy5, 3′-O-SS-dCTP-5-SS-Tetrazine, 3′-O-SS-dATP-7-SS-Biotin) to the immobilized primed DNA template enables incorporation of these nucleotide reversible terminators. Step 2, Addition of Therminator IX DNA polymerase and the four unlabeled reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to any primer strands not extended with the labeled reversible terminators. Step 3, after washing away the unincorporated nucleotide analogues at pH 5, fluorescence imaging will reveal those primers extended with either 3′-O-SS-dTTP-5-SS-Cy5 or 3′-O-SS-dGTP-7-SS-HCyC-646. Step 4, addition of streptavidin-Cy5 and TCO-HCyC-646 to label any incorporated 3′-O-SS-dATP-7-SS-Biotin or 3′-O-SS-dCTP-5-SS-Tetrazine analogues. Step 5, after washing away unused labeling reagents at pH 5, a second imaging step will reveal incorporation by either dA or dC. Step 6, after an additional washing step, this time at pH 9, imaging is performed for the third time. Because the ability of HCyC-646 to fluoresce is pH responsive, occurring below pH 6, it will not exhibit fluorescence at this step. Thus loss of fluorescence demonstrated in Step 3 will indicate extension of the dG analogue, while remaining fluorescence will indicate that dT analogue was incorporated. Similarly, loss of fluorescence first visualized in Step 5 will indicate dC analogue extension, and remaining fluorescence would be indicative of dA analogue incorporation. At this point, an optional chase step with the four 3′-O-azidomethyl dNTPs may be performed to ensure that nearly every primer has been extended either with one of the unlabeled or labeled NRT analogues. Step 7, cleavage of SS linkers by adding THP to the elongated DNA strands results in removal of all the dyes on the 3′-O-SS-dNTP terminator analogues and also restores the 3′-OH group 3′-O-SS-dNTP or 3′-O-azidomethyl-dNTPs. After washing away the cleaved dyes, an optional final round of imaging is performed. The DNA products are ready for the next cycle of the DNA sequencing reaction. Structures of nucleotides used in this scheme are presented in FIG. 23. In the imaging cartoons at each step, black indicates a positive fluorescent signal and white a backaground signal. The encoding in the summary cartoon at the end indicates the template sequence, not the incorporated nucleotides.

FIG. 26: Generalized set of dye and anchor labeled cleavable and uncleavable ddNTP analogues and labeling reagents for single color SBS with photobleaching: Two of the dideoxynucleotide analogues have an attached Cy5, one via a cleavable and one via an uncleavable linker. The other two dideoxynucleotide analogues have an attached biotin anchor, one via a cleavable and one via an uncleavable linker. The labeling molecule can bind specifically to one of the biotin anchors and has the same dye. A dye such as Cy5 which can be photobleached is needed. A requirement of this hybrid SBS method is a separate set of four unlabeled reversible terminators (e.g., 3′-O-azidomethyl dNTPs).

FIG. 27: Simplified presentation of scheme for single color SBS using cleavable and uncleavable nucleotide analogues such as those presented in FIG. 26. Two of the ddNTPs have Cy5 and the other two have Biotin attached via either an SS Linker or an Uncleavable Linker. The rectangles represent areas on a substrate containing numerous copies of attached primer-loop-template molecules (or other template-bound primer arrangements) in which the next base in the template strand, from left to right is C, G, T or A. After incubation with the four unlabeled nucleotide reversible terminators (NRTs, e.g., 3′-O-azidomethyl dNTPs) and Therminator IX to extend the majority of the primers, extension is carried out with Thermo Sequenase and four ddNTP analogues, ddATP attached to Biotin via an SS linker, ddTTP attached to Cy5 via an SS linker, ddGTP attached to Cy5 via an uncleavable linker, and ddCTP attached to biotin via an uncleavable linker. Imaging will reveal a positive signal in the left and right rectangular areas (representing extension of the primer strand with either G or T) and a background signal in the remaining areas. After labeling with Streptavidin-Cy5, imaging will reveal a new positive signal in the remaining areas, indicating incorporation of A or C. Treatment with THP cleaves the SS linkers on A and T ddNTP analogues and removes the azidomethyl group on any primers extended with NRTs in preparation for the next sequencing cycle. Finally, photobleaching is performed to destroy the remaining dyes attached to C and G, which do not have cleavable linkers. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (010 for A, 011 for C, 111 for G and 110 for T considering all three of these imaging steps; 00 for A, 01 for C, 11 for G and 10 for T considering just the first and last of these imaging steps).

FIG. 28: Example ddNTP Analogues Used for FIG. 29.

FIGS. 29A-29B: Single Color Sequencing by Synthesis Using Set of ddNTP Analogues, One with SS Linker and Cy5, One with SS Linker and Biotin, One with Uncleavable Linker and Cy5, and One with Uncleavable Linker and Biotin, with a Photobleaching Step. Use of ddNTP-Cleavable Linker-Dye (ddTTP-5-SS-Cy5), ddNTP-Cleavable Linker-Anchor (ddATP-7-SS-Biotin), ddNTP-Uncleavable Linker-Dye (ddGTP-7-Cy5), ddNTP-Uncleavable Linker-Dye (ddCTP-5-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddATP-7-SS-Biotin, ddTTP-5-SS-Cy5, ddCTP-5-Biotin, ddGTP-7-Cy5) to the immobilized primed DNA template enables incorporation of ddNTPs on most of the remaining primers. Step 3, after washing away the unincorporated nucleotide analogues, imaging for Cy5 fluorescence will reveal those primers extended with either ddTTP-5-SS-Cy5 or ddGTP-7-Cy5. Step 4, addition of streptavidin-Cy5 to label any incorporated ddATP-7-SS-Biotin or ddCTP-5-Biotin analogues. Step 5, after washing away unused labeling reagents, a second imaging step will reveal incorporation by either ddA or ddC. At this point, an optional chase step with the four 3′-O-azidomethyl dNTPs may be performed to ensure that nearly every primer has been extended either with one of the ddNTP or NRT analogues. Step 6, cleavage of SS linkers by adding THP to the elongated DNA strands results in removal of the dyes on the ddATP and ddTTP analogues and also restores the 3′-OH group on any growing strands extended with a 3′-O-azidomethyl-dNTPs. Step 7, after washing to remove THP, an imaging step is performed. Loss of Cy5 signal in the case of previously determined ddGTP or ddTTP analogue incorporation indicates ddT and remaining signal indicates ddG incorporation. Similarly, loss of Cy5 signal in the case of previously determined ddATP or ddCTP analogue incorporation indicates ddA and remaining signal indicates ddC incorporation. Step 8, a photobleaching step is performed to eliminate any fluorescence due to incorporation of either ddCTP or ddGTP analogues. The DNA products are ready for the next cycle of the DNA sequencing reaction. Structures of nucleotides used in this scheme are presented in FIG. 28. In the imaging cartoons at each step, black indicates a positive Cy5 signal and white a background signal. The encoding in the summary cartoon at the end indicates the template sequence, not the incorporated nucleotides.

FIG. 30: Example ddNTP Analogues Used for FIG. 31.

FIG. 31: Two Color Sequencing by Synthesis Using Set of ddNTP Analogues, Two with Alexa488 and Two with Cy5, with SS Linker only. Use of ddNTP-Cleavable Linker-Dye (ddTTP-5-SS-Cy5, ddATP-7-SS-Alexa488, ddGTP-7-SS-Cy5, ddCTP-5-SS-Alexa488) and 3′-O-azidomethyl dNTPs (3′-O-azidomethyl-dATP, 3′-O-azidomethyl-dCTP, 3′-O-azidomethyl-dGTP, 3′-O-azidomethyl-dTTP to perform 2-color DNA SBS. Step 1, Addition of Therminator IX DNA polymerase, two of the four ddNTP analogues (ddTTP-5-SS-Cy5, ddATP-7-SS-Alexa488) and an excess of the four 3′-blocked dNTPs (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide reversible terminators to the majority of growing DNA strands (>90%) and the ddA or ddT analogues to some of the remaining primers (opposite either a template T or A moiety) to terminate DNA synthesis. Step 2, after washing away the unincorporated nucleotide analogues, imaging for Cy5 and Alexa488 fluorescence will reveal those primers extended with ddTTP-5-SS-Cy5 and ddATP-7-Alexa488 specifically. Step 3, addition of Therminator IX DNA polymerase, the other two of the four ddNTP analogues (ddGTP-7-SS-Cy5, ddCTP-5-SS-Alexa488). and the other two of the four 3′-blocked dNTPs (3′-O-azidomethyl-dATP, 3′-O-azidomethyl-dTTP), to ensure incorporation fidelity, to the immobilized primed DNA template enables the incorporation of the ddC or ddG analogues to some of the remaining primers (opposite either a template G or C moiety) to terminate DNA synthesis. Step 4, after washing away unused labeling reagents, a second imaging step will reveal incorporation by either ddC or ddG. At this point, an optional chase step with the four 3′-O-azidomethyl dNTPs may be performed to ensure that nearly every primer has been extended either with one of the ddNTP or NRT analogues. Step 5, cleavage of SS linkers by adding THP to the elongated DNA strands results in removal of all the dyes on the ddNTP analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 30. Fluorescence due to Cy5 is shown as black squares and fluorescence due to Alexa488 is shown as black circles.

FIG. 32: Generalized set of dye and anchor labeled cleavable ddNTP analogues and labeling reagents for single color SBS using click-to-release chemistry: Two of the dideoxynucleotide analogues have an attached Cy5, one via an SS linker and one via a carbamyl TCO linker. The other two dideoxynucleotide analogues have an attached biotin anchor, one via an SS linker and one via a carbamyl TCO linker. The labeling molecule can bind specifically to one of the biotin anchors and has the same dye. Clicking of tetrazine to the TCO results in an elimination reaction that triggers dye or anchor cleavage. A requirement of this hybrid SBS method is a separate set of four unlabeled reversible terminators (e.g., 3′-O-azidomethyl dNTPs).

FIG. 33: Simplified presentation of scheme for single color SBS using cleavable nucleotide analogues such as those presented in FIG. 32. Two of the ddNTPs have Cy5 and the other two have biotin attached via either an SS linker or a carbamyl TCO Linker. The rectangles represent areas on a substrate containing numerous copies of attached primer-loop-template molecules (or other template-bound primer arrangements) in which the next base in the template strand, from left to right is T, G, C or A. After incubation with the four unlabeled nucleotide reversible terminators (NRTs, e.g., 3′-O-azidomethyl dNTPs) and Therminator IX to extend the majority of the primers, extension is carried out with Thermo Sequenase and four ddNTP analogues, ddATP attached to Cy5 via an SS linker, ddTTP attached to Cy5 via a carbamyl TCO linker, ddGTP attached to biotin via an SS linker, and ddCTP attached to biotin via a carbamyl TCO linker. Imaging will reveal a positive signal in the left and right rectangular areas (representing extension of the primer strand with either A or T) and a background signal in the remaining areas. After labeling with Streptavidin-Cy5, imaging will reveal a new positive signal in the remaining areas, indicating incorporation of C or G. Treatment with tetrazine cleaves the TCO linkers on ddC and ddT analogues. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 011 for G and 110 for T considering all three of these imaging steps; 11 for A, 00 for C, 01 for G and 10 for T considering just the first and last of these imaging steps).

FIG. 34: Example ddNTP Analogues Used for FIG. 35.

FIGS. 35A-35B: Single Color Sequencing by Synthesis Using Set of ddNTP Analogues, One with SS Linker and Cy5, One with SS Linker and Biotin, One with TCO-Carbamate Linker and Cy5, and One with TCO-Carbamate Linker and Biotin. Use of ddNTP-Cleavable Linker-Dyes (ddATP-7-SS-Cy5, ddTTP-5-TCO-Cy5), ddNTP-Cleavable Linker-Anchors (ddGTP-7-SS-Biotin, ddCTP-5-TCO-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. This is essentially identical to two-linker schemes presented in our recently filed patent application (Ju et al. PCT/US2019/022326) but instead of an azo linker cleavable by sodium dithionite, here a TCO carbamate linker which undergoes a click-and-release reaction with tetrazine is used. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddATP-7-SS-Cy5, ddTTP-5-TCO-Cy5, ddGTP-7-SS-Biotin, ddCTP-5-TCO-Biotin) to the immobilized primed DNA template enables incorporation of ddNTPs on most of the remaining template-loop-primers. Step 3, after washing away the unincorporated nucleotide analogues, imaging for Cy5 fluorescence will reveal those primers extended with either ddATP-7-SS-Cy5 or ddTTP-5-TCO-Cy5. Step 4, addition of streptavidin-Cy5 to label any incorporated ddGTP-7-SS-Biotin or ddCTP-5-TCO-Biotin analogues. Step 5, after washing away unused labeling reagents, a second imaging step will reveal incorporation by either ddC or ddG. At or just prior to this point, an optional chase step with the four 3′-O-azidomethyl dNTPs may be performed to ensure that nearly every primer has been extended either with one of the ddNTP or NRT analogues. Step 6, cleavage of SS linkers by adding tetrazine to the elongated DNA strands results in removal of the dyes on the ddCTP and ddTTP analogues and also restores the 3′-OH group on any growing strands extended with a 3′-O-azidomethyl-dNTPs. Step 7, after washing away excess tetrazine, imaging is carried out. Loss of Cy5 signal in the case of previously determined ddATP or ddTTP analogue incorporation indicates ddT and remaining signal indicates ddA incorporation. Similarly, loss of Cy5 signal in the case of previously determined ddCTP or ddGTP analogue incorporation indicates ddC and remaining signal indicates ddG incorporation. Step 8, cleavage of SS linker by adding THP to the elongated DNA strands results in removal of dyes on the ddATP and ddGTP analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 34. In the imaging cartoons at each step, black indicates a positive Cy5 signal and white a background signal. The encoding in the summary cartoon at the end indicates the template sequence, not the incorporated nucleotides.

FIG. 36: Generalized set of dye and anchor labeled cleavable ddNTP analogues, labeling and quenching reagents for single color SBS using quenching: One of the dideoxynucleotide analogues has Cy5 attached to the base, one has biotin attached, one has both biotin and TCO in branched chain configuration attached, and the last has Cy5 and TCO attached, also in branched chain configuration, all four via an SS linker. The labeling molecule can bind specifically to one of the biotin anchors and has the same dye. The quenching molecule (e.g., BHQ3) binds via the TCO anchor. A requirement of this hybrid SBS method is a separate set of four unlabeled reversible terminators (e.g., 3′-O-azidomethyl dNTPs).

FIG. 37: Simplified presentation of scheme for single color SBS using cleavable nucleotide analogues such as those presented in FIG. 36. Each type of ddNTP has one of the following, Cy5, biotin, biotin-TCO, or Cy5-TCO, attached via an SS linker. The rectangles represent areas on a substrate containing numerous copies of attached primer-loop-template molecules (or other template-bound primer arrangements) in which the next base in the template strand, from left to right is T, G, C or A. After incubation with the four unlabeled nucleotide reversible terminators (NRTs, e.g., 3′-O-azidomethyl dNTPs) and Therminator IX to extend the majority of the primers, extension is carried out with Thermo Sequenase and four ddNTP analogues, ddATP attached to Cy5, ddTTP attached to Cy5-TCO, ddGTP attached to biotin, and ddCTP attached to biotin-TCO. Imaging will reveal a positive signal in the left and right rectangular areas (representing extension of the primer strand with either A or T) and a background signal in the remaining areas. After labeling with Streptavidin-Cy5, imaging will reveal a new positive signal in the remaining areas, indicating incorporation of C or G. Treatment with tetrazine-BHQ3 quenches the Cy5 fluorescence on C and T ddNTP analogues. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 011 for G and 110 for T considering all three of these imaging steps; 11 for A, 00 for C, 01 for G and 10 for T considering just the first and last of these imaging steps).

FIG. 38: Example ddNTP Analogues and Quencher-Anchor Binding Molecule Used for FIG. 39.

FIGS. 39A-39B: Single Color Sequencing by Synthesis Using a Set of ddNTP Analogues, One with Cy5, One with Biotin, One with Cy5 and Biotin, and One with Biotin and TCO Anchors, all with SS Linkers, Taking Advantage of a Dye Quencher. Use of ddNTP-Cleavable Linker-Dye (ddATP-7-SS-Cy5), ddNTP-Cleavable Linker-Anchor1 (ddGTP-7-SS-Biotin), ddNTP-Cleavable Linker-Branched Anchors 1 and 2 (ddCTP-5-SS-Biotin/TCO), ddNTP-Cleavable Linker-Dye-Anchor (ddTTP-5-SS-Cy5-TCO), 3′-O-azidomethyl dNTPs (3′-O-azidomethyl-dATP, 3′-O-azidomethyl-dCTP, 3′-O-azidomethyl-dGTP, 3′-O-azidomethyl-dTTP), Anchor Binding Molecule-Dye (Streptavidin-Cy5), and Anchor Binding Molecule-Quencher (Tetrazine-BHQ) to perform 1-color DNA SBS. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddATP-7-SS-Cy5, ddGTP-7-SS-Biotin, ddCTP-5-SS-Biotin/TCO, ddTTP-5-SS-Cy5-TCO) to the immobilized primed DNA template enables incorporation of ddNTPs on most of the remaining primers. Step 3, after washing away the unincorporated nucleotide analogues, imaging for Cy5 fluorescence will reveal those primers extended with either ddATP-7-SS-Cy5 or ddTTP-5-SS-Cy5-TCO. Step 4, addition of streptavidin-Cy5 to label any incorporated ddGTP-7-SS-Biotin or ddCTP-5-SS-Biotin/TCO analogues. Step 5, after washing away unused labeling reagents, a second imaging step is performed, and new fluorescence signals will confirm incorporation by either ddC or ddG. At or just prior to this point, an optional chase step with the four 3′-O-azidomethyl dNTPs may be performed to ensure that nearly every primer has been extended either with one of the ddNTP or NRT analogues. Step 6, incubation with Tetrazine-BHQ to quench the fluorescence of the dyes on ddC or ddT analogues. Step 7, after washing to remove any free tetrazine-BHQ, a third imaging step is carried out. Substantial loss of Cy5 signal in the case of previously determined ddATP or ddTTP analogue incorporation indicates ddT and remaining signal indicates ddA incorporation. Similarly, substantial loss of Cy5 signal in the case of previously determined ddCTP or ddGTP analogue incorporation indicates ddC and remaining signal indicates ddG incorporation. Step 8, cleavage of SS linker by adding THP to the elongated DNA strands results in removal of dyes and quenchers on the nucleotide analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 38. In the imaging cartoons at each step, black indicates a positive Cy5 signal and white or light gray a background signal. The encoding in the summary cartoon at the end indicates the template sequence, not the incorporated nucleotides.

FIG. 40: Generalized set of dye and anchor labeled cleavable virtual terminator nucleotide analogues, labeling and quenching reagents for single color SBS using quenching: One of the virtual terminator analogues has Cy5 attached to the base, one has biotin attached, one has both biotin and TCO in branched chain configuration attached, and the last has Cy5 and TCO attached, also in branched chain configuration, all four via an SS linker. The labeling molecule can bind specifically to one of the biotin anchors and has the same dye. The quenching molecule binds via the TCO anchor. Chase reactions are performed with four unlabeled reversible terminators (e.g., 3′-O-azidomethyl dNTPs).

FIG. 41: Simplified presentation of scheme for single color SBS using cleavable nucleotide analogues such as those presented in FIG. 40. Each type of virtual terminator has one of the following, Cy5, biotin, biotin-TCO, or Cy5-TCO, attached via an SS linker. The rectangles represent areas on a substrate containing numerous copies of attached primer-loop-template molecules (or other template-bound primer arrangements) in which the next base in the template strand, from left to right is T, G, C or A. An extension is carried out with Thermo Sequenase and four virtual terminator analogues, dATP attached to Cy5, dTTP attached to Cy5-TCO, dGTP attached to biotin, and dCTP attached to biotin-TCO. A chase with the four unlabeled nucleotide reversible terminators (NRTs, e.g., 3′-O-azidomethyl dNTPs) and Therminator IX is performed to extend all the remaining primers. Imaging will reveal a positive signal in the left and right rectangular areas (representing extension of the primer strand with either A or T) and a background signal in the remaining areas. After labeling with Streptavidin-Cy5, imaging will reveal a new positive signal in the remaining areas, indicating incorporation of C or G. Treatment with tetrazine-BHQ quenches the Cy5 fluorescence on C and T virtual terminator analogues. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 011 for G and 110 for T considering all three of these imaging steps; 11 for A, 00 for C, 01 for G and 10 for T considering just the first and last of these imaging steps).

FIG. 42: Example dNTP Analogues (Virtual Terminators) and Quencher-Anchor Binding Molecule Used for FIG. 43.

FIGS. 43A-43B: Single Color Sequencing by Synthesis Using a Set of Virtual Terminator Nucleotide Analogues, One with Cy5, One with Biotin, One with Cy5 and Biotin, and One with Biotin and TCO Anchors, all with SS Linkers, Taking Advantage of a Dye Quencher. Use of dNTP-Cleavable Linker-Blocker-Dye (dATP-7-SS-Blocker-Cy5), dNTP-Cleavable Linker-Blocker Anchor (ddGTP-7-SS-Blocker-Biotin), dNTP-Cleavable Linker-Blocker-Branched Anchors 1 and 2 (dCTP-5-SS-Blocker-Biotin/TCO), ddNTP-Cleavable Linker-Blocker-Dye-Anchor (ddTTP-5-SS-Blocker-Cy5-TCO), 3′-O-azidomethyl dNTPs (3′-O-azidomethyl-dATP, 3′-O-azidomethyl-dCTP, 3′-O-azidomethyl-dGTP, 3′-O-azidomethyl-dTTP), Anchor Binding Molecule-Dye (Streptavidin-Cy5), and Anchor Binding Molecule-Quencher (Tetrazine-BHQ) to perform 1-color DNA SBS. Step 1, Addition of Thermo Sequenase and the four dNTP-Blocker virtual terminator analogues (dATP-7-SS-Blocker-Cy5, ddGTP-7-SS-Blocker-Biotin, dCTP-5-SS-Blocker-Biotin/TCO, ddTTP-5-SS-Blocker-Cy5-TCO) to the immobilized primed DNA template enables incorporation of these virtual terminators. Step 2, a chase with Therminator IX DNA polymerase and the four unlabeled reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to any primer strands not terminated with the virtual terminators. Step 3, after washing away the unincorporated nucleotide analogues, imaging for Cy5 fluorescence will reveal those primers extended with either ddATP-7-SS-Blocker-Cy5 or ddTTP-5-SS-Blocker-Cy5-TCO. Step 4, addition of streptavidin-Cy5 to label any incorporated ddGTP-7-SS-Blocker-Biotin or ddCTP-5-SS-Blocker-Biotin/TCO analogues. Step 5, after washing away unused labeling reagents, a second imaging step is performed, and new fluorescence signals will confirm incorporation by either C or G virtual terminators. If a chase step has not been performed earlier, at or just prior to this point, an optional chase step with the four 3′-O-azidomethyl dNTPs may be performed to ensure that nearly every primer has been extended either with one of the virtual terminator or NRT analogues. Step 6, incubation with Tetrazine-BHQ to quench the fluorescence of the dyes on C or T virtual terminators. Step 7, after washing to remove any free tetrazine-BHQ, a third imaging step is carried out. Substantial loss of Cy5 signal in the case of previously determined dATP or dTTP virtual terminator analogue incorporation indicates dT and remaining signal indicates dA incorporation. Similarly, substantial loss of Cy5 signal in the case of previously determined dCTP or dGTP virtual terminator analogue incorporation indicates dC and remaining signal indicates dG incorporation. Step 8, cleavage of SS linker by adding THP to the elongated DNA strands results in removal of dyes and quenchers on the nucleotide analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 42. In the imaging cartoons at each step, black indicates a positive Cy5 signal and white or light gray a background signal. The encoding in the summary cartoon at the end indicates the template sequence, not the incorporated nucleotides.

FIG. 44: Generalized set of dye and anchor labeled cleavable 3′-blocked nucleotide reversible terminator analogues, labeling and quenching reagents for single color SBS using quenching: One of the reversible terminator analogues has Cy5 attached to the base, one has biotin attached, one has both biotin and TCO in branched chain configuration attached, and the last has Cy5 and TCO attached, also in branched chain configuration, all four via an SS linker. The labeling molecule can bind specifically to one of the biotin anchors and has the same dye. The quenching molecule binds via the TCO anchor. Chase reactions are performed with four unlabeled reversible terminators (e.g., 3′-O-azidomethyl dNTPs).

FIG. 45: Simplified presentation of scheme for single color SBS using cleavable nucleotide analogues such as those presented in FIG. 44. Each type of nucleotide reversible terminator (A, C, G and T) has one of the following, Cy5, biotin, biotin and TCO, or Cy5-TCO, attached via an SS linker. The rectangles represent areas on a substrate containing numerous copies of attached primer-loop-template molecules (or other template-bound primer arrangements) in which the next base in the template strand, from left to right is T, G, C or A. extension is carried out with Therminator IX and four nucleotide reversible terminator analogues, 3′-O-t-Butyl-SS-ATP attached to Cy5, 3′-O-t-Butyl-SS-dTTP attached to Cy5-TCO, 3′-O-t-Butyl-SS-dGTP attached to biotin, and 3′-O-t-Butyl-SS-dCTP attached to both biotin and TCO, all via an SS linker. A chase with the four unlabeled nucleotide reversible terminators (NRTs, e.g., 3′-O-azidomethyl dNTPs) and Therminator IX is performed to extend all the remaining primers. Imaging will reveal a positive signal in the left and right rectangular areas (representing extension of the primer strand with either A or T) and a background signal in the remaining areas. After labeling with Streptavidin-Cy5, imaging will reveal a new positive signal in the remaining areas, indicating incorporation of C or G. Treatment with tetrazine-BHQ quenches the Cy5 fluorescence on C and T reversible terminator analogues. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 011 for G and 110 for T considering all three of these imaging steps; 11 for A, 00 for C, 01 for G and 10 for T considering just the first and last of these imaging steps).

FIG. 46: Example 3′-SS-dNTP Analogues (Reversible Terminators) and Quencher-Anchor Binding Molecule Used for FIG. 47.

FIGS. 47A-47B: Single Color Sequencing by Synthesis Using a Set of Nucleotide Reversible Terminator Analogues, One with Cy5, One with Biotin, One with Cy5 and Biotin, and One with Biotin and TCO Anchors, all with SS Linkers, Taking Advantage of a Dye Quencher. Use of 3′-SS-dNTP-Cleavable Linker-Dye (3′-O-SS-dATP-7-SS-Cy5), 3′-O-SS-dNTP-Cleavable Linker-Anchor (3′-O-SS-ddGTP-7-SS-Biotin), 3′-O-SS-dNTP-Cleavable Linker-Blocker-Branched Anchors 1 and 2 (3′-O-SS-dCTP-5-SS-Biotin/TCO), 3′-O-SS-ddNTP-Cleavable Linker-Dye-Anchor (3′-O-SS-dTTP-5-SS-Cy5-TCO), 3′-O-azidomethyl dNTPs (3′-O-azidomethyl-dATP, 3′-O-azidomethyl-dCTP, 3′-O-azidomethyl-dGTP, 3′-O-azidomethyl-dTTP), Anchor Binding Molecule-Dye (Streptavidin-Cy5), and Anchor Binding Molecule-Quencher (Tetrazine-BHQ) to perform 1-color DNA SBS. Step 1, Addition of Therminator IX and the four 3′-blocked reversible terminator analogues (3′-O-SS-dATP-7-SS-Cy5, 3′-O-SS-dGTP-7-SS-Biotin, 3′-O-SS-dCTP-5-SS-Biotin/TCO, 3′-O-SS-dTTP-5-SS-Cy5-TCO) to the immobilized primed DNA template enables incorporation of these virtual terminators. Step 2, a chase with Therminator IX DNA polymerase and the four unlabeled reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to any primer strands not terminated with the labeled reversible terminators. Step 3, after washing away the unincorporated nucleotide analogues, imaging for Cy5 fluorescence will reveal those primers extended with either 3′-O-SS-dATP-7-SS-Cy5 or 3′-O-SS-dTTP-5-SS-Cy5-TCO. Step 4, addition of streptavidin-Cy5 to label any incorporated 3′-O-SS-ddGTP-7-SS-Biotin or 3′-O-SS-dCTP-5-SS-Biotin/TCO analogues. Step 5, after washing away unused labeling reagents, a second imaging step is performed, and new fluorescence signals will confirm incorporation by either C or G reversible terminators. If a chase step has not been performed earlier, at or just prior to this point, an optional chase step with the four 3′-O-azidomethyl dNTPs may be performed to ensure that nearly every primer has been extended either with one of the unlabeled or labeled NRT analogues. Step 6, incubation with Tetrazine-BHQ to quench the fluorescence of the dyes on 3′-blocked reversible terminators C or T. Step 7, after washing to remove any free tetrazine-BHQ, a third imaging step is carried out. Substantial loss of Cy5 signal in the case of previously determined ATP or TTP reversible terminator analogue incorporation indicates T and remaining signal indicates A incorporation. Similarly, substantial loss of Cy5 signal in the case of previously determined CTP or GTP reversible terminator analogue incorporation indicates C and remaining signal indicates G incorporation. Step 8, cleavage of SS linker by adding THP to the elongated DNA strands results in removal of dyes on the reversible terminator analogues and also restores the 3′-OH group on any growing strands extended with either 3′-O-SS-dNTPs or 3′-O-azidomethyl-dNTPs. The DNA products are ready for the next cycle of the DNA sequencing reaction. Structures of nucleotides used in this scheme are presented in FIG. 46. In the imaging cartoons at each step, black indicates a positive Cy5 signal and white or light gray a background signal. The encoding in the summary cartoon at the end indicates the template sequence, not the incorporated nucleotides.

FIG. 48: Generalized set of dye labeled cleavable ddNTP analogues, labeling and quenching reagents for single color SBS using a pH responsive dye and quenching: One of the dideoxynucleotide analogues has Cy5 attached to the base, one has HCyC-646 attached, one has Cy5 and TCO attached in branched chain configuration, and the last has HCyC-646 and TCO attached, also in branched chain configuration, all via an SS linker. HCyC-646 exhibits pH responsive fluorescence. The quenching molecule binds via the TCO anchors. A requirement of this hybrid SBS method is a separate set of four unlabeled reversible terminators (e.g., 3′-O-azidomethyl dNTPs).

FIG. 49: Simplified presentation of scheme for single color SBS using cleavable nucleotide analogues such as those presented in FIG. 48. Each type of ddNTP has one of the following, Cy5, HCyC-646, Cy5-TCO or HCyC-646-TCO, attached via an SS linker. The rectangles represent areas on a substrate containing numerous copies of attached primer-loop-template molecules (or other template-bound primer arrangements) in which the next base in the template strand, from left to right is T, G, C or A. After incubation with the four unlabeled nucleotide reversible terminators (NRTs, e.g., 3′-O-azidomethyl dNTPs) and Therminator IX to extend the majority of the primers, extension is carried out with Thermo Sequenase and four ddNTP analogues, ddATP attached to Cy5, ddTTP attached to HCyC-646, ddGTP attached to Cy5-TCO, and ddCTP attached to HCyC-646-TCO. After washing at pH 9, imaging will reveal a positive signal in the first and third rectangular areas (representing extension of the primer strand with either A or G) and a background signal in the remaining areas. After washing at pH 5, imaging will reveal a new positive signal in the remaining areas due to protonation of HCyC-646, indicating incorporation of C or T. Treatment with tetrazine-BHQ quenches the fluorescence of HCyC-646 and Cy5 on C and G ddNTP analogues. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps; 11 for A, 00 for C, 10 for G and 01 for T considering just the first and last of these imaging steps).

FIG. 50: Example ddNTP Analogues and Quencher-Anchor Binding Molecule Used for FIG. 51.

FIGS. 51A-51B: Single Color Sequencing by Synthesis Using a Set of ddNTP Analogues, One with Cy5, One with pH-Responsive Fluor HCyC-646, One with Cy5 and TCO Anchor, and One with HCyC-646 and TCO Anchor, All Attached to Base Via SS Linkers, Using a Dye Quencher Attached to Tetrazine. Use of ddNTP-Cleavable Linker-Dyes (ddATP-7-SS-Cy5, ddTTP-5-SS-HCyC-646), ddNTP-Cleavable Linker-Dye-Anchors (ddGTP-7-SS-Cy5-TCO, ddCTP-5-SS-HCyC-646-TCO), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Quencher (Tetrazine-BHQ) to perform 1-color DNA SBS. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddATP-7-SS-Cy5, ddTTP-5-SS-HCyC-646, ddGTP-7-SS-Cy5-TCO, ddCTP-5-SS-HCyC-646) to the immobilized primed DNA template enables incorporation of ddNTPs on most of the remaining template-loop-primers. Step 3, after washing away the unincorporated nucleotide analogues at pH 9, imaging for Cy5 fluorescence will reveal those primers extended with either ddATP-7-SS-Cy5 or ddGTP-7-SS-Cy5-TCO. Step 4, a second wash at pH 5 will permit fluorescence of the HCyC-646 on the ddTTP-5-SS-HCyC-646 and the ddCTP-5-SS-HCyC-646-TCO. A second imaging step is performed at pH 5, and new fluorescence signals will confirm incorporation by either ddC or ddT. At or just prior to this point, an optional chase step with the four 3′-O-azidomethyl dNTPs may be performed to ensure that nearly every primer has been extended either with one of the ddNTP or NRT analogues. Step 5, incubation with Tetrazine-BHQ to quench dyes on ddG or ddC analogues. Step 6, after washing to remove any free tetrazine-BHQ, a third imaging step is carried out at pH 5. Loss of Cy5 signal in the case of previously determined ddATP or ddGTP analogue incorporation indicates ddG and remaining signal indicates ddA incorporation. Similarly, loss of fluorescence signal in the case of previously determined ddCTP or ddTTP analogue incorporation indicates ddC and remaining signal indicates ddT incorporation. Step 7, cleavage of SS linker by adding THP to the elongated DNA strands results in removal of dyes on the nucleotide analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 50. In the imaging cartoons at each step, black indicates a positive fluorescent signal and white or light gray a background signal. The encoding in the summary cartoon at the end indicates the template sequence, not the incorporated nucleotides.

FIG. 52: Generalized set of dye or anchor labeled cleavable ddNTP analogues and labeling reagents for single color SBS using a click-to-cleave linker and quenching: One of the dideoxynucleotide analogues has Cy5 attached to the base via an SS linker, one has biotin attached to the base via an SS linker, one has Cy5 attached to the base via a linker containing SS (shown as Cleavable Linker 1) and TCO (shown as Cleavable Linker 2), and the last has biotin attached to the base via an SS- and TCO-containing linker. The binding molecule is dye-labeled streptavidin, and the dyes can be released via a click-to-cleave reaction at Cleavable Linker 2 or standard cleavage at Cleavable Linker 1. A requirement of this hybrid SBS method is a separate set of four unlabeled reversible terminators (e.g., 3′-O-azidomethyl dNTPs).

FIG. 53: Simplified presentation of scheme for single color SBS using cleavable nucleotide analogues such as those presented in FIG. 52. Each type of ddNTP has one of the following, Cy5 or Biotin, attached via an SS linker or a linker with both an SS and a TCO group. The rectangles represent areas on a substrate containing numerous copies of attached primer-loop-template molecules (or other template-bound primer arrangements) in which the next base in the template strand, from left to right is T, G, C or A. After incubation with the four unlabeled nucleotide reversible terminators (NRTs, e.g., 3′-O-azidomethyl dNTPs) and Therminator IX to extend the majority of the primers, extension is carried out with Thermo Sequenase and four ddNTP analogues, ddATP attached to Cy5 via an SS linker, ddTTP attached to biotin via an SS linker, ddGTP attached to Cy5 via a linker containing both SS and TCO, and ddCTP attached to biotin via a linker containing both SS and TCO. After washing, imaging will reveal a positive signal in the first and third rectangular areas (representing extension of the primer strand with either A or G) and a background signal in the remaining areas. Treatment with Streptavidin-Cy5 will label ddCTP and ddTTP. After washing, imaging will reveal new positive signals in the second and fourth rectangular areas (representing extension of the primer strand with either C or T). Reaction of tetrazine with TCO will release the Cy5 on the ddCTP and ddGTP nucleotide analogues. After washing, loss of fluorescence will reveal incorporation by C and G specifically, while remaining fluorescence will reveal incorporation by A and T respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps; 11 for A, 00 for C, 10 for G and 01 for T considering just the first and last of these imaging steps).

FIG. 54: Example ddNTP Analogues Used for FIG. 55.

FIGS. 55A-55B: Single Color Sequencing by Synthesis Using a Set of Orthogonal ddNTP Analogues, Containing Either Cy5 or Biotin and Either SS Only Linker or SS Plus TCO Linkers, Using a Streptavidin-Cy5 Labeling Step. Use of ddNTP-Cleavable Linker-Dyes (ddATP-7-SS-Cy5, ddGTP-7-SS-TCO-Cy5), ddNTP-Cleavable Linker-Dye-Anchors (ddTTP-5-SS-Biotin, ddCTP-5-SS-TCO-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddATP-7-SS-Cy5, ddGTP-7-SS-TCO-Cy5, ddTTP-5-SS-Biotin, ddCTP-5-SS-TCO-Biotin) to the immobilized primed DNA template enables incorporation of ddNTPs on most of the remaining template-loop-primers. Step 3, after washing away the unincorporated nucleotide analogues, imaging for Cy5 fluorescence will reveal those primers extended with either ddATP-7-SS-Cy5 or ddGTP-7-SS-TCO-Cy5. Step 4, labeling with Streptavidin-Cy5 will attach Cy5 via the biotin anchor on ddTTP-5-SS-Biotin and ddCTP-5-SS-TCO-Biotin. Step 5, a second imaging step is performed, and new fluorescence signals will confirm incorporation by either ddC or ddT. At or just prior to this point, an optional chase step with the four 3′-O-azidomethyl dNTPs may be performed to ensure that nearly every primer has been extended either with one of the ddNTP or NRT analogues. Step 6, incubation with Tetrazine to cleave dyes on ddC or ddG analogues. Step 7, after washing to remove any free tetrazine, a third imaging step is carried out. Loss of Cy5 signal in the case of previously determined ddATP or ddGTP analogue incorporation indicates ddG and remaining signal indicates ddA incorporation. Similarly, loss of Cy5 signal in the case of previously determined ddCTP or ddTTP analogue incorporation indicates ddC and remaining signal indicates ddT incorporation. Step 8, cleavage of SS linker by adding THP to the elongated DNA strands results in removal dyes on the nucleotide analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 54. In the imaging cartoons at each step, black indicates a positive Cy5 signal and white a background signal. The encoding in the summary cartoon at the end indicates the template sequence, not the incorporated nucleotides.

FIG. 56: Generalized set of dye or anchor labeled cleavable virtual terminator nucleotide analogues and labeling reagent for single color SBS using a click-to-cleave linker and quenching: One of the virtual terminator analogues has Cy5 attached to the base via an SS linker, one has biotin attached to the base via an SS linker, one has Cy5 attached to the base via a linker containing SS (shown as Cleavable Linker 1) and TCO (shown as Cleavable Linker 2), and the last has biotin attached to the base via an SS- and TCO-containing linker. The binding molecule is dye-labeled streptavidin, and the dyes can be released via a click-to-cleave reaction at Cleavable Linker 2 or standard cleavage at Cleavable Linker 1. Chase reactions are performed with four unlabeled reversible terminators (e.g., 3′-O-azidomethyl dNTPs).

FIG. 57: Simplified presentation of scheme for single color SBS using cleavable nucleotide analogues such as those presented in FIG. 56. Each type of virtual terminator nucleotide analogue has one of the following, Cy5 or Biotin, attached via an SS linker or a linker with both an SS and a TCO group. The rectangles represent areas on a substrate containing numerous copies of attached primer-loop-template molecules (or other template-bound primer arrangements) in which the next base in the template strand, from left to right is T, G, C or A. An extension is carried out with Thermo Sequenase and four virtual terminator analogues, dATP attached to Cy5 via an SS linker, dTTP attached to biotin via an SS linker, dGTP attached to Cy5 via a linker containing both SS and TCO, and dCTP attached to biotin via a linker containing both SS and TCO. A chase with the four unlabeled nucleotide reversible terminators (NRTs, e.g., 3′-O-azidomethyl dNTPs) and Therminator IX is performed to extend all the remaining primers. After washing, imaging will reveal a positive signal in the first and third rectangular areas (representing extension of the primer strand with either A or G) and a background signal in the remaining areas. Treatment with Streptavidin-Cy5 will label dCTP and dTTP virtual terminator analogues. After washing, imaging will reveal new positive signals in the second and fourth rectangular areas (representing extension of the primer strand with either C or T). Reaction between tetrazine and TCO will release the Cy5 on the dCTP and dGTP virtual terminator analogues. After washing, loss of fluorescence will reveal incorporation by C and G specifically, while remaining fluorescence will reveal incorporation by A and T respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps; 11 for A, 00 for C, 10 for G and 01 for T considering just the first and last of these imaging steps).

FIG. 58: Example Virtual Terminator Nucleotide Analogues Used for FIG. 59.

FIGS. 59A-59B: Single Color Sequencing by Synthesis Using a Set of Orthogonal Virtual Terminator Nucleotide Analogues, Containing Either Cy5 or Biotin and Either SS Only Linker or SS Plus TCO Linkers, Using a Streptavidin-Cy5 Labeling Step. Use of dNTP-Blocker-Cleavable Linker-Dyes (dATP-7-SS-Blocker-Cy5, dGTP-7-SS-Blocker-TCO-Cy5), dNTP-Cleavable Linker-Blocker-Dye-Anchors (dTTP-5-SS-Blocker-Biotin, dCTP-5-SS-Blocker-TCO-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. Step 1, Addition of Thermo Sequenase and the four virtual terminator analogues (dATP-7-SS-Blocker-Cy5, dGTP-7-SS-Blocker-TCO-Cy5, dTTP-5-SS-Blocker-Biotin, dCTP-5-SS-Blocker-TCO-Biotin) to the immobilized primed DNA template enables incorporation of virtual terminators on the template-loop-primers (or other template-bound primer arrangements). Step 2, chase with Therminator IX DNA polymerase and the four unlabeled reversible terminators (3′-O-azidomethyl-dATP, 3′-O-azidomethyl-dCTP, 3′-O-azidomethyl-dGTP, 3′-O-azidomethyl-dTTP) enables the incorporation of the complementary nucleotide analogue to the remaining growing DNA strands. Step 3, after washing away the unincorporated nucleotide analogues, imaging for Cy5 fluorescence will reveal those primers extended with either dATP-7-SS-Blocker-Cy5 or dGTP-7-SS-Blocker-TCO-Cy5. Step 4, labeling with Streptavidin-Cy5 will attach Cy5 via the biotin anchor on dTTP-5-SS-Blocker-Biotin and dCTP-5-SS-Blocker-TCO-Biotin. Step 5, a second imaging step is performed, and new fluorescence signals will confirm incorporation by either the dC or dT virtual terminator. At or just prior to this point, an optional chase step with the four 3′-O-azidomethyl dNTPs may be performed to ensure that nearly every primer has been extended either with one of the virtual terminator or NRT analogues. Step 6, incubation with Tetrazine to cleave dyes on dC or dG virtual terminator analogues. Step 7, after washing to remove any free tetrazine, a third imaging step is carried out. Loss of Cy5 signal in the case of previously determined dATP or dGTP virtual terminator analogue incorporation indicates dG and remaining signal indicates dA incorporation. Similarly, loss of Cy5 signal in the case of previously determined dCTP or dTTP virtual terminator analogue incorporation indicates dC and remaining signal indicates dT incorporation. Step 8, cleavage of SS linker by adding THP to the elongated DNA strands results in removal dyes on the virtual terminator analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 58. In the imaging cartoons at each step, black indicates a positive Cy5 signal and white a background signal. The encoding in the summary cartoon at the end indicates the template sequence, not the incorporated nucleotides.

FIG. 60: Generalized set of dye or anchor cleavable nucleotide reversible terminator analogues and labeling reagent for single color SBS using a click-to-cleave linker: One of the nucleotide reversible terminator analogues has Cy5 attached to the base via an SS linker, one has biotin attached to the base via an SS linker, one has Cy5 attached to the base via a linker containing SS (shown as Cleavable Linker 1) and TCO (shown as Cleavable Linker 2), and the last has biotin attached to the base via an SS- and TCO-containing linker. The binding molecule is dye-labeled streptavidin, and the dyes can be released via a click-to-cleave reaction at Cleavable Linker 2 or standard cleavage at Cleavable Linker 1. Chase reactions are performed with four unlabeled reversible terminators (e.g., 3′-O-azidomethyl dNTPs).

FIG. 61: Simplified presentation of scheme for single color SBS using cleavable nucleotide analogues such as those presented in FIG. 60. Each type of nucleotide reversible terminator analogue has one of the following, Cy5 or Biotin, attached via an SS linker or a linker with both an SS and a TCO group. The rectangles represent areas on a substrate containing numerous copies of attached primer-loop-template molecules (or other template-bound primer arrangements) in which the next base in the template strand, from left to right is T, G, C or A. An extension is carried out with Therminator IX and four reversible terminator analogues, 3′-O-t-Butyl-SS-dATP attached to Cy5 via an SS linker, 3′-O-t-Butyl-SS-dTTP attached to biotin via an SS linker, 3′-O-t-Butyl-SS-dGTP attached to Cy5 via a linker containing both SS and TCO, and 3′-O-t-Butyl-SS-dCTP attached to biotin via a linker containing both SS and TCO. A chase with the four unlabeled nucleotide reversible terminators (NRTs, e.g., 3′-O-azidomethyl dNTPs) and Therminator IX is performed to extend all the remaining primers. After washing, imaging will reveal a positive signal in the first and third rectangular areas (representing extension of the primer strand with either A or G) and a background signal in the remaining areas. Treatment with Streptavidin-Cy5 will label dCTP and dTTP reversible terminator analogues. After washing, imaging will reveal new positive signals in the second and fourth rectangular areas (representing extension of the primer strand with either C or T). Reaction of tetrazine with TCO will release the Cy5 on the dCTP and dGTP reversible terminator analogues. After washing, loss of fluorescence will reveal incorporation by C and G specifically, while remaining fluorescence will reveal incorporation by A and T respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps; 11 for A, 00 for C, 10 for G and 01 for T considering just the first and last of these imaging steps).

FIG. 62: Example 3′-SS-dNTP Analogues (Reversible Terminators) Used for FIG. 63.

FIGS. 63A-63B: Single Color Sequencing by Synthesis Using a Set of Orthogonal Nucleotide Reversible Terminator Analogues, Containing Either Cy5 or Biotin and Either SS Only Linker or SS Plus TCO Linkers, Using a Streptavidin-Cy5 Labeling Step. Use of 3′-O-SS-dNTP-Cleavable Linker-Dyes (3′-O-SS-ATP-7-SS-Cy5, 3′-O-SS-dGTP-7-SS-TCO-Cy5), 3′-O-SS-dNTP-Cleavable Linker-Dye-Anchors (3′-O-SS-dTTP-5-SS-Biotin, 3′-O-SS-dCTP-5-SS-TCO-Biotin), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Dye (Streptavidin-Cy5) to perform 1-color DNA SBS. Step 1, Addition of Therminator IX and the four virtual terminator analogues (3′-O-SS-ATP-7-SS-Cy5, 3′-O-SS-dGTP-7-SS-TCO-Cy5, 3′-O-SS-dTTP-5-SS-Biotin, 3′-O-SS-dCTP-5-SS-TCO-Biotin) to the immobilized primed DNA template enables incorporation of 3′-blocked reversible terminators on the template-loop-primers. Step 2, chase with Therminator IX DNA polymerase and the four unlabeled reversible terminators (3′-O-azidomethyl-dATP, 3′-O-azidomethyl-dCTP, 3′-O-azidomethyl-dGTP, 3′-O-azidomethyl-dTTP) enables the incorporation of the complementary nucleotide analogue to the remaining growing DNA strands. Step 3, after washing away the unincorporated nucleotide analogues, imaging for Cy5 fluorescence will reveal those primers extended with either 3′-O-SS-ATP-7-SS-Cy5 or 3′-O-SS-dGTP-7-SS-TCO-Cy5. Step 4, labeling with Streptavidin-Cy5 will will attach Cy5 via the biotin anchor on 3′-O-SS-dTTP-5-SS-Biotin and 3′-O-SS-dCTP-5-SS-TCO-Biotin. Step 5, a second imaging step is performed, and new fluorescence signals will confirm incorporation by either the dC or dT reversible terminators. At or just prior to this point, an optional chase step with the four 3′-O-azidomethyl dNTPs may be performed to ensure that nearly every primer has been extended either with one of the labeled or unlabeled NRT analogues. Step 6, incubation with tetrazine to cleave dyes on dC or dG reversible terminator analogues. Step 7, after washing to remove any free tetrazine, a third imaging step is carried out. Loss of Cy5 signal in the case of previously determined dATP or dGTP reversible terminator analogue incorporation indicates dG and remaining signal indicates dA incorporation. Similarly, loss of Cy5 signal in the case of previously determined dCTP or dTTP reversible terminator analogue incorporation indicates dC and remaining signal indicates dT incorporation. Step 8, cleavage of SS linker by adding THP to the elongated DNA strands results in removal dyes on the dATP and dGTP reversible terminator analogues and also restores the 3′-OH group on any growing strands extended with 3′-O-SS-dNTPs or 3′-O-azidomethyl-dNTPs. The DNA products are ready for the next cycle of the DNA sequencing reaction. Structures of nucleotides used in this scheme are presented in FIG. 62. In the imaging cartoons at each step, black indicates a positive Cy5 signal and white a background signal. The encoding in the summary cartoon at the end indicates the template sequence, not the incorporated nucleotides.

FIG. 64: Generalized set of dye labeled cleavable ddNTP analogues for single color SBS using a click-to-cleave linker and a pH responsive dye: One of the dideoxynucleotide analogues has Cy5 attached to the base via an SS linker, one has HCyC-646 attached to the base via an SS linker, one has Cy5 attached to the base via a linker containing SS (shown as Cleavable Linker 1) and TCO (shown as Cleavable Linker 2), and the last has HCyC-646 attached to the base via an SS- and TCO-containing linker. HCyC-646 is a pH-responsive dye that fluoresces below pH 6. The dyes can be released via a click-to-cleave reaction at Cleavable Linker 2 or standard cleavage at Cleavable Linker 1. A requirement of this hybrid SBS method is a separate set of four unlabeled reversible terminators (e.g., 3′-O-azidomethyl dNTPs).

FIG. 65: Simplified presentation of scheme for single color SBS using cleavable nucleotide analogues such as those presented in FIG. 64. Each type of ddNTP has one of the following, Cy5 or HCyC-646, attached via an SS linker or a linker with both an SS and a TCO group. The rectangles represent areas on a substrate containing numerous copies of attached primer-loop-template molecules (or other template-bound primer arrangements) in which the next base in the template strand, from left to right is T, G, C or A. After incubation with the four unlabeled nucleotide reversible terminators (NRTs, e.g., 3′-O-azidomethyl dNTPs) and Therminator IX to extend the majority of the primers, extension is carried out with Thermo Sequenase and four ddNTP analogues, ddATP attached to Cy5 via an SS linker, ddTTP attached to HCyC-646 via an SS linker, ddGTP attached to Cy5 via a linker containing both SS and TCO, and ddCTP attached to HCyC-646 via a linker containing both SS and TCO. After washing at pH 9, imaging will reveal a positive signal due to Cy5 fluorescence in the first and third rectangular areas (representing extension of the primer strand with either A or G) and a background signal in the remaining areas. After washing at pH 5, imaging will reveal new positive signals in the second and fourth rectangular areas due to the ability of HCyC-646 to fluoresce below pH 6 (representing extension of the primer strand with either C or T). Reaction of tetrazine with TCO will release the Cy5 on the ddCTP and ddGTP nucleotide analogues. After washing, loss of fluorescence will reveal incorporation by C and G specifically, while remaining fluorescence will reveal incorporation by A and T respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps; 11 for A, 00 for C, 10 for G and 01 for T considering just the first and last of these imaging steps).

FIG. 66: Example ddNTP Analogues Used for FIG. 67.

FIGS. 67A-67B: Single Color Sequencing by Synthesis Using a Set of Orthogonal ddNTP Analogues, Containing Either Cy5 or HCyC-646 and Either SS Only Linker or SS Plus TCO Linkers. Use of ddNTP-Cleavable Linker-Dyes (ddATP-7-SS-Cy5, ddGTP-7-SS-TCO-Cy5, ddTTP-5-SS-HCyC-646, ddCTP-5-SS-TCO-HCyC-646) and 3′-O-azidomethyl dNTPs (3′-O-azidomethyl-dATP, 3′-O-azidomethyl-dCTP, 3′-O-azidomethyl-dGTP, 3′-O-azidomethyl-dTTP) to perform 1-color DNA SBS. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddATP-7-SS-Cy5, ddGTP-7-SS-TCO-Cy5, ddTTP-SS-HCyC-646, ddCTP-SS-TCO-HCyC-646) to the immobilized primed DNA template enables incorporation of ddNTPs on most of the remaining primers. Step 3, after washing away the unincorporated nucleotide analogues at pH 9, imaging for Cy5 fluorescence will reveal those primers extended with either ddATP-7-SS-Cy5 or ddGTP-7-SS-TCO-Cy5. Step 4, washing at pH 5 will permit fluorescence of the HCyC-646 dyes on ddTTP-5-SS-HCyC-646 and ddCTP-5-SS-TCO-HCyC-646. A second imaging step at pH 5 is performed, and new fluorescence signals will confirm incorporation by either ddC or ddT. At or just prior to this point, an optional chase step with the four 3′-O-azidomethyl dNTPs may be performed to ensure that nearly every primer has been extended either with one of the ddNTP or NRT analogues. Step 5, incubation with Tetrazine to cleave dyes on ddC or ddG analogues. Step 6, after washing at pH 5 to remove any free tetrazine, a third imaging step is carried out at pH 5. Loss of HCyC-646 fluorescence signal in the case of previously determined ddATP or ddGTP analogue incorporation indicates ddG and remaining signal indicates ddA incorporation. Similarly, loss of Cy5 signal in the case of previously determined ddCTP or ddTTP analogue incorporation indicates ddC and remaining signal indicates ddT incorporation. Step 7, cleavage of SS linker by adding THP to the elongated DNA strands results in removal of and remaining blockers and dyes on virtual terminators nucleotide analogues and also restores the 3′-OH group on any growing strands extended with 3′-O-azidomethyl-dNTPs. The DNA products are ready for the next cycle of the DNA sequencing reaction. Structures of nucleotides used in this scheme are presented in FIG. 66. In the imaging cartoons at each step, black indicates a positive fluorescence signal and white a background signal. The encoding in the summary cartoon at the end indicates the template sequence, not the incorporated nucleotides.

FIG. 68: Generalized set of dye labeled cleavable dNTP-Blocker (Virtual Terminator) analogues for single color SBS using a click-to-cleave linker and a pH responsive dye: One of the virtual terminator analogues has Cy5 attached to the base via an SS linker, one has HCyC-646 attached to the base via an SS linker, one has Cy5 attached to the base via a linker containing SS (shown as Cleavable Linker 1) and TCO (shown as Cleavable Linker 2), and the last has HCyC-646 attached to the base via an SS- and TCO-containing linker. HCyC-646 is a pH-responsive dye that fluoresces below pH 6. The dyes can be released via a click-to-cleave reaction at Cleavable Linker 2 or standard cleavage at Cleavable Linker 1. A chase step with four unlabeled reversible terminators (e.g., 3′-O-azidomethyl dNTPs) is also needed.

FIG. 69: Simplified presentation of scheme for single color SBS using cleavable nucleotide analogues such as those presented in FIG. 68. Each type of virtual terminator has one of the following, Cy5 or HCyC-646, attached via an SS linker or a linker with both an SS and a TCO group. The rectangles represent areas on a substrate containing numerous copies of attached primer-loop-template molecules (or other template-bound primer arrangements) in which the next base in the template strand, from left to right is T, G, C or A. An extension is carried out with Thermo Sequenase and four virtual terminator analogues, dATP attached to Cy5 via an SS linker, dTTP attached to HCyC-646 via an SS linker, dGTP attached to Cy5 via a linker containing both SS and TCO, and dCTP attached to HCyC-646 via a linker containing both SS and TCO. A chase with the four unlabeled nucleotide reversible terminators (NRTs, e.g., 3′-O-azidomethyl dNTPs) and Therminator IX is performed to extend all the remaining primers. After washing at pH 9, imaging will reveal a positive signal due to Cy5 fluorescence in the first and third rectangular areas (representing extension of the primer strand with either A or G) and a background signal in the remaining areas. After washing at pH 5, imaging will reveal new positive signals in the second and fourth rectangular areas due to the ability of HCyC-646 to fluoresce below pH 6 (representing extension of the primer strand with either C or T). Reaction of tetrazine with TCO will release the Cy5 on the dCTP and dGTP nucleotide analogues. After washing, loss of fluorescence will reveal incorporation by C and G specifically, while remaining fluorescence will reveal incorporation by A and T respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps; 11 for A, 00 for C, 10 for G and 01 for T considering just the first and last of these imaging steps).

FIG. 70: Example dNTP Analogues Used for FIG. 71.

FIGS. 71A-71B: Single Color Sequencing by Synthesis Using a Set of Orthogonal dNTP-Blocker (Virtual Terminator) Analogues, Containing Either Cy5 or HCyC-646 and Either SS Only Linker or SS Plus TCO Linkers. Use of dNTP-Cleavable Linker-Blocker-Dyes (dATP-7-SS-Blocker-Cy5, dGTP-7-SS-Blocker-TCO-Cy5, dTTP-5-SS-Blocker-HCyC-646, dCTP-5-SS-Blocker-TCO-HCyC-646) and 3′-O-azidomethyl dNTPs (3′-O-azidomethyl-dATP, 3′-O-azidomethyl-dCTP, 3′-O-azidomethyl-dGTP, 3′-O-azidomethyl-dTTP) to perform 1-color DNA SBS. Step 1, Addition of Thermo Sequenase and the four dNTP-Blocker virtual terminator analogues (dATP-7-SS-Blocker-Cy5, dGTP-7-SS-Blocker-TCO-Cy5, dTTP-5-SS-Blocker-HCyC-646, dCTP-5-SS-Blocker-TCO-HCyC-646) to the immobilized primed DNA template enables incorporation of these dNTPs to the 3′ end of the template-loop-primers (or primers in other template-bound primer arrangements) opposite the complementary base on the template strand. Step 2, Chase with Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to most of the remaining growing DNA strands to terminate DNA synthesis. Step 3, after washing away the unincorporated nucleotide analogues at pH 9, imaging for Cy5 fluorescence will reveal those primers extended with either dATP-7-SS-Blocker-Cy5 or dGTP-7-SS-Blocker-TCO-Cy5. Step 4, washing at pH 5 will permit fluorescence of the HCyC-646 dyes on dTTP-5-SS-Blocker-HCyC-646 and dCTP-5-SS-Blocker-TCO-HCyC-646. A second imaging step at pH 5 is performed, and new fluorescence signals will confirm incorporation by either dC or dT. At or just prior to this point, an optional chase step with the four 3′-O-azidomethyl dNTPs may be performed to ensure that nearly every primer has been extended either with one of the virtual terminator or NRT analogues. Step 5, incubation with Tetrazine to cleave dyes on dC or dG analogues. Step 6, after washing at pH 5 to remove any free tetrazine, a third imaging step is carried out at pH 5. Loss of HCyC-646 fluorescence signal in the case of previously determined dATP or dGTP analogue incorporation indicates dG and remaining signal indicates dA incorporation. Similarly, loss of Cy5 signal in the case of previously determined dCTP or dTTP analogue incorporation indicates dC and remaining signal indicates dT incorporation. Step 7, cleavage of SS linker by adding THP to the elongated DNA strands results in removal of any remaining blockers and dyes from the incorporated virtual terminators and also restores the 3′-OH group on any growing strands extended with 3′-O-azidomethyl-dNTPs. The DNA products are ready for the next cycle of the DNA sequencing reaction. Structures of nucleotides used in this scheme are presented in FIG. 70. In the imaging cartoons at each step, black indicates a positive fluorescence signal and white a background signal. The encoding in the summary cartoon at the end indicates the template sequence, not the incorporated nucleotides.

FIG. 72: Generalized set of dye labeled cleavable 3′-blocked reversible terminator analogues for single color SBS using a click-to-cleave linker and a pH responsive dye: One of the reversible terminator analogues has Cy5 attached to the base via an SS linker, one has HCyC-646 attached to the base via an SS linker, one has Cy5 attached to the base via a linker containing SS (shown as Cleavable Linker 1) and TCO (shown as Cleavable Linker 2), and the last has HCyC-646 attached to the base via an SS- and TCO-containing linker. HCyC-646 is a pH-responsive dye that fluoresces below pH 6. The dyes can be released via a click-to-cleave reaction at Cleavable Linker 2 or standard cleavage at Cleavable Linker 1. A chase step with four unlabeled reversible terminators (e.g., 3′-O-azidomethyl dNTPs) is also needed.

FIG. 73: Simplified presentation of scheme for single color SBS using cleavable nucleotide analogues such as those presented in FIG. 72. Each type of 3′-blocked nucleotide reversible terminator has one of the following, Cy5 or HCyC-646, attached via an SS linker or a linker with both an SS and a TCO group. The rectangles represent areas on a substrate containing numerous copies of attached primer-loop-template molecules (or other template-bound primer arrangements) in which the next base in the template strand, from left to right is T, G, C or A. An extension is carried out with Therminator IX and four reversible terminator analogues, dATP attached to Cy5 via an SS linker, dTTP attached to HCyC-646 via an SS linker, dGTP attached to Cy5 via a linker containing both SS and TCO, and dCTP attached to HCyC-646 via a linker containing both SS and TCO. A chase with the four unlabeled nucleotide reversible terminators (NRTs, e.g., 3′-O-azidomethyl dNTPs) and Therminator IX is performed to extend all the remaining primers. After washing at pH 9, imaging will reveal a positive signal due to Cy5 fluorescence in the first and third rectangular areas (representing extension of the primer strand with either A or G) and a background signal in the remaining areas. After washing at pH 5, imaging will reveal new positive signals in the second and fourth rectangular areas due to the ability of HCyC-646 to fluoresce below pH 6 (representing extension of the primer strand with either C or T). Reaction of tetrazine with TCO will release the Cy5 on the dCTP and dGTP nucleotide reversible terminator analogues. After washing, loss of fluorescence will reveal incorporation by C and G specifically, while remaining fluorescence will reveal incorporation by A and T respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps; 11 for A, 00 for C, 10 for G and 01 for T considering just the first and last of these imaging steps).

FIG. 74: Example 3′-SS-dNTP Analogues Used for FIG. 75.

FIGS. 75A-75B: Single Color Sequencing by Synthesis Using a Set of Orthogonal 3′-O-Blocked Nucleotide Reversible Terminator Analogues, Containing Either Cy5 or HCyC-646 and Either SS Only Linker or SS Plus TCO Linkers. Use of 3′-O-SS-dNTP-Cleavable Linker-Dyes (3′-O-SS-dATP-7-SS-Cy5, 3′-O-SS-dGTP-7-SS-TCO-Cy5, 3′-O-SS-dTTP-5-SS-HCyC-646, 3′-O-SS-dCTP-5-SS-TCO-HCyC-646) and 3′-O-azidomethyl dNTPs (3′-O-azidomethyl-dATP, 3′-O-azidomethyl-dCTP, 3′-O-azidomethyl-dGTP, 3′-O-azidomethyl-dTTP) to perform 1-color DNA SBS. Step 1, Addition of Therminator IX and the four 3′-O-SS-dNTP analogues (3′-O-SS-dATP-7-SS-Cy5, 3′-O-SS-dGTP-7-SS-TCO-Cy5, 3′-O-SS-dTTP-5-SS-HCyC-646, 3′-O-SS-dCTP-5-SS-TCO-HCyC-646) to the immobilized primed DNA template enables incorporation of these dye-labeled reversible terminators to the 3′ end of the template-loop-primers (or primers in other template-bound primer arrangements) opposite the complementary base on the template strand. Step 2, Chase with Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to most of the remaining growing DNA strands to terminate DNA synthesis. Step 3, after washing away the unincorporated nucleotide analogues at pH 9, imaging for Cy5 fluorescence will reveal those primers extended with either 3′-O-SS-dATP-7-SS-Cy5 or 3′-O-SS-dGTP-7-SS-TCO-Cy5. Steop 4, washing at pH 5 will permit fluorescence of the HCyC-646 dyes on 3′-O-SS-dTTP-5-SS-HCyC-646 and 3′-O-SS-dCTP-5-SS-TCO-HCyC-646. A second imaging step at pH 5 is performed, and new fluorescence signals will confirm incorporation by either dC or dT. At or just prior to this point, an optional chase step with the four 3′-O-azidomethyl dNTPs may be performed to ensure that nearly every primer has been extended either with one of the dye-labeled nucleotide reversible terminator or unlabeled NRT analogues. Step 5, incubation with Tetrazine to cleave dyes on dC or dG analogues. Step 6, after washing at pH 5 to remove any free tetrazine, a third imaging step is carried out. Loss of HCyC-646 fluorescence signal in the case of previously determined dATP or dGTP analogue incorporation indicates dG and remaining signal indicates dA incorporation. Similarly, loss of Cy5 signal in the case of previously determined dCTP or dTTP analogue incorporation indicates dC and remaining signal indicates dT incorporation. Step 7, cleavage of SS linker by adding THP to the elongated DNA strands results in removal of remaining dyes on the nucleotide analogues and also restores the 3′-OH group on any growing strands extended with 3′-O-SS-dNTPs or 3′-O-azidomethyl-dNTPs. The DNA products are ready for the next cycle of the DNA sequencing reaction. Structures of nucleotides used in this scheme are presented in FIG. 74. In the imaging cartoons at each step, black indicates a positive fluorescence signal and white a background signal. The encoding in the summary cartoon at the end indicates the template sequence, not the incorporated nucleotides.

FIG. 76: Generalized set of dye labeled cleavable ddNTP analogues for single color SBS using a a pH responsive dye: Two of the dideoxynucleotide analogues have Cy5 attached to the base via an SS linker and the other two have HCyC-646 attached to the base via an SS linker. HCyC-646 is a pH-responsive dye that fluoresces below pH 6. A requirement of this hybrid SBS method is a separate set of four unlabeled reversible terminators (e.g., 3′-O-azidomethyl dNTPs).

FIG. 77: Simplified presentation of scheme for single color SBS using cleavable nucleotide analogues such as those presented in FIG. 76. Each type of ddNTP has one of the following, Cy5 or HCyC-646, attached via an SS linker. The rectangles represent areas on a substrate containing numerous copies of attached primer-loop-template molecules (or other template-bound primer arrangements) in which the next base in the template strand, from left to right is T, G, C or A. Extension is carried out with Therminator IX and two of the ddNTP analogues, ddATP attached to Cy5 via an SS linker and ddTTP attached to HCyC-646 via an SS linker, along with an excess of the 3′-O-azidomethyl dNTPs. After a wash at pH 5, imaging will reveal a positive signal in the first and fourth rectangular areas, due to Cy5 or HCyC-646 fluorescence, indicating incorporation of A or T. Next, incubation with ddGTP attached to Cy5 via an SS linker, and ddCTP attached to HCyC-646 via an SS linker, along with an excess of 3′-O-azidomethyl-dATP and 3′-O-azidomethyl-dTTP, to ensure fidelity and washing at pH 5, will result in new positive signals in the second and third rectangular area, indicating C or G incorporation. After washing at pH 9, imaging will reveal loss of positive signals in the second and fourth rectangular areas due to the ability of HCyC-646 to fluoresce below pH 6 but not at pH 9. Thus loss of fluorescence will indicate incorporation by T if it was formerly determined that either A or T were incorporated and incorporation of C if it was formerly determined that either C or G was incorporated. Remaining fluorescence indicates incorporation of A and G respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the four indicated imaging steps, a positive signal indicated by a 1 and a background signal indicated by a 0. Incorporation of each of the four possible nucleotide analogues will be revealed by a unique digital code (111 for A, 010 for C, 011 for G and 110 for T) considering all four of these imaging steps.

FIG. 78: Example ddNTP Analogues Used for FIG. 79.

FIGS. 79A-79B: Single Color Sequencing by Synthesis Using a Set of ddNTP Analogues, Containing Either Cy5 or HCyC-646. Use of ddNTP-Cleavable Linker-Dyes (ddATP-7-SS-Cy5, ddGTP-7-SS-Cy5, ddTTP-5-SS-HCyC-646, ddCTP-5-SS-HCyC-646) and 3′-O-azidomethyl dNTPs (3′-O-azidomethyl-dATP, 3′-O-azidomethyl-dCTP, 3′-O-azidomethyl-dGTP, 3′-O-azidomethyl-dTTP) to perform 1-color DNA SBS. Step 1, Addition of Therminator IX DNA polymerase, two of the ddNTP-Cleavable Linker-Dyes (ddATP-7-SS-Cy5, ddTTP-5-SS-HCyC-646) and an excess of the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary 3′-O-azidomethyl-dNTP to the majority of growing DNA strands (>95%) and the ddATP-7-SS-Cy5, ddTTP-5-SS-HCyC-646 on most of the remaining primers to terminate DNA synthesis. Step 2, after washing away the unincorporated nucleotide analogues at pH 5, imaging for Cy5 or HCyC-646 fluorescence (the two dyes absorb and emit light at essentially the same wavelengths as each other) will reveal those primers extended with either ddATP-7-SS-Cy5 or ddT-5-SS-HCyC-646. Step 3, Addition of Therminator IX DNA polymerase, the remaining two ddNTP-Cleavable Linker-Dyes (ddGTP-7-SS-Cy5, ddCTP-SS-HCyC-646) and the other two reversible terminators (3′-O-azidomethyl-dATP, 3′-O-azidomethyl-dTTP) to the immobilized primed DNA template enables high fidelity incorporation of ddCTP-5-SS-HCyC-646 and ddGTP-7-SS-Cy5. Step 4, After washing away the unincorporated nucleotides at pH 5, a second imaging step is performed to reveal Cy5 or HCyC-646 fluorescence, and new fluorescence signals will confirm incorporation by ddC or ddG. Step 5, after washing at pH 9 to eliminate fluorescence of the HCyC-646 dye on ddCTP-SS-HCyC-646 and ddTTP-SS-HCyC-646, a third imaging step will reveal which nucleotide was incorporated. Thus if it was determined that ddA or ddT was added in Imaging Step 2, loss of the fluorescence signal indicates incorporation by T and remaining signal indicates incorporation by A. If it was determined that ddC or ddG was added in Imaging Step 4, loss of fluorescence signal indicates incorporation by C and remaining signal incorporation by G. At or just prior to this point, an optional chase step with the four 3′-O-azidomethyl dNTPs may be performed to ensure that nearly every primer has been extended either with one of the ddNTP or NRT analogues. Step 6, cleavage of SS linker by adding THP to the elongated DNA strands results in removal dyes on the nucleotide analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 78. In the imaging cartoons at each step, black indicates a positive fluorescent signal and white a background signal. The encoding in the summary cartoon at the end indicates the template sequence, not the incorporated nucleotides.

FIG. 80: Generalized set of dye labeled cleavable ddNTP analogues for single color SBS using a pH responsive dye and an anchor for attachment of a dye quencher molecule: Two of the dideoxynucleotide analogues have Cy5 attached to the base via an SS linker and the other two have HCyC-646 attached to the base via an SS linker. An anchor for attachment of a quencher is present on one of the ddNTPs containing Cy5 and one containing HCyC-646. HCyC-646 is a pH-responsive dye that fluoresces below pH 6. A requirement of this hybrid SBS method is a separate set of four unlabeled reversible terminators (e.g., 3′-O-azidomethyl dNTPs).

FIG. 81: Simplified presentation of scheme for single color SBS using cleavable nucleotide analogues such as those presented in FIG. 80. Each type of ddNTP has one of the following, Cy5, Cy5-Tetrazine, HCyC-646, or HCyC-646-Tetrazine, attached via an SS linker. The rectangles represent areas on a substrate containing numerous copies of attached primer-loop-template molecules (or other template-bound primer arrangements) in which the next base in the template strand, from left to right is T, G, C or A. After incubation with the four unlabeled nucleotide reversible terminators (NRTs, e.g., 3′-O-azidomethyl dNTPs) and Therminator IX to extend the majority of the primers, extension is carried out with Thermo Sequenase and the four ddNTP analogues, ddATP attached to Cy5, ddTTP attached to HCyC-646, ddGTP attached to both tetrazine and Cy5, and ddCTP attached to both tetrazine and HCyC-646. After a wash at pH 9, imaging will reveal a positive signal in the first and third rectangular areas, due to Cy5 fluorescence, indicating incorporation of A or G. After switching to pH 5, imaging will reveal new fluorescence in the second and fourth rectangular areas, due to the low pH dependency of HCyC-646 fluorescence, indicating incorporation of C or T. Incubation with TCO-BHQ3 will attach the quencher to the tetrazine anchor on ddCTP and ddGTP, and after washing at pH 5, imaging will reveal fluorescence quenching resulting in substantially reduced fluorescence for these two nucleotide analogues, indicating incorporation of C or G specifically, whereas no loss of fluorescence will indicate incorporation of A or T specifically. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 110 for G and 011 for T considering all three of these imaging steps, or 11 for A, 00 for C, 10 for G and 01 for T considering only the first and third imaging step).

FIG. 82: Example ddNTP Analogues and Quencher-Anchor Binding Molecule Used for FIG. 83.

FIGS. 83A-83B: Single Color Sequencing by Synthesis Using a Set of Orthogonal ddNTP Analogues, Containing Either Cy5, HCyC-646, Tetrazine-Cy5, or Tetrazine-HCyC-646, and Quenching with TCO-BHQ3. Use of ddNTP-Cleavable Linker-Dyes (ddATP-7-SS-Cy5, ddGTP-7-SS-Tetrazine/Cy5, ddTTP-5-SS-HCyC-646, ddCTP-5-SS-Tetrazine/HCyC-646), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and Anchor Binding Molecule-Quencher (TCO-BHQ3), to perform 1-color DNA SBS. Step 1, Addition of Therminator IX DNA polymerase and the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary nucleotide analogue to the majority of growing DNA strands (>90%) to terminate DNA synthesis. Step 2, Addition of Thermo Sequenase and the four ddNTP analogues (ddATP-7-SS-Cy5, ddGTP-7-SS-Tetrazine/Cy5, ddTTP-5-SS-HCyC-646, ddCTP-5-SS-Tetrazine/HCyC-646) to the immobilized primed DNA template enables incorporation of ddNTPs on most of the remaining template-loop-primers. Step 3, after washing away the unincorporated nucleotide analogues at pH 9, imaging for Cy5 fluorescence will reveal those primers extended with either ddATP-7-SS-Cy5 or ddGTP-7-SS-Tetrazine/Cy5. Step 4, washing at pH 5 will permit fluorescence of the HCyC-646 dyes on ddTTP-5-SS-HCyC-646 and ddCTP-5-SS-Tetrazine/HCyC-646. A second imaging step is performed at pH 5, and new fluorescence signals will confirm incorporation by either ddC or ddT. At or just prior to this point, an optional chase step with the four 3′-O-azidomethyl dNTPs may be performed to ensure that nearly every primer has been extended either with one of the ddNTP or NRT analogues. Step 5, incubation with TCO-BHQ3 will attach the BHQ quencher to the tetrazine anchors on ddC and ddG. Step 6, after washing at pH 5 to remove any free TCO-BHQ, a third imaging step is carried out. Substantial reduction of fluorescence signal in the case of previously determined ddATP or ddGTP analogue incorporation indicates ddG and remaining signal indicates ddA incorporation. Similarly, substantial loss of fluorescence signal in the case of previously determined ddCTP or ddTTP analogue incorporation indicates ddC and remaining signal indicates ddT incorporation. Step 7, cleavage of SS linker by adding THP to the elongated DNA strands results in removal of dyes on the ddNTP analogues and also restores the 3′-OH group on any growing strands extended with 3′-O-azidomethyl-dNTPs. The DNA products are ready for the next cycle of the DNA sequencing reaction. Structures of nucleotides used in this scheme are presented in FIG. 82. In the imaging cartoons at each step, black indicates a positive Cy5 signal and white or light gray a background signal. The encoding in the summary cartoon at the end indicates the template sequence, not the incorporated nucleotides.

FIG. 84: Generalized set of dye labeled cleavable ddNTP analogues for single color SBS and anchor for attachment of a dye quencher molecule: All of the dideoxynucleotide analogues have Cy5 attached to the base via an SS linker, two of which have an anchor for attachment of a dye quencher. A requirement of this hybrid SBS method is a separate set of four unlabeled reversible terminators (e.g., 3′-O-azidomethyl dNTPs).

FIG. 85: Simplified presentation of scheme for single color SBS using cleavable nucleotide analogues such as those presented in FIG. 84. Each type of ddNTP has one of the following, Cy5 or Cy5 and Tetrazine, attached via an SS linker. The rectangles represent areas containing numerous copies of attached primer-loop-template molecules (or other template-bound primer arrangements) in which the next base in the template strand, from left to right is T, G, C or A. Extension is carried out with Therminator IX and two of the ddNTP analogues, ddATP attached to Cy5 via an SS linker and ddTTP attached to both tetrazine and Cy5 via an SS linker, along with an excess of the 3′-O-azidomethyl dNTPs. After washing, imaging will reveal a positive signal in the first and fourth rectangular areas, due to Cy5 fluorescence, indicating incorporation of A or T. Next, incubation with ddGTP attached to Cy5 via an SS linker, and ddCTP attached to both tetrazine and Cy5 via an SS linker, along with an excess of 3′-O-azidomethyl-dATP and 3′-O-azidomethyl-dTTP, followed by washing and imaging, will result in positive signal in the second and third rectangular areas, indicating incorporation of C or G. Incubation with TCO-BHQ3 will attach the quencher to the tetrazine anchor on ddCTP and ddTTP to ensure fidelity, and after washing, imaging will reveal fluorescence quenching resulting in substantially reduced fluorescence (in the second and fourth rectangular areas) for these two nucleotide analogues, indicating incorporation of C or T specifically, whereas no loss of fluorescence (in the first and third rectangular areas) will indicate incorporation of A or G specifically. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 010 for C, 011 for G and 110 for T considering all three of these imaging steps, or 11 for A, 00 for C, 01 for G and 10 for T considering only the first and third imaging step).

FIG. 86: Example ddNTP Analogues and Quencher-Anchor Binding Molecule Used for FIG. 87.

FIG. 87A-87B: Single Color Sequencing by Synthesis Using a Set of ddNTP Analogues, Containing Either Cy5 or Cy5 Plus Anchor, and a Quenching Step. Use of ddNTP-Cleavable Linker-Dyes (ddATP-7-SS-Cy5, ddGTP-7-SS-Cy5, ddTTP-5-SS-Tetrazine/Cy5, ddCTP-5-SS-Tetrazine/Cy5), 3′-O-azidomethyl dNTPs (3′-O-azidomethyl-dATP, 3′-O-azidomethyl-dCTP, 3′-O-azidomethyl-dGTP, 3′-O-azidomethyl-dTTP), and anchor binding molecule-quencher (TCO-BHQ3) to perform 1-color DNA SBS. Step 1, Addition of Therminator IX DNA polymerase, two of the ddNTP-Cleavable Linker-Dyes (ddATP-7-SS-Cy5, ddTTP-5-SS-Tetrazine/Cy5) and an excess of the four reversible terminators (3′-O-azidomethyl-dATP, 3′-O-azidomethyl-dCTP, 3′-O-azidomethyl-dGTP, 3′-O-azidomethyl-dTTP) to the immobilized primed DNA template enables the incorporation of the complementary 3′-O-azidomethyl-dNTP to the majority of growing DNA strands (>95%) and the ddATP-7-SS-Cy5, ddTTP-5-SS-Tetrazine/Cy5 on most of the remaining primers to terminate DNA synthesis. Step 2, after washing away the unincorporated nucleotide analogues, imaging for Cy5 fluorescence will reveal primers extended with either ddATP-7-SS-Cy5 or ddTTP-5-SS-Tetrazine/Cy5. Step 3, Addition of Therminator IX DNA polymerase, the remaining two ddNTP-Cleavable Linker-Dyes (ddGTP-7-SS-Cy5, ddCTP-5-SS-Tetrazine/Cy5) and the other two reversible terminators (3′-O-azidomethyl-dATP, 3′-O-azidomethyl-dTTP) to the immobilized primed DNA template enables accurate incorporation of ddGTP-7-SS-Cy5 and ddCTP-5-SS-Tetrazine/Cy5. Step 4, After washing away the unincorporated nucleotides, a third imaging step is performed to reveal Cy5 fluorescence, and new fluorescence signals will confirm incorporation by ddC or ddG. At or just prior to this point, an optional chase step with the four 3′-O-azidomethyl dNTPs may be performed to ensure that nearly every primer has been extended either with one of the ddNTP or NRT analogues. Step 5, incubation with TCO-BHQ3 will attach the BHQ quencher to the tetrazine anchors on ddC and ddT. Step 6, after washing to remove any free tetrazine-BHQ, a third imaging step is carried out. Substantial reduction of Cy5 signal in the case of previously determined ddATP or ddTTP analogue incorporation indicates ddT and remaining signal indicates ddA incorporation. Similarly, substantial loss of Cy5 signal in the case of previously determined ddCTP or ddGTP analogue incorporation indicates ddC and remaining signal indicates ddG incorporation. Step 7, cleavage of SS linker by adding THP to the elongated DNA strands results in removal of dyes on the ddATP and ddGTP analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 86. In the imaging cartoons at each step, black indicates a positive Cy5 signal and white or light gray a background signal. The encoding in the summary cartoon at the end indicates the template sequence, not the incorporated nucleotides.

FIG. 88: Synthesis of ddNTP-SS-Dye-TCO (ddGTP-7-SS-Cy5-TCO as example). Compounds of this type are used in Examples 6 and 7.

FIG. 89: Synthesis of ddNTP-SS-Dye-TCO (short linker version) (ddGTP-7-SS-Cy5-TCO as example). Compounds of this type are used in Example 6.

FIG. 90: Synthesis of ddNTP-SS-Anchor-TCO (ddCTP-5-SS-Biotin-TCO as example). Compounds of this type are used in Example 6.

FIG. 91: Synthesis of Binding Molecule-Quencher (Tetrazine-BHQ3 as example). Compounds of this type are used in Examples 6 and 7.

FIG. 92: Synthesis of Binding Molecule-Dye (TCO-HCyC-646 as example). Compounds of this type are used in Example 2.

FIG. 93: Synthesis of Binding Molecule-Quencher (TCO-BHQ3 as example). Compounds of this type are used in Examples 11 and 12.

FIG. 94: Synthesis of HCyC-646 NHS Ester. Compounds of this type are used in Examples 2, 7 and 10.

FIG. 95: Synthesis of ddNTP-SS-Dye (ddTTP-5-SS-HCyc-646 as example).

Compounds of this type are used in Examples 2, 7 and 10.

FIG. 96: Synthesis of Dye-TCO Linker-NHS Ester (Cy5-TCO-NHS Ester as example). Compounds of this type are used in Examples 5, 8 and 9.

FIG. 97: Synthesis of ddNTP-TCO Linker-Anchor (ddCTP-5-TCO-Biotin as example). Compounds of this type are used in Example 5.

FIG. 98: Synthesis of ddNTP-SS-Dye-Tetrazine (ddGTP-7-SS-Cy5-Tetrazine as example). Compounds of this type are used in Examples 11 and 12.

FIG. 99: Synthesis of ddNTP-SS-Dye-Tetrazine (ddGTP-7-SS-Cy5-Tetrazine as example). Compounds of this type are used in Examples 11 and 12.

FIG. 100: Synthesis of dNTP-SS-Blocker-TCO-Anchor (dCTP-SS-Blocker-TCO-Biotin as example, base can be A, C, T or G). Compounds of this type are used in Example 8.

FIG. 101: Synthesis of dNTP-SS-Blocker-TCO-Dye (dCTP-SS-Blocker-TCO-HCyC-646 as example, base can be either A,C,T or G, Dye can be Cy5). Compounds of this type are used in Examples 8 and 9.

FIG. 102: Synthesis of 3′-SS-dNTP-SS-TCO-Dye (3′-SS-dGTP-SS-TCO-HCyC-646 as example, base can be either A,C,T or G, Dye can also be Cy5). Compounds of this type are used in Example 8.

FIG. 103: Synthesis of 3′-SS-dNTP-SS-TCO-Anchor (3′-SS-dCTP-SS-TCO-Biotin as example, base can be either A, C, T or G). Compounds of this type are used in Examples 8 and 9.

FIG. 104: Thirteen cycles of single-color sequencing by synthesis results using ddCTP-5-SS-Cy5, ddGTP-7-SS-Cy5, ddATP-7-SS-biotin, ddTTP-5-SS-biotin, streptavidin-Cy5, and the four 3′-O-azidomethyl dNTPs for Two Different Immobilized DNA Templates. Each cycle consisted of the following steps (with intermediate wash steps): (1) extension with the four 3′-O-azidomethyl dNTPs using Therminator IX DNA polymerase to extend ˜95% of the primer-loop-template molecules in each spot on the slide; (2) extension with ddCTP-SS-Cy5, ddATP-SS-biotin, 3′-O-azidomethyl-dGTP and 3′-O-azidomethyl-dTTP (“E-ddAddC”) (3) Incubation with streptavidin-Cy5 (1st “Labeling” step); (4) Extension with ddGTP-SS-Cy5, ddTTP-SS-biotin, 3′-O-azidomethyl-dATP and 3′-O-azidomethyl-dCTP (“E-ddGddT”); (5) Incubation with streptavidin-Cy5 (2nd “Labeling” step); (6) Chase with the four 3′-O-azidomethyl dNTPs; (7) Treatment with THP to remove dyes and restore 3′-OH group on incorporated reversible terminators (3′-O-azidomethyl dNTPs) (“Cleavage”). Signals below 700 are considered background and encoded “0”; signals above 850 are considered positive and encoded “1”. In the bar graphs, each group of 4 bars represents 1 cycle, and the bars from left to right represent fluorescence images in arbitrary units for 1st extension, 1st labeling, 2nd extension and 2nd labeling, respectively. Examining the top bar graph, imaging results for Cycle 1 (0011) indicate incorporation of G, Cycle 2 (0111) indicate A, Cycle 3 (0011) indicate G, Cycle 4 (1111) indicate C, Cycle 5 (0111) indicate A, Cycle 6 (0001) indicate T, and so on, revealing the first 13 bases of the template sequence as 3′-CTCGTAGTTCAAA-5′ (SEQ ID NO: 1), exactly matching the expected template sequence attached to the surface in that area of the slide. Similarly, examining the bottom bar graph, the sequence obtained is 3′-GTAGTTCAAACCC-5′ (SEQ ID NO: 2), exactly as expected for the template attached to the surface in that area of the slide. These results demonstrate that the SBS approach described in Example 1 of this application can be used to successfully and accurately sequence DNA.

FIG. 105: The set of nucleotide analogues (ddCTP-SS-Cy5, ddGTP-SS-Cy5, ddTTP-SS-HCyC-646, ddATP-SS-HCyC-646, 3′-O—CH₂—N₃-dATP, 3′-O—CH₂—N₃-dCTP, 3′-O—CH₂—N₃-dGTP and 3′-O—CH₂—N₃-dTTP to Achieve 1-Color Sequencing by Synthesis without the Need for a Labeling Step as in Example 2.

FIG. 106: Scheme for 1-color sequencing by synthesis using the set of nucleotides shown in FIG. 105.

FIG. 107: Synthesis of pH-responsive dye HCyC-646 and conjugation of HCyC-646 NHS to 5-amino-SS-dTTP. The detailed protocol is described under Example 10 in the text.

FIG. 108: Attachment of an HCyC-646 NHS to 7-amino-SS-dATP. The detailed protocol is described under Example 10 in the text.

FIG. 109: MALDI-TOF-MS spectrum for ddTTP-5-SS-HCyC-646 synthesized and purified as described under Example 10. Expected MW (1298 Da); obtained (1302 Da).

FIG. 110: MALDI-TOF-MS spectrum for ddA-7-SS-HCyC-646 synthesized and purified as described under Example 10. Expected MW (1321 Da); obtained (1326 Da).

FIG. 111: Example of protonated and deprotonated forms of HCyC-646 attached to a ddNTP (ddATP shown).

FIG. 112: MALDI-TOF-MS spectrum for ddTTP-5-SS-HCyC-646 extended primer. The protocol is described under Example 10 in the text. Expected product size (6286 Da); obtained (6287 Da). This indicates that the ddTTP-5-SS-HCyC-646 nucleotide is recognized by DNA polymerase (Therminator IX in this case).

FIG. 113: Use of dTTP-5-SS-CyC-646 for sequencing in the style of Example 10. Three cycles of extension, pH washes and cleavage were performed. The protocol is described in detail under Example 10 and the various steps are indicated in the figure. Four images are shown for Cycle 1, and two images each for Cycles 2 and 3. Note in particular the loss of fluorescence when the slides previously washed at low pH buffer (below 7) are then washed using high pH buffers (above 9). The expected sequences for the first 3 positions of the templates are TAG in the left rectangular area of the slide, GAG for the 2nd area, CAT for the 3rd area, and ATT for the right-most area of the slide. Thus, using dTTP-5-SS-CyC-646, we would expect it to be incorporated in the left area in Cycle 1, the right area in Cycle 2, and the two right-most areas of the slide in Cycle 3, exactly what is observed. A similar successful characterization of dATP-SS-HCyC-646 was also performed.

FIG. 114: Example of cycle of sequencing using scheme illustrated in FIG. 106 using ddCTP-5-SS-Cy5, ddATP-7-SS-HCyC-646, ddGTP-7-SS-Cy5, ddTTP-5-SS-HCyC-646 and the four 3′-O-azidomethyl dNTPs. The simplified protocol is indicated in the boxes at the left of the figure, and the detailed protocol is provided in the text accompanying Example 10. As expected, a positive fluorescence signal for either Cy5 or HCyC-646 is obtained after extension with ddCTP-5-SS-Cy5, ddATP-7-SS-HCyC-646, along with 3′-O-azidomethyl dGTP and 3′-O-azidomethyl dTTP and a pH 5 wash, indicating incorporation of either C or A. A second extension with ddGTP-7-SS-Cy5, ddTTP-5-SS-HCyC-646, along with 3′-O-azidomethyl dCTP and 3′-O-azidomethyl dATP, followed by a pH 5 wash, resulting in positive fluorescence signals due to incorporation of either G or T. Washing with a pH 9 buffer eliminates the signals due to fluorescence of HCyC-646. Thus, if it was previously determined that either A or C was incorporated, loss of fluorescence indicates incorporation of A while remaining fluorescence indicates incorporation by C. Similarly, if it was previously determined that either G or T was incorporated, loss of fluorescence indicates incorporation of T while remaining fluorescence indicates incorporation by G. Finally, THP treatment cleaves the disulfide bonds, resulting in removal of the dyes from the ddNTP analogues, indicated by only background fluorescence, and restoration of the 3′-OH group on any incorporated 3′-O-azidomethyl dNTPs.

FIG. 115: Four-cycle sequencing by synthesis using ddCTP-5-SS-Cy5, ddATP-7-SS-HCyC-646, ddGTP-7-SS-Cy5, ddTTP-5-SS-HCyC-646 and the four 3′-O-azidomethyl dNTPs for four different templates, two of which were duplicated in different portions of the slide. The procedure described in FIG. 106 and illustrated for one cycle in FIG. 114 was performed for 4 continuous cycles of sequencing by synthesis. The bar graph at top left shows the results for the first cycle. The six sets of 3 bars represent the different templates. The first bar of each set (E-ddAC5) represents the fluorescence results obtained after the first extension with ddATP-7-SS-HCyC-646 and ddCTP-5-SS-Cy5 and a pH 5 wash. The second bar of each set (E-ddGT5) represents the fluorescence results obtained after the second extension with ddTTP-7-SS-HCyC-646 and ddGTP-5-SS-Cy5 and a pH 5 wash. The last bar of each set (pH9) represents the fluorescence results obtained after switching to a pH 9 buffer to substantially reduce fluorescence due to the pH-sensitive dye HCyC-646. As an example, background fluorescence (below 700 arbitrary units) after the first extension, positive fluorescence after the second extension and background fluorescence after the shift to pH 9 is recorded digitally as 010 and indicates incorporation by T. The digital readouts 011, 111 and 110 are indicative of G, C and A incorporation respectively. The bar graph at the bottom left indicates the results for the 2nd cycle of SBS, the lower right bar graph the results for the 3rd cycle of SBS, and the upper right bar graph the results for the 4th cycle of SBS. The correct results were obtained in each cycle for these 4 template DNAs.

FIG. 116: Generalized set of anchor and dye labeled nucleotide analogues with blockers on the base (virtual terminators) for use in single molecule energy transfer SBS using a donor dye and an anchor for attachment of either a pH responsive or pH-unresponsive dye acceptor molecule. All of the nucleotide analogues have Cy3 and either a biotin or tetrazine anchor attached to the base via an SS linker. The anchor binding molecules streptavidin and TCO are attached to Cy5 and HCyC-646 respectively. The latter is a pH-responsive dye that fluoresces below pH 6.

FIG. 117: Simplified presentation of scheme for single molecule energy transfer SBS using nucleotide analogues with blockers on the base (virtual terminators) such as those presented in FIG. 116. Each type of nucleotide analogue has both Cy3 and either a biotin or tetrazine anchor attached to the base via an SS linker. The rectangles represent areas on a substrate containing single template DNA molecules consisting of attached primer-loop-template molecules (or other template-bound primer arrangements) in which the next base in the template strand, from left to right is T, G, C or A. Extension is carried out with Thermo Sequenase and two of the dNTP analogues, dATP attached to Cy3 and biotin, and dTTP attached to Cy3 and tetrazine. Labeling is then performed with both streptavidin-Cy5 and TCO-HCyC-646. After a wash at pH 5, excitation of Cy3 and imaging will reveal a positive signal in the first and fourth rectangular areas, due to energy transfer from Cy3 to either the Cy5 or HCyC-646 dye, indicating incorporation of A or T. A second extension is carried out with Thermo Sequenase and the remaining two nucleotide analogues, dCTP attached to Cy3 and biotin, and dGTP attached to Cy3 and tetrazine. Labeling is again performed with both streptavidin-Cy5 and TCO-HCyC-646. After a wash at pH 5, excitation of Cy3 and imaging will reveal new fluorescence in the second and third rectangular areas, due to energy transfer to Cy5 or HCyC-646, indicating incorporation of C or G. After washing at pH 9, imaging will reveal substantially reduced fluorescence of HCyC-646 on the T and G nucleotide analogues (third and fourth rectangular areas on the slides) but no loss of fluorescence of Cy5 on the A and C nucleotide analogues (first and second rectangular areas). Finally, treatment with THP cleaves off the remaining dyes and restores the 3′-OH group on these nucleotides. The 1, 2 and 3 numerical codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 011 for C, 010 for G and 110 for T considering all three of these imaging steps, or 11 for A, 01 for C, 00 for G and 10 for T considering only the first and third imaging step).

FIG. 118: Example dNTP virtual terminator analogs attached with both donor dye (Cy3) and anchor molecule (either Tetrazine or Biotin), and the corresponding binding molecule (TCO or Streptavidin)-Acceptor Dye (Cy5 or HCyc-646) conjugates used for FIG. 119.

FIG. 119A-D: Single Molecule Energy Transfer Sequencing by Synthesis Using a Set of Virtual Terminator Analogues, Containing Cy3 and Either Biotin or Tetrazine, for Attachment of Cy5 or the pH-Responsive Dye HCyC-646. Use of dNTP-Blocker-Cleavable Linker-Anchor/Dyes (dATP-7-SS-Blocker-Biotin/Cy3, dTTP-5-SS-Blocker-Tetrazine/Cy3, dCTP-5-SS-Blocker-Biotin/Cy3 and dGTP-7-SS-Blocker-Tetrazine/Cy3) and Anchor Binding Molecule-Dye Molecules (Streptavidin-Cy5 and TCO-HCyC-646), to perform single molecule energy transfer DNA SBS. Step 1, Addition of Thermo Sequenase DNA polymerase and two of the four virtual terminator analogues (dATP-7-SS-Blocker-Biotin/Cy3 and dTTP-5-SS-Blocker-Tetrazine/Cy3) to the immobilized primed DNA template. Step 2, after washing away any unincorporated nucleotides, Streptavidin-Cy5 and TCO-HCyC-646 are added together to label the dATP and dTTP nucleotide analogues via their biotin and tetrazine anchors. Step 3, after a wash at pH 5 and excitation of Cy3, fluorescence of Cy5 and HCyC-646 due to energy transfer from the Cy3 will reveal those primers extended with either dATP-7-SS-Blocker-Biotin/Cy3 or dTTP-5-SS-Blocker-Tetrazine/Cy3. Step 4, Addition of Thermo Sequenase DNA polymerase and the remaining virtual terminator analogues (dCTP-5-SS-Blocker-Biotin/Cy3 and dGTP-7-SS-Blocker-Tetrazine/Cy3) to the immobilized primed DNA template. Step 5, after washing away any unincorporated nucleotides, Streptavidin-Cy5 and TCO-HCyC-646 are again added together to label the dCTP and dGTP nucleotide analogues via their biotin and tetrazine anchors. Step 6, after a wash at pH 5 and excitation of Cy3, appearance of new Cy5 and HCyC-646 fluorescence signals due to energy transfer from the Cy3 will reveal those primers extended with either dCTP-5-SS-Blocker-Biotin/Cy3 or dGTP-7-SS-Blocker-Tetrazine/Cy3. Step 7, after washing at pH 9, to obtain positive Cy5 fluorescence signals but only background HCyC-646 fluorescence, a third imaging step is carried out. Substantial reduction of fluorescence signal in the case of previously determined dATP or dTTP analogue incorporation indicates dT and remaining signal indicates dA incorporation. Similarly, substantial loss of fluorescence signal in the case of previously determined dCTP or dGTP analogue incorporation indicates dG and remaining signal indicates dC incorporation. Step 8, cleavage of SS linker by adding THP to the elongated DNA strands results in removal of dyes on the reversible terminator analogues and also restores their 3′-OH group. The DNA products are ready for the next cycle of the DNA sequencing reaction. Structures of nucleotides used in this scheme are presented in FIG. 118. In the imaging cartoons at each step, black indicates a positive Cy5 or HCyC-646 signal and white a background signal. The encoding in the summary cartoon at the end indicates the template sequence, not the incorporated nucleotides.

FIG. 120: Generalized set of anchor and dye labeled cleavable 3′-blocked dNTP analogues (reversible terminators) for use in single molecule energy transfer SBS using a donor dye and an anchor for attachment of either a pH responsive or pH-unresponsive dye acceptor molecule. All of the nucleotide analogues have Cy3 and either a biotin or tetrazine anchor attached to the base via an SS linker. The anchor binding molecules streptavidin and TCO are attached to Cy5 and HCyC-646 respectively. The latter is a pH-responsive dye that fluoresces below pH 6.

FIG. 121: Simplified presentation of scheme for single molecule energy transfer SBS using cleavable 3′-blocked nucleotide analogues such as those presented in FIG. 120. Each type of dNTP has both Cy3 and either a biotin or tetrazine anchor attached to the base via an SS linker. The rectangles represent areas on a substrate containing single template DNA molecules consisting of attached primer-loop-template molecules (or other template-bound primer arrangements) in which the next base in the template strand, from left to right is T, G, C or A. Extension is carried out with Therminator IX and two of the dNTP analogues, 3′-SS-dATP attached to Cy3 and biotin, and 3′-SS-dTTP attached to Cy3 and tetrazine. Labeling is then performed with both streptavidin-Cy5 and TCO-HCyC-646. After a wash at pH 5, excitation of Cy3 and imaging will reveal a positive signal in the first and fourth rectangular areas, due to energy transfer from Cy3 to either the Cy5 or HCyC-646 dye, indicating incorporation of A or T. A second extension is carried out with Therminator IX and the remaining two dNTP analogues, 3′-SS-dCTP attached to Cy3 and biotin, and 3′-SS-dGTP attached to Cy3 and tetrazine. Labeling is again performed with both streptavidin-Cy5 and TCO-HCyC-646. After a wash at pH 5, excitation of Cy3 and imaging will reveal new fluorescence in the second and third rectangular areas, due to energy transfer to Cy5 or HCyC-646, indicating incorporation of C or G. After washing at pH 9, imaging will reveal substantially reduced fluorescence of HCyC-646 on the T and G nucleotide analogues (third and fourth rectangular areas on the slide) but no loss of fluorescence of Cy5 on the A and C nucleotide analogues (first and second rectangular areas). Finally, treatment with THP cleaves off the remaining dyes and restores the 3′-OH group on these nucleotides. The 1, 2 and 3 numerical codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 011 for C, 010 for G and 110 for T considering all three of these imaging steps, or 11 for A, 01 for C, 00 for G and 10 for T considering only the first and third imaging step).

FIG. 122: Example 3′-SS-dNTP reversible terminator analogues attached with both donor dye (Cy3) and anchor molecule (either Tetrazine or Biotin), and the corresponding binding molecule (TCO or Streptavidin)-Acceptor Dye (Cy5 or HCyc-646) conjugates used for FIG. 123.

FIG. 123A-D: Single Molecule Energy Transfer Sequencing by Synthesis Using a Set of Nucleotide Reversible Terminator Analogues, Containing Cy3 and Either Biotin or Tetrazine, for Attachment of Cy5 or the pH-Responsive Dye HCyC-646. Use of 3′-blocked reversible terminator analogues (3′-O-SS-dATP-7-SS-Biotin/Cy3, 3′-O-SS-dGTP-7-SS-Tetrazine/Cy3, 3′-O-SS-dTTP-5-SS-Tetrazine/Cy3, 3′-O-SS-dCTP-5-SS-Biotin/Cy3), and Anchor Binding Molecule-Dye Molecules (Streptavidin-Cy5 and TCO-HCyC-646), to perform single molecule energy transfer DNA SBS. Step 1, Addition of Therminator IX DNA polymerase and two of the four 3′-blocked dNTP analogues (3′-O-SS-dATP-7-SS-Biotin/Cy3 and 3′-O-SS-dTTP-5-SS-Tetrazine/Cy3) to the immobilized primed DNA template. Step 2, after washing away any unincorporated nucleotides, Streptavidin-Cy5 and TCO-HCyC-646 are added together to label the dATP and dTTP nucleotide analogues via their biotin and tetrazine anchors. Step 3, after a wash at pH 5 and excitation of Cy3, fluorescence of Cy5 and HCyC-646 due to energy transfer from the Cy3 will reveal those primers extended with either 3′-O-SS-dATP-7-SS-Biotin/Cy3 or 3′-O-SS-dTTP-5-SS-Tetrazine/Cy3. Step 4, Addition of Therminator IX DNA polymerase and the remaining 3′-blocked dNTP analogues (3′-O-SS-dCTP-5-SS-Biotin/Cy3 and 3′-O-SS-dGTP-7-SS-Tetrazine/Cy3) to the immobilized primed DNA template. Step 5, after washing away any unincorporated nucleotides, Streptavidin-Cy5 and TCO-HCyC-646 are again added together to label the dCTP and dGTP nucleotide analogues via their biotin and tetrazine anchors. Step 6, after a wash at pH 5 and excitation of Cy3, appearance of new Cy5 and HCyC-646 fluorescence signals due to energy transfer from the Cy3 will reveal those primers extended with either 3′-O-SS-dCTP-5-SS-Biotin/Cy3 or 3′-O-SS-dGTP-7-SS-Tetrazine/Cy3. Step 7, after washing at pH 9, to obtain positive Cy5 fluorescence signals but only background HCyC-646 fluorescence, a third imaging step is carried out. Substantial reduction of fluorescence signal in the case of previously determined dATP or dTTP analogue incorporation indicates dT and remaining signal indicates dA incorporation. Similarly, substantial loss of fluorescence signal in the case of previously determined dCTP or dGTP analogue incorporation indicates dG and remaining signal indicates dC incorporation. Step 8, cleavage of SS linker by adding THP to the elongated DNA strands results in removal of dyes on the reversible terminator analogues and also restores their 3′-OH group. The DNA products are ready for the next cycle of the DNA sequencing reaction. Structures of nucleotides used in this scheme are presented in FIG. 122. In the imaging cartoons at each step, black indicates a positive Cy5 or HCyC-646 signal and white a background signal. The encoding in the summary cartoon at the end indicates the template sequence, not the incorporated nucleotides.

FIG. 124: Generalized set of anchor and dye labeled nucleotide analogues with blockers on the base (virtual terminators) for use in single molecule energy transfer SBS using a donor dye and an anchor for attachment of either a pH responsive or pH-unresponsive dye acceptor molecule. Two of the nucleotide analogues have Cy3 and either a biotin or tetrazine anchor attached to the base via an SS linker. The other two nucleotide analogues have Cy3 and either a biotin or tetrazine anchor attached to the base via an azo (N═N) linker. The anchor binding molecules streptavidin and TCO are attached to Cy5 and HCyC-646 respectively. The latter is a pH-responsive dye that fluoresces below pH 6.

FIG. 125: Simplified presentation of scheme for single molecule energy transfer SBS using nucleotide analogues with blockers on the base (virtual terminators) such as those presented in FIG. 124. Each type of nucleotide analogue has both Cy3 and either a biotin or tetrazine anchor attached to the base via an SS or Azo linker, with all combinations of linkers and anchors, SS and biotin on A, SS and tetrazine on C, Azo and tetrazine on G, and Azo and biotin on T. The rectangles represent areas on a substrate containing single template DNA molecules consisting of attached primer-loop-template molecules (or other template-bound primer arrangements) in which the next base in the template strand, from left to right is T, G, C or A. Extension is carried out with Thermo Sequenase and the four dNTP analogues. Labeling is then performed with both streptavidin-Cy5 and TCO-HCyC-646. After a wash at pH 5, excitation of Cy3 and imaging will reveal a positive signal in all four rectangular areas, due to energy transfer from Cy3 to either the Cy5 or HCyC-646 dye, indicating incorporation of A, C, G or T. After a wash at pH 9, excitation of Cy3 and imaging will reveal loss of fluorescence in the second and third rectangular areas, due to the low-pH dependency of HCyC-646 fluorescence, indicating incorporation of C or G. Remaining fluorescence due to Cy5, which is not pH responsive, indicates incorporation of A or T. Treatment with sodium dithionite will cleavage the Azo linkers on G and T, removing the dyes attached to these nucleotides. Thus, fluorescence signals will only be present in the first and second rectangular areas. Finally, treatment with THP cleaves off the remaining dyes and restores the 3′-OH group on these nucleotides. The 1, 2 and 3 numerical codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 101 for C, 100 for G and 110 for T considering all three of these imaging steps, or 11 for A, 01 for C, 00 for G and 10 for T considering only the second and third imaging step).

FIG. 126: Example 3′-dNTP virtual terminator analogs attached with both donor dye (Cy3) and anchor molecule (either Tetrazine or Biotin) through orthogonally cleavable linkers (SS and Azo linker), and the corresponding binding molecule (TCO and Streptavidin)-Acceptor Dye (Cy5 and HCyc-646) conjugates used for FIG. 127.

FIG. 127A-D: Single Molecule Energy Transfer Sequencing by Synthesis Using an Orthogonal Set of Virtual Terminator Analogues, Containing Cy3 and Either Biotin or Tetrazine, Attached to the Base Via an SS or Azo Linker, for Conjugation of Cy5 or the pH-Responsive Dye HCyC-646. Use of dNTP-Blocker-Cleavable Linker-Anchor/Dyes (dATP-7-SS-Blocker-Biotin/Cy3, dTTP-5-SS-Blocker-Azo-Biotin/Cy3, dCTP-5-SS-Blocker-Tetrazine/Cy3 and dGTP-7-SS-Blocker-Azo-Tetrazine/Cy3) and Anchor Binding Molecule-Dye Molecules (Streptavidin-Cy5 and TCO-HCyC-646), to perform single molecule energy transfer DNA SBS. Step 1, Addition of Thermo Sequenase DNA polymerase and the four virtual terminator analogues (dATP-7-SS-Blocker-Biotin/Cy3, dTTP-5-SS-Blocker-Azo-Biotin/Cy3, dCTP-5-SS-Blocker-Tetrazine/Cy3 and dGTP-7-SS-Blocker-Azo-Tetrazine/Cy3) to the immobilized primed DNA template. Step 2, after washing away any unincorporated nucleotides, Streptavidin-Cy5 and TCO-HCyC-646 are added together to label the nucleotide analogues via their biotin and tetrazine anchors. Step 3, after a wash at pH 5 and excitation of Cy3, fluorescence of Cy5 and HCyC-646 due to energy transfer from the Cy3 will reveal those primers extended with any of the four virtual terminator nucleotide analogues. Step 4, after a wash at pH 9 and excitation of Cy3, substantial loss of fluorescence signals due to energy transfer from the Cy3 will reveal those primers extended with either the dC or dG nucleotide analogues which were labeled with HCyC-646, while remaining fluorescence due to Cy5 will indicate incorporation by the dA and dT nucleotide analogues. Step 5, cleavage of the azo group in the linkers attaching the dyes and anchors to the base on the dG and dT nucleotide analogues will result in removal of the dyes on these nucleotides. Step 6, excitation of Cy3 and imaging for Cy5 or HCyC-646 fluorescence is carried out. Substantial reduction of fluorescence signal in the case of previously determined dATP or dTTP analogue incorporation indicates dT and remaining signal indicates dA incorporation. Similarly, substantial loss of fluorescence signal in the case of previously determined dCTP or dGTP analogue incorporation indicates dG and remaining signal indicates dC incorporation. Step 7, cleavage of SS linker by adding THP to the elongated DNA strands results in removal of dyes on the reversible terminator analogues and also restores their 3′-OH group. The DNA products are ready for the next cycle of the DNA sequencing reaction. Structures of nucleotides used in this scheme are presented in FIG. 126. In the imaging cartoons at each step, black indicates a positive Cy5 or HCyC-646 signal and white a background signal. The encoding in the summary cartoon at the end indicates the template sequence, not the incorporated nucleotides.

FIG. 128: Generalized set of anchor and dye labeled cleavable 3′-blocked dNTP analogues (reversible terminators) for use in single molecule energy transfer SBS using a donor dye and an anchor for attachment of either a pH responsive or pH-unresponsive dye acceptor molecule. Two of the nucleotide analogues have Cy3 and either a biotin or tetrazine anchor attached to the base via an SS linker. The other two nucleotide analogues have Cy3 and either a biotin or tetrazine anchor attached to the base via an azo (N═N) linker. The anchor binding molecules, streptavidin and TCO, are attached to Cy5 and HCyC-646 respectively. The latter is a pH-responsive dye that fluoresces below pH 6.

FIG. 129: Simplified presentation of scheme for single molecule energy transfer SBS using cleavable 3′-blocked nucleotide analogues such as those presented in FIG. 128. Each type of dNTP has both Cy3 and either a biotin or tetrazine anchor attached to the base via either an SS or azo linker, with all combinations of linkers and anchors, SS and biotin on A, SS and tetrazine on C, Azo and tetrazine on G, and Azo and biotin on T. The rectangles represent areas on a substrate containing single template DNA molecules consisting of attached primer-loop-template molecules (or other template-bound primer arrangements) in which the next base in the template strand, from left to right is T, G, C or A. Extension is carried out with Therminator IX and the four dNTP analogues. Labeling is then performed with both streptavidin-Cy5 and TCO-HCyC-646. After a wash at pH 5, excitation of Cy3 and imaging will reveal a positive signal in all four rectangular areas, due to energy transfer from Cy3 to either the Cy5 or HCyC-646 dye, indicating incorporation of A, C, G or T. After a wash at pH 9, excitation of Cy3 and imaging will reveal loss of fluorescence in the second and third rectangular areas, due to the low-pH dependency of HCyC-646 fluorescence, indicating incorporation of C or G. Remaining fluorescence due to Cy5, which is not pH responsive, indicates incorporation of A or T. Treatment with sodium dithionite will cleavage the Azo linkers on G and T, removing the dyes attached to these nucleotides. Thus, fluorescence signals will only be present in the first and second rectangular areas. Finally, treatment with THP cleaves off the remaining dyes and restores the 3′-OH group on these nucleotides. The 1, 2 and 3 numerical codes at the left represent the cumulative signals at each of the three indicated imaging steps, a positive signal indicated by a 1 and a background signal indicated by a 0. Incorporation of each of the four possible nucleotide analogues will be revealed by a unique digital code (111 for A, 101 for C, 100 for G and 110 for T considering all three of these imaging steps, or 11 for A, 01 for C, 00 for G and 10 for T considering only the second and third imaging step).

FIG. 130: Example 3′-SS-dNTP reversible terminator analogues attached with both donor dye (Cy3) and anchor molecule (Tetrazine and Biotin) through orthogonally cleavable linkers (SS and Azo linker), and the corresponding binding molecule (TCO and Streptavidin)-Acceptor Dye (Cy5 and HCyc-646) conjugates used for FIG. 131.

FIG. 131A-D: Single Molecule Energy Transfer Based Sequencing by Synthesis Using an Orthogonal Set of Nucleotide Reversible Terminator Analogues, Containing Cy3 and Either Biotin or Tetrazine Attached to the Base Via an SS or Azo Linker, for Conjugation of Cy5 or the pH-Responsive Dye HCyC-646. Use of 3′-blocked reversible terminator analogues (3′-O-SS-dATP-7-SS-Biotin/Cy3, 3′-O-SS-dGTP-7-Azo-Tetrazine/Cy3, 3′-O-SS-dTTP-5-Azo-Biotin/Cy3, 3′-O-SS-dCTP-5-SS-Tetrazine/Cy3), and Anchor Binding Molecule-Dye Molecules (Streptavidin-Cy5 and TCO-HCyC-646), to perform single molecule energy transfer DNA SBS. Step 1, Addition of Therminator IX DNA polymerase and the four 3′-blocked dNTP analogues (3′-O-SS-dATP-7-SS-Biotin/Cy3, 3′-O-SS-dGTP-7-Azo-Tetrazine/Cy3, 3′-O-SS-dTTP-5-Azo-Biotin/Cy3 and 3′-O-SS-dCTP-5-SS-Tetrazine/Cy3) to the immobilized primed DNA template. Step 2, after washing away any unincorporated nucleotides, Streptavidin-Cy5 and TCO-HCyC-646 are added together to label the nucleotide reversible terminator analogues via their biotin and tetrazine anchors. Step 3, after a wash at pH 5 and excitation of Cy3, fluorescence of Cy5 and HCyC-646 due to energy transfer from the Cy3 will reveal primers extended with any of the four nucleotide reversible terminator analogues. Step 4, after a wash at pH 9 and excitation of Cy3, substantial loss of fluorescence signals due to energy transfer from the Cy3 will reveal those primers extended with either the dC or dG nucleotide analogues which were labeled with HCyC-646, while remaining fluorescence due to Cy5 will indicate incorporation by the dA and dT nucleotide analogues. Step 5, cleavage of the azo group in the linkers attaching the dyes and anchors to the base on the dG and dT nucleotide analogues will result in removal of the dyes on these nucleotides. Step 6, excitation of Cy3 and imaging for Cy5 or HCyC-646 fluorescence is carried out. Substantial reduction of fluorescence signal in the case of previously determined dATP or dTTP analogue incorporation indicates dT and remaining signal indicates dA incorporation. Similarly, substantial loss of fluorescence signal in the case of previously determined dCTP or dGTP analogue incorporation indicates dG and remaining signal indicates dC incorporation. Step 7, cleavage of SS linker by adding THP to the elongated DNA strands results in removal of dyes on the reversible terminator analogues and also restores their 3′-OH group. The DNA products are ready for the next cycle of the DNA sequencing reaction. Structures of nucleotides used in this scheme are presented in FIG. 130. In the imaging cartoons at each step, black indicates a positive Cy5 or HCyC-646 signal and white a background signal. The encoding in the summary cartoon at the end indicates the template sequence, not the incorporated nucleotides.

FIG. 132: Generalized set of dye labeled cleavable reversible terminators for single color SBS using a pH responsive dye: Two of the nucleotide analogues have Cy5 attached to the base via an SS linker and the other two have HCyC-646 attached to the base via an SS linker. HCyC-646 is a pH-responsive dye that fluoresces below pH 6. A requirement of this hybrid SBS method is a separate set of four unlabeled reversible terminators (e.g., 3′-O-azidomethyl dNTPs).

FIG. 133: Simplified presentation of scheme for single color SBS using cleavable nucleotide analogues such as those presented in FIG. 132. Each type of reversible nucleotide has one of the following, Cy5 or HCyC-646, attached via an SS linker. The rectangles represent areas on a substrate containing numerous copies of attached primer-loop-template molecules (or other template-bound primer arrangements) in which the next base in the template strand, from left to right, is T, G, C or A. Extension is carried out with Therminator IX, two of the dNTP analogues, 3′-tBu-dATP attached to HCyC-646 via an SS linker and 3′-tBu-dCTP attached to Cy5 via an SS linker, along with a small amount of 3′-O-azidomethyl-dGTP and 3′-O-azidomethyl-dTTP to increase fidelity. After a wash at pH 5, imaging will reveal a positive signal in the first and second rectangular areas, due to Cy5 or HCyC-646 fluorescence, indicating incorporation of A or C. Next, incubation with 3′-tBu-dGTP attached to Cy5 via an SS linker, 3′-tBu-dTTP attached to HCyC-646 via an SS linker, 3′-O-azidomethyl-dATP and 3′-O-azidomethyl-dCTP to increase fidelity, and washing at pH 5, will result in new positive signals in the third and fourth rectangular areas, indicating G or T incorporation.

After washing at pH 9, imaging will reveal loss of positive signals in the first and fourth rectangular areas due to the ability of HCyC-646 to fluoresce below pH 6 but not at pH 9. Thus loss of fluorescence will indicate incorporation by A if it was formerly determined that either A or C were incorporated and incorporation of T if it was formerly determined that either G or T was incorporated. Remaining fluorescence indicates incorporation of C and G respectively. Finally, treatment with THP cleaves off the remaining dyes and removes the azidomethyl group on any primers extended with NRTs in preparation for the next sequencing cycle. The 1, 2 and 3 numeral codes at the left represent the cumulative signals at each of the indicated imaging steps, a positive signal indicated by a 1 and a background signal indicated by a 0. Incorporation of each of the four possible nucleotide analogues will be revealed by a unique digital code (110 for A, 111 for C, 011 for G and 010 for T) considering all three of these imaging steps.

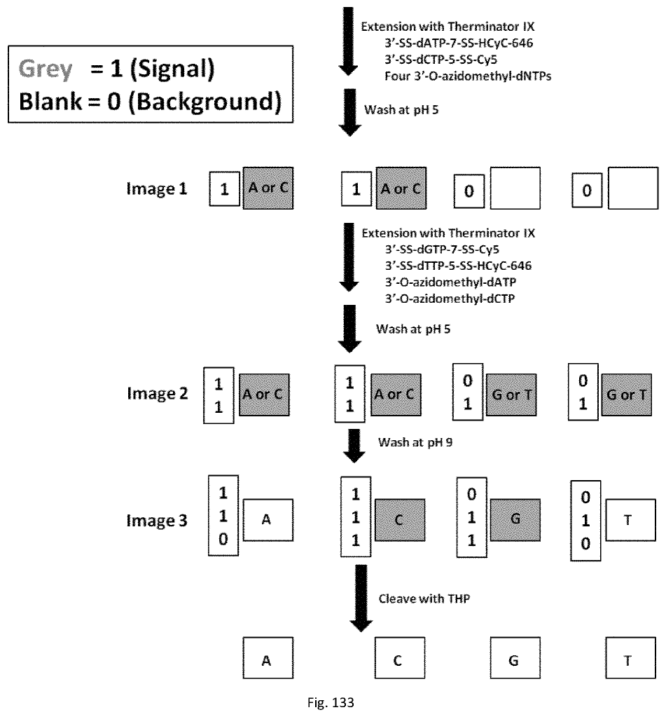

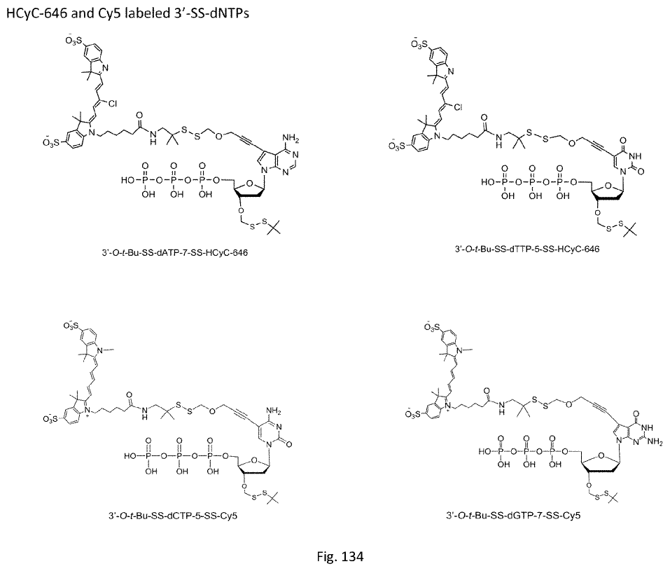

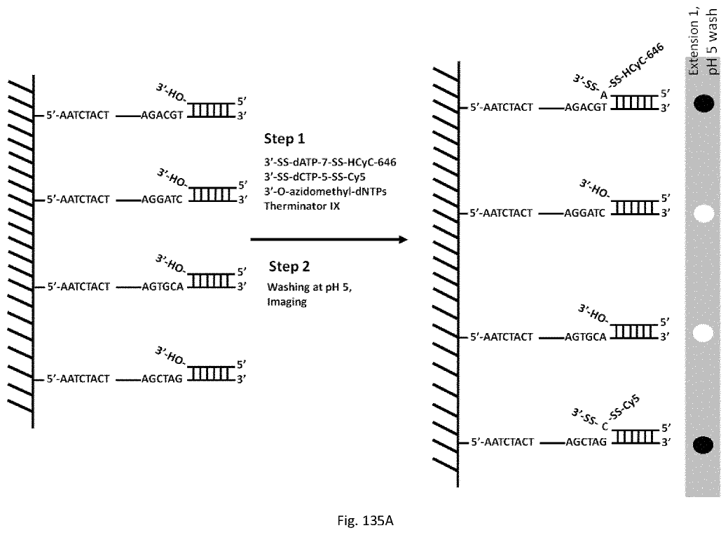

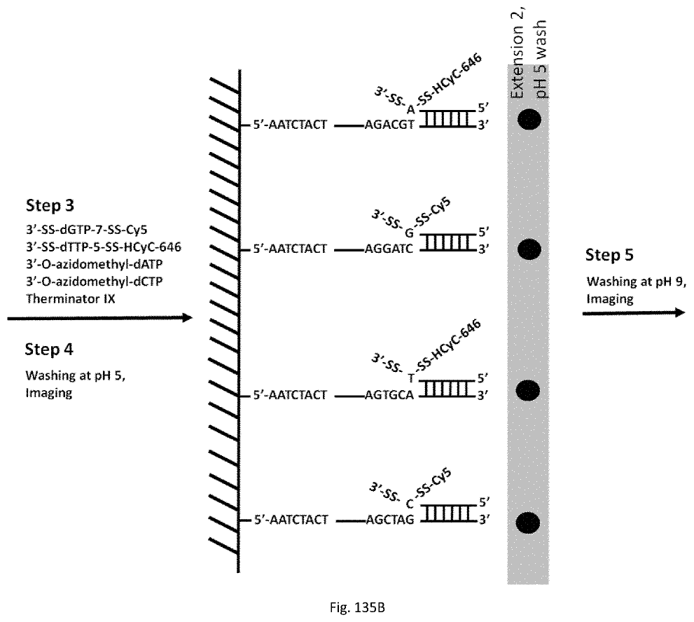

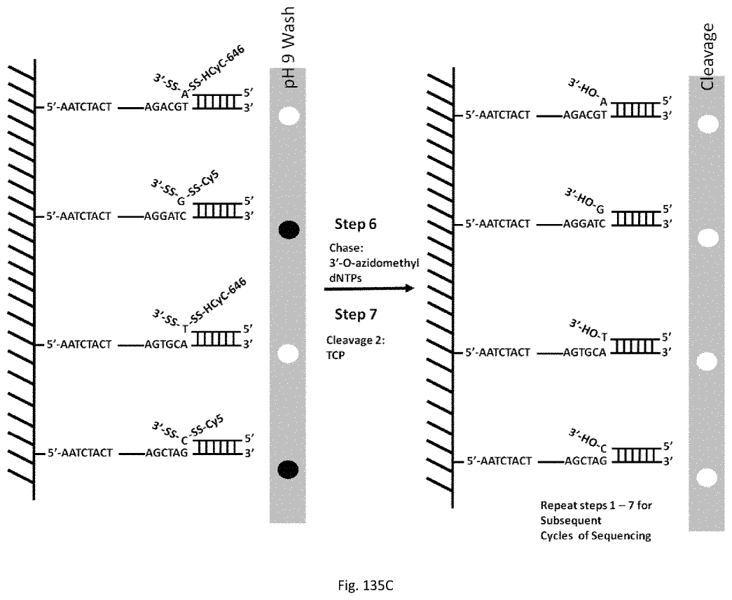

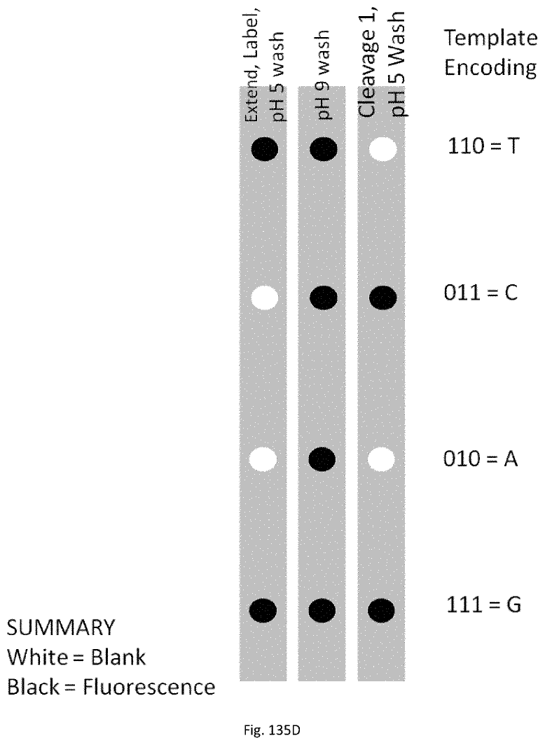

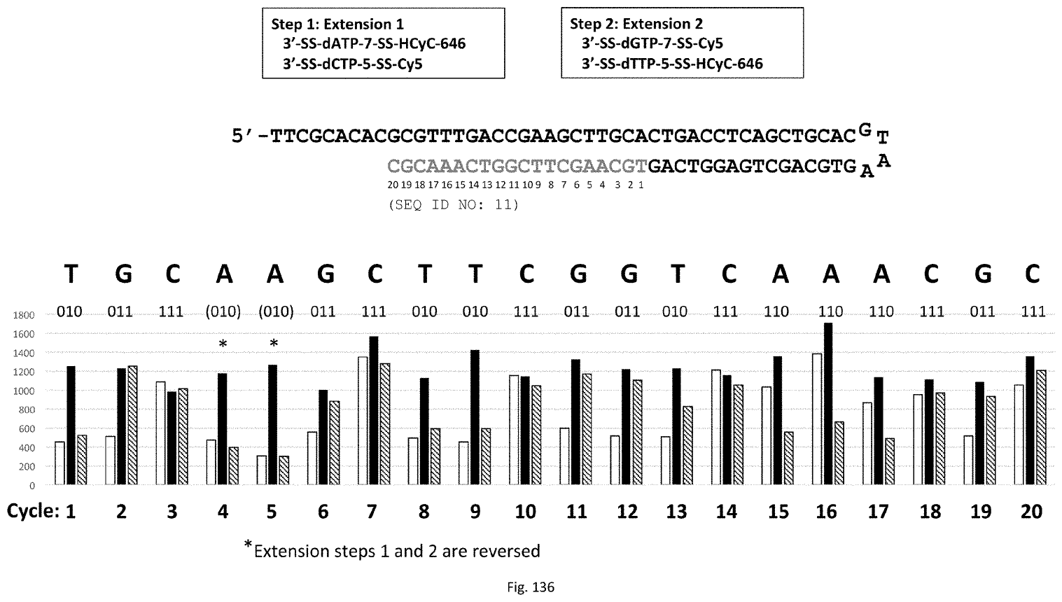

FIG. 134: Example Structures of Reversible Terminators Used for FIG. 133.

FIG. 135A-D: Single Color Sequencing by Synthesis Using a Set of Fluorescent 3′-t-Butyl-SS Nucleotide Analogues, Containing Either Cy5 or HCyC-646. Use of 3′-tBu-SS-dNTP-Cleavable Linker-Dyes (3′-tBu-SS-dATP-7-SS-HCyC-646, 3′-tBu-SS-dCTP-5-SS-Cy5, 3′-tBu-SS-dGTP-7-SS-Cy5, 3′-tBu-SS-dTTP-5-SS-HCyC-646) and 3′-O-azidomethyl dNTPs (3′-O-azidomethyl-dATP, 3′-O-azidomethyl-dCTP, 3′-O-azidomethyl-dGTP, 3′-O-azidomethyl-dTTP) to perform 1-color DNA SBS. Step 1, Addition of Therminator IX DNA polymerase, two of the 3′-t-Butyl-SS-dNTP-Cleavable Linker-Dyes (3′-tBu-SS-dATP-7-SS-HCyC-646 and 3′-tBu-SS-dCTP-5-SS-Cy5), along with a small amount of 3′-O-azidomethyl-dGTP and 3′-O-azidomethyl-dTTP to increase fidelity, to the immobilized primed DNA template enables the incorporation of 3′-tBu-SS-dATP-7-SS-HCyC-646 and 3′-tBu-SS-dCTP-5-SS-Cy5, or the 3′-O-azidomethyl-dNTPs, to terminate DNA synthesis. Step 2, after washing away the unincorporated nucleotide analogues at pH 5, imaging for Cy5 or HCyC-646 fluorescence (the two dyes absorb and emit light at essentially the same wavelengths as each other) will reveal those primers extended with either 3′-tBu-SS-dATP-7-SS-HCyC-646 and 3′-tBu-SS-dCTP-5-SS-Cy5. Step 3, Addition of Therminator IX DNA polymerase, the remaining two 3′-t-Butyl-SS-Cleavable Linker-Dyes (3′-tBu-SS-dGTP-7-SS-Cy5 and 3′-tBu-SS-dTTP-5-SS-HCyC-646), along with 3′-O-azidomethyl-dATP and 3′-O-azidomethyl-dCTP to increase fidelity, to the immobilized primed DNA template enables incorporation of 3′-tBu-SS-dGTP-7-SS-Cy5 and 3′-tBu-SS-dTTP-5-SS-HCyC-646, or the 3′-O-azidomethyl-dNTPs. Step 4, After washing away the unincorporated nucleotides at pH 5, a second imaging step is performed to reveal Cy5 or HCyC-646 fluorescence, and new fluorescence signals will confirm incorporation by G or T. Step 5, after washing at pH 9 to reduce fluorescence of the HCyC-646 dye on 3′-tBu-SS-dATP-7-SS-HCyC-646 or 3′-tBu-SS-dTTP-5-SS-HCyC-646, a third imaging step will reveal which nucleotide was incorporated. Thus if it was determined that A or C was added in Imaging Step 1, loss of the fluorescence signal indicates incorporation by A and remaining signal indicates incorporation by C. If it was determined that G or T was added in Imaging Step 2, loss of fluorescence signal indicates incorporation by T and remaining signal incorporation by G. At or just prior to this point, a chase step with the four 3′-O-azidomethyl dNTPs is performed to ensure that nearly every primer has been extended either with one of the dye-labeled 3′-SS-dNTPs or NRT analogues, especially if 3′-O-azidomethyl dNTPs were not added in the first and second extension reactions. Step 6, cleavage of SS linker by adding THP to the elongated DNA strands results in removal of dyes on the nucleotide analogues and also restores the 3′-OH group on any growing strands extended with a 3′-O-azidomethyl-dNTPs. The DNA products are ready for the next cycle of the DNA sequencing reaction. Structures of nucleotides used in this scheme are presented in FIG. 134. In the imaging cartoons at each step, black indicates a positive fluorescent signal and white a background signal. The encoding in the summary cartoon at the end indicates the template sequence, not the incorporated nucleotides.

FIG. 136: Twenty continuous cycles of sequencing by synthesis using 3′-tBu-SS-dATP-7-SS-HCyC-646, 3′-tBu-SS-dCTP-5-SS-Cy5, 3′-tBu-SS-dGTP-7-SS-Cy5, 3′-tBu-SS-dTTP-5-SS-HCyC-646 and the four 3′-O-azidomethyl dNTPs. The procedure, described in FIGS. 133 and 135, was performed for 20 continuous cycles with the template and primer shown at the top of the figure. However, 3′-O-azidomethyl-dNTPs were not added in steps 1 and 3, just during the chase in Step 6. The white bar in each cycle represents the fluorescence result following extension with the first 2 nucleotide analogues (3′-tBu-SS-dATP-7-SS-HCyC-646 and 3′-tBu-SS-dCTP-5-SS-Cy5) and a pH 5 wash, the black bar represents the fluorescence result following extension of the second 2 nucleotide analogues (3′-tBu-SS-dGTP-7-SS-Cy5 and 3′-tBu-SS-dTTP-5-SS-HCyC-646) and a pH 5 wash, and the hatched bar represents the fluorescence results following a pH 9 wash. The expected encoding (010 for T, 011 for G, 111 for C, and 110 for A) was obtained, indicating successful sequencing at each cycle. Treatment with THP at the end of each cycle brings the fluorescence to background levels (not shown). Note that in cycles 4 and 5, the first extension was carried out with 3′-tBu-SS-dGTP-7-SS-Cy5 and 3′-tBu-SS-dTTP-5-SS-HCyC-646 and the second extension was carried out with 3′-tBu-SS-dATP-7-SS-HCyC-646 and 3′-tBu-SS-dCTP-5-SS-Cy5. This is the reverse of the order of addition in the other 18 cycles, and results in different encoding for cycles 4 and 5 (010 for A, 011 for C, 111 for G and 110 for T).

### DETAILED DESCRIPTION

The currently widely used high-throughput SBS technology (Bentley et al. 2008) uses cleavable fluorescent nucleotide reversible terminator (NRT) sequencing chemistry developed previously (Ju et al. 2003; Ju et al. 2006). These cleavable fluorescent NRTs were designed based on the following rationale: each of the four nucleotides (A, C, G, T) is modified by attaching a unique cleavable fluorophore to the specific location of the base and capping the 3′-OH group with a small reversible moiety so that they are still recognized by DNA polymerase as substrates. Thus, the cleavable fluorescent NRTs involve two site modifications (Ju et al. 2003; Ju et al. 2006): a fluorescent dye to serve as a reporter group on the base and a small chemical moiety to cap the 3′-OH group to temporarily terminate the polymerase reaction after nucleotide incorporation for sequence determination. After incorporation and signal detection, the fluorophore is cleaved and the 3′-OH capping moiety removed to resume the polymerase reaction in the next cycle. These cleavable fluorescent NRTs have proved to be good substrates for reengineered polymerases and have been used extensively in next generation DNA sequencing systems (Ju et al. 2006; Bentley et al. 2008). Moreover, they enable accurate determination of homopolymer sequences, since only one base is identified in each cycle.

An SBS approach using cleavable fluorescent nucleotide analogues as reversible terminators to sequence surface-immobilized DNA has been used (Ju et al. 2003; Li et al. 2003; Ruparel et al. 2005; Ju et al. 2006; Wu et al. 2007; Guo et al. 2008). In this approach, the nucleotides are modified at two specific locations so that they are still recognized by DNA polymerase as substrates: (i) a different fluorophore with a distinct fluorescent emission is linked to the specific location of each of the four bases through a cleavable linker and (ii) the 3′-OH group is capped by a small chemically reversible moiety. DNA polymerase incorporates only a single nucleotide analogue complementary to the base on a DNA template covalently linked to a surface. After incorporation, a unique fluorescence emission is detected to identify the incorporated nucleotide. The fluorophore is subsequently removed and 3′-OH group is chemically regenerated, which allows the next cycle of the polymerase reaction to proceed. Because the large surface on a DNA chip can have a high density of different DNA templates spotted, each cycle can identify many bases in parallel, allowing the simultaneous sequencing of a large number of DNA molecules. Previous research efforts have firmly established the molecular level strategy to rationally modify the nucleotides by attaching a cleavable fluorescent dye to the base and reversibly capping the 3′-OH with a small moiety for SBS.

A class of nucleotide analogues with unprotected 3′-OH and a cleavable disulfide linker attached between the base and fluorescent dye has been reported (Turcatti et al. 2008; Mitra et al. 2003). However, after DNA polymerase catalyzed extension reaction on the primer/template and imaging the incorporated base, the cleavage of the disulfide linkage generates a free reactive —SH group which has to be capped with alkylating agent, iodoacetamide, before the second extension reaction can be carried out. This capping step not only adds an extra step in the process but also limits the addition of multiple nucleotides in a row because of the long remnant tail on the nucleotide base moiety. With this approach the sequencing read length is limited to only 10 bases (Turcatti et al. 2008). Other disulfide-based approaches require a similar capping reaction to render the free SH group unreactive (Mitra et al. 2003).

The invention provides a nucleotide analogue having structure:

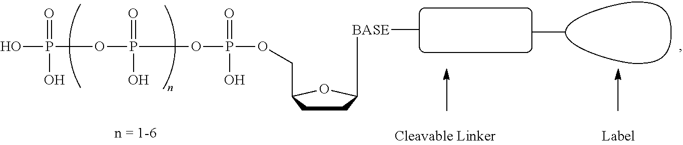

wherein:
   BASE comprises adenine, guanine, cytosine, thymine, uracil, hypoxanthine or analog thereof;
   Cleavable Linker comprises DTM, Azo, 2-Nitrobenzyl, Allyl, Azidomethyl, or TCO Derivative, and is attached to the base via 5 position of pyrimidines (C, U) or 7 position of deazapurines (A, G, I); and
   Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye.

The invention provides a nucleotide analogue having the structure:

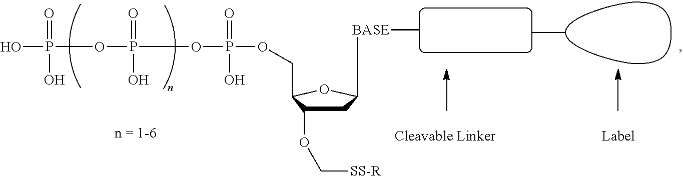

wherein:
   BASE comprises adenine, guanine, cytosine, thymine, uracil, hypoxanthine or analog thereof;
   R comprises methyl, ethyl, propyl, t-butyl, aryl, alkyl aryl;
   Cleavable Linker comprises DTM, Azo, 2-Nitrobenzyl, Allyl, Azidomethyl or TCO Derivative; and
   Label comprises a fluorescent dye, a pH responsive fluorescent dye, a cluster of a fluorescent dye, a cluster of a pH responsive fluorescent dye, an anchor for attachment of a fluorescent dye, a cluster of an anchor for attachment of fluorescent dyes, or an anchor and dye.

The invention provides a nucleotide analogue having structure:

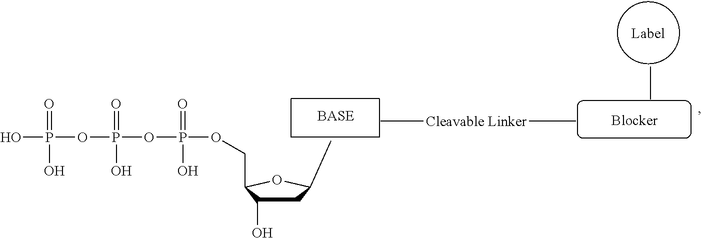

wherein:
   BASE comprises adenine, guanine, cytosine, thymine, uracil, hypoxanthine or analog thereof;
   Cleavable Linker comprises DTM, Azo, 2-Nitrobenzyl, Allyl, Azidomethyl, or TCO Derivative, or more than one of these cleavable linkers, including the special case where one cleavable linker is present between the base and the blocker and a second different cleavable linker is present between the blocker and the label;
   Blocker is a nucleotide or oligonucleotide comprising 2-50 monomer units of abasic sugars or modified nucleosides or a combination thereof; and blocker is connected to the 5-position of pyrimidines (C, U) and 7-position of deazapurines (A, G, I) via a cleavable linker;
   wherein a Blocker is a moiety that, after incorporation, prevents further incorporation of additional nucleotides or nucleotide analogues into a primer strand; and
   Label comprises a fluorescent dye, a pH responsive fluorescent dye, a cluster of a fluorescent dye, a cluster of a pH responsive fluorescent dye, an anchor for attachment of a fluorescent dye, a cluster of an anchor for attachment of fluorescent dyes, or an anchor and dye, wherein the label is attached to the blocker.

The invention provides a nucleotide analogue having structure:

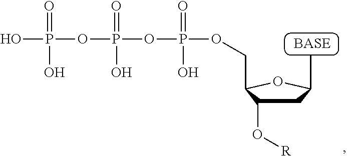

wherein:
   BASE comprises adenine, guanine, cytosine, uracil, thymine, hypoxanthine or analogue thereof; and
   R is a cleavable chemical group comprising alkyl DTM, Azo, 2-Nitrobenzyl, Allyl and Azidomethyl Derivatives.

The invention provides a nucleotide analogue having structure:

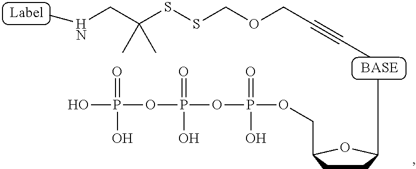

wherein:
   BASE comprises adenine, guanine, cytosine, thymine, uracil, hypoxanthine or analog thereof; and
   Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye.

The invention provides a nucleotide analogue having structure:

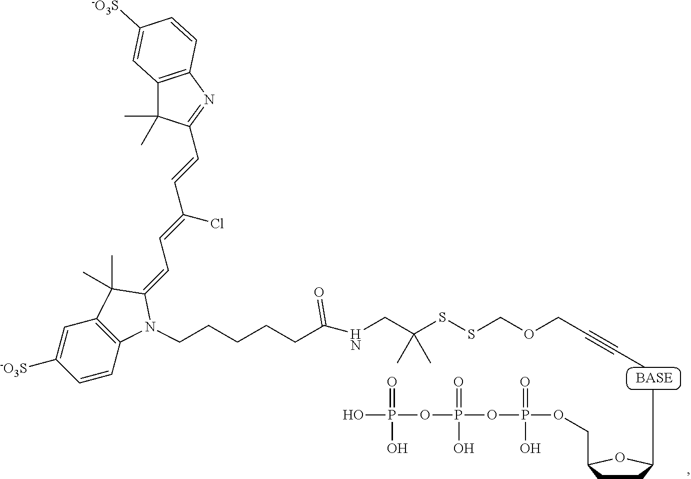

wherein BASE comprises adenine, guanine, cytosine, thymine, uracil, hypoxanthine or analog thereof.

The invention provides a nucleotide analogue having structure:

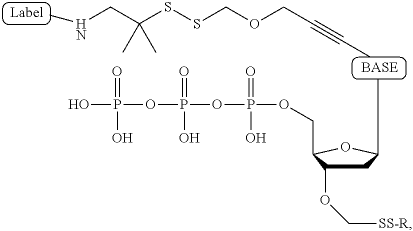

wherein:
   BASE comprises adenine, guanine, cytosine, thymine, uracil, hypoxanthine or analog thereof;
   Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye; and
   R comprises methyl, ethyl, propyl, t-butyl, aryl, alkyl aryl.

The invention provides a nucleotide analogue having structure:

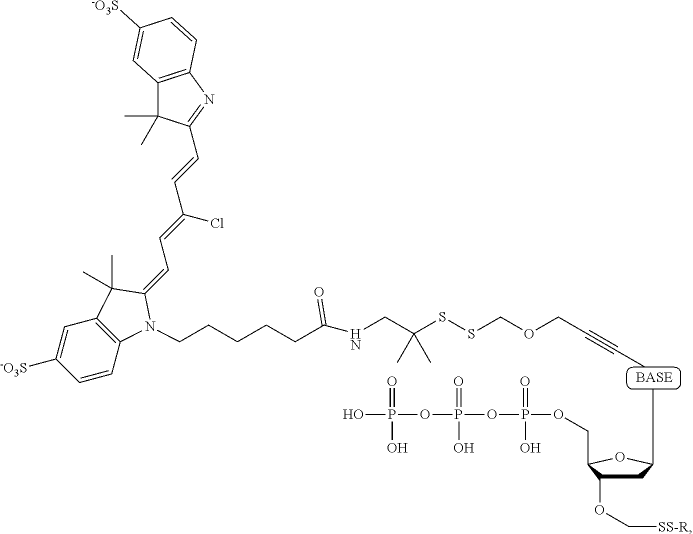

wherein:
   BASE comprises adenine, guanine, cytosine, thymine, uracil, hypoxanthine or analog thereof; and
   R comprises methyl, ethyl, propyl, t-butyl, aryl, alkyl aryl.

The invention provides a nucleotide analogue having structure:

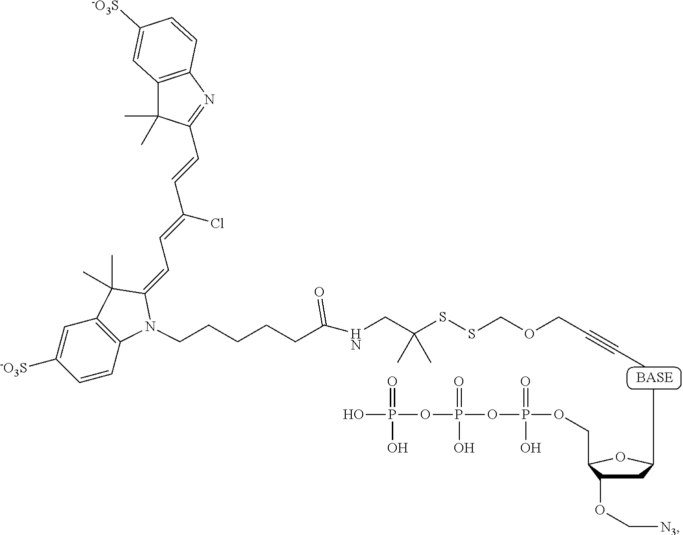

wherein BASE comprises adenine, guanine, cytosine, thymine, uracil, hypoxanthine or analog thereof.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) (A) a fluorescently labeled dideoxynucleotide analogue comprising a base and a fluorescent label linked to the base via a cleavable linker and (B) an anchor labeled dideoxynucleotide analogue comprising a base and an anchor attached to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) an anchor labeled nucleotide analogue comprising a base and an anchor attached to the base via a cleavable linker and a blocking group at the 3′-OH position, wherein said blocking group prevents incorporation of a subsequent nucleotide analogue into the extended primer strand,
      wherein the cleavable linkers and 3′-OH group are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) an anchor labeled nucleotide analogue comprising a base, a blocking group linked to the base via a cleavable linker, and an anchor linked to the base distal to the blocking group, wherein said blocking group prevents or greatly reduces incorporation of a subsequent nucleotide analogue into the extended primer strand,
      wherein the cleavable linkers are cleavable by an identical cleaving agent;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the anchor of the provided anchor labeled nucleotide analogue of step (b), wherein said anchor binding group comprises a fluorescent label identical to the fluorescent label of the fluorescently labeled nucleotide analogue of step (b);
f) identifying any fluorescence signal due to the binding of the anchor binding group to the anchor of any incorporated anchor labeled nucleotide analogue of step (b);
g) repeating steps (b)-(f) with two different labeled nucleotide analogues that are different from the two different labeled nucleotide analogues from the previous iteration of step (b); h) cleaving the cleavable linkers from the incorporated nucleotide analogue, thereby removing any label, anchor, or blocking group from the incorporated nucleotide analogue of step (b);
i) cleaving the 3′-O blocking group from any incorporated nucleotide analogue from step (c); and
j) iteratively repeating steps (b) to (i) for each residue of the nucleic acid to be sequenced,
   thereby obtaining the sequence of the nucleic acid.

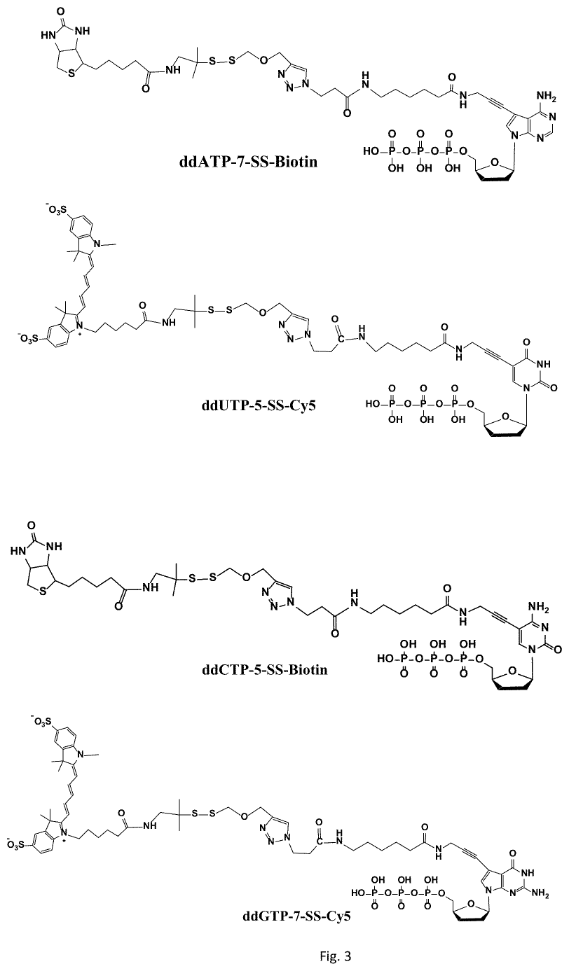

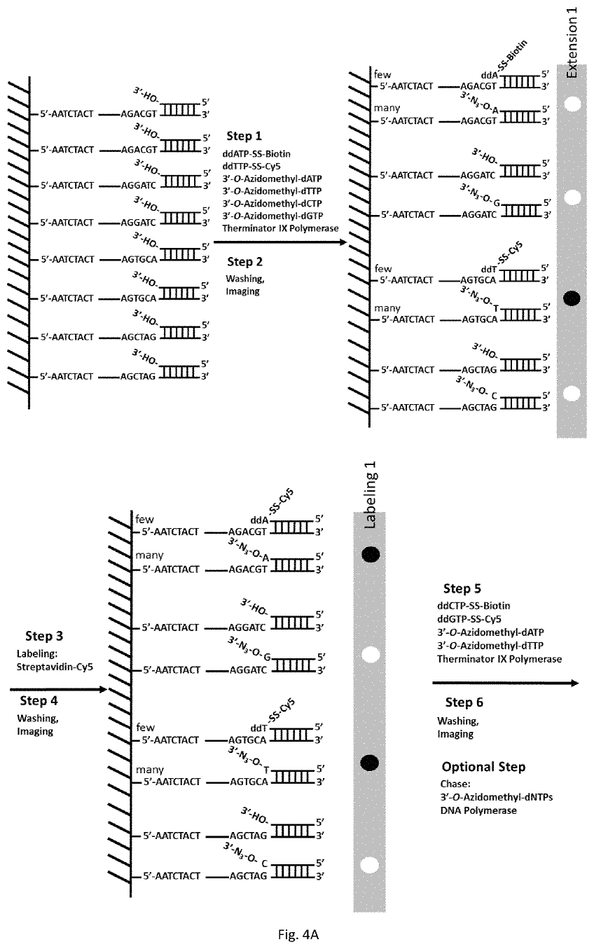

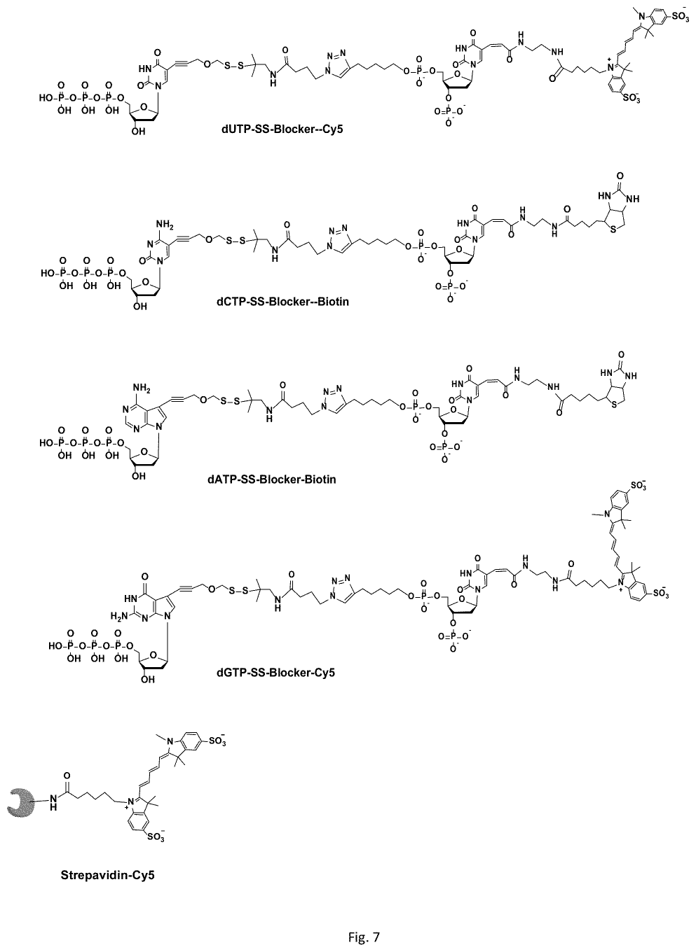

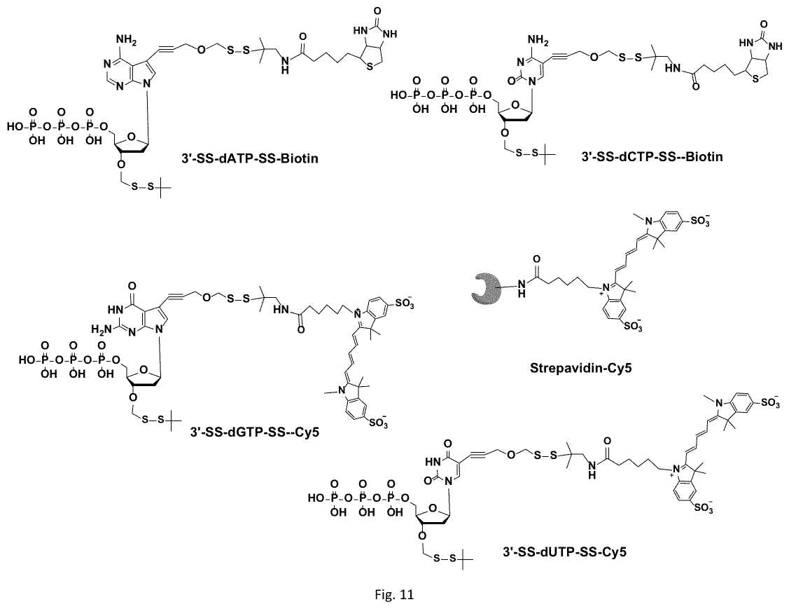

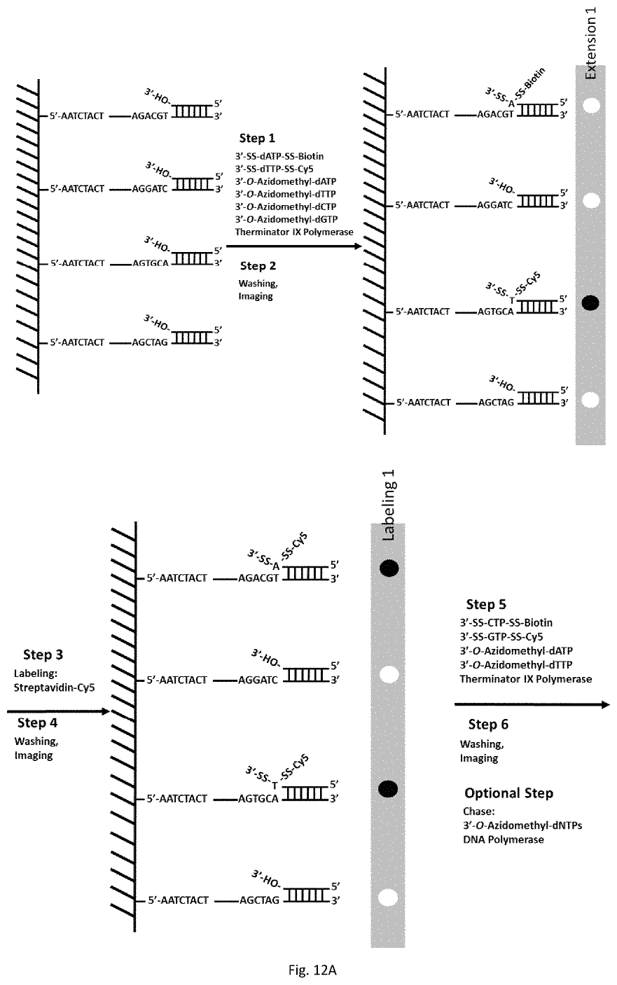

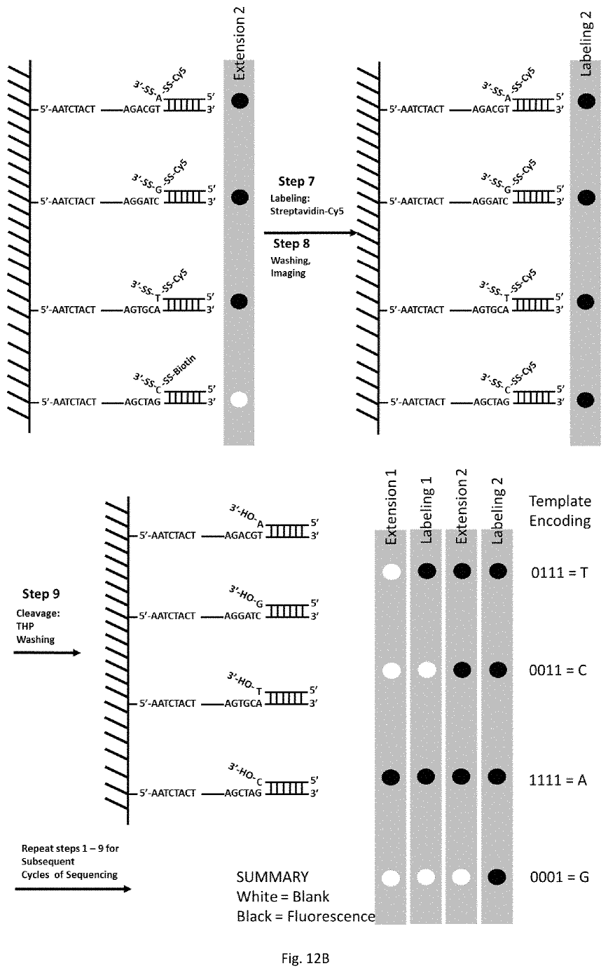

In an embodiment, the nucleotide analogues of step (b) are selected from the group consisting of the nucleotide analogues of FIG. 3, FIG. 7, or FIG. 11.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) fluorescently labeled dideoxynucleotide analogue comprising a base and a fluorescent label linked to the base via a cleavable linker, (B) pH-responsive fluorescently labeled dideoxynucleotide analogue comprising a base and a pH-responsive fluorescent label linked to the base via a cleavable linker, (C) two different anchor labeled dideoxynucleotide analogues, wherein each analogue comprises a different anchor attached to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a pH-responsive fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (C) two different anchor labeled nucleotide analogues comprising a base and an anchor attached to the base via a cleavable linker and a blocking group at the 3′-OH position, wherein said blocking group prevents incorporation of a subsequent nucleotide analogue into the extended primer strand, wherein each analogue comprises a different anchor attached to the base via a cleavable linker,
      wherein the cleavable linkers and 3′-O blocking groups are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand, and (C) two different anchor labeled nucleotide analogues comprising a base, a blocking group linked to the base via a cleavable linker, and an anchor linked to the base distal to the blocking group, wherein said blocking group prevents or greatly reduces incorporation of a subsequent nucleotide analogue into the extended primer strand,
      wherein each analogue comprises a different anchor attached to the base via a cleavable linker,
      wherein the cleavable linkers are cleavable by an identical cleaving agent;
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label has same or similar absorption and emission profile as the fluorescent label on the fluorescently labeled nucleotide analogue and identifying any fluorescence signal due to incorporation of a labeled nucleotide analogue from step (b);
e) contacting the incorporated nucleotide analogue from step (b) with (A) an anchor binding group that binds to the anchor of only one of the anchor labeled nucleotide analogues of step (b), wherein the anchor binding group comprises the same fluorescent label as the fluorescently labeled nucleotide analogue of step (b), and (B) an anchor binding group that binds only to the anchor of the remaining anchor labeled nucleotide analogue, wherein the anchor binding group comprises the same pH-responsive fluorescent label as the pH-responsive fluorescently labeled nucleotide analogue of step (b);
f) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of an anchor labeled nucleotide analogue from step (b);
g) washing the incorporated nucleotide analogue from step (b) at a pH at which the pH-responsive fluorescent label no longer has the same or similar absorption and emission profile as the fluorescent label on the fluorescently labeled nucleotide analogue and identifying any fluorescence signal due to incorporation of a labeled nucleotide analogue from step (b);
h) cleaving the cleavable linker from the incorporated nucleotide analogue, thereby removing any label, anchor, or blocking group from the incorporated nucleotide analogue of step (b);
i) cleaving the 3′-O blocking group from any incorporated nucleotide analogue from step (c); and
j) iteratively repeating steps (b) to (i) for each residue of the nucleic acid to be sequenced,
   thereby obtaining the sequence of the nucleic acid.

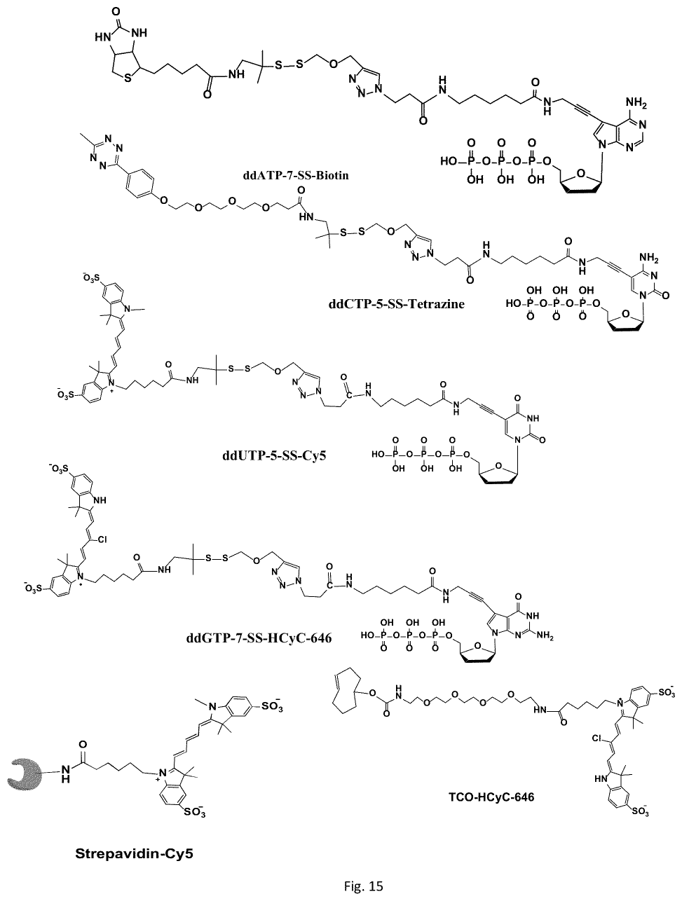

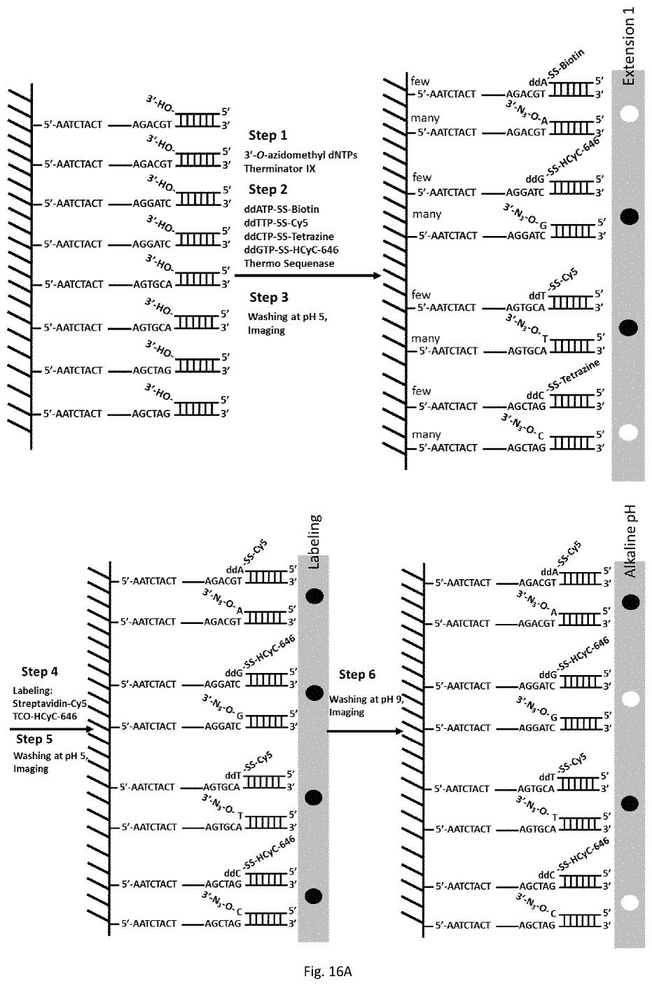

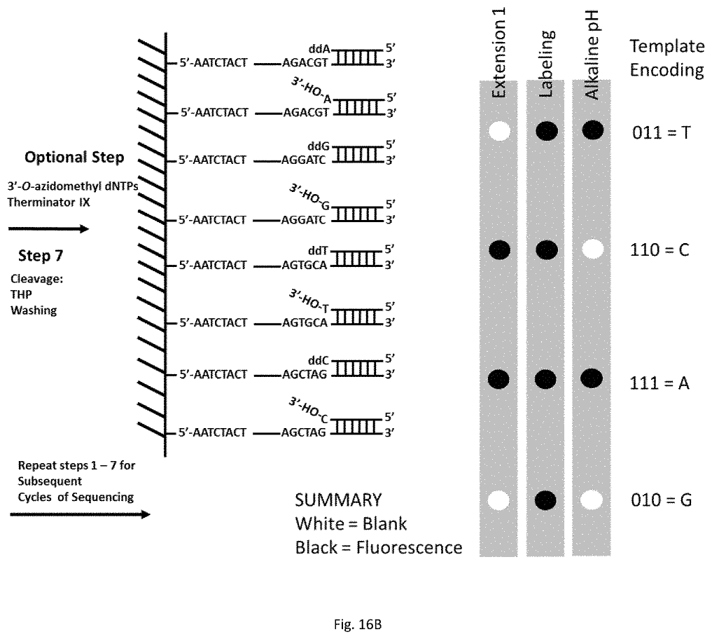

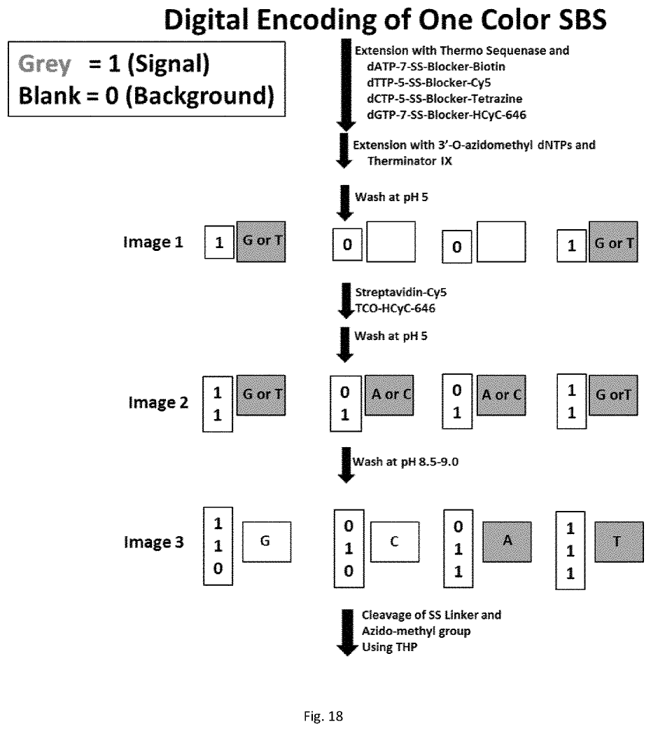

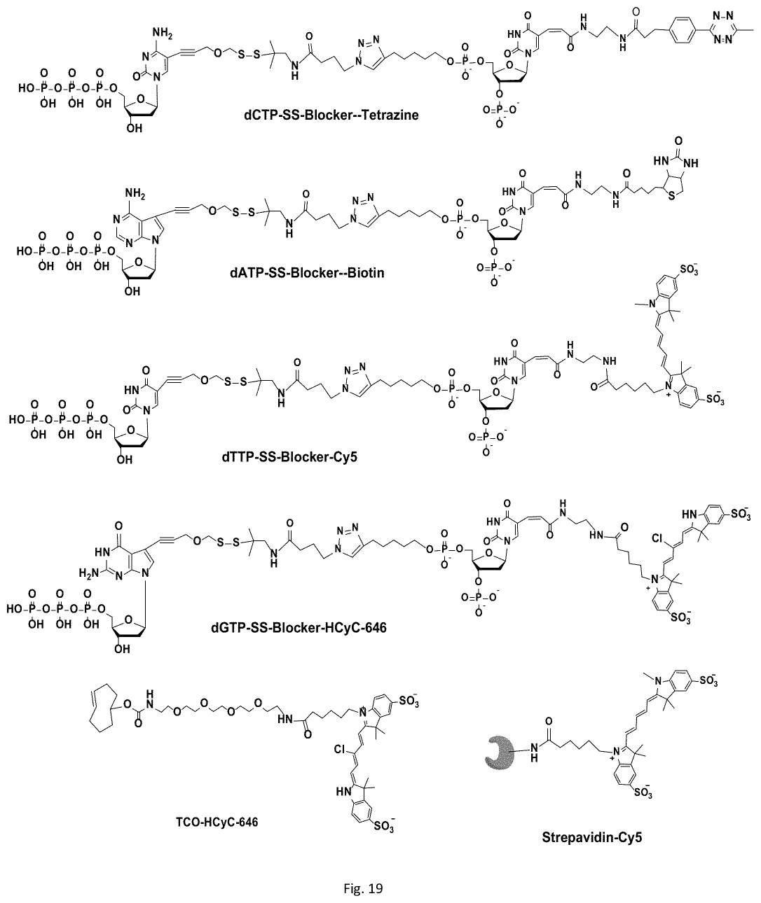

In an embodiment, the nucleotide analogues of step (b) are selected from the group consisting of the nucleotide analogues of FIG. 15, FIG. 18, or FIG. 19. In an embodiment, the label with pH-responsive fluorescence is HCyC-646 and the label with pH-inresponsive fluorescence is Cy5.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) (A) a fluorescently labeled dideoxynucleotide analogue comprising a base and a fluorescent label linked to the base via a cleavable linker and (B) pH-responsive fluorescently labeled dideoxynucleotide analogue comprising a base and a pH-responsive fluorescent label linked to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a pH-responsive fluorescently labeled nucleotide analogue comprising a base and a pH-responsive fluorescent label linked to the base via a cleavable linker and a blocking group at the 3′-OH position, wherein said blocking group prevents incorporation of a subsequent nucleotide analogue into the extended primer strand,
      wherein the cleavable linkers and the 3′-O blocking group are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand,
      wherein the cleavable linkers are cleavable by an identical cleaving agent;
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label has same or similar absorption and emission profile as the fluorescent label on the fluorescently labeled nucleotide analogue and identifying any fluorescence signal due to incorporation of a labeled nucleotide analogue from step (b);
e) repeating steps (b)-(d) with two different labeled nucleotide analogues that are different from the two different labeled nucleotide analogues from the previous iteration of step (b);
f) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of a labeled nucleotide analogue from step (b),
   wherein steps (e) and (f) may be performed in the reverse order;
g) cleaving the cleavable linkers from the incorporated nucleotide analogue, thereby removing any label or blocking group from the incorporated nucleotide analogue of step (b);
h) cleaving the 3′-O blocking group from any incorporated nucleotide analogue from step (c); and
i) iteratively repeating steps (b) to (h) for each residue of the nucleic acid to be sequenced,
   thereby obtaining the sequence of the nucleic acid.

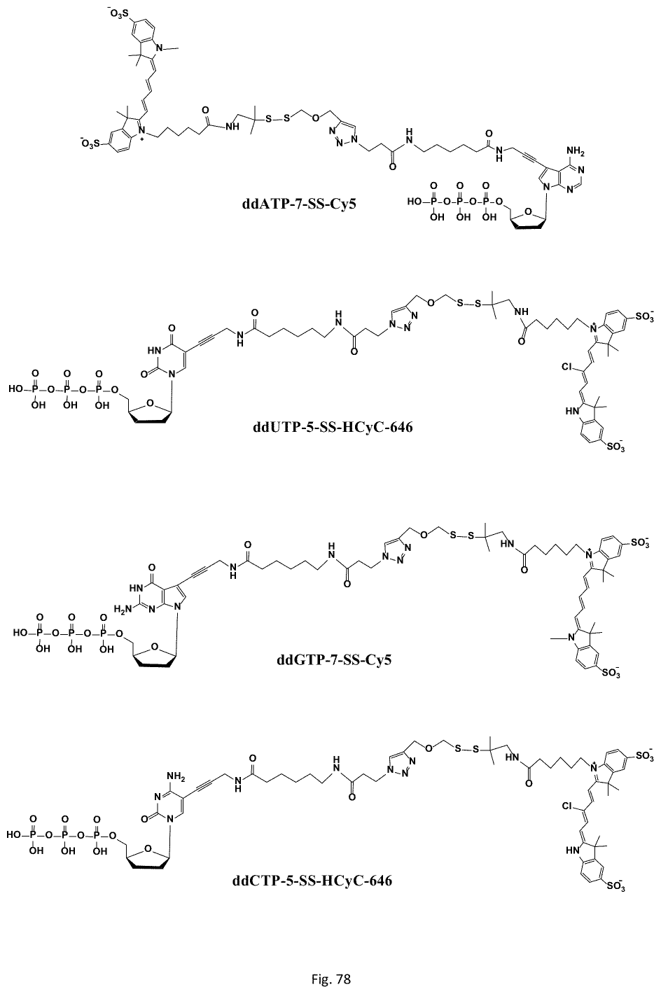

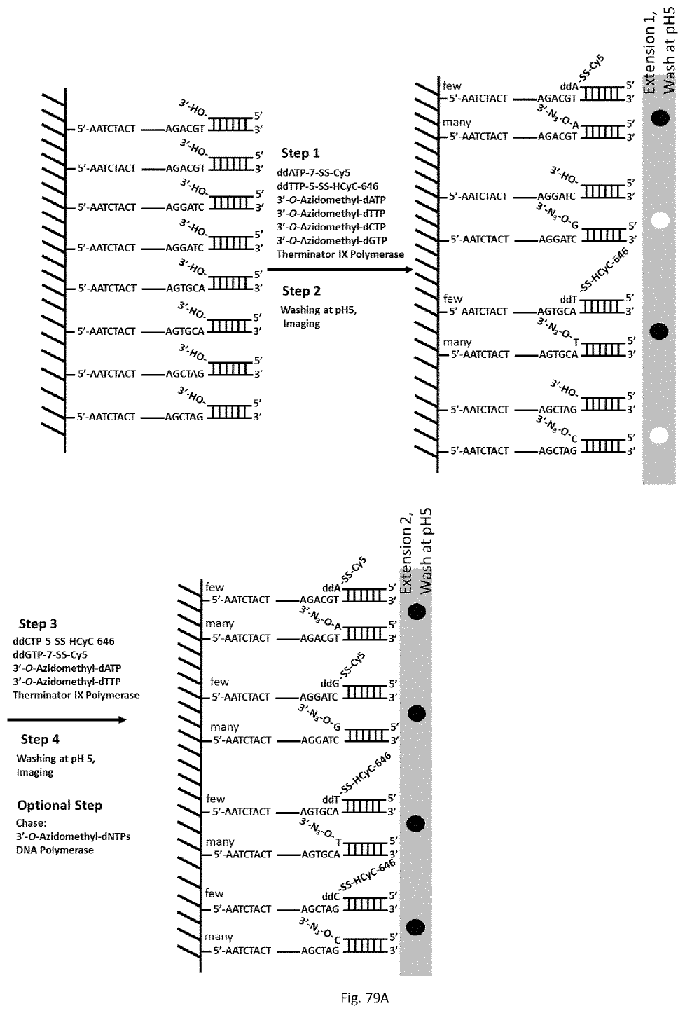

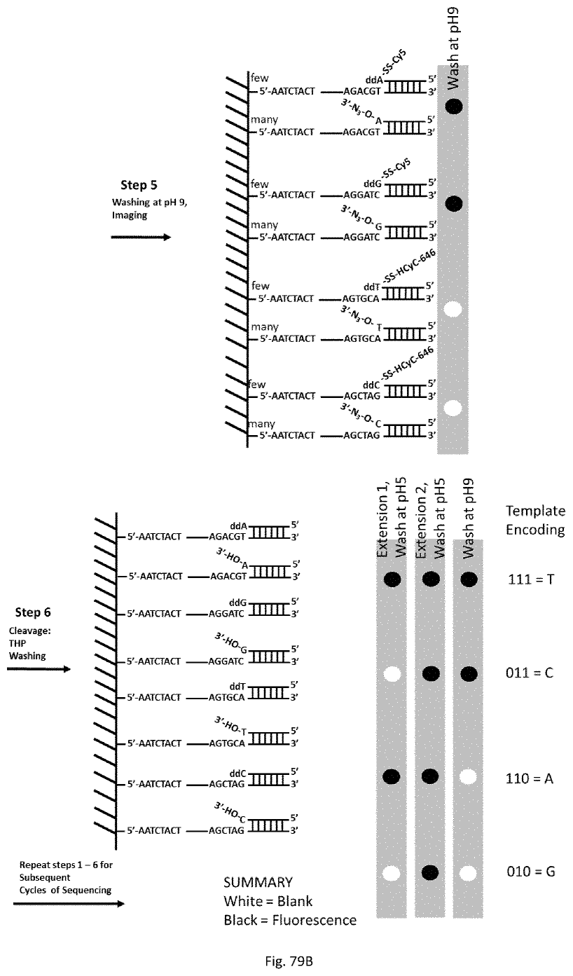

In an embodiment, the nucleotide analogues of step (b) are selected from the group consisting of the nucleotide analogues of FIG. 78. In an embodiment, the label with pH-responsive fluorescence is HCyC-646 and the label with pH-unresponsive fluorescence is Cy5.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via a carbamyl TCO linker, (C) an anchor labeled dideoxynucleotide analogue comprising a base and an anchor attached to the base via the first cleavable linker, and (D) an anchor labeled dideoxynucleotide analogue comprising a base and an anchor attached to the base via a carbamyl TCO linker;
   (ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via a carbamyl TCO linker, and a blocking group at the 3′-OH position, wherein said blocking group prevents incorporation of a subsequent nucleotide analogue into the extended primer strand, (C) an anchor labeled nucleotide analogue comprising a base and an anchor attached to the base via the first cleavable linker, and a blocking group at the 3′-OH position, wherein said blocking group prevents incorporation of a subsequent nucleotide analogue into the extended primer strand, and (D) an anchor labeled nucleotide analogue comprising a base and an anchor attached to the base via a carbamyl TCO linker, and a blocking group at the 3′-OH position, wherein said blocking group prevents incorporation of a subsequent nucleotide analogue into the extended primer strand, wherein the 3′-O blocking group and the first cleavable linker are cleavable by the same cleaving agent; or
   (iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a carbamyl TCO linker, and a fluorescent label linked to the base distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a first cleavable linker, and an anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a carbamyl TCO linker, and an anchor linked to the base distal to the blocking group, wherein said blocking group prevents or greatly reduces incorporation of a subsequent nucleotide analogue into the extended primer strand,
      wherein the fluorescent labels on each analogue are the same,
      wherein the anchors on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the anchor of the provided anchor labeled nucleotide analogues of step (b), wherein said anchor binding group comprises a fluorescent label identical to the fluorescent label of the fluorescently labeled nucleotide analogues of step (b);
f) identifying any fluorescence signal due to the binding of the anchor binding group to the anchor of any incorporated anchor labeled nucleotide analogue of step (b);
g) contacting the incorporated nucleotide analogue with a tetrazine derivative to click to the TCO moiety of the carbamyl TCO linker to release any label or anchor linked by a carbamyl TCO linker and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced,
   thereby obtaining the sequence of the nucleic acid.

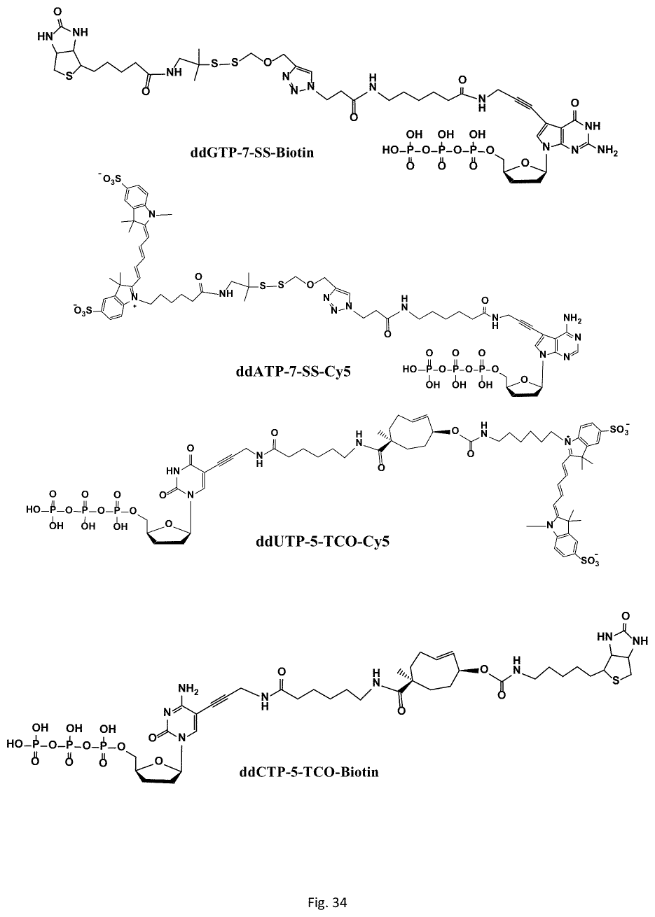

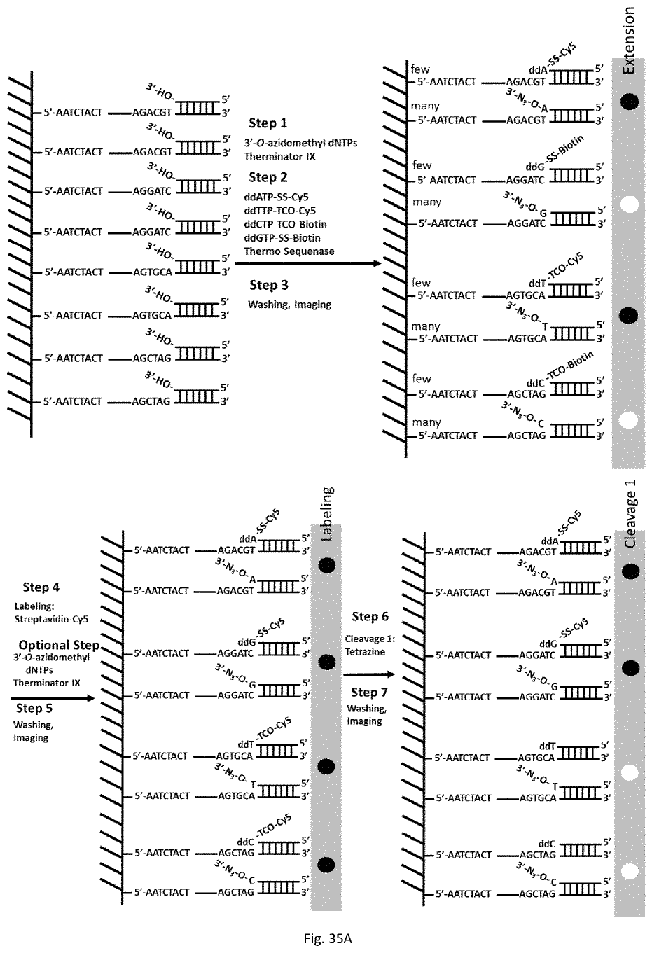

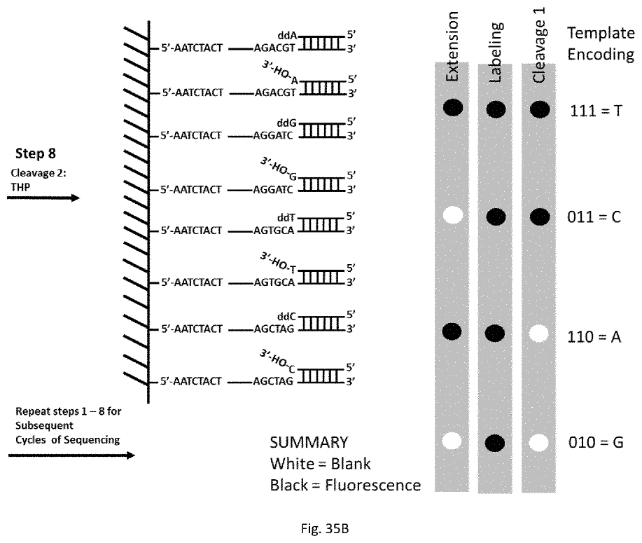

In an embodiment, the nucleotide analogues of step (b) are selected from the group consisting of the nucleotide analogues of FIG. 34.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a cleavable linker, (B) a fluorescently labeled dideoxynucleotide analogue comprising a base and a fluorescent label and a first anchor linked to the base via a cleavable linker, (C) an anchor labeled dideoxynucleotide analogue comprising a base and the first anchor and a second anchor attached to the base via a cleavable linker, and (D) an anchor labeled dideoxynucleotide analogue comprising a base and the second anchor attached to the base via a cleavable linker, wherein the cleavable linkers are cleavable by an identical cleavage agent;
   (ii) (A) a fluorescently labeled nucleotide analogue comprising a base and a fluorescent label linked to the base via a cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label and a first anchor linked to the base via a cleavable linker, and a blocking group at the 3′-OH position, wherein said blocking group prevents incorporation of a subsequent nucleotide analogue into the extended primer strand, (C) an anchor labeled nucleotide analogue comprising a base and the first anchor and a second anchor attached to the base via the cleavable linker, and a blocking group at the 3′-OH position, wherein said blocking group prevents incorporation of a subsequent nucleotide analogue into the extended primer strand, and (D) an anchor labeled nucleotide analogue comprising a base and the second anchor attached to the base via a cleavable linker, and a blocking group at the 3′-OH position, wherein said blocking group prevents incorporation of a subsequent nucleotide analogue into the extended primer strand, wherein the cleavable linkers and the 3′-O blocking group are cleavable by an identical cleavage agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a cleavable linker, and a fluorescent label and a first anchor linked to the base distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a cleavable linker, and the first anchor and a second anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a cleavable linker, and the second anchor linked to the base distal to the blocking group, wherein said blocking group prevents or greatly reduces incorporation of a subsequent nucleotide analogue into the extended primer strand, wherein the cleavable linkers are cleavable by an identical cleavage agent,
   wherein the fluorescent labels on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the second anchor of the nucleotide analogues of step (b), wherein said anchor binding group comprises a fluorescent label identical to fluorescent label of the fluorescently labeled nucleotide analogues of step (b);
f) identifying any fluorescence signal due to the binding of the anchor binding group to the anchor of any incorporated nucleotide analogue of step (b);
g) contacting the incorporated nucleotide analogue with a second anchor binding group that binds to the first anchor of the nucleotide analogues of step (b) and comprises a moiety that quenches the fluorescent signal of any fluorescent label attached to the nucleotide analogue to which the anchor binding group attaches, and identifying any fluorescence signal due to incorporation of a fluorescently labeled nucleotide analogue;
h) contacting the incorporated nucleotide analogue with a cleaving agent that cleaves the cleavable linker and any 3′-O blocking group; and
i) iteratively repeating steps (b) to (h) for each residue of the nucleic acid to be sequenced,
   thereby obtaining the sequence of the nucleic acid.

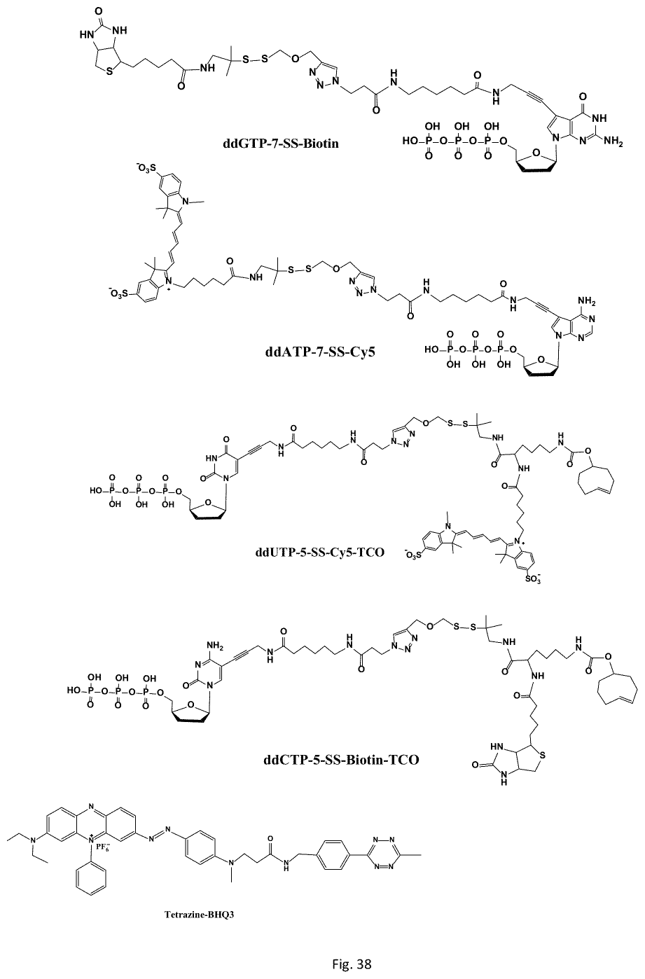

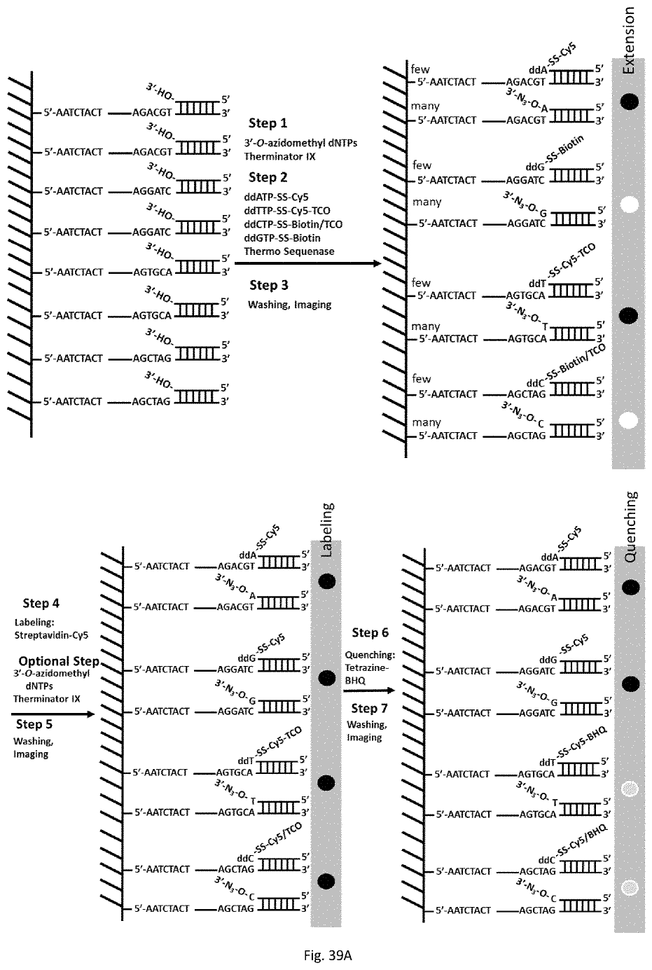

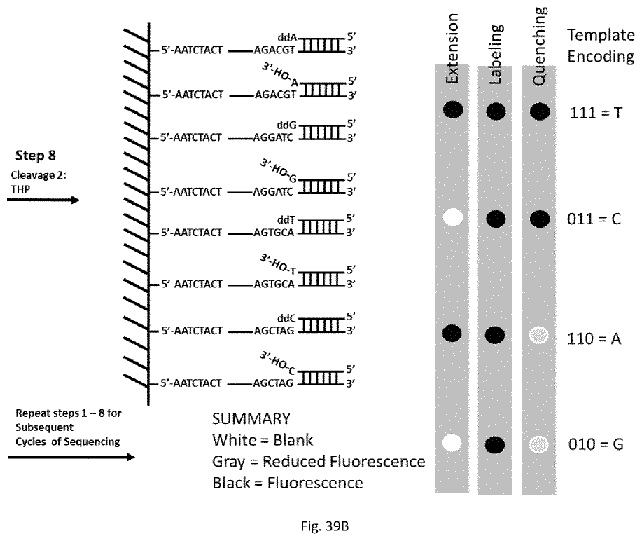

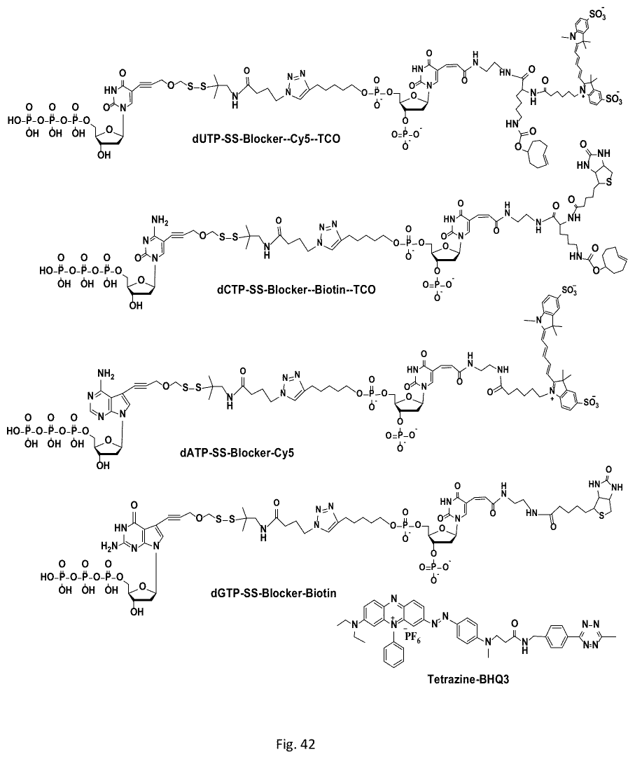

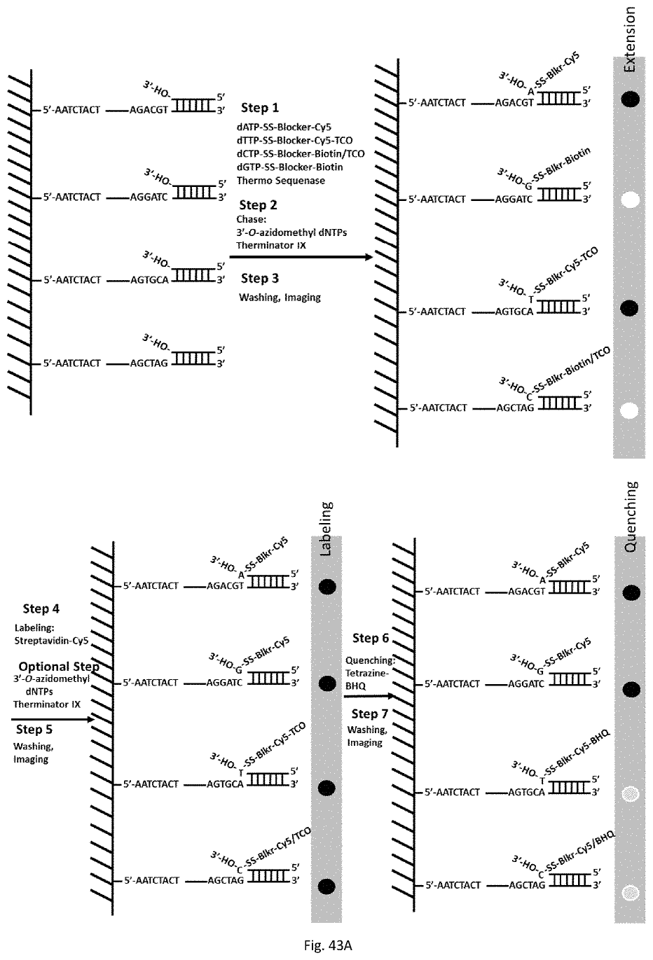

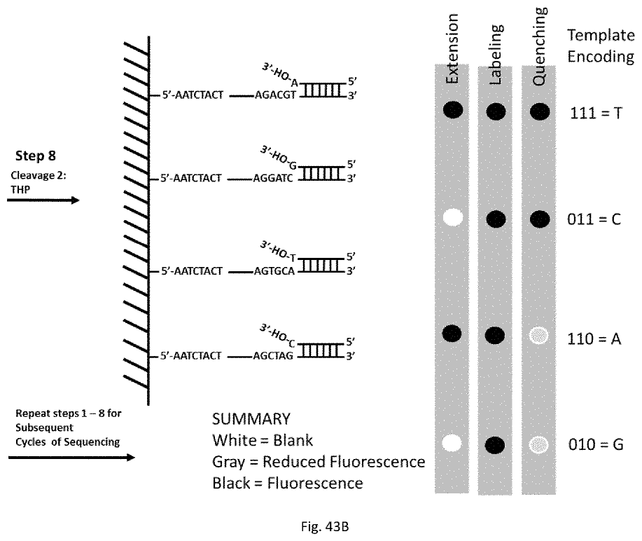

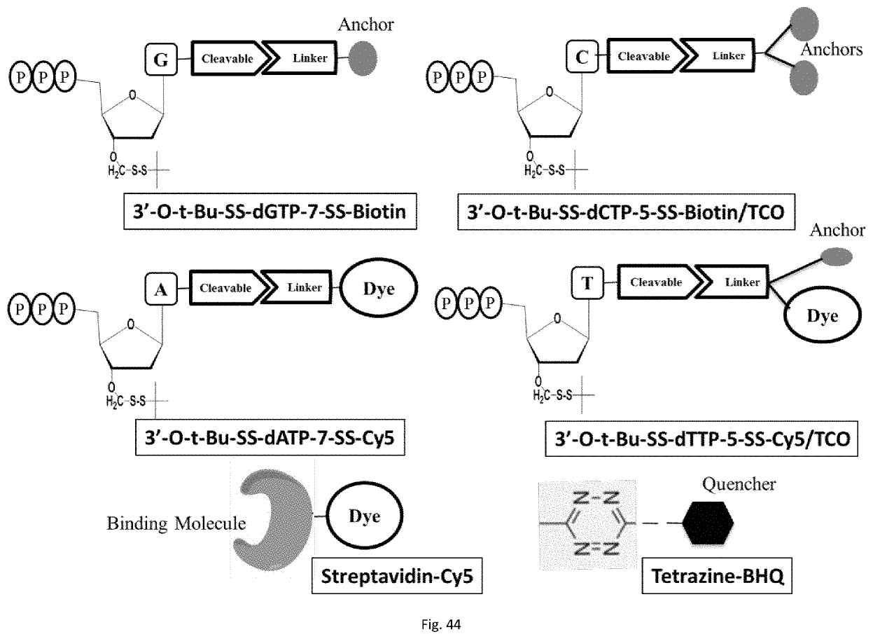

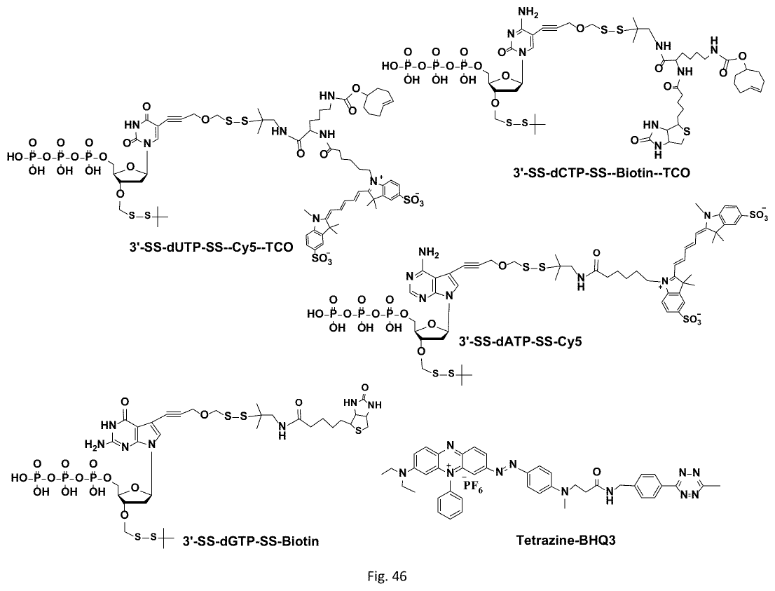

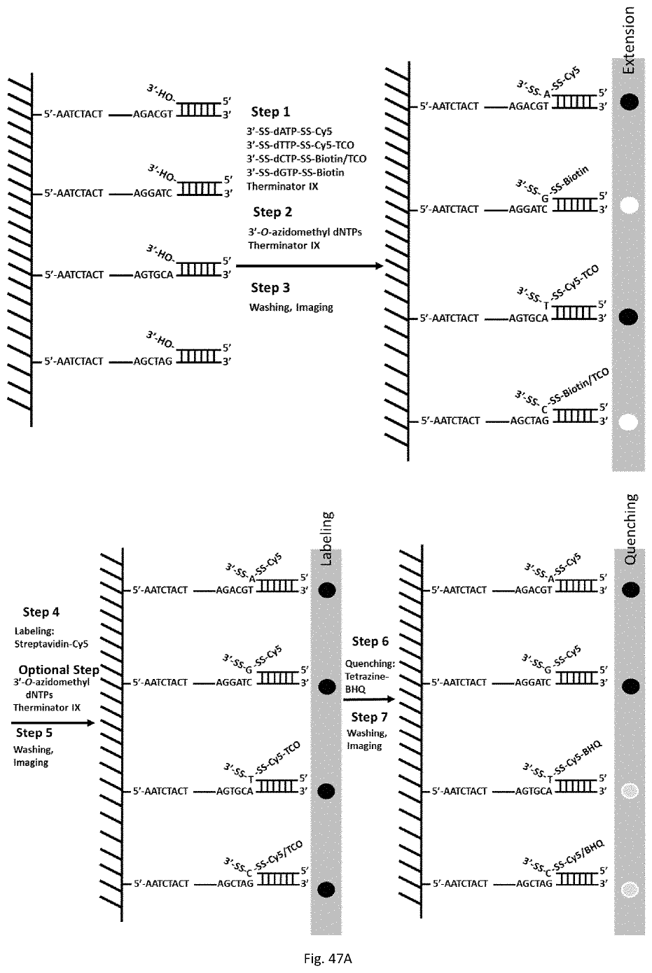

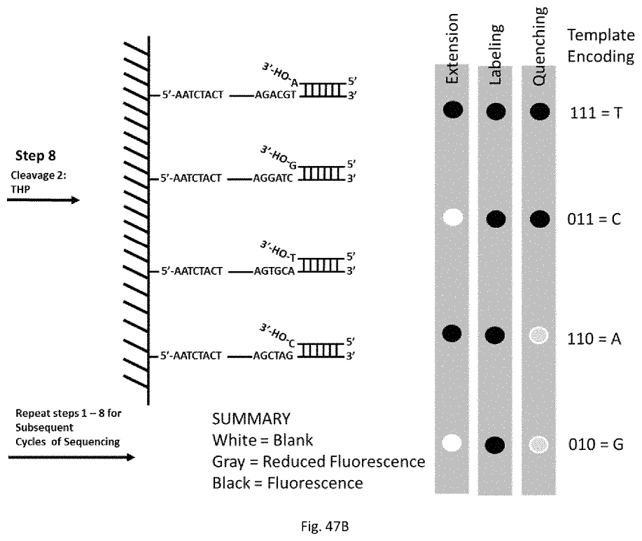

In an embodiment, the nucleotide analogues of step (b) are selected from the group consisting of the nucleotide analogues of FIG. 38, FIG. 42 or FIG. 46.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via the first cleavable linker and a carbamyl TCO linker attached distal to the first cleavable linker, (C) an anchor labeled dideoxynucleotide analogue comprising a base and an anchor linked to the base via the first cleavable linker and a carbamyl TCO linker attached distal to the first cleavable linker, and (D) an anchor labeled dideoxynucleotide analogue comprising a base and an anchor attached to the base via the first cleavable linker;
   (ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, (C) an anchor labeled nucleotide analogue comprising a base and an anchor attached to the base via the first cleavable linker, and a blocking group at the 3′-OH position, wherein said blocking group prevents incorporation of a subsequent nucleotide analogue into the extended primer strand, and (D) an anchor labeled nucleotide analogue comprising a base and an anchor linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, wherein the 3′-OH blocking group and the first cleavable linker are cleavable by the same agent; or
   (iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via the first cleavable linker, and a fluorescent label linked to the base via a carbamyl TCO linker distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a first cleavable linker, and an anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a the first cleavable linker, and an anchor linked to the base via a carbamyl TCO linker distal to the blocking group, wherein said blocking group prevents or greatly reduces incorporation of a subsequent nucleotide analogue into the extended primer strand,
      wherein the fluorescent labels on each analogue are the same,
      wherein the anchors on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue in step (b);
e) contacting the incorporated nucleotide analogue from step (b) with an anchor binding group that binds to the anchor of the provided anchor labeled nucleotide analogues of step (b), wherein said anchor binding group comprises a fluorescent label identical to the fluorescent label of the fluorescently labeled nucleotide analogues of step (b);
f) identifying any fluorescence signal due to the binding of the anchor binding group to the anchor of any incorporated anchor labeled nucleotide analogue of step (b);
g) contacting the incorporated nucleotide analogue with a tetrazine derivative to click to the TCO moiety of the carbamyl TCO linker to release any label or anchor linked by a carbamyl TCO linker and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced,
   thereby obtaining the sequence of the nucleic acid.

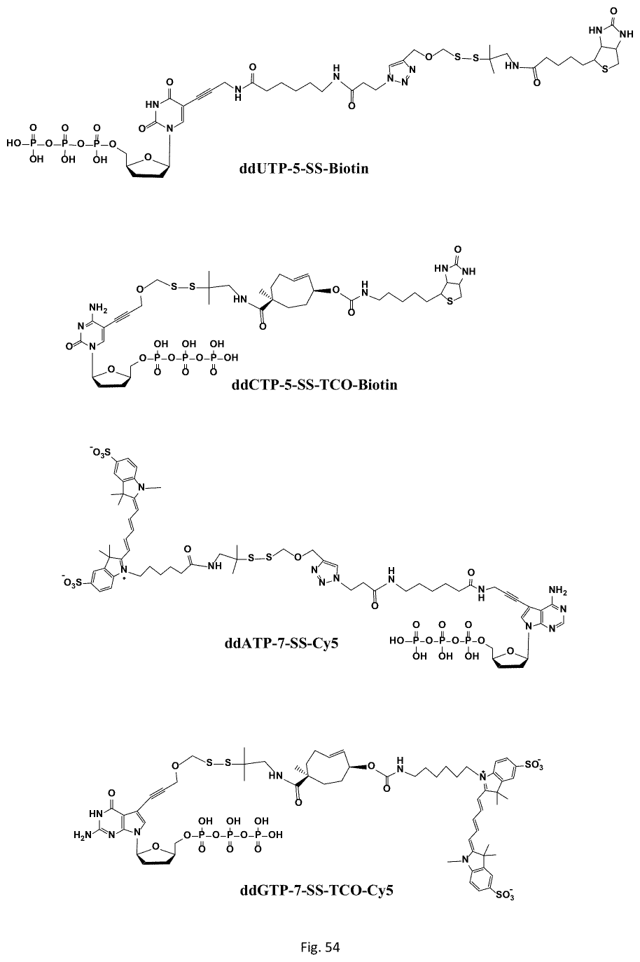

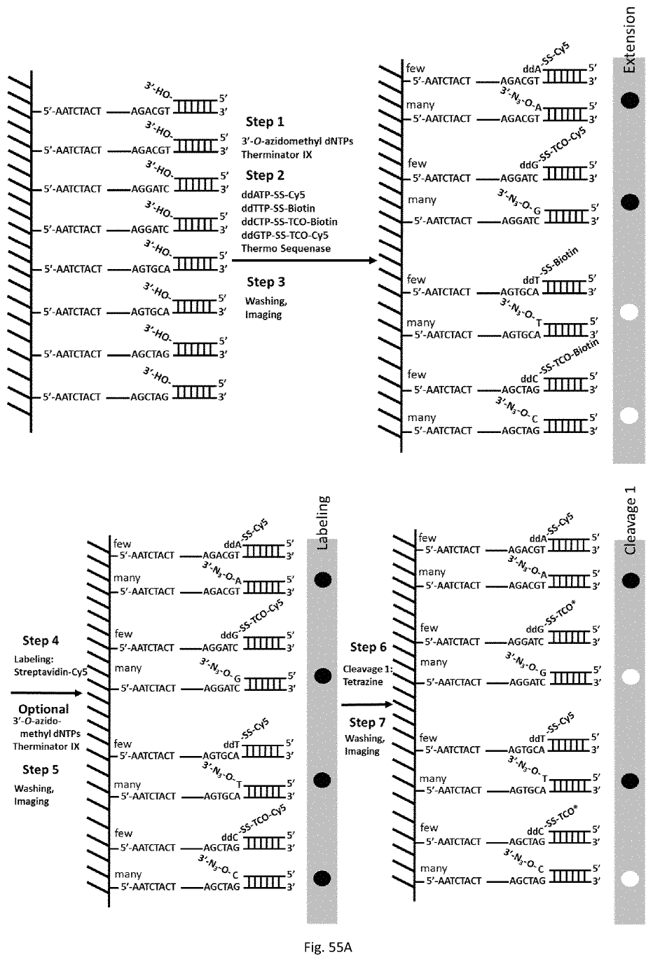

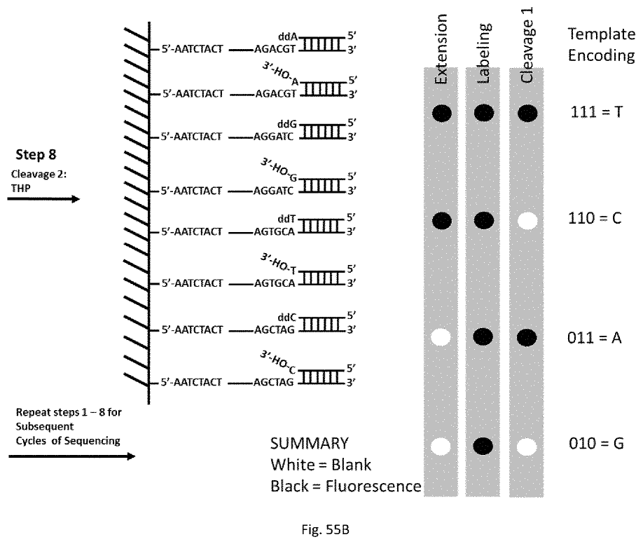

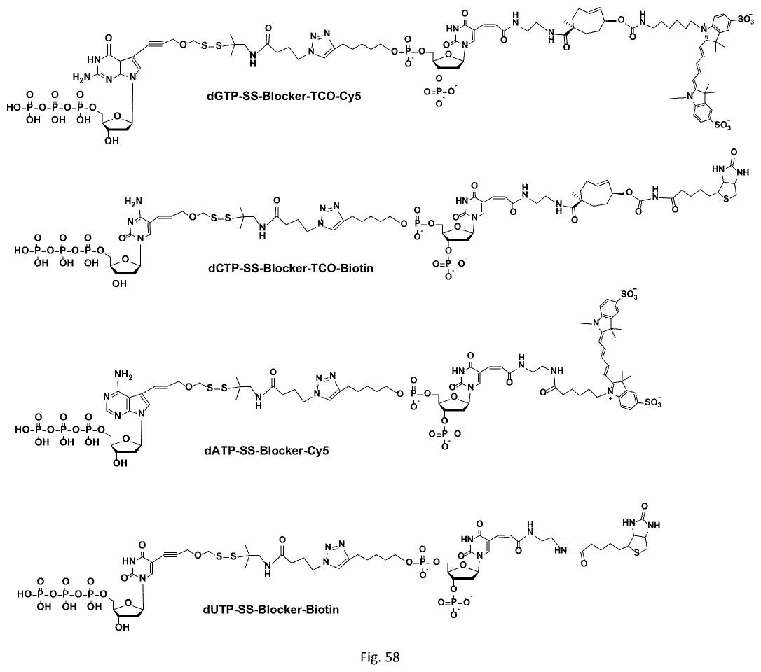

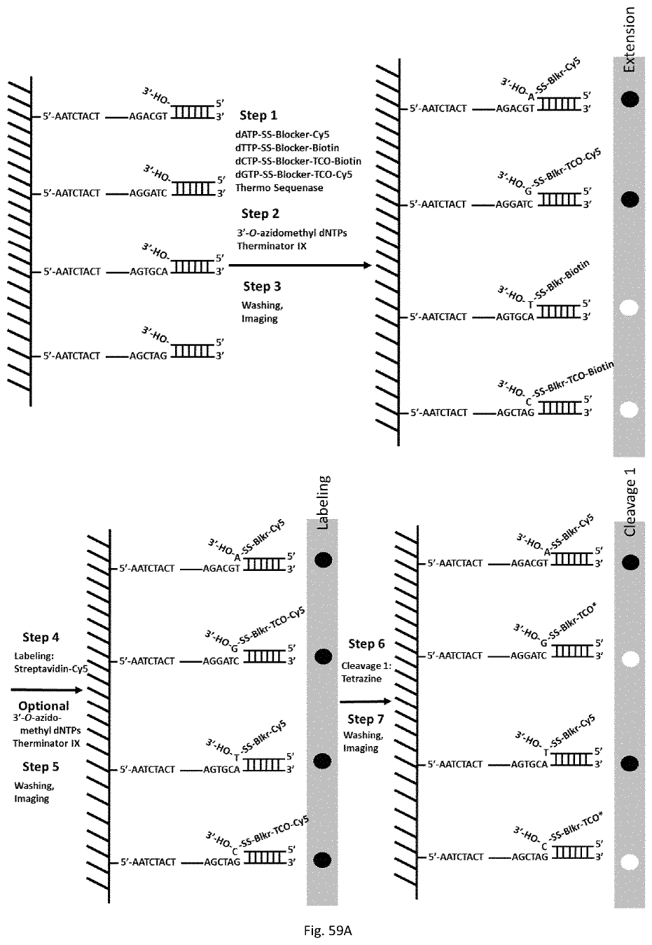

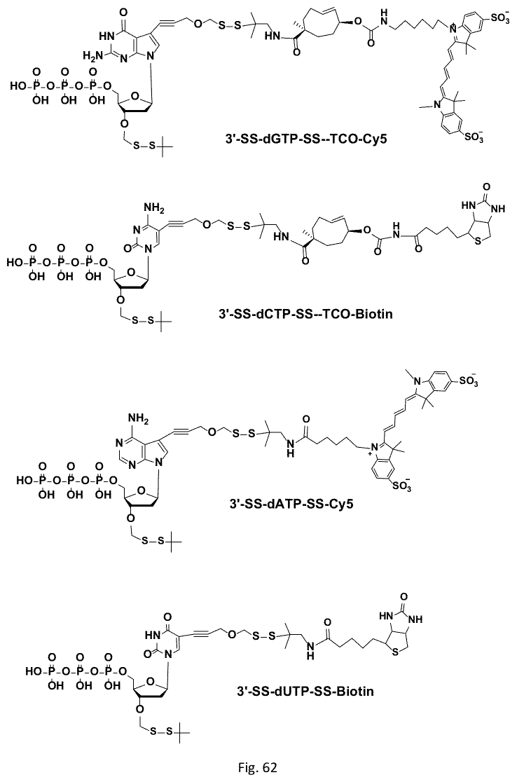

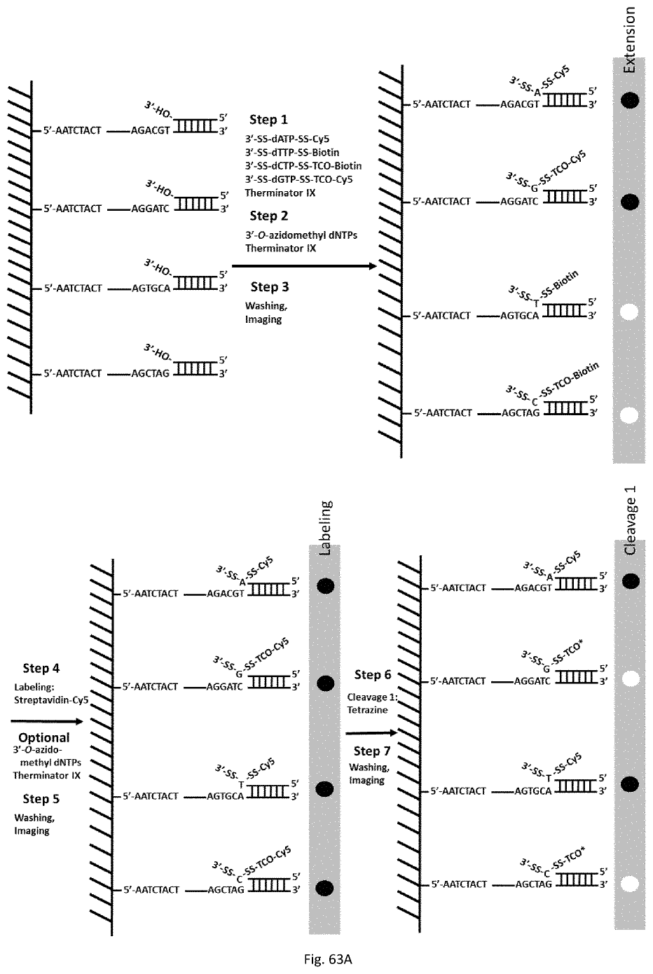

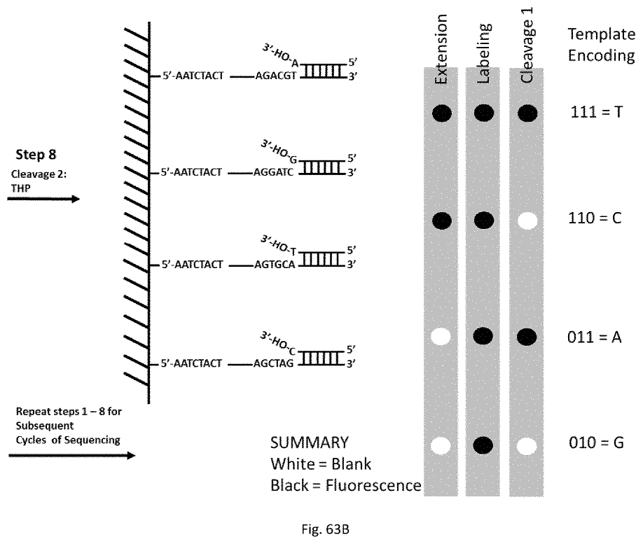

In an embodiment, the nucleotide analogues of step (b) are selected from the group consisting of the nucleotide analogues of FIG. 54, FIG. 58 or FIG. 62.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via the first cleavable linker and a carbamyl TCO linker attached distal to the first cleavable linker, (C) a pH-responsive fluorescently labeled dideoxynucleotide analogue comprising a base and a pH-responsive fluorescent label linked to the base via the first cleavable linker and a carbamyl TCO linker attached distal to the first cleavable linker, and (D) a pH-responsive fluorescently labeled dideoxynucleotide analogue comprising a base and a pH-responsive fluorescent label attached to the base via the first cleavable linker;
   (ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, (C) a pH-responsive fluorescently labeled nucleotide analogue comprising a base and a pH-responsive fluorescent label attached to the base via the first cleavable linker, and a blocking group at the 3′-OH position, wherein said blocking group prevents incorporation of a subsequent nucleotide analogue into the extended primer strand, and (D) a pH-responsive fluorescently labeled nucleotide analogue comprising a base and a pH-responsive fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, wherein the 3′-OH blocking group and the first cleavable linker are cleavable by the same agent; or
   (iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via the first cleavable linker, and a fluorescent label linked to the base via a carbamyl TCO linker distal to the blocking group, wherein said blocking group prevents or greatly reduces incorporation of a subsequent nucleotide analogue into the extended primer strand, (C) a pH-responsive fluorescently labeled nucleotide analogue nucleotide analogue comprising a base, a blocking group linked to the base via a first cleavable linker, and a pH-responsive fluorescent label linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a the first cleavable linker, and a pH-responsive fluorescent label linked to the base via a carbamyl TCO linker distal to the blocking group, wherein said blocking group prevents or greatly reduces incorporation of a subsequent nucleotide analogue into the extended primer strand,
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label does not have the same or similar absorption and emission profile as the fluorescent label on the fluorescently labeled nucleotide analogues and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b), wherein steps (d) and (e) may be performed in the reverse order;
f) contacting the incorporated nucleotide analogue with tetrazine to click the TCO moiety of the carbamyl TCO linker to release any label linked by a carbamyl TCO linker;
g) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced,
   thereby obtaining the sequence of the nucleic acid.

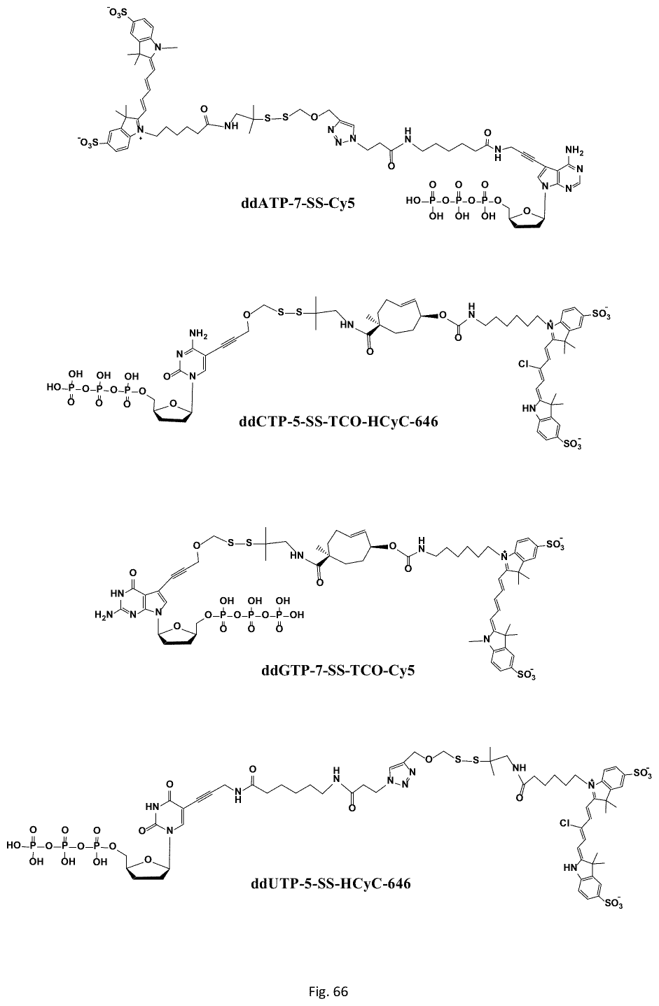

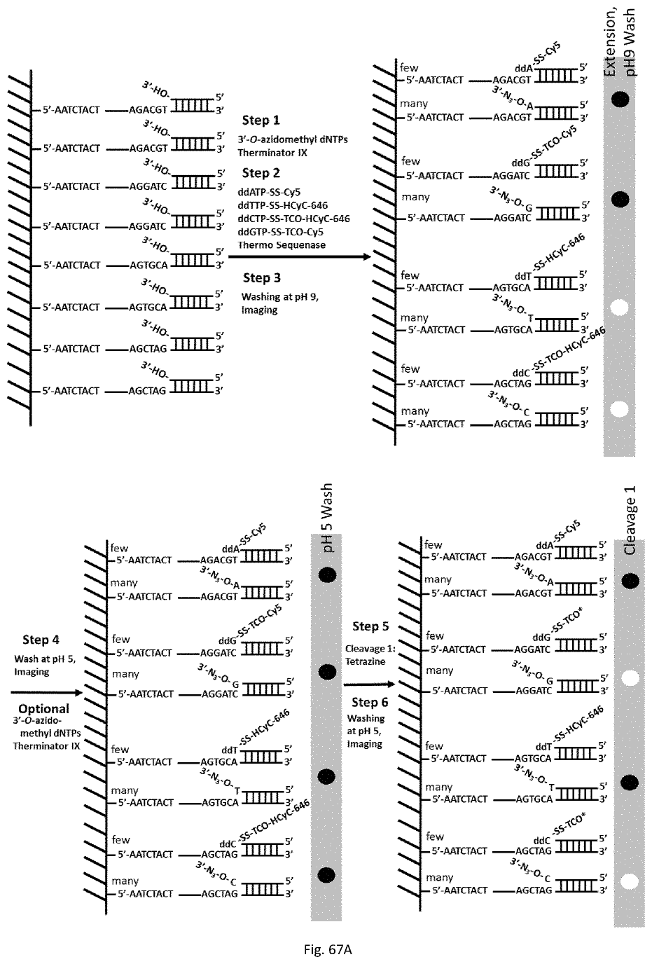

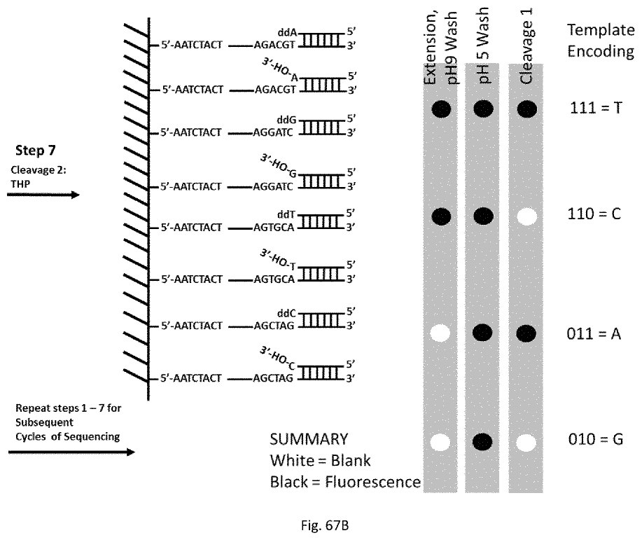

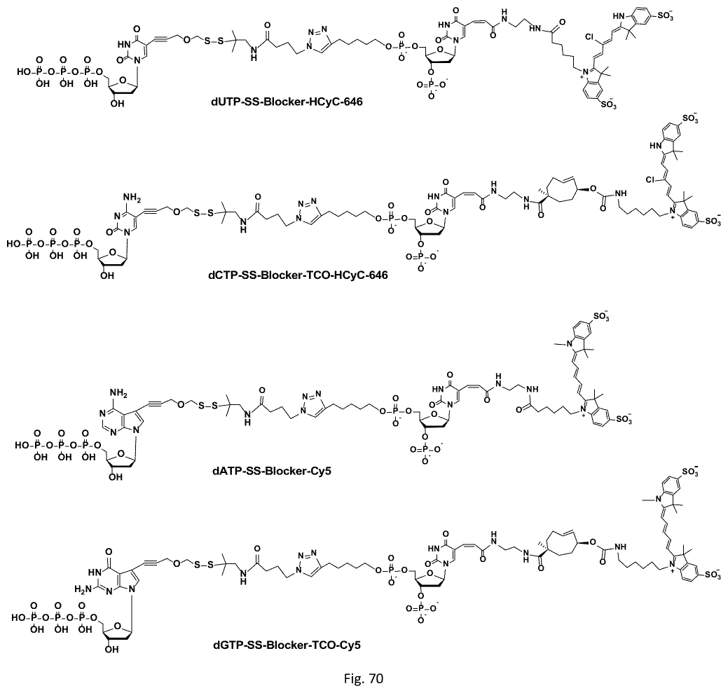

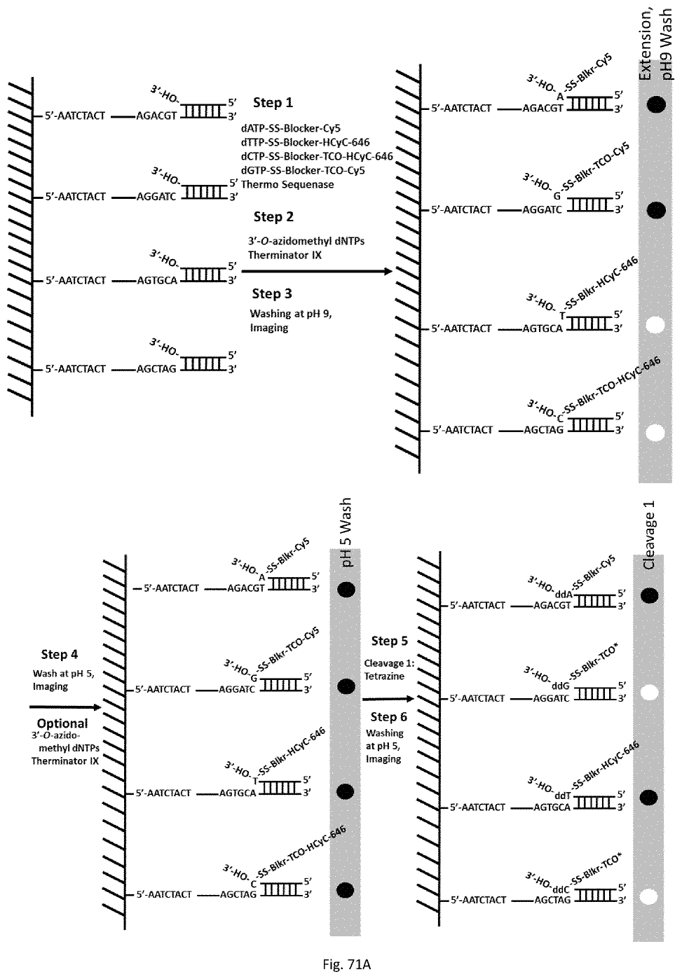

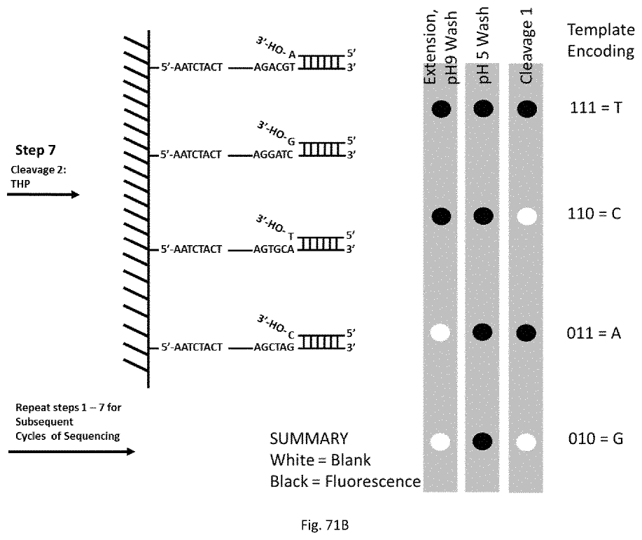

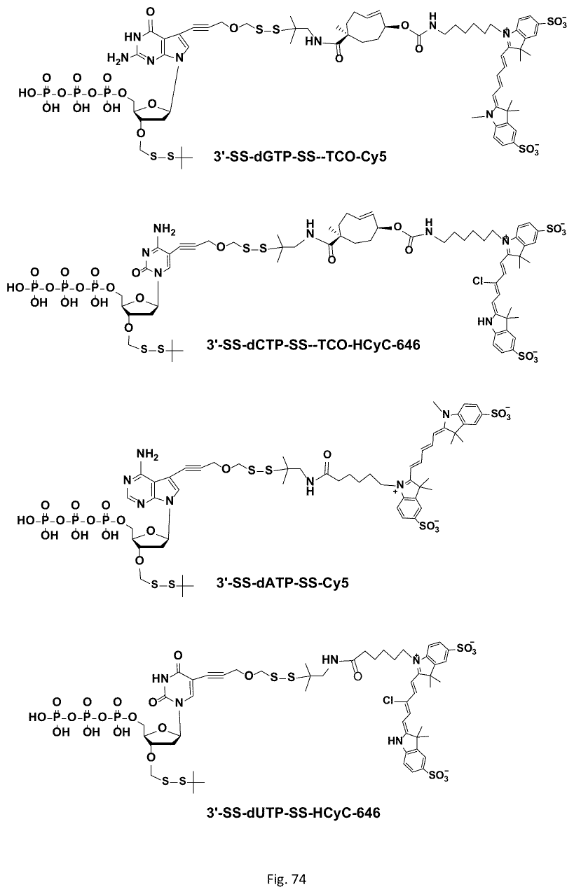

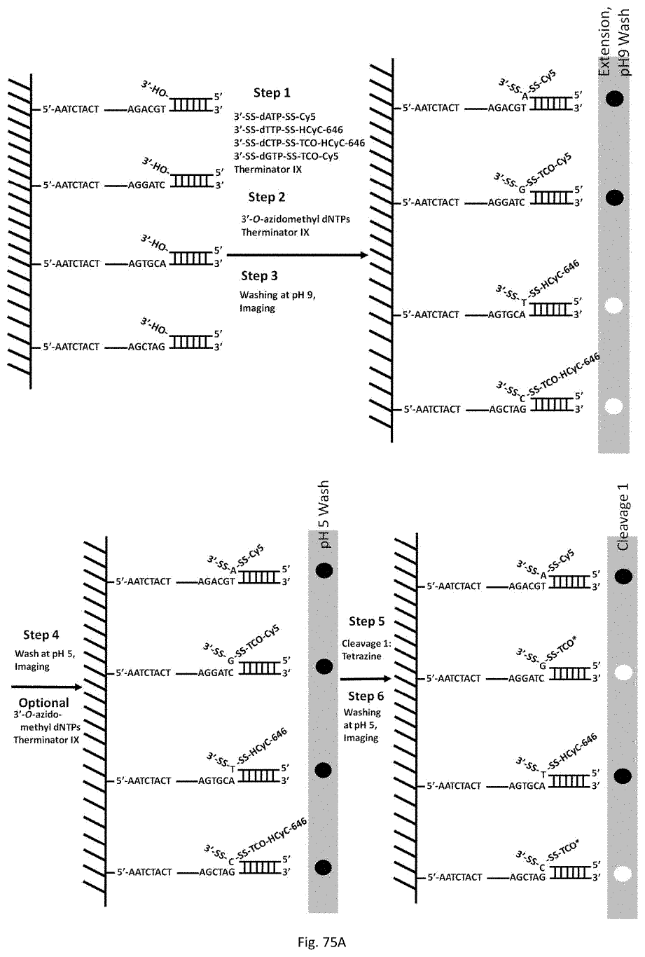

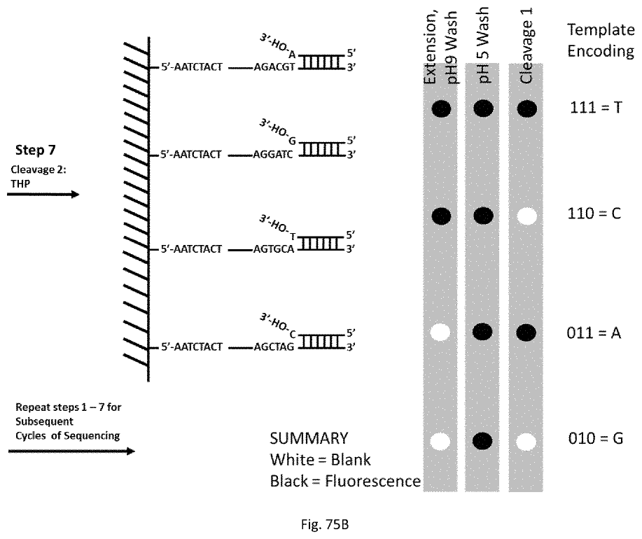

In an embodiment, the nucleotide analogues of step (b) are selected from the group consisting of the nucleotide analogues of FIG. 66, FIG. 70 or FIG. 74. In an embodiment, the label with pH-responsive fluorescence is HCyC-646 and the label with pH-unresponsive fluorescence is Cy5.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a cleavable linker, (B) a fluorescently labeled dideoxynucleotide analogue comprising a base and a fluorescent label and an anchor linked to the base via a cleavable linker, (C) a pH-responsive fluorescently labeled dideoxynucleotide analogue comprising a base and a pH-responsive fluorescent label linked to the base via a cleavable linker, and (D) a pH-responsive fluorescently labeled dideoxynucleotide analogue comprising a base and pH-responsive fluorescent label and anchor attached to the base via a cleavable linker, wherein the cleavable linkers are cleavable by an identical cleaving agent;
   (ii) (A) a fluorescently labeled nucleotide analogue comprising a base and a fluorescent label linked to the base via a cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label and an anchor linked to the base via a cleavable linker, and a blocking group at the 3′-OH position, wherein said blocking group prevents incorporation of a subsequent nucleotide analogue into the extended primer strand, (C) a pH-responsive fluorescently labeled nucleotide analogue comprising a base and pH-responsive fluorescent label linked to the base via a cleavable linker, and a blocking group at the 3′-OH position, wherein said blocking group prevents incorporation of a subsequent nucleotide analogue into the extended primer strand, and (D) a pH-responsive fluorescently labeled nucleotide analogue comprising a base and a pH-responsive fluorescent label and anchor attached to the base via a cleavable linker, and a blocking group at the 3′-OH position, wherein said blocking group prevents incorporation of a subsequent nucleotide analogue into the extended primer strand, wherein the cleavable linkers and the 3′-O blocking group are cleavable by an identical cleaving agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, (C) a pH-responsive fluorescently labeled nucleotide analogue comprising a base, a blocking group linked to the base via a cleavable linker, a pH-responsive fluorescent label linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a cleavable linker, and a pH-responsive fluorescent label and an anchor linked to the base distal to the blocking group, wherein said blocking group prevents or greatly reduces incorporation of a subsequent nucleotide analogue into the extended primer strand, wherein the cleavable linkers are cleavable by an identical cleavage agent,
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label does not have the same or similar absorption and emission profile as the fluorescent label on the fluorescently labeled nucleotide analogues and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b), wherein steps (d) and (e) may be performed in the reverse order;
f) contacting the incorporated nucleotide analogue from step (b) with an anchor binding group that binds to the anchor of the nucleotide analogues of step (b), wherein said anchor binding group comprises a moiety that quenches the fluorescent label of the fluorescently labeled nucleotide analogues of step (b);
g) washing away any unbound anchor binding group comprising a quenching moiety at a pH at which the pH-responsive fluorescent label has same or similar absorption and emission profile as the fluorescent label on the fluorescently labeled nucleotide analogues and identifying any fluorescence signal due to incorporation of a labeled nucleotide analogue from step (b);
h) contacting the incorporated nucleotide analogue with a cleaving agent that cleaves the cleavable linker and any 3′-O blocking group; and
i) iteratively repeating steps (b) to (h) for each residue of the nucleic acid to be sequenced,
   thereby obtaining the sequence of the nucleic acid.

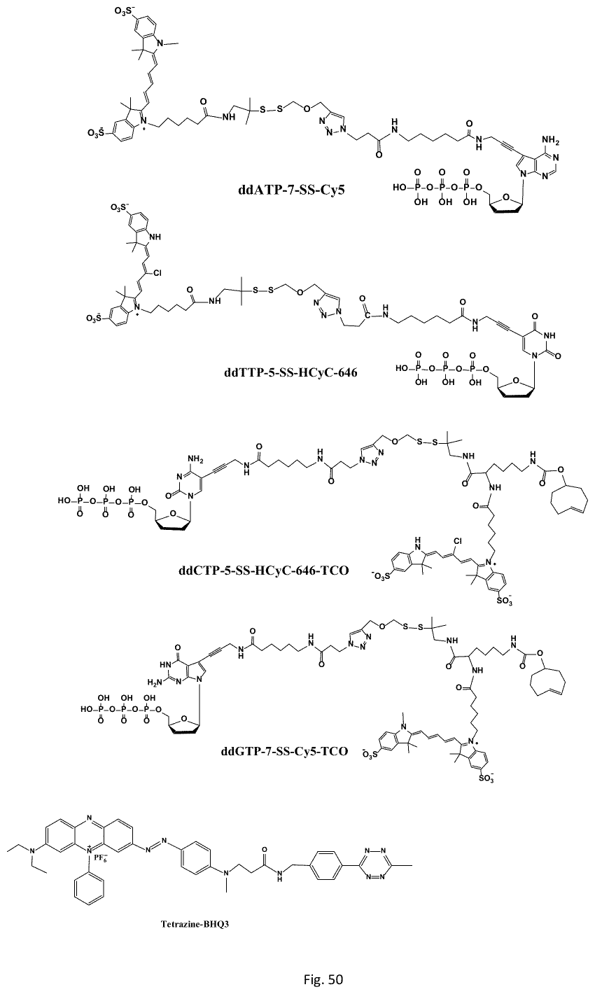

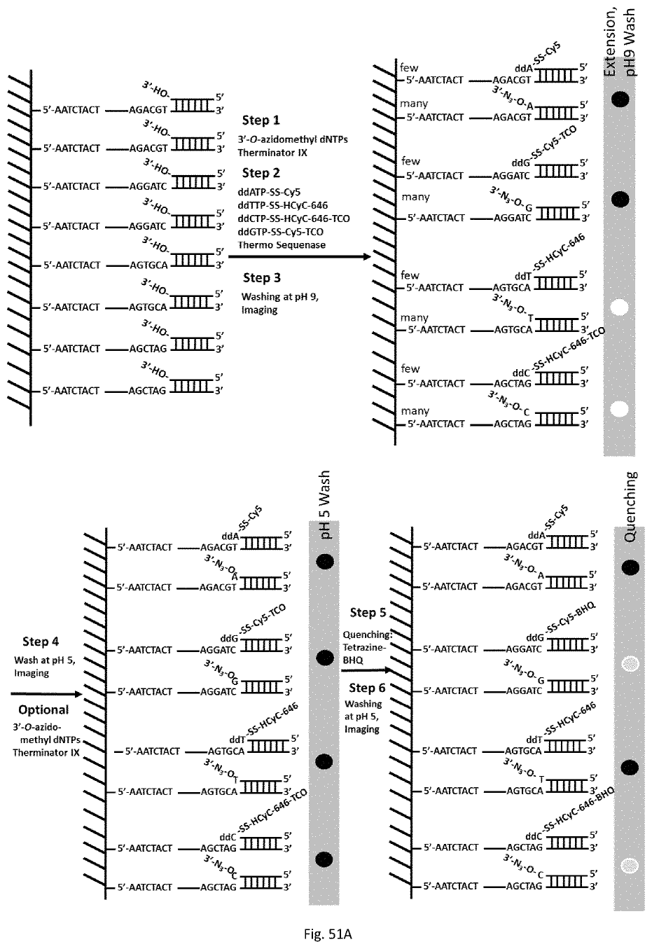

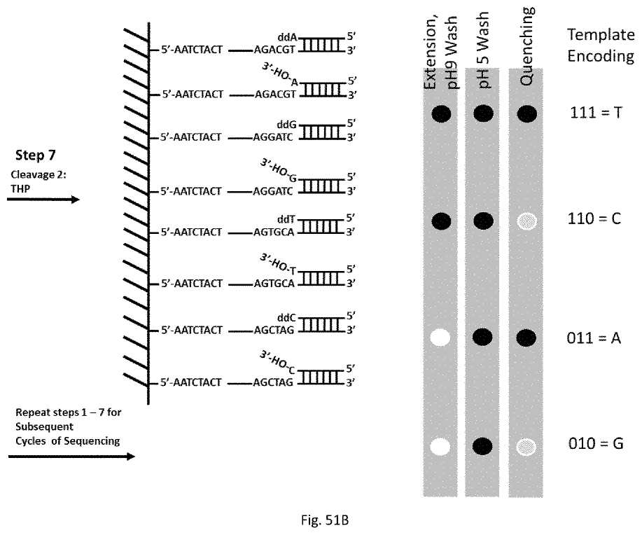

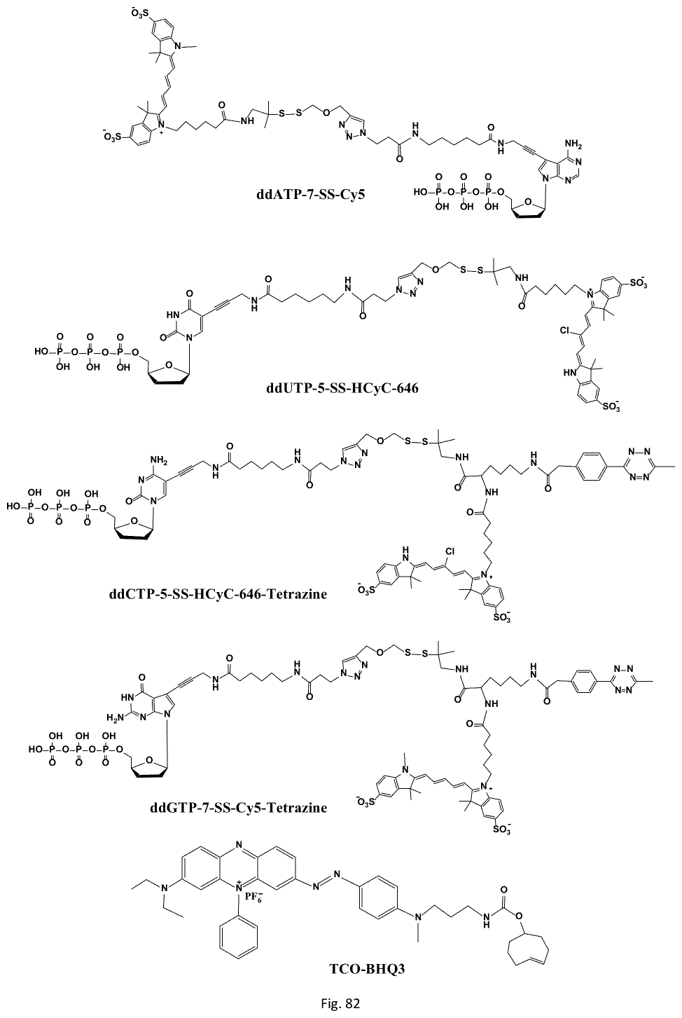

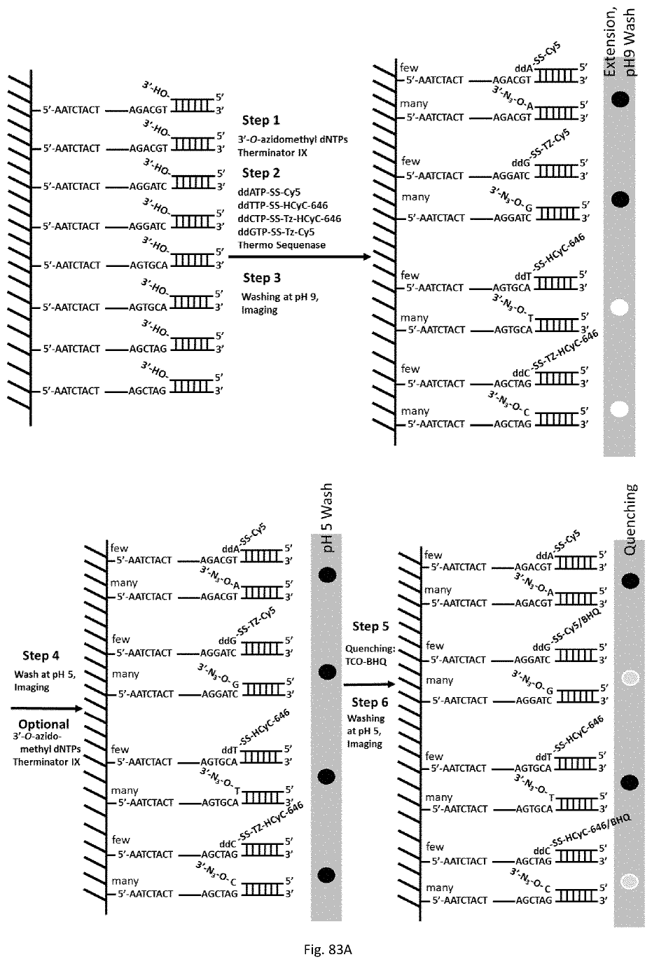

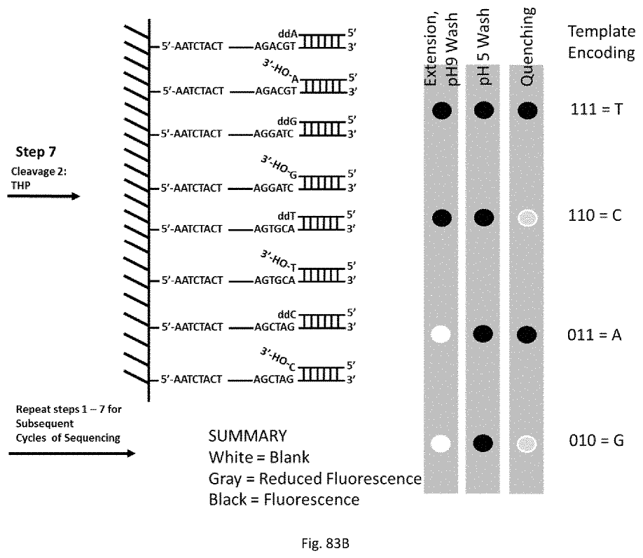

In an embodiment, the nucleotide analogues of step (b) are selected from the group consisting of the nucleotide analogues of FIG. 50 or FIG. 82. In an embodiment, the label with pH-responsive fluorescence is HCyC-646 and the label with pH-unresponsive fluorescence is Cy5.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) (A) a fluorescently labeled dideoxynucleotide analogue comprising a base and a fluorescent label linked to the base via a cleavable linker and (B) a fluorescently labeled dideoxynucleotide analogue comprising a base and a fluorescent label and anchor linked to the base via a cleavable linker, wherein the cleavable linkers are cleavable by the identical cleavage agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a fluorescently labeled nucleotide analogue comprising a base and a fluorescent label and anchor attached to the base via a cleavable linker and a blocking group at the 3′-OH position, wherein said blocking group prevents incorporation of a subsequent nucleotide analogue into the extended primer strand, wherein the cleavable linkers and 3′-O blocking group are cleavable by the identical cleavage agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, wherein the cleavable linkers are cleavable by the identical cleavage agent,
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) repeating steps (b)-(d) with two different labeled nucleotide analogues that are different from the two different labeled nucleotide analogues from the previous iteration of step (b);
f) contacting the incorporated nucleotide analogue from step (b) with an anchor binding group that binds to the anchor of the nucleotide analogues of step (b), wherein said anchor binding group comprises a moiety that quenches the fluorescent label of the fluorescently labeled nucleotide analogues of step (b);
g) identifying any fluorescence signal due to incorporation of a fluorescently labeled nucleotide analogue in step (b);
h) cleaving the cleavable linkers from the incorporated nucleotide analogue, thereby removing any label, anchor, or blocking group from the incorporated nucleotide analogue of step (b);
i) cleaving the 3′-O blocking group from any incorporated nucleotide analogue from step (c); and
j) iteratively repeating steps (b) to (i) for each residue of the nucleic acid to be sequenced,
   thereby obtaining the sequence of the nucleic acid.

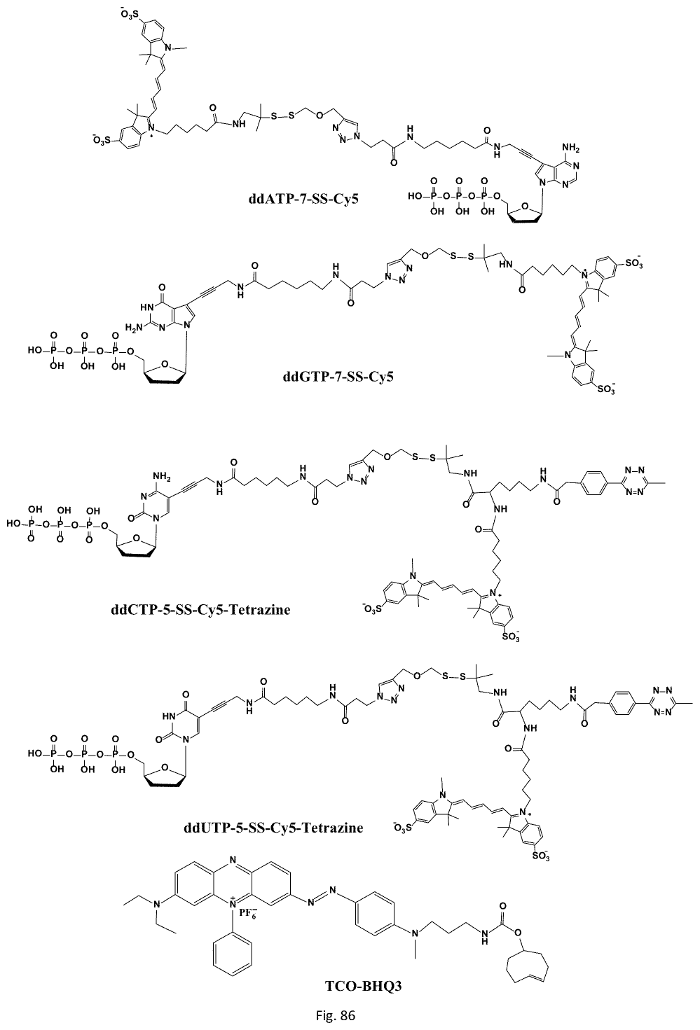

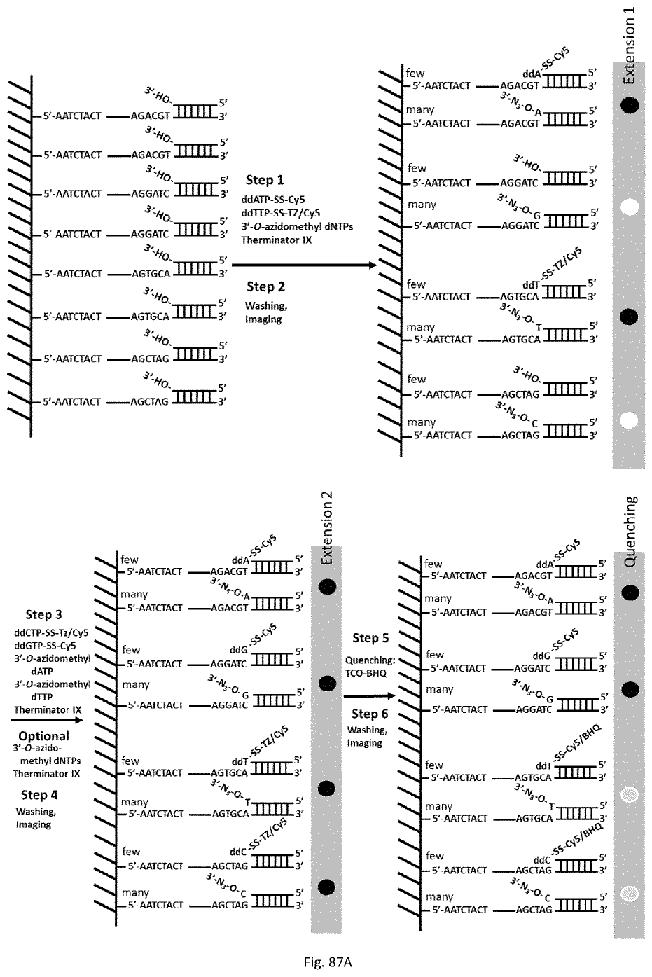

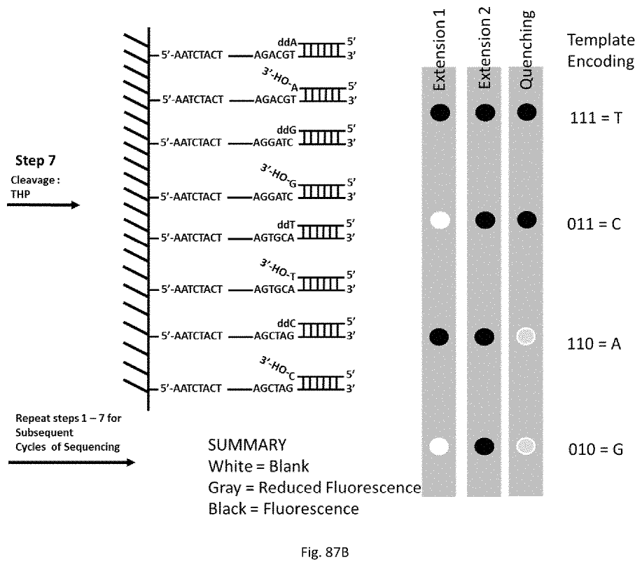

In an embodiment, the nucleotide analogues of step (b) are selected from the group consisting of the nucleotide analogues of FIG. 86.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are:
   (A) fluorescently labeled dideoxynucleotide analogue comprising a base and a fluorescent label linked to the base via a cleavable linker, (B) fluorescently labeled dideoxynucleotide analogue comprising a base and a fluorescent label linked to the base via an uncleavable linker, (C) an anchor labeled dideoxynucleotide analogue comprising a base and an anchor attached to the base via a cleavable linker, and (D) an anchor labeled dideoxynucleotide analogue comprising a base and an anchor attached to the base via an uncleavable linker;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue in step (b);
e) contacting the incorporated nucleotide analogue from step (b) with an anchor binding group that binds to the anchor of the anchor labeled nucleotide analogues of step (b);
f) identifying any fluorescence signal due to incorporation of a fluorescently labeled nucleotide analogue in step (b);
g) contacting the incorporated nucleotide analogue of step (b) with an agent that cleaves the cleavable linker of the nucleotide analogues of step (b) and cleaves the 3′-O blocking group of the nucleotide analogues of step (c);
h) identifying any fluorescence signal due to incorporation of a fluorescently labeled nucleotide analogue in step (b);
i) photobleaching the incorporated nucleotide analogue of step (b) to thereby photobleach any remaining fluorescent label; and
j) iteratively repeating steps (b) to (i) for each residue of the nucleic acid to be sequenced,
   thereby obtaining the sequence of the nucleic acid.

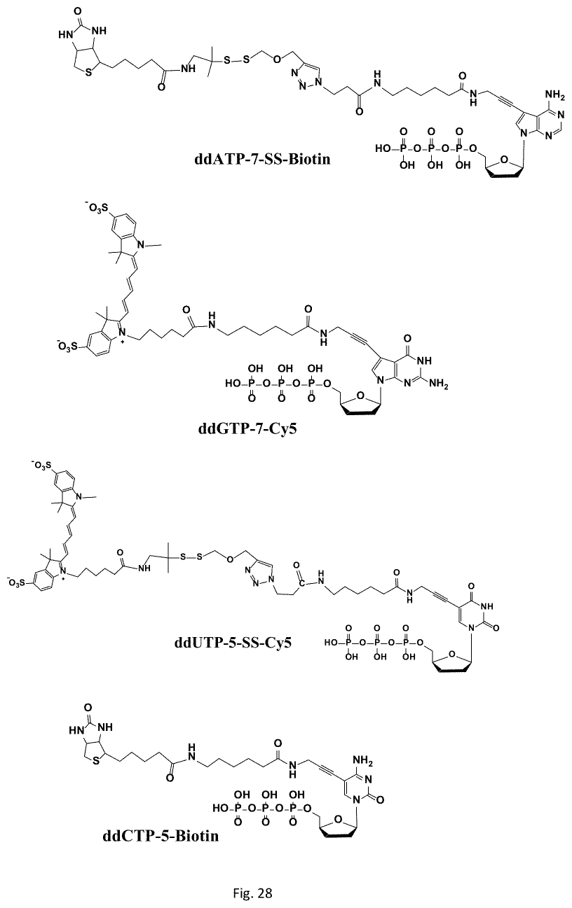

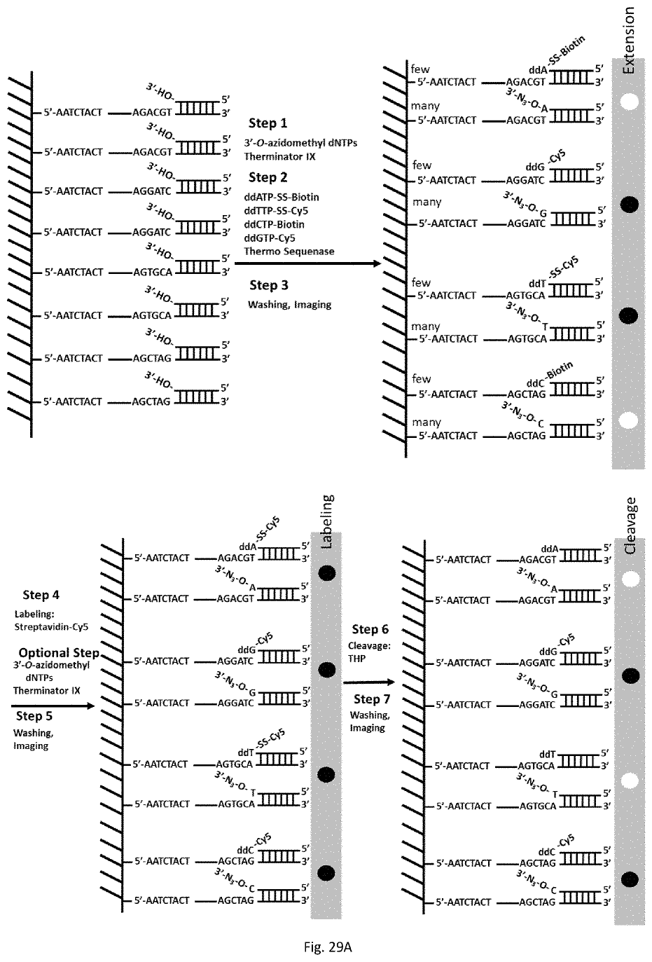

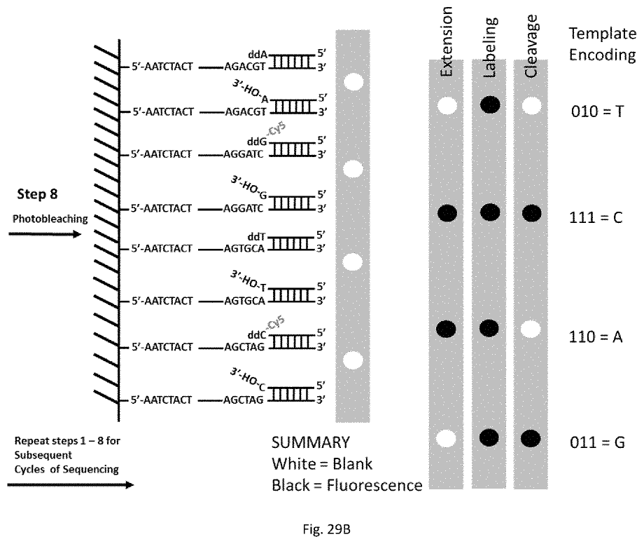

In an embodiment, the nucleotide analogues of step (b) are selected from the group consisting of the nucleotide analogues of FIG. 28. In an embodiment, step (c) occurs before step (b). In an embodiment, step (c) occurs after step (b). In an embodiment, the nucleotide analogues added in step (c) are incorporated into primers of greater than 90% the nucleic acid templates. In an embodiment, the nucleotide analogues added in step (c) are incorporated into primers of greater than 95% the nucleic acid templates.

In an embodiment, if there is an anchor present, the anchor comprises biotin, TCO, tetrazine, or DBCO, and the corresponding anchor binding molecule comprises streptavidin, azide, tetrazine and TCO. In an embodiment, the fluorescent dye comprises organic dyes comprising xanthine, cyanine and ATTO dyes, quantum dots and clusters of organic dyes and quantum dots.

The invention provides a method of sequencing a nucleic acid comprising:
a) providing a plurality of nucleic acid templates each hybridized to a primer and a nucleic acid polymerase, wherein each template has the same sequence as the nucleic acid to be sequenced;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) a fluorescently labeled dideoxynucleotide analogue comprising a base and a fluorescent label linked to the base via a cleavable linker and (B) a fluorescently labeled dideoxynucleotide analogue comprising a base and a different fluorescent label linked to the base via a cleavable linker, wherein the cleavable linkers are cleavable by an identical cleavage agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a fluorescently labeled nucleotide analogue comprising a base and a different fluorescent label attached to the base via a cleavable linker and a blocking group at the 3′-OH position, wherein the cleavable linkers and 3′-O blocking groups are cleavable by the identical cleavage agent; or
   (iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, and (B) a fluorescently labeled nucleotide analogue comprising a base, a blocking group linked to the base via a cleavable linker, and a different fluorescent label linked to the base distal to the blocking group, wherein said blocking group prevents or greatly reduces incorporation of a subsequent nucleotide analogue into the extended primer strand, wherein the cleavable linkers are cleavable by an identical cleavage agent;
c) contacting the nucleic acid templates with unlabeled nucleotide analogues (A, C, T, G) without any base modifications and comprising a 3′-O blocking group, wherein said 3′-O blocking groups are cleavable by an identical cleavage agent to the cleavable linkers and and/or blocking groups of the two labeled nucleotide analogues of step (b), and extending any unextended primers with said unlabeled nucleotide analogues, wherein step (c) occurs before, simultaneously, or after step (b);
d) identifying any fluorescence signal due to incorporation of a fluorescently labeled nucleotide analogue;
e) repeating steps (b)-(d) with two different labeled nucleotide analogues that are different from the two different labeled nucleotide analogues from the previous iteration of step (b), but with only two unlabeled nucleotides comprising a 3′-O blocking group different from the two labeled nucleotide analogues added in this step;
f) cleaving the cleavable linkers from the incorporated nucleotide analogue, thereby removing any label or blocking group from the incorporated nucleotide analogue of step (b) and (c);
g) identifying any fluorescence signal due to incorporation of a fluorescently labeled nucleotide analogue in step (b); and
h) iteratively repeating steps (b) to (g) for each residue of the nucleic acid to be sequenced,
   thereby obtaining the sequence of the nucleic acid.

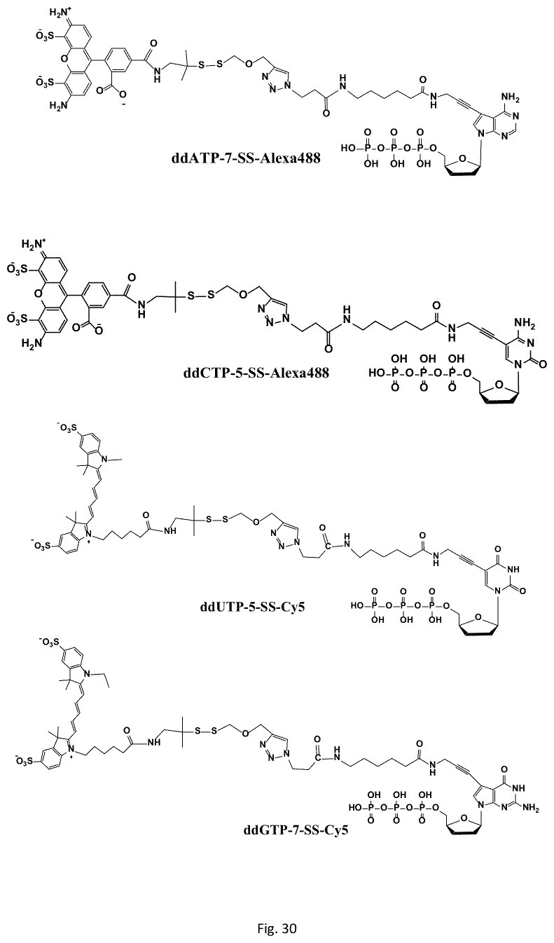

In an embodiment, the nucleotide analogues of step (b) are selected from the group consisting of the nucleotide analogues of FIG. 30.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) (A) a fluorescently labeled dideoxynucleotide analogue comprising a base and a fluorescent label linked to the base via a cleavable linker, (B) a pH-responsive fluorescently labeled dideoxynucleotide analogue comprising a base and a pH-responsive fluorescent label linked to the base via a cleavable linker, (C) a fluorescently labeled dideoxynucleotide analogue comprising a base and a fluorescent label and anchor linked to the base via a cleavable linker, and (D) a pH-responsive fluorescently labeled dideoxynucleotide analogue comprising a base and a pH-responsive fluorescent label and identical anchor linked to the base via a cleavable linker,
      wherein the cleavable linkers are cleavable by an identical cleaving agent;
   (ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, (B) a pH-responsive fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, (C) a fluorescently labeled nucleotide analogue comprising a base and a fluorescent label and anchor linked to the base via a cleavable linker and a blocking group at the 3′-OH position, wherein said blocking group prevents incorporation of a subsequent nucleotide analogue into the extended primer strand, and (D) a pH-responsive fluorescently labeled nucleotide analogue comprising a base and a fluorescent label and identical anchor linked to the base via a cleavable linker and a blocking group at the 3′-OH position, wherein said blocking group prevents incorporation of a subsequent nucleotide analogue into the extended primer strand,
      wherein the cleavable linkers and 3′-O blocking groups are cleavable by an identical cleaving agent; or
   (iii) a fluorescently labeled nucleotide analogue comprising a base, a blocking group linked to the base via a cleavable linker, and a fluorescent label linked to the base linker distal to the blocking group, wherein said blocking group prevents or greatly reduces incorporation of a subsequent nucleotide analogue into the extended primer strand, (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand, (C) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a cleavable linker, and a pH-responsive fluorescent label and identical anchor linked to the base distal to the blocking group, wherein said blocking group prevents or greatly reduces incorporation of a subsequent nucleotide analogue into the extended primer strand,
      wherein the cleavable linkers are cleavable by an identical cleaving agent;
c) extending unextended primers with a nucleotide analogue without any base modifications and comprising a 3′-O blocking group, wherein step (c) occurs before, simultaneously or after step (b);
d) washing away any unincorporated nucleotide analogues at a pH at which the pH-responsive fluorescent label does not have the same or similar absorption and emission profile as the fluorescent label on the fluorescently labeled nucleotide analogue and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of an anchor labeled nucleotide analogue from step (b);
f) contacting the incorporated nucleotide analogue with an anchor binding group that binds to the anchor of the nucleotide analogues of step (b) and comprises a moiety that quenches the fluorescent signal of any fluorescent label attached to the nucleotide analogue to which the anchor binding group attaches, and identifying any fluorescence signal due to incorporation of a fluorescently labeled nucleotide analogue;
g) contacting the incorporated nucleotide analogue with a cleaving agent that cleaves the cleavable linker and any 3′-O blocking group; and
h) iteratively repeating steps (b) to (g) for each residue of the nucleic acid to be sequenced,
   thereby obtaining the sequence of the nucleic acid.

In an embodiment, the nucleotide analogues of step (b) are selected from the group consisting of the nucleotide analogues of FIG. 50.

The invention provides a method of sequencing a nucleic acid comprising:
a) providing a plurality of nucleic acid templates each hybridized to a primer;
b) contacting the nucleic acid templates with two different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the two different labeled nucleotide analogues are either:
   (i) two fluorescently labeled nucleotide analogues comprising a base and a fluorescent label serving as an energy transfer donor linked to the base via a cleavable linker, an anchor for attachment of an energy transfer acceptor label, and a blocking group at the 3′-OH position, wherein said blocking group prevents incorporation of a subsequent nucleotide analogue into the extended primer strand,
      wherein the cleavable linkers and 3′-O blocking groups are cleavable by an identical cleaving agent;
      wherein each of the nucleotide analogues has a different anchor; or
   (ii) two fluorescently labeled nucleotide analogues comprising a base, a blocking group linked to the base via a cleavable linker, and a fluorescent energy transfer donor label and an anchor for attachment of energy transfer acceptor label linked to the base distal to the blocking group, wherein said blocking group prevents or greatly reduces incorporation of a subsequent nucleotide analogue into the extended primer strand,
      wherein the cleavable linkers are cleavable by an identical cleaving agent, and
      wherein each of the nucleotide analogues has a different anchor;
c) washing away any unincorporated nucleotide analogues and contacting the incorporated nucleotide analogue with two anchor binding groups that bind specifically to each of the anchors of the nucleotide analogues of step (b) and comprises a moiety that serves as an energy transfer acceptor,
   wherein said energy transfer acceptor on one of the anchor binding groups is a pH-unresponsive label and said energy transfer acceptor on the other anchor binding groups is a pH-responsive label;
d) washing away any free labels at a pH at which the pH-responsive fluorescent energy transfer acceptor dye label has the same or similar absorption and emission profile as the pH-unresponsive fluorescent energy transfer acceptor label;
e) exposing the incorporated nucleotides to a wavelength that can excite the energy transfer donor dye, and identifying any fluorescence signal due to energy transfer and emission of the energy transfer acceptor dyes attached to the nucleotide analogues due to the labeling reaction performed in step (c);
f) repeating steps (b) to (e) with two different labeled nucleotide analogues than the two different labeled nucleotide analogues in (b), but otherwise having all other properties described in (b);
g) changing the buffer to a pH at which the pH-responsive fluorescent label does not have the same or similar absorption and emission profile as the pH-unresponsive fluorescent label on the fluorescently labeled nucleotide analogue and identifying any fluorescence signal due to incorporation of an anchor labeled nucleotide analogue from steps (b) or (f), wherein the order of steps (e) and (g) may be reversed;
h) contacting the incorporated nucleotide analogue with a cleaving agent that cleaves the cleavable linker and the 3′-O blocking group; and
i) iteratively repeating steps (b) to (h) for each residue of the nucleic acid to be sequenced,
   thereby obtaining the sequence of the nucleic acid.

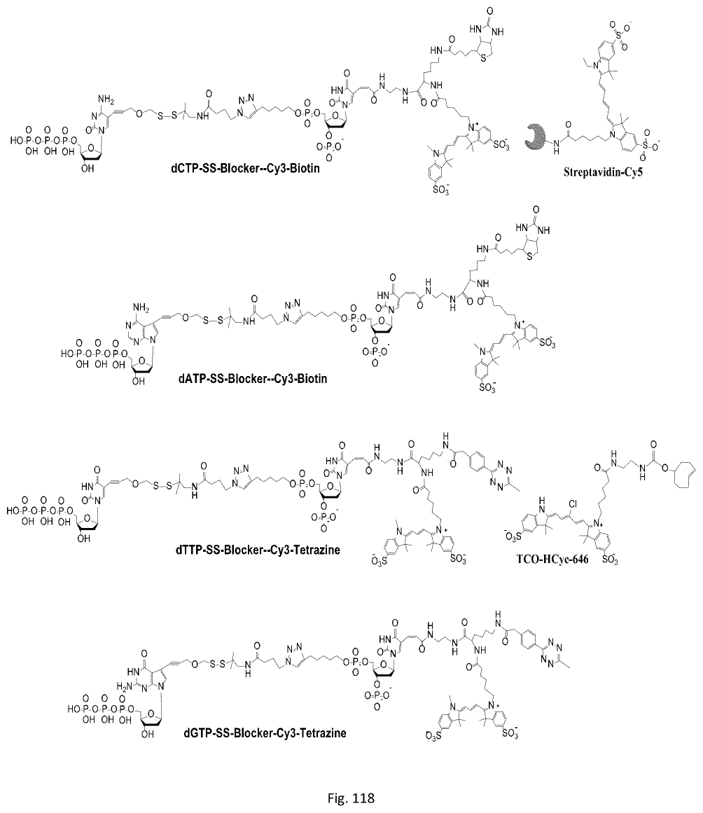

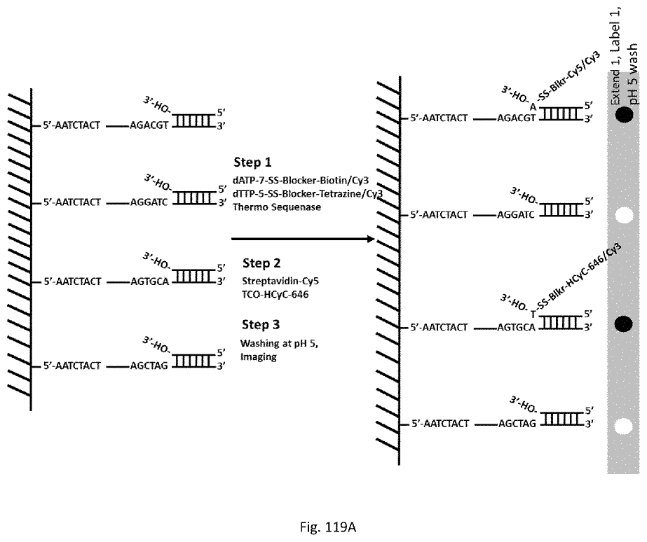

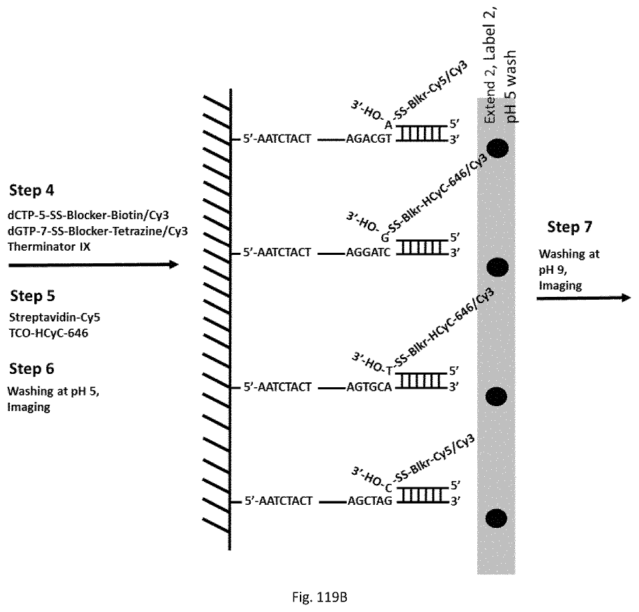

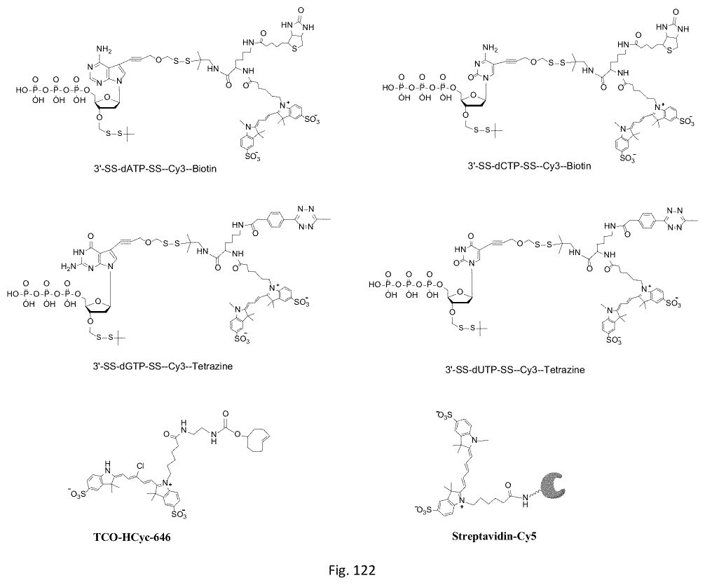

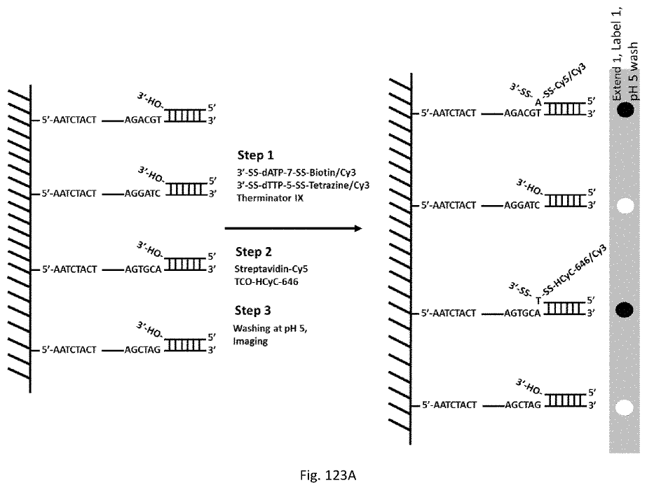

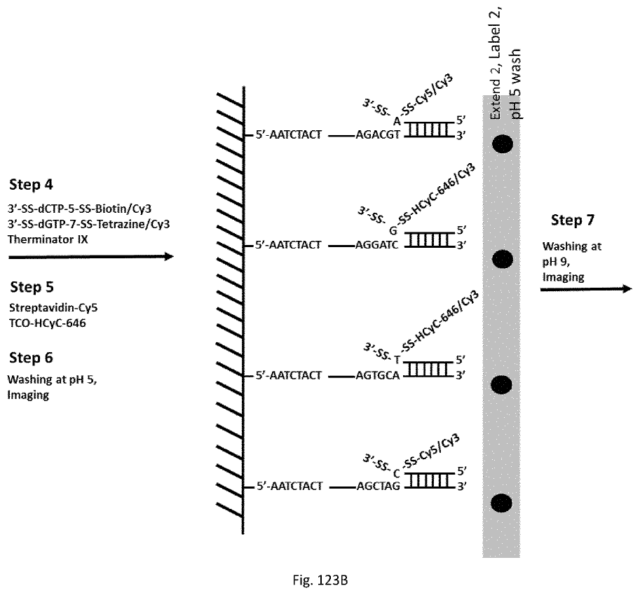

In an embodiment, the nucleotide analogues of step (b) are selected from the group consisting of the nucleotide analogues of FIG. 118 or FIG. 122.

The invention provides a method of sequencing a nucleic acid comprising:
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, G, T)) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
   (i) a fluorescently labeled nucleotide analogue comprising a base and a fluorescent label serving as an energy transfer donor and an anchor (anchor 1) for attachment of a pH unresponsive energy transfer acceptor label, linked to the base via a first cleavable linker (cleavable linker 1), and a blocking group at the 3′-OH position, wherein said blocking group prevents incorporation of a subsequent nucleotide analogue into the extended primer strand, (B) a fluorescently labeled nucleotide analogue comprising a base and both a fluorescent label serving as an energy transfer donor and a second anchor (anchor 2) for attachment of a pH-responsive energy transfer acceptor label linked to the base via the same cleavable linker (cleavable linker 1), and a blocking group at the 3′-OH position, wherein said blocking group prevents incorporation of a subsequent nucleotide analogue into the extended primer strand, (C) a fluorescently labeled nucleotide analogue comprising a base and both a fluorescent label serving as an energy transfer donor and the first anchor (anchor 1) for attachment of a pH-unresponsive energy transfer acceptor label linked to the base via a second cleavable linker (cleavable linker 2), and a blocking group at the 3′-OH position, wherein said blocking group prevents incorporation of a subsequent nucleotide analogue into the extended primer strand, and (D) a fluorescently labeled nucleotide analogue comprising a base and both a fluorescent label serving as an energy transfer donor linked to the base via the second cleavable linker (cleavable linker 2), the second anchor (anchor 2) for attachment of a pH-responsive energy transfer acceptor label, and a blocking group at the 3′-OH position, wherein said blocking group prevents incorporation of a subsequent nucleotide analogue into the extended primer strand,
      wherein the first cleavable linker and 3′-O blocking groups are cleavable by an identical cleaving agent, and the second cleavable linker is cleavable by a different cleaving agent; or
   (ii) (A) a fluorescently labeled nucleotide analogue comprising a base, a blocking group linked to the base via a first cleavable linker (cleavable linker 1), and a fluorescent energy transfer donor label and an anchor (anchor 1) for attachment of a pH-unresponsive energy transfer acceptor label linked to the base linker distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a first cleavable linker (cleavable linker 1), and a fluorescent energy transfer donor label and a second anchor (anchor 2) for attachment of a pH-responsive energy transfer acceptor label linked to the base linker distal to the blocking group, wherein said blocking group prevents or greatly reduces incorporation of a subsequent nucleotide analogue into the extended primer strand, (C) a fluorescently labeled nucleotide analogue comprising a base, a blocking group linked to the base via a second cleavable linker (cleavable linker 2), and a fluorescent energy transfer donor label and the first anchor (anchor 1) for attachment of a pH-unresponsive energy transfer acceptor label linked to the base linker distal to the blocking group, wherein said blocking group prevents or greatly reduces incorporation of a subsequent nucleotide analogue into the extended primer strand, and (D) a fluorescently labeled nucleotide analogue comprising a base, a blocking group linked to the base via a second cleavable linker (cleavable linker 2), and a fluorescent energy transfer donor label and the second anchor (anchor 2) for attachment of a pH-responsive energy transfer acceptor label linked to the base linker distal to the blocking group, wherein said blocking group prevents or greatly reduces incorporation of a subsequent nucleotide analogue into the extended primer strand,
      wherein each cleavable linker is cleavable by a different cleaving agent;
c) washing away any unincorporated nucleotide analogues and contacting the incorporated nucleotide analogue with two anchor binding groups that bind specifically to each of the anchors of the nucleotide analogues of step (b) and comprises a moiety that serves as an energy transfer acceptor,
   wherein said energy transfer acceptor on one of the anchor binding groups is a pH-unresponsive label and said energy transfer acceptor on the other anchor binding groups is a pH-responsive label;
d) washing away any free labels at a pH at which the pH-responsive fluorescent energy transfer acceptor dye label has the same or similar absorption and emission profile as the pH-unresponsive fluorescent energy transfer acceptor label;
e) exposing the incorporated nucleotides to a wavelength that can excite the energy transfer donor dye, and identifying any fluorescence signal due to energy transfer and emission of the energy transfer acceptor dyes attached to the nucleotide analogues incorporated in step (b) due to the labeling reaction performed in step (c);
f) changing the buffer to a pH at which the pH-responsive fluorescent label does not have the same or similar absorption and emission profile as the pH-unresponsive fluorescent label on the fluorescently labeled nucleotide analogue and identifying any fluorescence signal due to incorporation of an anchor labeled nucleotide analogue from step (b) due to the labeling reaction performed in step (c), wherein steps (d) and (f) may be reversed;
g) contacting the incorporated nucleotide analogue with a cleaving agent that cleaves the second cleavable linker;
h) washing away the cleaving agent and released labels at a pH at which the pH-responsive fluorescent energy transfer acceptor dye label has the same or similar absorption and emission profile as the pH-unresponsive fluorescent energy transfer acceptor label;
i) repeating step (e);
j) contacting the incorporated nucleotide analogue with a cleaving agent that cleaves the first cleavable linker and the 3′-O blocking group; and
k) iteratively repeating steps (b) to (j) for each residue of the nucleic acid to be sequenced,
   thereby obtaining the sequence of the nucleic acid.

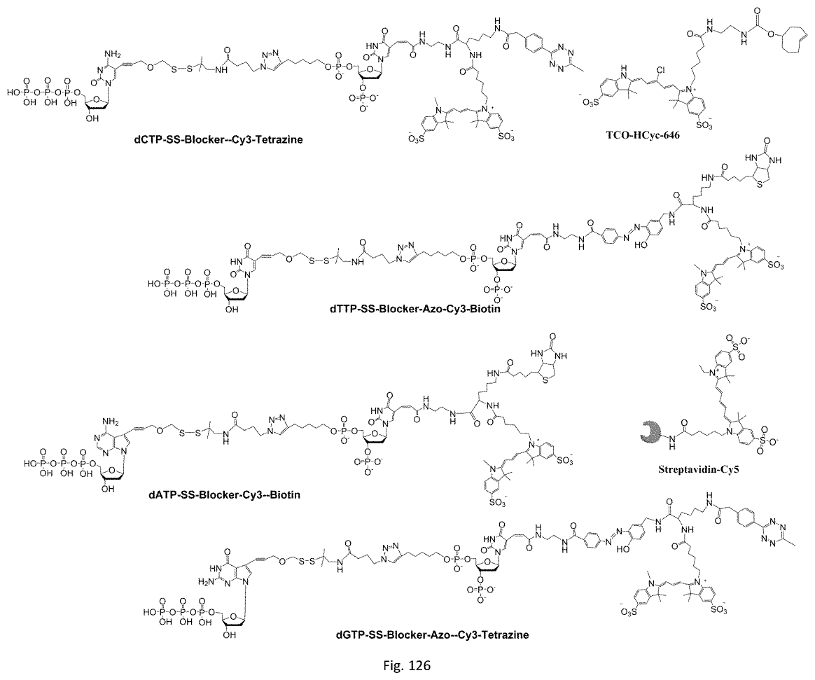

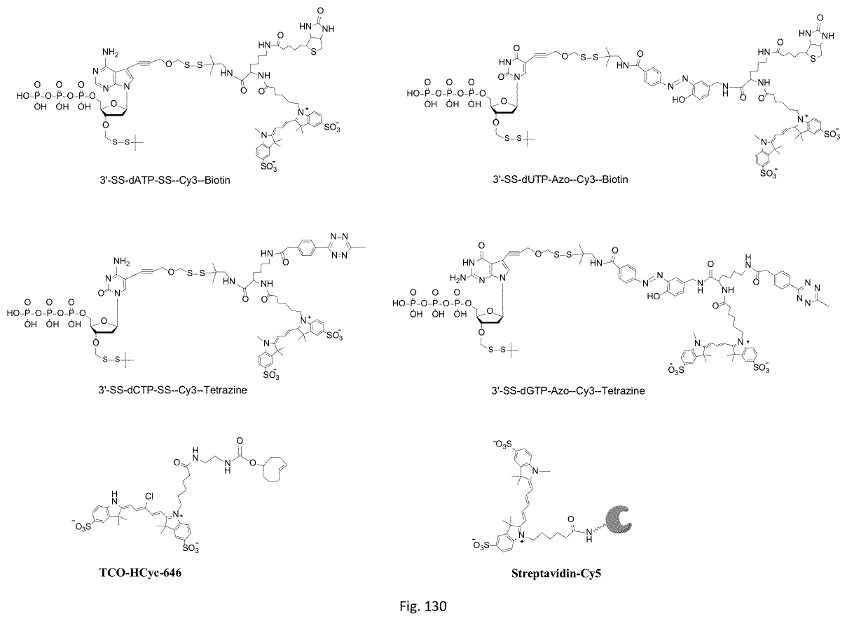

In an embodiment, the nucleotide analogues of step (b) are selected from the group consisting of the nucleotide analogues of FIG. 126 or FIG. 130.

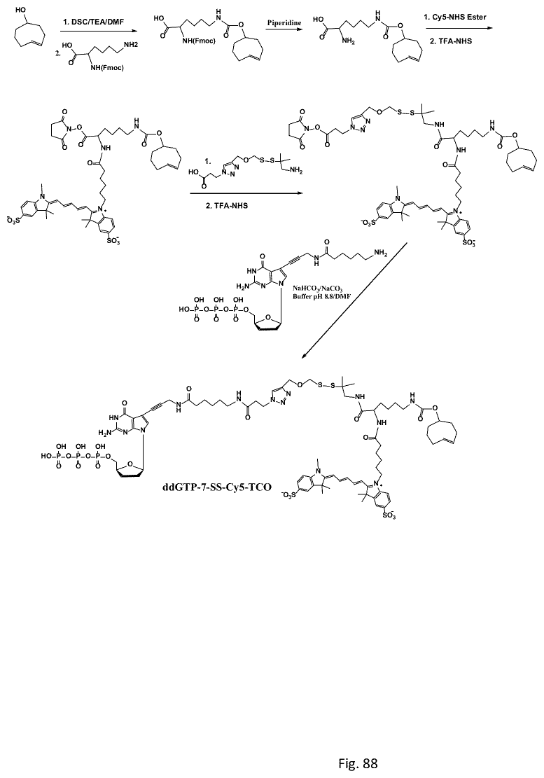

The invention provides a method for synthesizing a nucleotide analogue according to the protocol of any one of FIGS. 88-103.

### Terms

The abbreviations used herein have their conventional meaning within the chemical and biological arts. The chemical structures and formulae set forth herein are constructed according to the standard rules of chemical valency known in the chemical arts.

As used herein, and unless stated otherwise, each of the following terms shall have the definition set forth below.

### A—Adenine;

### C—Cytosine;

### G—Guanine;

### T—Thymine;

### U—Uracil;

DNA—Deoxyribonucleic acid;
RNA—Ribonucleic acid;

Throughout this application, U and T are sometimes used interchangeably, depending on context, e.g., U is generally used when referring to chemical structure drawings, whereas T is generally used in the figures and in the schemes.

“Nucleic acid” shall mean, unless otherwise specified, any nucleic acid molecule, including, without limitation, DNA, RNA and hybrids thereof. In an embodiment the nucleic acid bases that form nucleic acid molecules can be the bases A, C, G, T and U, as well as derivatives thereof.

“Derivatives” or “analogues” of these bases are well known in the art, and are exemplified in PCR Systems, Reagents and Consumables (Perkin Elmer Catalogue 1996-1997, Roche Molecular Systems, Inc., Branchburg, N.J., USA).

A “nucleotide residue” is a single nucleotide in the state it exists after being incorporated into, and thereby becoming a monomer of, a polynucleotide. Thus, a nucleotide residue is a nucleotide monomer of a polynucleotide, e.g. DNA, which is bound to an adjacent nucleotide monomer of the polynucleotide through a phosphodiester bond at the 3′ position of its sugar and is bound to a second adjacent nucleotide monomer through its phosphate group, with the exceptions that (i) a 3′ terminal nucleotide residue is only bound to one adjacent nucleotide monomer of the polynucleotide by a phosphodiester bond from its phosphate group, and (ii) a 5′ terminal nucleotide residue is only bound to one adjacent nucleotide monomer of the polynucleotide by a phosphodiester bond from the 3′ position of its sugar.

“Substrate” or “Surface” shall mean any suitable medium present in the solid phase to which a nucleic acid or an agent may be affixed. Non-limiting examples include chips, beads, nanopore structures and columns. In an embodiment the solid substrate can be present in a solution, including an aqueous solution, a gel, or a fluid.

“Hybridize” shall mean the annealing of one single-stranded nucleic acid to another nucleic acid based on the well-understood principle of sequence complementarity. In an embodiment the other nucleic acid is a single-stranded nucleic acid. The propensity for hybridization between nucleic acids depends on the temperature and ionic strength of their milieu, the length of the nucleic acids and the degree of complementarity. The effect of these parameters on hybridization is well known in the art (see Sambrook J, Fritsch E F, Maniatis T. 1989.

Molecular cloning: a laboratory manual. Cold Spring Harbor Laboratory Press, New York). As used herein, hybridization of a primer sequence, or of a DNA extension product, to another nucleic acid shall mean annealing sufficient such that the primer, or DNA extension product, respectively, is extendable by creation of a phosphodiester bond with an available nucleotide or nucleotide analog capable of forming a phosphodiester bond.

As used herein, unless otherwise specified, a base which is “unique” or “different from” another base or a recited list of bases shall mean that the base has a different structure from the other base or bases. For example, a base that is “unique” or “different from” adenine, thymine, and cytosine would include a base that is guanine or a base that is uracil.

As used herein, unless otherwise specified, a label or tag moiety which is “different” from the label or tag moiety of a referenced molecule means that the label or tag moiety has a different chemical structure from the chemical structure of the other/referenced label or tag moiety.

As used herein, unless otherwise specified, “primer” means an oligonucleotide that upon forming a duplex with a polynucleotide template, is capable of acting as a point of polymerase incorporation and extension from its 3′ end along the template, thereby resulting in an extended duplex. Throughout this application, “template-loop-primers” are often referred to. However, other template-primer arrangements are also included within the scope of the invention (e.g., linear primers bound to surface attached linear or circular templates).

As used herein, “alkyl” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C1-C10 means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

As used herein, “alkenyl” refers to a non-aromatic hydrocarbon group, straight or branched, containing at least 1 carbon to carbon double bond, and up to the maximum possible number of non-aromatic carbon-carbon double bonds may be present, and may be unsubstituted or substituted. For example, “C2-C5 alkenyl” means an alkenyl group having 2, 3, 4, or 5, carbon atoms, and up to 1, 2, 3, or 4, carbon-carbon double bonds respectively. Alkenyl groups include ethenyl, propenyl, and butenyl.

The term “alkynyl” refers to a hydrocarbon group straight or branched, containing at least 1 carbon to carbon triple bond, and up to the maximum possible number of non-aromatic carbon-carbon triple bonds may be present, and may be unsubstituted or substituted. Thus, “C2-C5 alkynyl” means an alkynyl group having 2 or 3 carbon atoms and 1 carbon-carbon triple bond, or having 4 or 5 carbon atoms and up to 2 carbon-carbon triple bonds. Alkynyl groups include ethynyl, propynyl and butynyl.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The term “substituted” refers to a functional group as described above such as an alkyl, or a hydrocarbyl, in which at least one bond to a hydrogen atom contained therein is replaced by a bond to non-hydrogen or non-carbon atom, provided that normal valences are maintained and that the substitution(s) result(s) in a stable compound. Substituted groups also include groups in which one or more bonds to a carbon(s) or hydrogen(s) atom are replaced by one or more bonds, including double or triple bonds, to a heteroatom. Non-limiting examples of substituents include the functional groups described above, and for example, N, e.g. so as to form —CN.

A “detectable agent” or “detectable compound” or “detectable label” or “detectable moiety” is a composition detectable by spectroscopic, photochemical, biochemical, immunochemical, chemical, magnetic resonance imaging, or other physical means. For example, detectable agents include 18F, 32P, 33P, 45Ti, 47Sc, 52Fe, 59Fe, 62Cu, 64Cu, 67Cu, 67Ga, 68Ga, 77As, 86Y, 90Y. 89Sr, 89Zr, 94Tc, 94Tc, 99mTc, 99Mo, 105Pd, 105Rh, 111Ag, 111In, 123I, 124I, 125I, 131I, 142Pr, 143Pr, 149Pm, 153Sm, 154-1581Gd, 161Tb, 166Dy, 166Ho, 169Er, 175Lu, 177Lu, 186Re, 188Re, 189Re, 194Ir, 198Au, 199Au, 211At, 211Pb, 212Bi, 212Pb, 213Bi, 223Ra, 225Ac, Cr, V, Mn, Fe, Co, Ni, Cu, La, Ce, Pr, Nd, Pm, Sm, Eu, Gd, Tb, Dy, Ho, Er, Tm, Yb, Lu, 32P, fluorophore (e.g. fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes.

In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescein isothiocyanate moiety, tetramethylrhodamine-5-(and 6)-isothiocyanate moiety, Cy2 moeity, Cy3 moiety, Cy5 moiety, Cy7 moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, the detectable moiety is a Indo-1, Ca saturated moiety, Indo-1 Ca2+ moiety, Cascade Blue BSA pH 7.0 moiety, Cascade Blue moiety, LysoTracker Blue moiety, Alexa 405 moiety, LysoSensor Blue pH 5.0 moiety, LysoSensor Blue moiety, DyLight 405 moiety, DyLight 350 moiety, BFP (Blue Fluorescent Protein) moiety, Alexa 350 moiety, 7-Amino-4-methylcoumarin pH 7.0 moiety, Amino Coumarin moiety, AMCA conjugate moiety, Coumarin moiety, 7-Hydroxy-4-methylcoumarin moiety, 7-Hydroxy-4-methylcoumarin pH 9.0 moiety, 6,8-Difluoro-7-hydroxy-4-methylcoumarin pH 9.0 moiety, Hoechst 33342 moiety, Pacific Blue moiety, Hoechst 33258 moiety, Hoechst 33258-DNA moiety, Pacific Blue antibody conjugate pH 8.0 moiety, PO-PRO-1 moiety, PO-PRO-1-DNA moiety, POPO-1 moiety, POPO-1-DNA moiety, DAPI-DNA moiety, DAPI moiety, Marina Blue moiety, SYTOX Blue-DNA moiety, CFP (Cyan Fluorescent Protein) moiety, eCFP (Enhanced Cyan Fluorescent Protein) moiety, 1-Anilinonaphthalene-8-sulfonic acid (1,8-ANS) moiety, Indo-1, Ca free moiety, 1,8-ANS (1-Anilinonaphthalene-8-sulfonic acid) moiety, BO-PRO-1-DNA moiety, BOPRO-1 moiety, BOBO-1-DNA moiety, SYTO 45-DNA moiety, evoglow-Ppl moiety, evoglow-Bsl moiety, evoglow-Bs2 moiety, Auramine O moiety, DiO moiety, LysoSensor Green pH 5.0 moiety, Cy 2 moiety, LysoSensor Green moiety, Fura-2, high Ca moiety, Fura-2 Ca2+sup> moiety, SYTO 13-DNA moiety, YO-PRO-1-DNA moiety, YOYO-1-DNA moiety, eGFP (Enhanced Green Fluorescent Protein) moiety, LysoTracker Green moiety, GFP (S65T) moiety, BODIPY FL, MeOH moiety, Sapphire moiety, BODIPY FL conjugate moiety, MitoTracker Green moiety, MitoTracker Green FM, MeOH moiety, Fluorescein 0.1 M NaOH moiety, Calcein pH 9.0 moiety, Fluorescein pH 9.0 moiety, Calcein moiety, Fura-2, no Ca moiety, Fluo-4 moiety, FDA moiety, DTAF moiety, Fluorescein moiety, CFDA moiety, FITC moiety, Alexa Fluor 488 hydrazide-water moiety, DyLight 488 moiety, 5-FAM pH 9.0 moiety, Alexa 488 moiety, Rhodamine 110 moiety, Rhodamine 110 pH 7.0 moiety, Acridine Orange moiety, BCECF pH 5.5 moiety, PicoGreendsDNA quantitation reagent moiety, SYBR Green I moiety, Rhodaminen Green pH 7.0 moiety, CyQUANT GR-DNA moiety, NeuroTrace 500/525, green fluorescent Nissl stain-RNA moiety, DansylCadaverine moiety, Fluoro-Emerald moiety, Nissl moiety, Fluorescein dextran pH 8.0 moiety, Rhodamine Green moiety, 5-(and-6)-Carboxy-2′, 7′-dichlorofluorescein pH 9.0 moiety, DansylCadaverine, MeOH moiety, eYFP (Enhanced Yellow Fluorescent Protein) moiety, Oregon Green 488 moiety, Fluo-3 moiety, BCECF pH 9.0 moiety, SBFI-Na+ moiety, Fluo-3 Ca2+ moiety, Rhodamine 123 MeOH moiety, FlAsH moiety, Calcium Green-1 Ca2+ moiety, Magnesium Green moiety, DM-NERF pH 4.0 moiety, Calcium Green moiety, Citrine moiety, LysoSensor Yellow pH 9.0 moiety, TO-PRO-1-DNA moiety, Magnesium Green Mg2+ moiety, Sodium Green Na+ moiety, TOTO-1-DNA moiety, Oregon Green 514 moiety, Oregon Green 514 antibody conjugate pH 8.0 moiety, NBD-X moiety, DM-NERF pH 7.0 moiety, NBD-X, MeOH moiety, CI-NERF pH 6.0 moiety, Alexa 430 moiety, CI-NERF pH 2.5 moiety, Lucifer Yellow, CH moiety, LysoSensor Yellow pH 3.0 moiety, 6-TET, SE pH 9.0 moiety, Eosin antibody conjugate pH 8.0 moiety, Eosin moiety, 6-Carboxyrhodamine 6G pH 7.0 moiety, 6-Carboxyrhodamine 6G, hydrochloride moiety, Bodipy R6G SE moiety, BODIPY R6G MeOH moiety, 6 JOE moiety, Cascade Yellow moiety, mBanana moiety, Alexa 532 moiety, Erythrosin-5-isothiocyanate pH 9.0 moiety, 6-HEX, SE pH 9.0 moiety, mOrange moiety, mHoneydew moiety, Cy 3 moiety, Rhodamine B moiety, DiI moiety, 5-TAMRA-MeOH moiety, Alexa 555 moiety, DyLight 549 moiety, BODIPY TMR-X, SE moiety, BODIPY TMR-X MeOH moiety, PO-PRO-3-DNA moiety, PO-PRO-3 moiety, Rhodamine moiety, POPO-3 moiety, Alexa 546 moiety, Calcium Orange Ca2+ moiety, TRITC moiety, Calcium Orange moiety, Rhodaminephalloidin pH 7.0 moiety, MitoTracker Orange moiety, MitoTracker Orange MeOH moiety, Phycoerythrin moiety, Magnesium Orange moiety, R-Phycoerythrin pH 7.5 moiety, 5-TAMRA pH 7.0 moiety, 5-TAMRA moiety, Rhod-2 moiety, FM 1-43 moiety, Rhod-2 Ca2+ moiety, FM 1-43 lipid moiety, LOLO-1-DNA moiety, dTomato moiety, DsRed moiety, Dapoxyl(2-aminoethyl)sulfonamide moiety, Tetramethylrhodamine dextran pH 7.0 moiety, Fluor-Ruby moiety, Resorufin moiety, Resorufin pH 9.0 moiety, mTangerine moiety, LysoTracker Red moiety, Lissaminerhodamine moiety, Cy 3.5 moiety, Rhodamine Red-X antibody conjugate pH 8.0 moiety, Sulforhodamine 101 EtOH moiety, JC-1 pH 8.2 moiety, JC-1 moiety, mStrawberry moiety, MitoTracker Red moiety, MitoTracker Red, MeOH moiety, X-Rhod-1 Ca2+ moiety, Alexa 568 moiety, 5-ROX pH 7.0 moiety, 5-ROX (5-Carboxy-X-rhodamine, triethylammonium salt) moiety, BO-PRO-3-DNA moiety, BOPRO-3 moiety, BOBO-3-DNA moiety, Ethidium Bromide moiety, ReAsH moiety, Calcium Crimson moiety, Calcium Crimson Ca2+ moiety, mRFP moiety, mCherry moiety, HcRed moiety, DyLight 594 moiety, Ethidium homodimer-1-DNA moiety, Ethidiumhomodimer moiety, Propidium Iodide moiety, SYPRO Ruby moiety, Propidium Iodide-DNA moiety, Alexa 594 moiety, BODIPY TR-X, SE moiety, BODIPY TR-X, MeOH moiety, BODIPY TR-X phallacidin pH 7.0 moiety, Alexa Fluor 610 R-phycoerythrin streptavidin pH 7.2 moiety, YO-PRO-3-DNA moiety, Di-8 ANEPPS moiety, Di-8-ANEPPS-lipid moiety, YOYO-3-DNA moiety, Nile Red-lipid moiety, Nile Red moiety, DyLight 633 moiety, mPlum moiety, TO-PRO-3-DNA moiety, DDAO pH 9.0 moiety, Fura Red high Ca moiety, Allophycocyanin pH 7.5 moiety, APC (allophycocyanin) moiety, Nile Blue, EtOH moiety, TOTO-3-DNA moiety, Cy 5 moiety, BODIPY 650/665-X, MeOH moiety, Alexa Fluor 647 R-phycoerythrin streptavidin pH 7.2 moiety, DyLight 649 moiety, Alexa 647 moiety, Fura Red Ca2+ moiety, Atto 647 moiety, Fura Red, low Ca moiety, Carboxynaphthofluorescein pH 10.0 moiety, Alexa 660 moiety, Cy 5.5 moiety, Alexa 680 moiety, DyLight 680 moiety, Alexa 700 moiety, FM 4-64, 2% CHAPS moiety, or FM 4-64 moiety. In embodiments, the detectable moiety is a moiety of 1,1-Diethyl-4,4-carbocyanine iodide, 1,2-Diphenylacetylene, 1,4-Diphenylbutadiene, 1,4-Diphenylbutadiyne, 1,6-Diphenylhexatriene, 1,6-Diphenylhexatriene, 1-anilinonaphthalene-8-sulfonic acid, 2,7-Dichlorofluorescein, 2,5-DIPHENYLOXAZOLE, 2-Di-1-ASP, 2-dodecylresorufin, 2-Methylbenzoxazole, 3,3-Diethylthiadicarbocyanine iodide, 4-Dimethylamino-4-Nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein, 5-(and-6)-carboxy-2,7-dichlorofluorescein, 5-(N-hexadecanoyl)aminoeosin, 5-(N-hexadecanoyl)aminoeosin, 5-chloromethylfluorescein, 5-FAM, 5-ROX, 5-TAMRA, 5-TAMRA, 6,8-difluoro-7-hydroxy-4-methylcoumarin, 6,8-difluoro-7-hydroxy-4-methylcoumarin, 6-carboxyrhodamine 6G, 6-HEX, 6-JOE, 6-JOE, 6-TET, 7-aminoactinomycin D, 7-Benzylamino-4-Nitrobenz-2-Oxa-1,3-Diazole, 7-Methoxycoumarin-4-Acetic Acid, 8-Benzyloxy-5,7-diphenylquinoline, 8-Benzyloxy-5,7-diphenylquinoline, 9,10-Bis(Phenylethynyl)Anthracene, 9,10-Diphenylanthracene, 9-METHYLCARBAZOLE, (CS)2Ir((μ-Cl)2Ir(CS)2, AAA, Acridine Orange, Acridine Orange, Acridine Yellow, Acridine Yellow, Adams Apple Red 680, Adirondack Green 520, Alexa Fluor 350, Alexa Fluor 405, Alexa Fluor 430, Alexa Fluor 430, Alexa Fluor 480, Alexa Fluor 488, Alexa Fluor 488, Alexa Fluor 488 hydrazide, Alexa Fluor 500, Alexa Fluor 514, Alexa Fluor 532, Alexa Fluor 546, Alexa Fluor 546, Alexa Fluor 555, Alexa Fluor 555, Alexa Fluor 568, Alexa Fluor 594, Alexa Fluor 594, Alexa Fluor 594, Alexa Fluor 610, Alexa Fluor 610-R-PE, Alexa Fluor 633, Alexa Fluor 635, Alexa Fluor 647, Alexa Fluor 647, Alexa Fluor 647-R-PE, Alexa Fluor 660, Alexa Fluor 680, Alexa Fluor 680-APC, Alexa Fluor 680-R-PE, Alexa Fluor 700, Alexa Fluor 750, Alexa Fluor 790, Allophycocyanin, AmCyan1, Aminomethylcoumarin, Amplex Gold (product), Amplex Red Reagent, Amplex UltraRed, Anthracene, APC, APC-Seta-750, AsRed2, ATTO 390, ATTO 425, ATTO 430LS, ATTO 465, ATTO 488, ATTO 490LS, ATTO 495, ATTO 514, ATTO 520, ATTO 532, ATTO 550, ATTO 565, ATTO 590, ATTO 594, ATTO 610, ATTO 620, ATTO 633, ATTO 635, ATTO 647, ATTO 647N, ATTO 655, ATTO 665, ATTO 680, ATTO 700, ATTO 725, ATTO 740, ATTO Oxa12, ATTO Rho3B, ATTO Rho6G, ATTO Rholl, ATTO Rho12, ATTO Rho13, ATTO Rho14, ATTO Rhol01, ATTO Thio12, Auramine O, Azami Green, Azami Green monomeric, B-phycoerythrin, BCECF, BCECF, Bex1, Biphenyl, Birch Yellow 580, Blue-green algae, BO-PRO-1, BO-PRO-3, BOBO-1, BOBO-3, BODIPY 630 650-X, BODIPY 650/665-X, BODIPY FL, BODIPY FL, BODIPY R6G, BODIPY TMR-X, BODIPY TR-X, BODIPY TR-X Ph 7.0, BODIPY TR-X phallacidin, BODIPY-DiMe, BODIPY-Phenyl, BODIPY-TMSCC, C3-Indocyanine, C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, C545T, C-Phycocyanin, Calcein, Calcein red-orange, Calcium Crimson, Calcium Green-1, Calcium Orange, Calcofluor white 2MR, Carboxy SNARF-1 pH 6.0, Carboxy SNARF-1 pH 9.0, Carboxynaphthofluorescein, Cascade Blue, Cascade Yellow, Catskill Green 540, CBQCA, CellMask Orange, CellTrace BODIPY TR methyl ester, CellTrace calcein violet, CellTrace™ Far Red, CellTracker Blue, CellTracker Red CMTPX, CellTracker Violet BMQC, CF405M, CF405S, CF488A, CF543, CF555, CFP, CFSE, CF™ 350, CF™ 485, Chlorophyll A, Chlorophyll B, Chromeo 488, Chromeo 494, Chromeo 505, Chromeo 546, Chromeo 642, Citrine, Citrine, C10H butoxy aza-BODIPY, ClOH C12 aza-BODIPY, CM-H2DCFDA, Coumarin 1, Coumarin 6, Coumarin 6, Coumarin 30, Coumarin 314, Coumarin 334, Coumarin 343, Coumarine 545T, Cresyl Violet Perchlorate, CryptoLight CF1, CryptoLight CF2, CryptoLight CF3, CryptoLight CF4, CryptoLight CF5, CryptoLight CF6, Crystal Violet, Cumarin153, Cy2, Cy3, Cy3, Cy3.5, Cy3B, Cy3B, Cy3Cy5 ET, Cy5, Cy5, Cy5.5, Cy7, Cyanine3 NHS ester, Cyanine5 carboxylic acid, Cyanine5 NHS ester, Cyclotella meneghiniana KUtzing, CypHer5, CypHer5 pH 9.15, CyQUANT GR, CyTrak Orange, Dabcyl SE, DAF-FM, DAMC (Weiss), dansyl cadaverine, Dansyl Glycine (Dioxane), DAPI, DAPI, DAPI, DAPI, DAPI (DMSO), DAPI (H2O), Dapoxyl (2-aminoethyl)sulfonamide, DCI, DCM, DCM, DCM (acetonitrile), DCM (MeOH), DDAO, Deep Purple, di-8-ANEPPS, DiA, Dichlorotris(1,10-phenanthroline) ruthenium(II), DiClOH C12 aza-BODIPY, DiClOHbutoxy aza-BODIPY, DiD, DiI, DiIC18(3), DiO, DiR, Diversa Cyan-FP, Diversa Green-FP, DM-NERF pH 4.0, DOCI, Doxorubicin, DPP pH-Probe 590-7.5, DPP pH-Probe 590-9.0, DPP pH-Probe 590-11.0, DPP pH-Probe 590-11.0, Dragon Green, DRAQ5, DsRed, DsRed, DsRed, DsRed-Express, DsRed-Express2, DsRed-Express T1, dTomato, DY-350XL, DY-480, DY-480XL MegaStokes, DY-485, DY-485XL MegaStokes, DY-490, DY-490XL MegaStokes, DY-500, DY-500XL MegaStokes, DY-520, DY-520XL MegaStokes, DY-547, DY-549Pi, DY-549Pi, DY-554, DY-555, DY-557, DY-557, DY-590, DY-590, DY-615, DY-630, DY-631, DY-633, DY-635, DY-636, DY-647, DY-649P1, DY-649P1, DY-650, DY-651, DY-656, DY-673, DY-675, DY-676, DY-680, DY-681, DY-700, DY-701, DY-730, DY-731, DY-750, DY-751, DY-776, DY-782, Dye-28, Dye-33, Dye-45, Dye-304, Dye-1041, DyLight 488, DyLight 549, DyLight 594, DyLight 633, DyLight 649, DyLight 680, E2-Crimson, E2-Orange, E2-Red/Green, EBFP, ECF, ECFP, ECL Plus, eGFP, ELF 97, Emerald, Envy Green, Eosin, Eosin Y, epicocconone, EqFP611, Erythrosin-5-isothiocyanate, Ethidium bromide, ethidium homodimer-1, Ethyl Eosin, Ethyl Eosin, Ethyl Nile Blue A, Ethyl-p-Dimethylaminobenzoate, Ethyl-p-Dimethylaminobenzoate, Eu203 nanoparticles, Eu (Soini), Eu(tta)3DEADIT, EvaGreen, EVOblue-30, EYFP, FAD, FITC, FITC, FlAsH (Adams), Flash Red EX, FlAsH-CCPGCC, FlAsH-CCXXCC, Fluo-3, Fluo-4, Fluo-5F, Fluorescein, Fluorescein 0.1 NaOH, Fluorescein-Dibase, fluoro-emerald, Fluorol 5G, FluoSpheres blue, FluoSpheres crimson, FluoSpheres dark red, FluoSpheres orange, FluoSpheres red, FluoSpheres yellow-green, FM4-64 in CTC, FM4-64 in SDS, FM 1-43, FM 4-64, Fort Orange 600, Fura Red, Fura Red Ca free, fura-2, Fura-2 Ca free, Gadodiamide, Gd-Dtpa-Bma, Gadodiamide, Gd-Dtpa-Bma, GelGreen™, GelRed™, H9-40, HcRedl, Hemo Red 720, HiLyte Fluor 488, HiLyte Fluor 555, HiLyte Fluor 647, HiLyte Fluor 680, HiLyte Fluor 750, HiLyte Plus 555, HiLyte Plus 647, HiLyte Plus 750, HmGFP, Hoechst 33258, Hoechst 33342, Hoechst-33258, Hoechst-33258, Hops Yellow 560, HPTS, HPTS, HPTS, HPTS, HPTS, indo-1, Indo-1 Ca free, Ir(Cn)2(acac), Ir(Cs)2(acac), IR-775 chloride, IR-806, Ir-OEP—CO-Cl, IRDye® 650 Alkyne, IRDye® 650 Azide, IRDye® 650 Carboxylate, IRDye® 650 DBCO, IRDye® 650 Maleimide, IRDye® 650 NHS Ester, IRDye® 680LT Carboxylate, IRDye® 680LT Maleimide, IRDye® 680LT NHS Ester, IRDye® 680RD Alkyne, IRDye® 680RD Azide, IRDye® 680RD Carboxylate, IRDye® 680RD DBCO, IRDye® 680RD Maleimide, IRDye® 680RD NHS Ester, IRDye® 700 phosphoramidite, IRDye® 700DX, IRDye® 700DX, IRDye® 700DX Carboxylate, IRDye® 700DX NHS Ester, IRDye® 750 Carboxylate, IRDye® 750 Maleimide, IRDye® 750 NHS Ester, IRDye® 800 phosphoramidite, IRDye® 800CW, IRDye® 800CW Alkyne, IRDye® 800CW Azide, IRDye® 800CW Carboxylate, IRDye® 800CW DBCO, IRDye® 800CW Maleimide, IRDye® 800CW NHS Ester, IRDye® 800RS, IRDye® 800RS Carboxylate, IRDye® 800RS NHS Ester, IRDye® QC-1 Carboxylate, IRDye® QC-1 NHS Ester, Isochrysis galbana—Parke, JC-1, JC-1, JOJO-1, Jonamac Red Evitag T2, Kaede Green, Kaede Red, kusabira orange, Lake Placid 490, LDS 751, Lissamine Rhodamine (Weiss), LOLO-1, lucifer yellow CH, Lucifer Yellow CH, lucifer yellow CH, Lucifer Yellow CH Dilitium salt, Lumio Green, Lumio Red, Lumogen F Orange, Lumogen Red F300, Lumogen Red F300, LysoSensor Blue DND-192, LysoSensor Green DND-153, LysoSensor Green DND-153, LysoSensor Yellow/Blue DND-160 pH 3, LysoSensor YellowBlue DND-160, LysoTracker Blue DND-22, LysoTracker Blue DND-22, LysoTracker Green DND-26, LysoTracker Red DND-99, LysoTracker Yellow HCK-123, Macoun Red Evitag T2, Macrolex Fluorescence Red G, Macrolex Fluorescence Yellow 10GN, Macrolex Fluorescence Yellow 10GN, Magnesium Green, Magnesium Octaethylporphyrin, Magnesium Orange, Magnesium Phthalocyanine, Magnesium Phthalocyanine, Magnesium Tetramesitylporphyrin, Magnesium Tetraphenylporphyrin, malachite green isothiocyanate, Maple Red-Orange 620, Marina Blue, mBanana, mBBr, mCherry, Merocyanine 540, Methyl green, Methyl green, Methyl green, Methylene Blue, Methylene Blue, mHoneyDew, MitoTracker Deep Red 633, MitoTracker Green FM, MitoTracker Orange CMTMRos, MitoTracker Red CMXRos, monobromobimane, Monochlorobimane, Monoraphidium, mOrange, mOrange2, mPlum, mRaspberry, mRFP, mRFP1, mRFP1.2 (Wang), mStrawberry (Shaner), mTangerine (Shaner), N,N-Bis(2,4,6-trimethylphenyl)-3,4:9,10-perylenebis(dicarboximide), NADH, Naphthalene, Naphthalene, Naphthofluorescein, Naphthofluorescein, NBD-X, NeuroTrace 500525, Nilblau perchlorate, nile blue, Nile Blue, Nile Blue (EtOH), nile red, Nile Red, Nile Red, Nile red, Nileblue A, NIR1, NIR2, NIR3, NIR4, NIR820, Octaethylporphyrin, OH butoxy aza-BODIPY, OHC12 aza-BODIPY, Orange Fluorescent Protein, Oregon Green 488, Oregon Green 488 DHPE, Oregon Green 514, Oxazinl, Oxazin 750, Oxazine 1, Oxazine 170, P4-3, P-Quaterphenyl, P-Terphenyl, PA-GFP (post-activation), PA-GFP (pre-activation), Pacific Orange, Palladium(II) meso-tetraphenyl-tetrabenzoporphyrin, PdOEPK, PdTFPP, PerCP-Cy5.5, Perylene, Perylene, Perylene bisimide pH-Probe 550-5.0, Perylene bisimide pH-Probe 550-5.5, Perylene bisimide pH-Probe 550-6.5, Perylene Green pH-Probe 720-5.5, Perylene Green Tag pH-Probe 720-6.0, Perylene Orange pH-Probe 550-2.0, Perylene Orange Tag 550, Perylene Red pH-Probe 600-5.5, Perylenediimid, Perylne Green pH-Probe 740-5.5, Phenol, Phenylalanine, pHrodo, succinimidyl ester, Phthalocyanine, PicoGreen dsDNA quantitation reagent, Pinacyanol-Iodide, Piroxicam, Platinum(II) tetraphenyltetrabenzoporphyrin, Plum Purple, PO-PRO-1, PO-PRO-3, POPO-1, POPO-3, POPOP, Porphin, PPO, Proflavin, PromoFluor-350, PromoFluor-405, PromoFluor-415, PromoFluor-488, PromoFluor-488 Premium, PromoFluor-488LSS, PromoFluor-500LSS, PromoFluor-505, PromoFluor-510LSS, PromoFluor-514LSS, PromoFluor-520LSS, PromoFluor-532, PromoFluor-546, PromoFluor-555, PromoFluor-590, PromoFluor-610, PromoFluor-633, PromoFluor-647, PromoFluor-670, PromoFluor-680, PromoFluor-700, PromoFluor-750, PromoFluor-770, PromoFluor-780, PromoFluor-840, propidium iodide, Protoporphyrin IX, PTIR475/UF, PTIR545/UF, PtOEP, PtOEPK, PtTFPP, Pyrene, QD525, QD565, QD585, QD605, QD655, QD705, QD800, QD903, QD PbS 950, QDot 525, QDot 545, QDot 565, Qdot 585, Qdot 605, Qdot 625, Qdot 655, Qdot 705, Qdot 800, QpyMe2, QSY 7, QSY 7, QSY 9, QSY 21, QSY 35, quinine, Quinine Sulfate, Quinine sulfate, R-phycoerythrin, R-phycoerythrin, ReAsH-CCPGCC, ReAsH-CCXXCC, Red Beads (Weiss), Redmond Red, Resorufin, resorufin, rhod-2, Rhodamin 700 perchlorate, rhodamine, Rhodamine 6G, Rhodamine 6G, Rhodamine 101, rhodamine 110, Rhodamine 123, rhodamine 123, Rhodamine B, Rhodamine B, Rhodamine Green, Rhodamine pH-Probe 585-7.0, Rhodamine pH-Probe 585-7.5, Rhodamine phalloidin, Rhodamine Red-X, Rhodamine Red-X, Rhodamine Tag pH-Probe 585-7.0, Rhodol Green, Riboflavin, Rose Bengal, Sapphire, SBFI, SBFI Zero Na, Scenedesmus sp., SensiLight PBXL-1, SensiLight PBXL-3, Seta 633-NHS, Seta-633-NHS, SeTau-380-NHS, SeTau-647-NHS, Snake-Eye Red 900, SNIR1, SNIR2, SNIR3, SNIR4, Sodium Green, Solophenyl flavine 7GFE 500, Spectrum Aqua, Spectrum Blue, Spectrum FRed, Spectrum Gold, Spectrum Green, Spectrum Orange, Spectrum Red, Squarylium dye III, Stains All, Stilben derivate, Stilbene, Styryl8 perchlorate, Sulfo-Cyanine3 carboxylic acid, Sulfo-Cyanine3 carboxylic acid, Sulfo-Cyanine3 NHS ester, Sulfo-Cyanine5 carboxylic acid, Sulforhodamine 101, sulforhodamine 101, Sulforhodamine B, Sulforhodamine G, Suncoast Yellow, SuperGlo BFP, SuperGlo GFP, Surf Green EX, SYBR Gold nucleic acid gel stain, SYBR Green I, SYPRO Ruby, SYTO 9, SYTO 11, SYTO 13, SYTO 16, SYTO 17, SYTO 45, SYTO 59, SYTO 60, SYTO 61, SYTO 62, SYTO 82, SYTO RNASelect, SYTO RNASelect, SYTOX Blue, SYTOX Green, SYTOX Orange, SYTOX Red, T-Sapphire, Tb (Soini), tCO, tdTomato, Terrylen, Terrylendiimid, testdye, Tetra-t-Butylazaporphine, Tetra-t-Butylnaphthalocyanine, Tetracen, Tetrakis(o-Aminophenyl)Porphyrin, Tetramesitylporphyrin, Tetramethylrhodamine, tetramethylrhodamine, Tetraphenylporphyrin, Tetraphenylporphyrin, Texas Red, Texas Red DHPE, Texas Red-X, ThiolTracker Violet, Thionin acetate, TMRE, TO-PRO-1, TO-PRO-3, Toluene, Topaz (Tsienl998), TOTO-1, TOTO-3, Tris(2,2-Bipyridyl)Ruthenium(II) chloride, Tris(4,4-diphenyl-2,2-bipyridine) ruthenium(II) chloride, Tris(4,7-diphenyl-1,10-phenanthroline) ruthenium(II) TMS, TRITC (Weiss), TRITC Dextran (Weiss), Tryptophan, Tyrosine, Vex1, Vybrant DyeCycle Green stain, Vybrant DyeCycle Orange stain, Vybrant DyeCycle Violet stain, WEGFP (post-activation), WellRED D2, WellRED D3, WellRED D4, WtGFP, WtGFP (Tsienl998), X-rhod-1, Yakima Yellow, YFP, YO-PRO-1, YO-PRO-3, YOYO-1, YoYo-1, YoYo-1 dsDNA, YoYo-1 ssDNA, YOYO-3, Zinc Octaethylporphyrin, Zinc Phthalocyanine, Zinc Tetramesitylporphyrin, Zinc Tetraphenylporphyrin, ZsGreenl, or ZsYellowl.

In embodiments, the detectable label is a fluorescent dye. In embodiments, the detectable label is a fluorescent dye capable of exchanging energy with another fluorescent dye (e.g., fluorescence resonance energy transfer (FRET) chromophores).

In embodiments, the detectable label is a fluorescent dye capable of displaying absorbance and emission under one condition but not under a different condition. Examples are pH-responsive dyes.

In embodiments, the detectable moiety is a moiety of a derivative of one of the detectable moieties described immediately above, wherein the derivative differs from one of the detectable moieties immediately above by a modification resulting from the conjugation of the detectable moiety to a compound described herein.

The term “cyanine” or “cyanine moiety” as described herein refers to a compound containing two nitrogen groups separated by a polymethine chain. In embodiments, the cyanine moiety has 3 methine structures (i.e. cyanine 3 or Cy3). In embodiments, the cyanine moiety has 5 methine structures (i.e. cyanine 5 or Cy5). In embodiments, the cyanine moiety has 7 methine structures (i.e. cyanine 7 or Cy7). “Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The terms “streptavidin” and “ [symbol] ” refer to a tetrameric protein (including homologs, isoforms, and functional fragments thereof) capable of binding biotin. The term includes any recombinant or naturally-occurring form of streptavidin variants thereof that maintain streptavidin activity (e.g. within at least 30%, 40%, 50%, 60%, 70%, 80%, 90%, 95%, or 100% activity compared to wildtype streptavidin).

The term “anchor moiety” as used herein refers to a chemical moiety capable of interacting (e.g., covalently or non-covalently) with a second, optionally different, chemical moiety (e.g., complementary anchor moiety binder). In embodiments, the anchor moiety is a bioconjugate reactive group capable of interacting (e.g., covalently) with a complementary bioconjugate reactive group (e.g., complementary anchor moiety reactive group). In embodiments, an anchor moiety is a click chemistry reactant moiety. In embodiments, the anchor moiety (an “affinity anchor moiety”) is capable of non-covalently interacting with a second chemical moiety (e.g., complementary affinity anchor moiety binder). Non-limiting examples of an anchor moiety include biotin, azide, trans-cyclooctene (TCO) and phenyl boric acid (PBA). In embodiments, an affinity anchor moiety (e.g., biotin moiety) interacts non-covalently with a complementary affinity anchor moiety binder (e.g., streptavidin moiety). In embodiments, an anchor moiety (e.g., azide moiety, trans-cyclooctene (TCO) moiety, phenyl boric acid (PBA) moiety) covalently binds a complementary anchor moiety binder (e.g., dibenzocyclooctyne (DBCO) moiety, tetrazine (TZ) moiety, salicylhydroxamic acid (SHA) moiety).

The terms “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na2S2O4), hydrazine (N2H4)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation).

A photocleavable linker (e.g., including or consisting of a o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of an reducing agent (e.g., Tris(3-hydroxypropyl)phosphine). In embodiments, the cleavable linker is a dialkylketal linker, an azo linker, an allyl linker, a cyanoethyl linker, a 1-(4,4-dimethyl-2,6-dioxocyclohex-1-ylidene)ethyl linker, or a nitrobenzyl linker.

The term “orthogonally cleavable linker” or “orthogonal cleavable linker” as used herein refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent. In embodiments, an orthogonally is a cleavable linker that following cleavage the two separated entities (e.g., fluorescent dye, bioconjugate reactive group) do not further react and form a new orthogonally cleavable linker.

The term “orthogonal binding group” or “orthogonal binding molecule” as used herein refer to a binding group (e.g. anchor moiety or complementary anchor moiety binder) that is capable of binding a first complementary binding group (e.g., complementary anchor moiety binder or anchor moiety) in a mixture of two or more different complementary binding groups and is unable to bind any other different complementary binding group in the mixture of two or more complementary binding groups. For example, two different binding groups are both orthogonal binding groups when a mixture of the two different binding groups are reacted with two complementary binding groups and each binding group binds only one of the complementary binding groups and not the other complementary binding group. An example of a set of four orthogonal binding groups and a set of orthogonal complementary binding groups are the binding groups biotin, azide, trans-cyclooctene (TCO) and phenyl boric acid (PBA), which specifically and efficiently bind or react with the complementary binding groups streptavidin, dibenzocyclooctyne (DBCO), tetrazine (TZ) and salicylhydroxamic acid (SHA) respectively.

The term “orthogonal detectable label” or “orthogonal detectable moiety” as used herein refer to a detectable label (e.g. fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye.

Orthogonal detectable labels may be separately identified by different absorbance or emission intensities of the orthogonal detectable labels compared to each other and not only be the absolute presence of absence of a signal. An example of a set of four orthogonal detectable labels is the set of Rox-Labeled Tetrazine, Alexa488-Labeled SHA, Cy5-Labeled Streptavidin, and R6G-Labeled Dibenzocyclooctyne.

Throughout this application, many of the nucleotide analogues used in the various schemes contain dithiomethyl (DTM(SS)) blocking groups at the 3′ O position and often contain cleavable DTM(SS) groups in the linkers between the base and the dye or anchor molecules. Previous methods have placed SS groups between the base and dye but after cleavage a free, reactive —SH group is formed which has to be capped with iodoacetamide before the second extension reaction can be carried out (Mitra et al 2003, Turcatti et al 2008). This limits the length of sequencing reads. The new DTM based linker between the base and the fluorophore disclosed in this application does not require capping of the resulting free SH group after cleavage with THP as the cleaved product instantaneously collapses to the stable OH group.

The use of disulfide linker-based nucleotides as reversible terminators for DNA sequencing has been previously described (Ju et al WO 2017/058953 A1; Ju et al WO2017/205336 A1). Though the 3′-blocking group in all the examples shown in this section is t-butyl-dithiomethyl, other alkyl groups such as methyl-dithiomethyl or ethyl-dithiomethyl could also be used.

Wherever DTM is referred to in this patent application, it may refer to the dithiomethyl group or the various alkyl or other substituted dithiomethyl groups attached to the 3′-O position. Other blocking groups (azo, allyl, 2-nitrobenzyl, azidomethyl) may also be used, particularly if that group is present as the general cleavable group in all the linkers between the bases and dyes or anchors. In addition to nucleoside triphosphate analogues, nucleoside tetraphosphate, nucleoside pentaphosphate, nucleoside hexaphosphate and higher nucleoside polyphosphate analogues are feasible alternatives.

In some figures, positive fluorescence signals are indicated by the number 1, a gray rectangle, a black circle or the word “Signal”. In some figures, background signals are indicated by the number 0, a white rectangle and the words “Blank” or “Background”.

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention.

All combinations of the various elements described herein are within the scope of the invention. All sub-combinations of the various elements described herein are also within the scope of the invention.

Ju et al., PCT/US2019/022326, which is hereby incorporated by reference in its entirety, recently reported several single-color sequencing-by-synthesis (SBS) methods. Among those methods, included: (1) approaches using sets of nucleotides comprising two with identical anchors and two with identical dyes, with orthogonal arrangements of cleavable linkers between the base and the dye or anchor; and (2) approaches using sets of nucleotides comprising two with one species of anchor and two with a different species of anchor, with orthogonal arrangements of cleavable linkers between the base and the two anchors. Other approaches disclosed therein included those using more than two anchors or more than two cleavable linkers, approaches involving dye or anchor clusters, approaches using quantum dots, etc. Furthermore, the approaches above included: (a) those using only nucleotide analogues with dye or anchor attached to the base and reversibly blocked 3′-OH groups (nucleotide reversible terminators or NRTs), (b) those using dideoxynucleotide analogues with dye or anchor attached to the base in combination with non-fluorescent NRTs, and (c) those using nucleotide analogues with blocking groups (blockers) between the base and the dye/anchor but with a free 3′-OH group (referred to as “virtual terminators”).

The invention disclosed herein includes eleven novel single-color SBS schemes (Examples 1-3 and 5-12) involving the use of ddNTP analogues and one additional two-color scheme using ddNTP analogues (Example 4). The invention further includes the equivalent schemes for Examples 1, 2, 6, 8 and 9 using 3′-blocked nucleotide reversible terminators or virtual terminators. Though not explicitly presented herein, Examples 4, 5, 7, 10, 11 and 12 can also be performed using reversible terminators or virtual terminators. These schemes may differ by their use of, for example, different numbers of anchors and cleavable linkers, use of TCO linkers that can undergo click-to-cleave reactions (Examples 5, 8, 9), use of quenching (Examples 6, 7, 11, 12), use of photobleaching (Example 3), use of dyes with pH-responsive fluorescence (Examples 2, 7, 9, 10, 11), multiple extensions (Examples 1, 10, 12), and various combinations thereof (Examples 7, 9, 10, 11, 12). Each scheme has particular advantages, some of which are described in the introductory section to each of the examples. The invention also provides novel nucleotide analogues for use in the SBS schemes, for which example structures are provided. Two additional examples (Examples 13 and 14) describe a single molecule energy transfer approach, utilizing dyes with pH-responsive and pH-unresponsive fluorescence as acceptors. The use of energy transfer dyes for single molecule sequencing has been described previously (U.S. Pat. No. 6,627,748, US 2019/0153527 A1). Also disclosed herein, and provided within the scope of the invention, are several synthetic schemes for the synthesis of some of these nucleotide analogues.

In the various examples presented, and the associated embodiments and claims, different orders of addition of dye or anchor labeled nucleotides (ddNTPs or 3′-O blocked dNTPs, or dNTPs with blocking groups attached to the bases) relative to unlabeled 3′-O blocked nucleotides is described, but these should not be interpreted as exclusive orders of addition. Thus, in many examples the unlabeled 3′-O blocked nucleotides are said to be added “before or after” the labeled nucleotides, but this is not meant to exclude their being added at the same time. Similarly, in some examples, both sets of nucleotide analogues are shown added together, but this does not preclude their being added consecutively in either order. It will be apparent to those skilled in the art that the ratio of the labeled nucleotides and the 3′-O blocked nucleotides is adjusted depending on the order of addition, both to ensure synchronous sequencing by synthesis over many cycles and to ensure sufficient label detection in each cycle of sequencing by synthesis.

### Example 1: Single-Color Fluorescent Sequencing by Synthesis (SBS) Using Hybrid Approach with Unlabeled NRTs and Fluorescent ddNTPs, One Anchor and One Cleavable Linker

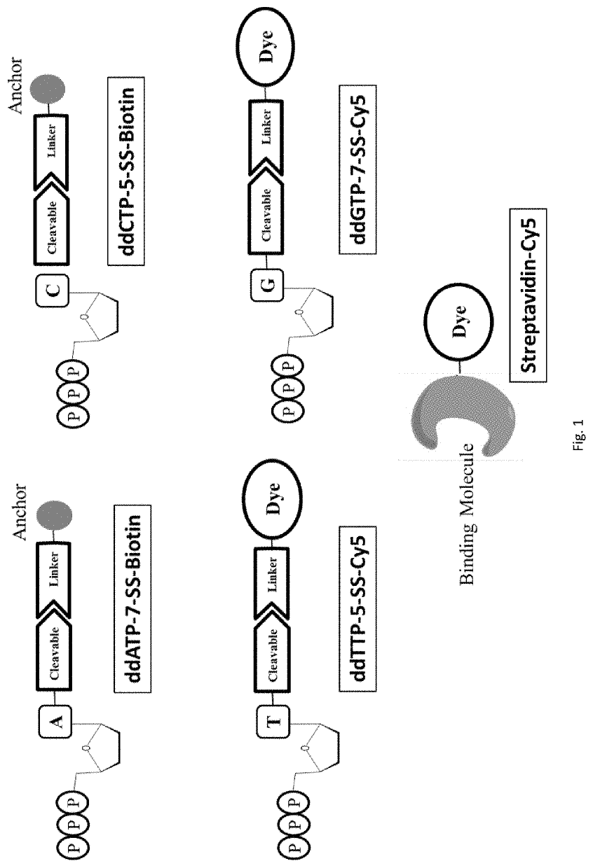

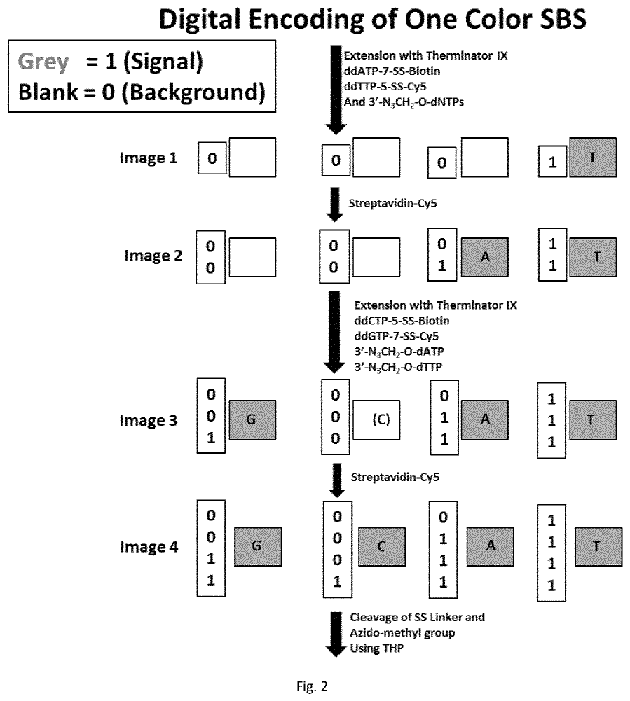

An exemplary schematic of this method is the one-color scheme presented in FIG. 2, which is shown using four azidomethyl-dNTPs (NRTs) and four ddNTP analogues, Cy5 is attached to two of the ddNTP analogues via an SS linker, and biotin is attached to the other two ddNTP analogues, also via an SS linker. The general structures of these ddNTP analogues are presented in FIG. 1. In this case, there are two extension steps and two labeling steps. For ease of presentation, the ddNTP analogues are presented as ddATP-SS-Biotin, ddTTP-SS-Cy5, ddCTP-SS-Biotin and ddGTP-SS-Cy5. In the first extension step, only ddATP-SS-Biotin and ddTTP-SS-Cy5 are added in the presence of an excess of the four azidomethyl-dNTPs (FIG. 3).

This is followed by the first imaging step, where a positive signal will reveal incorporation of T. Next labeling with streptavidin-Cy5 is carried out. A second imaging step is performed and the appearance of a new fluorescence signal will reveal incorporation of A. Next, a second extension step is performed with ddCTP-SS-Biotin and ddGTP-SS-Cy5 in the presence of higher concentrations of azidomethyl-dATP and azidomethyl-dTTP. A third imaging step is performed; a new positive signal will indicate incorporation of G.

Next, labeling is again carried out with streptavidin-Cy5. A fourth imaging step is performed; a new positive signal will indicate incorporation of C. Finally, cleavage with THP will be performed to cleave all the linkers, removing any Cy5 and also cleaving the azidomethyl blocking group on the NRTs.

A preponderance of NRTs are incorporated (>95%), with a sufficient amount of the ddNTP analogues to obtain a satisfactory signal, as any primers extended with the ddNTP analogues are lost to further cycles of sequencing. The advantage of this approach is that only one cleavage reagent is required, avoiding the potential of any cross-cleavage, which if high enough could potentially lead to incorrect base calls. THP cleavage of SS bonds has been previously described (Ju et al. US2018/0274024; Ju et al. PCT/US2019/022326).

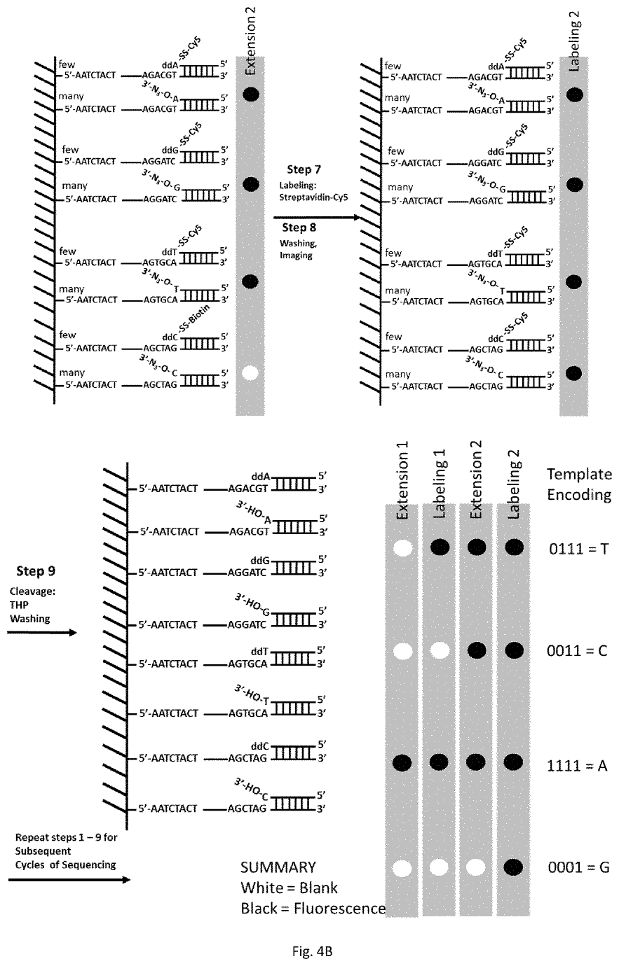

Another detailed embodiment of this SBS scheme is presented in FIG. 4. Though Cy5 is used in this example, many other fluorescent dyes could be used. A variety of anchors may be used in place of biotin such as those described in Ju et al. US2018/0274024 and Ju et al.

PCT/US2019/022326, each of which is incorporated herein in its entirety by reference. Though the cleavable linker in the example contains an SS group, alternative cleavable groups may be present in the linker, including allyl, 2-nitrobenzyl and others previously described, such as those in Ju et al. US2018/0274024 and Ju et al. PCT/US2019/022326.

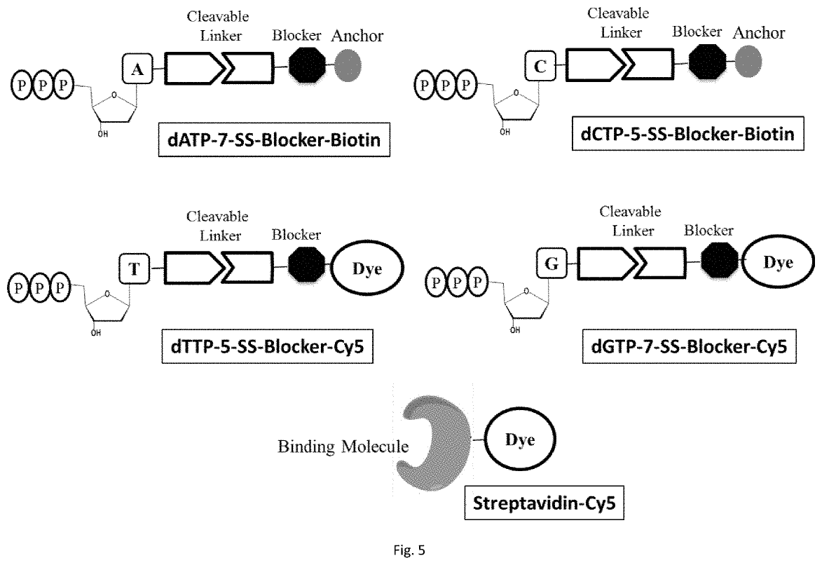

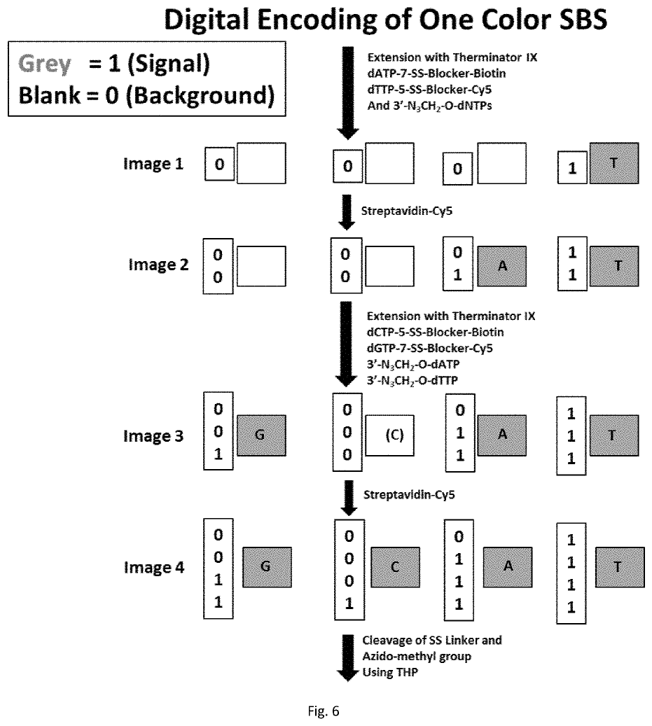

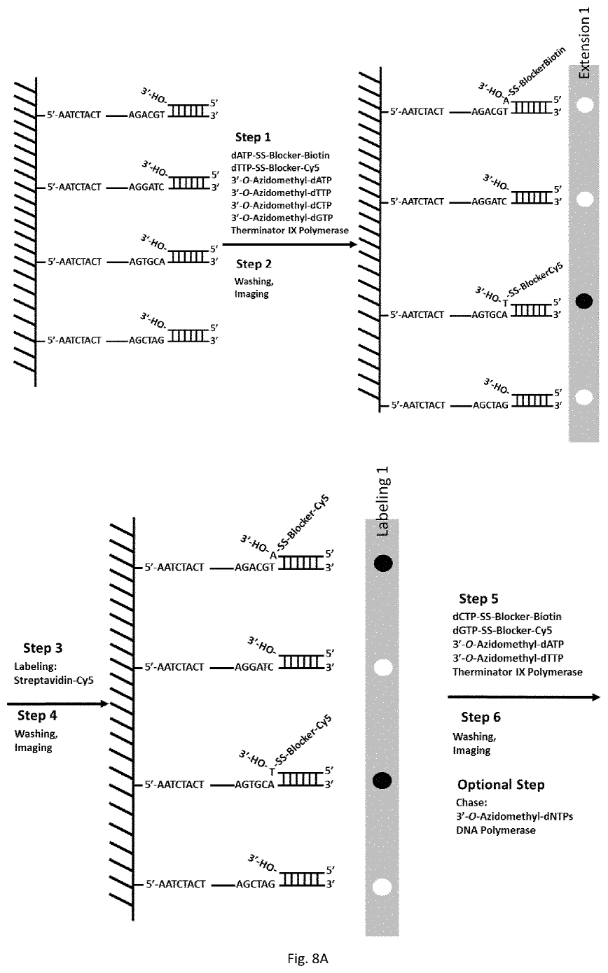

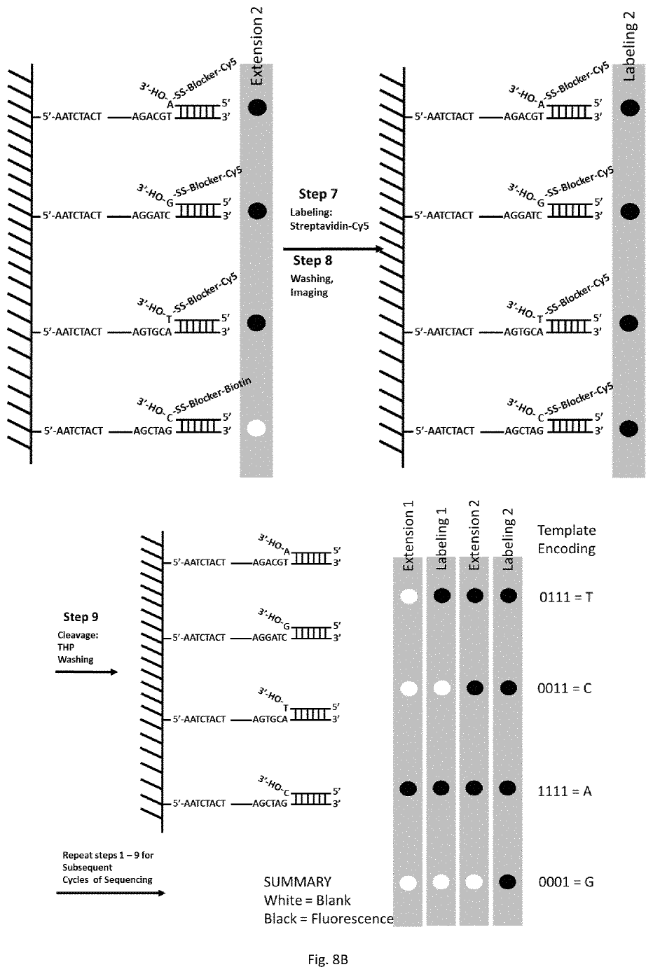

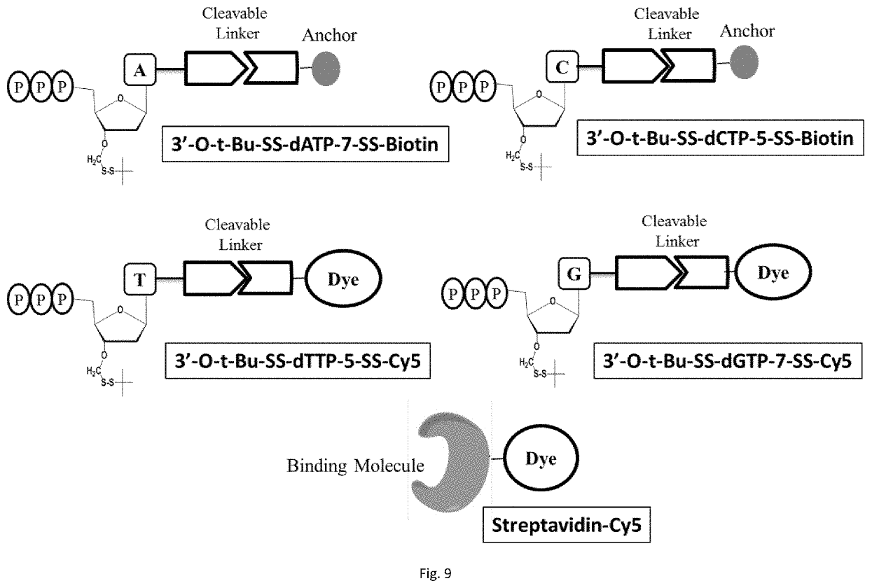

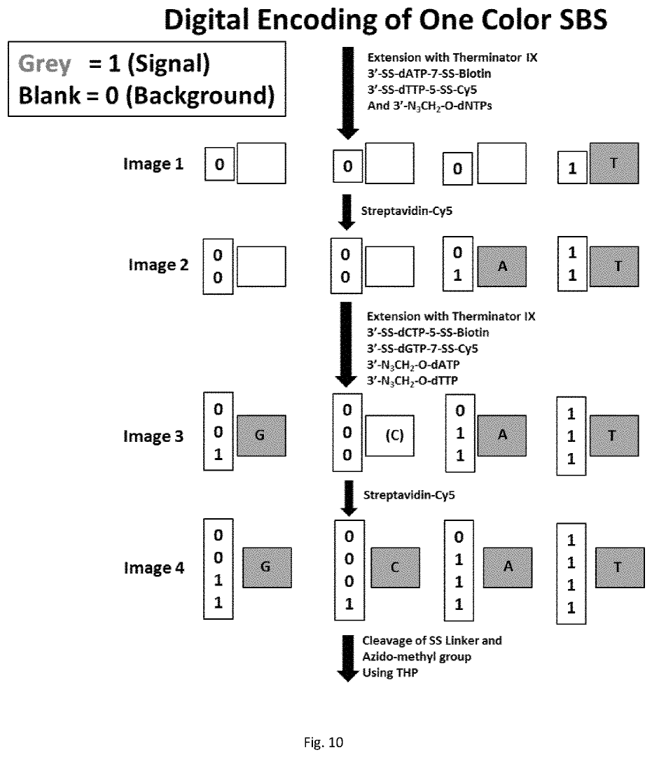

In other embodiments of the invention disclosed herein, a TCO cleavable linker that relies on a click-to-cleave strategy is used. Also disclosed herein, and provided by the subject invention, is a similar approach using virtual terminators (nucleotides containing a chemically cleavable blocking group connected to the base to inhibit the polymerase reaction) as shown in FIGS. 5-8, and another similar approach using 3′-blocked nucleotide reversible terminators as shown in FIGS. 9-12. In these cases, an optimal amount of unlabeled nucleotide reversible terminators is added together with the labeled nucleotide analogues to maintain fidelity of the polymerase reaction.

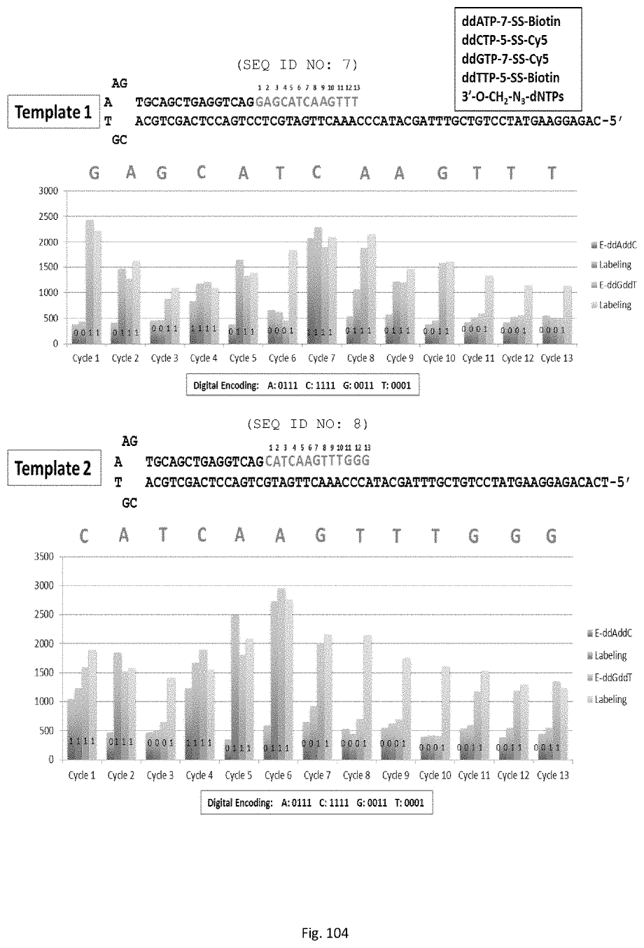

Example 1: Experimental Results (FIG. 104): Demonstration of the ability to conduct sequencing by synthesis with the same cleavable linker attached to either a dye (Cy5 in this case) or an anchor (biotin in this case) by conducting the following steps: (1) first extension: adding Therminator IX DNA polymerase and 3′-blocked reversible terminators; (2) second extension: adding Thermo Sequenase, one of the Cy5-labeled ddNTPs, one of the biotin-labeled ddNTPs, and the other two reversible terminators to maintain fidelity; (3) first labeling: adding Cy5-anchor binding molecule to label any DNA primers extended with the biotin-labeled ddNTP; (4) third extension: repeating extension step 2 for the remaining Cy5-labeled and biotin-labeled nucleotide analogue; (5) second labeling: repeating labeling step 3; (6) chase: repeating extension step 1; and (7) using THP to cleave the SS linkers to remove the dye and restore the 3′-OH group on incorporated reversible terminators.

Though in most aspects identical to the schemes presented in FIG. 2 and FIG. 4, in this instance, all four unlabeled 3′-O-t-butyldithiomethyl reversible terminators were added in the incorporation step with the first pair of labeled ddNTPs and two of the reversible terminators in the second incorporation step. The following four labeled ddNTP nucleotides (ddCTP-5-SS-Cy5, ddGTP-7-SS-Cy5, ddATP-7-SS-Biotin and ddTTP-5-SS-Biotin) were synthesized using the protocols described previously (Ju et al 2017a, b, 2018, 2019). A set of 3′-O-azidomethyl dNTPs (purchased commercially) was used to assure synchronization at every extension step. Primer-loop-templates with the following sequences, in which the next available positions in the template strand are shown in bold, were attached to a microscope slide as described in Ju et al. PCT/US2019/022326.

```
  AG
A TGCAGCTGAGGTCAG (SEQ ID NO: 3)
T ACGTCGACTCCAGTCCTCGTAGTTCAAACCCATACGATTTGCTGTCCTATGAAGGAGAC-5′
  GC
  AG
A TGCAGCTGAGGTCAG (SEQ ID NO: 4)
T ACGTCGACTCCAGTCGTAGTTCAAACCCATACGATTTGCTGTCCTATGAAGGAGACACT-5′
  GC
```

The following detailed SBS protocol was executed: First, three solutions were prepared.
Solution A: 5 μl 3′-O—CH₂—N₃-dATP (100 μM), 5 μl 3′-O—CH₂—N₃-dCTP (100 μM), 5 μl 3′-O—CH₂—N₃-dGTP (100 μM), 5 μl 3′-O—CH₂—N₃-dTTP (100 μM), 80 μl water.
Solution B: 4 μl ddATP-SS-Biotin (2 μM), 4 μl ddCTP-SS-Cy5 (2 μM), 5 μl 3′-O—CH₂—N₃-dGTP (2 μM), 5 μl 3′-O—CH₂—N₃-dTTP (2 μM).
Solution C: 4 μl ddTTP-SS-Biotin (2 μM), 4 μl ddGTP-SS-Cy5 (2 μM), 5 μl 3′-O—CH₂—N₃-dATP (2 μM), 5 μl 3′-O—CH₂—N₃-dCTP (2 μM).

For the first extension, a solution consisting of 40 μl Solution A, 6 μl 10× ThermoPol Buffer, 6 μl Therminator IX (1 unit/μl) and 8 μl water was added to the DNA on the slide, and incubation allowed to proceed for 10 min at 65° C. This step was designed to allow incorporation of the nucleotide reversible terminators in ˜95% of the growing primer strands.

Next, after washing the slides by dipping in a solution of 1×PBS pH 7.4, 0.1% Tween 20 for 10 min at 37° C. and thorough rinsing with water, the second extension step was carried out for 10 min at 65° C. using by adding a solution consisting of 5 μl Solution B, 6 μl 10× Thermo Sequenase Buffer, 6 μl Thermo Sequenase (1 unit/μl) and 43 μl water to the DNA on the slide. Another washing step was performed, the slide dried and imaged (633 nm laser and emission window centering around 670 nm) to reveal any Cy5 fluorescence due to incorporation of C.

Next, the first labeling step was carried out for 6 min at 37° C. by adding to the DNA on the slides a solution consisting of 6 μl Streptavidin-Cy5 (2 μM), 6 μl 10×PBS pH 7.4 and 48 μl water. The slide was washed as above, dried and re-imaged using the same conditions to reveal any Cy5 fluorescence due to incorporation and labeling of A.

The third extension step was performed for 10 min at 65° C. by adding a solution consisting of 5 μl Solution C, 6 μl 10× Thermo Sequenase Buffer, 6 μl Thermo Sequenase (1 unit/μl) and 43 μl water to the DNA on the slides. The slide was washed as above, dried and re-imaged using the same conditions to reveal any Cy5 fluorescence due to incorporation of G.

Next a second labeling step was performed in the same way as the first labeling step, followed by identical washing and imaging protocols to reveal any Cy5 fluorescence due to incorporation and labeling of T. The chase step to ensure essentially every DNA primer was extended was performed for 10 min at 65° C. by adding to the DNA on the slide a solution consisting of 40 μl Solution A, 6 μl 10× ThermoPol Buffer, 6 μl Therminator IX (1 unit/μl) and 8 μl water. The slide was washed as above.

Finally cleavage of the SS groups in the linkers of the ddNTPs to remove the dye and restoration of the 3′-OH on any incorporated 3′-O-azidomethyl dNTPs was accomplished by adding to the DNA on the slide a solution consisting of 6 μl THP (50 mM), 6 μl NaCl (200 mM), 12 μl Na Borate (0.1 M, pH 9) and 36 μl water and incubating for 5 min at 65° C. The slide was washed as above, dried and re-imaged using the same conditions as described previously to verify removal of dyes from the dye- or dye-anchor-labeled ddNTPs.

After repeating this protocol for 13 cycles for the above two primer-loop-templates covalently attached in separate areas of a slide, we obtained the results shown in FIG. 104. A background signal (0) or a positive signal (1) was determined in each imaging step (after extension with A or C, after the first labeling, after extension with G or T, after the second labeling, and after THP cleavage). The encoding for the first 13 steps are 1111 for C, 0111 for A, 0011 for G, and 0001 for T. The correct ddNTP analogue was incorporated in each cycle, based on the known sequence of the template strand.

### Example 2: Single-Color Fluorescent Sequencing by Synthesis (SBS) Using Hybrid Approach with Unlabeled NRTs and Fluorescent ddNTPs, with Two Anchors, One Cleavable Linker, and Two Dyes with the Same or Similar Fluorescence Spectra, One or which is pH Responsive

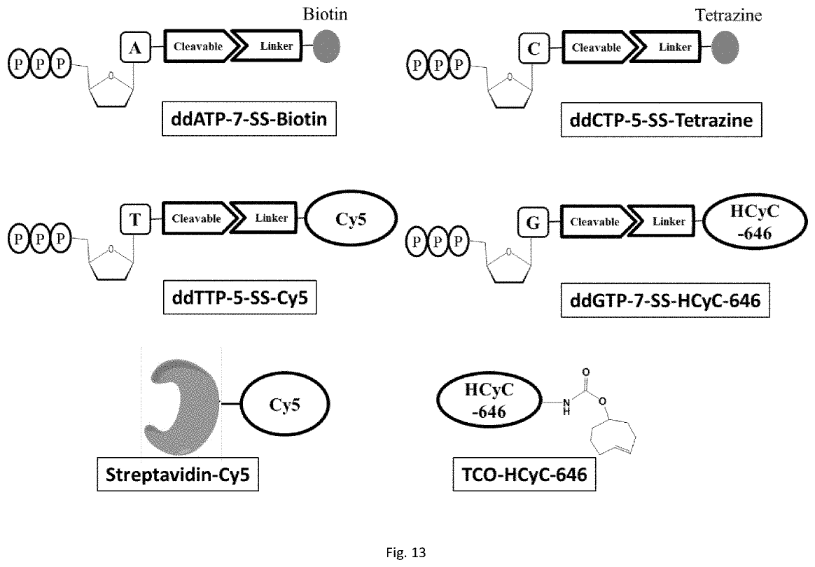

This scheme takes advantage of two dyes, Cy5 and HCyC-646, with very similar spectral properties, i.e., essentially identical absorption and emission profiles. However, while Cy5 will fluoresce over a wide pH range, HCyC-646 will only have substantial fluorescence in its protonated form below pH6 (Hilderbrand et al 2008). This pH-responsive property of HCyc-646 is used to develop a different “single-color” fluorescent SBS method utilizing a set of ddNTP analogues with either of two anchors (e.g., biotin and Tetrazine), either of two dyes (Cy5 and HCyC-646) (FIG. 13), and a single cleavable linker between the bases and the dyes or anchors.

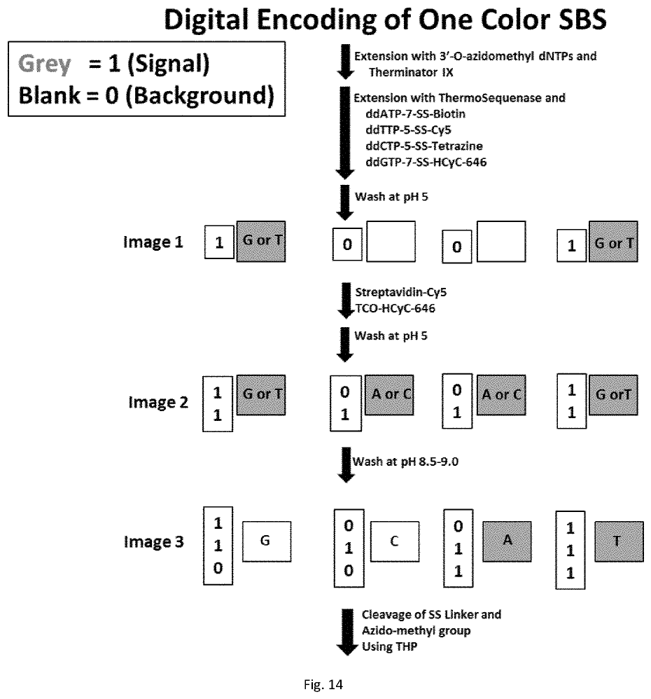

Following the logic of exemplary FIG. 14, which is based on the hybrid ddNTP/NRT approach, initially an extension reaction is performed with the four reversible terminators (e.g., 3′-O-azidomethyl-dNTPs or 3′-O-alkyldithiomethyl-dNTPs where the alkyl groups include methyl, ethyl or t-butyl moieties) under conditions leading to extension of at least 90% and up to about 98% of the amplified template molecules. Next, a second extension is performed with ddATP-7-SS-Biotin, ddTTP-5-SS-Cy5, ddCTP-5-SS-Tetrazine and ddGTP-7-SS-HCyC-646. After a wash at pH 5, imaging will reveal positive fluorescent signals for incorporation of the G and T analogues, but not which one was incorporated. Next labeling is performed with Streptavidin-Cy5 and TCO-HCyC-646. Another pH 5 wash followed by imaging will result in new signals for A and C, but again it will not be possible at this step to tell whether A or C was incorporated. Next a wash is carried out at pH 8.5-9, which will reverse the pH-induced fluorescence for the two ddNTP analogues now bearing HCyc-646, i.e., the C and G analogues.

Thus, if it was previously determined that either A or C was incorporated and the fluorescence remains, it indicates A incorporation; if the fluorescence is lost, it indicates C was incorporated. Similarly, if it was previously determined that either G or T was incorporated and the fluorescence remains, it indicates T was incorporated; if the fluorescence is lost, it indicates G was incorporated.

Cleavage with THP will be performed to cleave all the linkers, removing any dye and also cleaving the azidomethyl blocking group on the NRTs. If a positive signal is indicated by the integer 1 and a background signal by a 0, then based on imaging after extension with ddNTP analogues and a low pH wash, after labeling and a low pH wash and after the subsequent high pH wash, A will be encoded by 011, C by 010, G by 110 and T by 111.

The middle imaging step is not essential, as the first and third imaging steps will be sufficient to distinguish A (01), C (00), G (10) and T (11). Structures of the four nucleotide analogues are presented in FIG. 15, and a detailed embodiment of the general SBS scheme provided by the invention is presented in FIG. 16.

Any pair of dyes having essentially identical spectral properties to each other can be used, so long as one of them fluoresces conditionally, e.g., at a particular pH. Though biotin and tetrazine are used in this example, other pairs of anchors and anchor binding molecules can be used. Though the cleavable linker in the example contains an SS group, alternative cleavable groups may be present in the linker.

In a variant of this approach, the second pH 5 wash and the pH 9 wash are reversed. In this case, imaging after the pH 9 wash will indicate incorporation by either of the two nucleotide analogues labeled with Cy5, either directly or via labeling (the ddA and ddT analogues). Imaging after the subsequent pH 5 wash will result in gain of fluorescence due to HCyC-646 on ddC and ddT analogues, with remaining fluorescence due to Cy5 on ddA and ddG analogues. All other steps and the ultimate determination of which nucleotide analogue was incorporated are the same as described above in this example. With this variant, the digital encoding from the three imaging steps will be 101 for G, 001 for C, 011 for A and 111 for T, and actually only the images after the initial pH 5 wash after extension and the pH 9 wash after labeling are sufficient for sequence determination (10 for G, 00 for C, 01 for A and 11 for T).

The biotin and tetrazine anchors can be replaced with alternate anchors, so long as the desired dye molecule is attached to the appropriate anchor binding molecule. For instance, if the anchor is phenyldiboronic acid, salicyclhydroxamic acid-HCyC-646 or salicyclhydroxamic acid-Cy5 can be used, or if the anchor is an azide, dibenzocyclooctyne-HCyC-646 or dibenzocyclooctyne-Cy5 can be used.

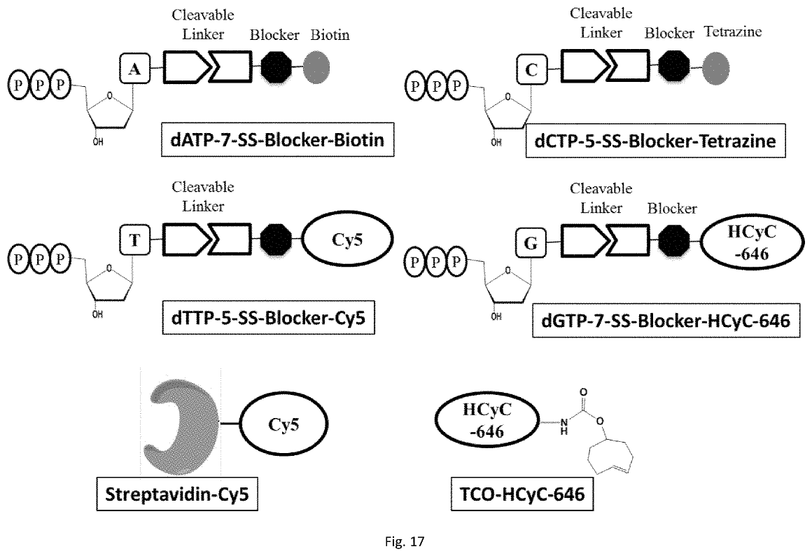

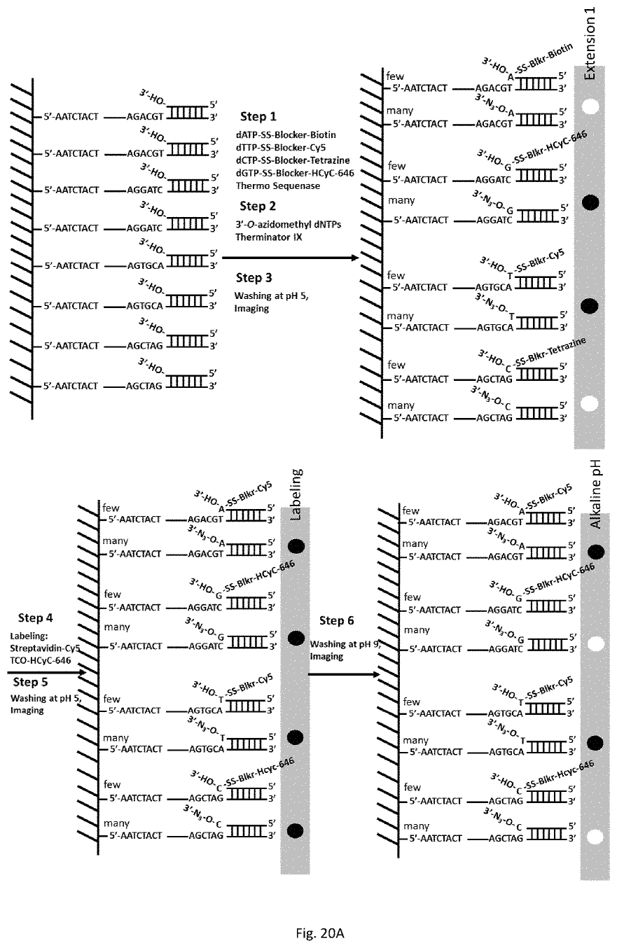

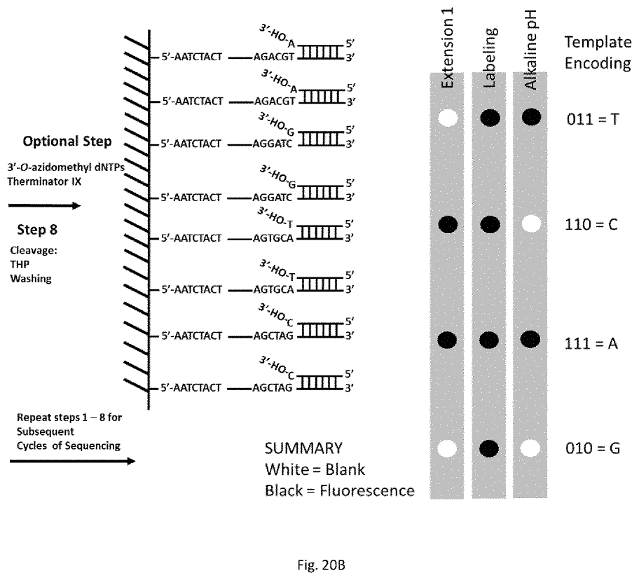

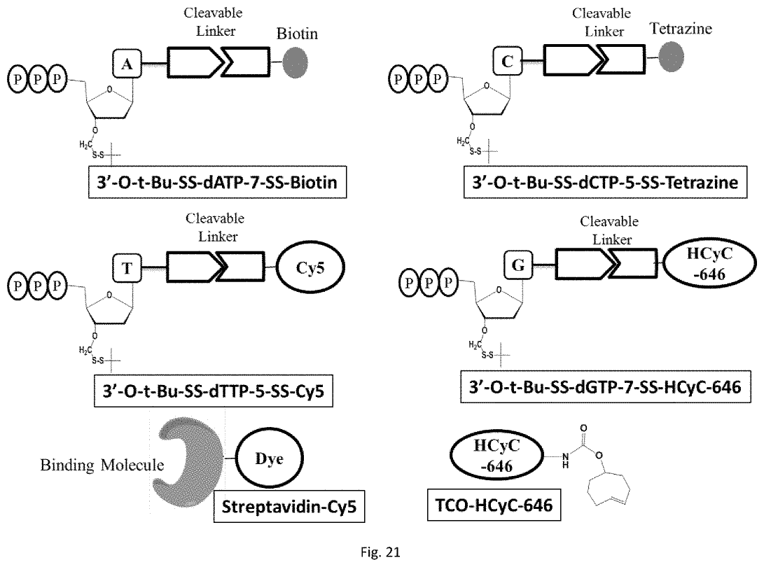

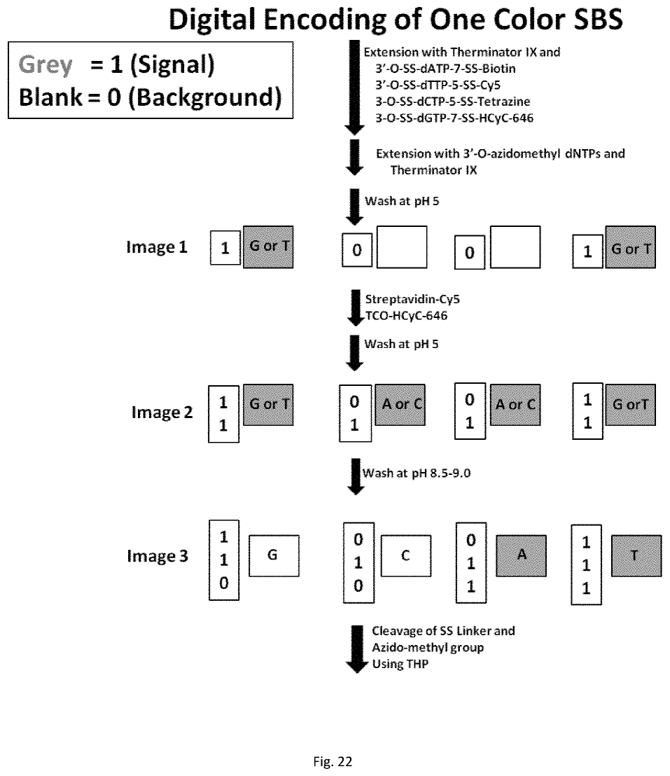

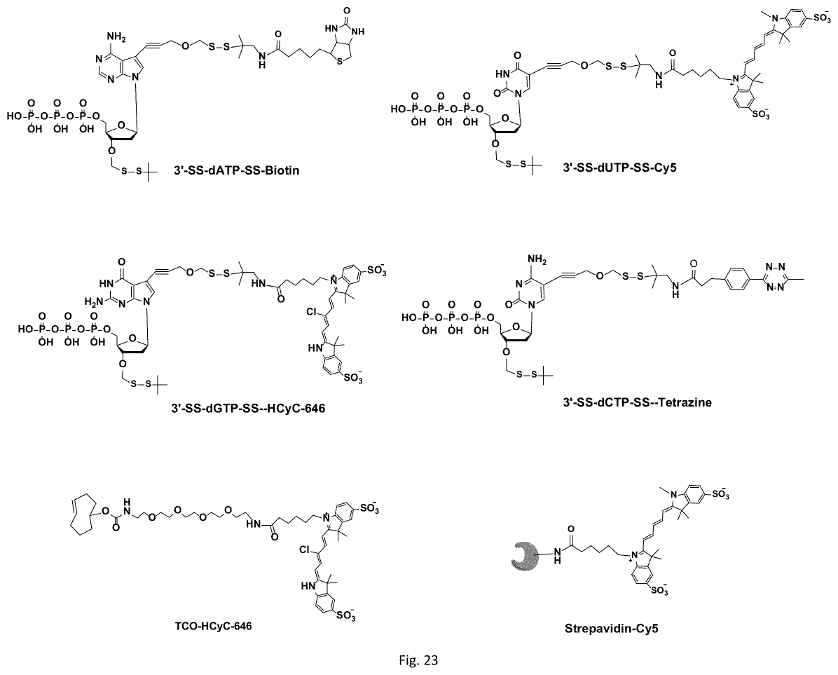

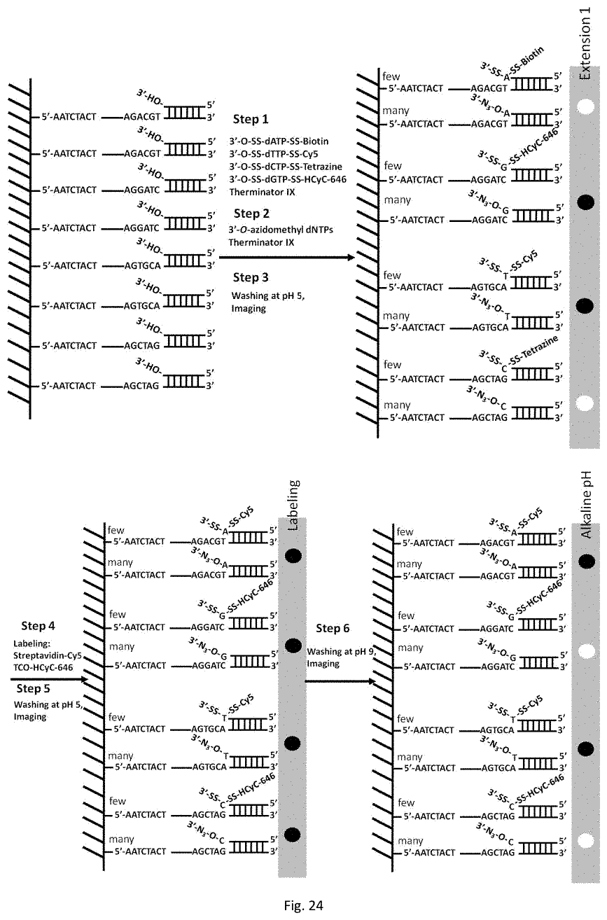

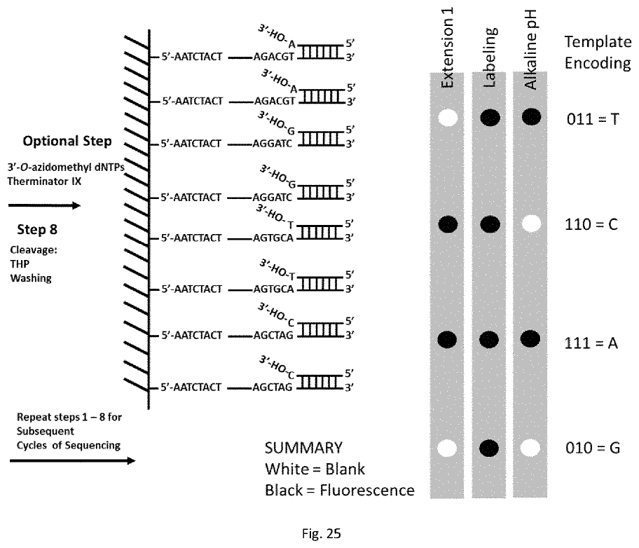

Another exemplary embodiment of this SBS approach using virtual terminators is disclosed in FIGS. 17-20. A similar approach using 3′-blocked nucleotide reversible terminators is disclosed in FIGS. 21-25.

### Example 3: Single-Color Fluorescent Sequencing by Synthesis (SBS) Using Hybrid Approach with Unlabeled NRTs and Fluorescent ddNTPs, Two with Cleavable Linkers and Two with Uncleavable Linkers, with Photobleaching

In addition to eliminating fluorescence by cleavage of a linker, thereby removing the dye, two other methods can also result in loss of a fluorescent signal—photobleaching and quenching. The use of quenching approaches in single-color SBS are described in some of the subsequent examples of the invention (Examples 6, 7, 11 and 12).

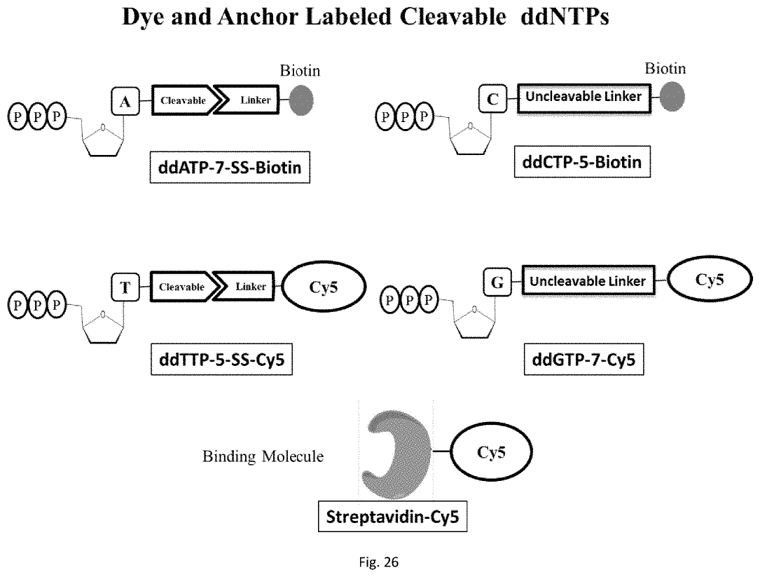

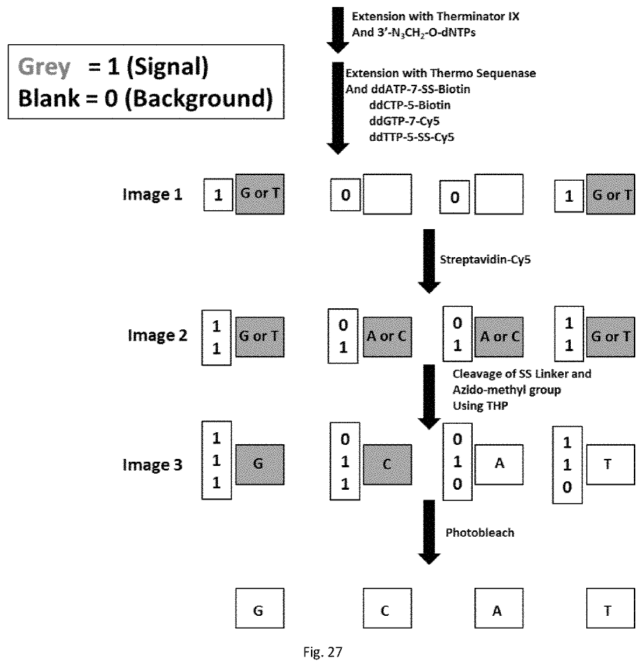

An example of this method provided by the invention presents a single-color SBS approach using photobleaching as shown in the exemplary scheme in FIG. 26. The general structures of the nucleotides for this scheme are presented in FIG. 27.

In this example, two of the nucleotides have either biotin or Cy5 attached via an SS linker; the other two have either biotin or Cy5 attached via an uncleavable linker. For ease of presentation the ddNTP analogues are presented as ddATP-SS-Biotin, ddTTP-SS-Cy5, ddCTP-Biotin and ddGTP-Cy5. The majority of template-loop-primers (or primers in other template-bound primer arrangements) on the surface are extended with reversible terminators, e.g., 3′-O-azidomethyl-dNTPs or 3′-O-alkyldithiomethyl-dNTPs where the alkyl groups include methyl, ethyl or t-butyl moieties, after which extension is carried out with the above four ddNTP analogues.

Imaging will reveal Cy5 fluorescence for incorporation by either the ddG or ddT analogues. Following a subsequent labeling reaction with streptavidin-Cy5 to link Cy5 to biotin on ddA and ddC analogues, a second round of imaging will result in cumulative fluorescence for all four nucleotides. Next, the disulfide bonds are cleaved to remove the dye molecules from the ddA and ddT analogues and restore the 3′-OH group on primers extended with the reversible terminators. Imaging will reveal Cy5 fluorescence only in the case where ddC or ddG analogues were incorporated. Finally, photobleaching is performed using the same laser emission but at higher power or for longer time. This will eliminate fluorescence of the dyes remaining on the ddC and ddG analogues. The system is now set for the second cycle of SBS.

Using the typical encoding scheme where 1 indicates a positive Cy5 signal and 0 a background signal, with imaging after the extension, labeling and quenching steps, incorporation of A will be encoded by 010, C by 011, G by 111, and T by 110; with consideration of just the first and third of these imaging steps, the encoding will be 00 for A, 01 for C, 11 for G, and 10 for T.

Examples of ddNTP analogues are presented in FIG. 28, and a detailed embodiment of the method provided by the invention is presented in FIG. 29. Any dye that can be effectively photobleached can be used for this method. Though the cleavable linker in the example contains an SS group, alternative cleavable groups may be present in the linker.

### Example 4: Two Color Fluorescent Sequencing by Syntheses (SBS) Using Hybrid Approach with Unlabeled NRTs and Fluorescent ddNTPs, and One Cleavable Linker

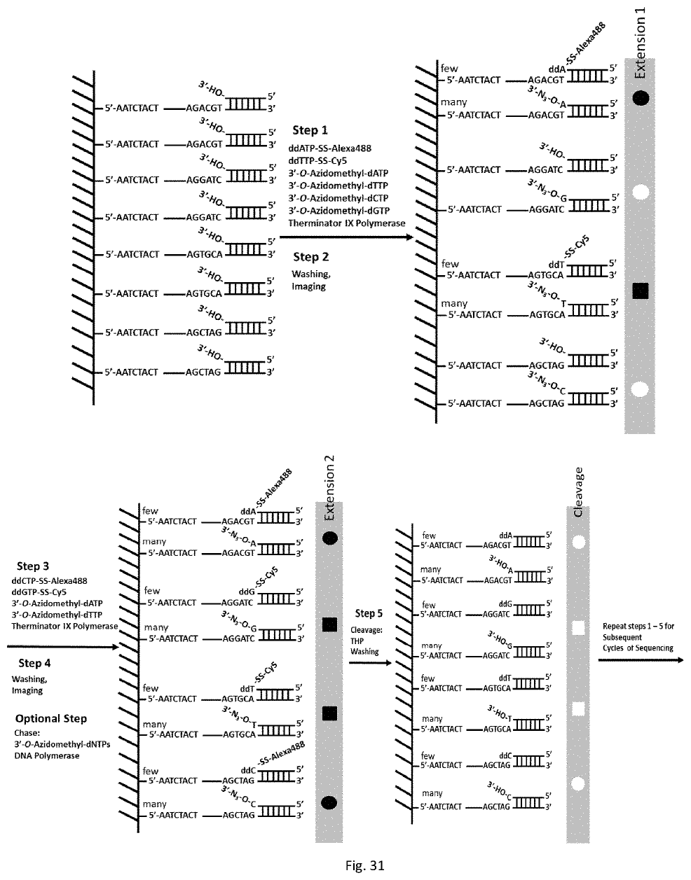

An example of this two-color SBS approach is shown in exemplary FIG. 31, and is essentially identical to the general method of the invention provided in Example 1, except that instead of biotin on two of the ddNTP analogues, a second dye, having different absorbance and emission spectra compared to the first dye, is attached. The general structures of such nucleotides are presented in FIG. 30. Imaging is only necessary after two sequential extension steps. For ease of presentation, the ddNTP analogues are ddATP-SS-Alexa488, ddTTP-SS-Cy5, ddCTP-SS-Alexa488 and ddGTP-SS-Cy5. In the first extension step, only ddATP-SS-Alexa488 and ddTTP-SS-Cy5 are added in the presence of an excess of the four azidomethyl-dNTPs (or other nucleotide reversible terminators). This is followed by the first imaging step, where a positive signal for Alexa488 will reveal incorporation of A and a positive signal for Cy5 will reveal T incorporation. A second extension step is performed with ddCTP-SS-Alexa488 and ddGTP-SS-Cy5 in the presence of higher concentrations of azidomethyl-dATP and azidomethyl-dTTP.

A second imaging step is performed; an Alexa488 signal will indicate incorporation of C and a Cy5 signal will indicate incorporation of G. Cleavage with THP will be performed to cleave all the linkers, removing any dye and also cleaving the azidomethyl blocking group on the NRTs. As in Example 1, a preponderance of the primers are extended by NRTs (>95%), with a sufficient number of primers having the ddNTP analogues incorporated to obtain a satisfactory signal, as any primers extended with the ddNTP analogues are lost to further cycles of sequencing.

This approach shares many of the same advantages as Example 1. Among these advantages are eliminating the use of a second cleavable group and the requirement for labeling steps, though this method requires two labels. The ddNTP analogues used in the example are presented in FIG. 30.

Other pairs of dyes (e.g., Cy5 and BodipyFL) can also be used for this two-color approach. Though the cleavable linker in the example contains an SS group, alternative cleavable groups may be present in the linker.

The same design can be used also with NRTs in a standard SBS design or with virtual terminators. In these cases, an optimal amount of unlabeled unlabeled 3′-O-azidomethyl dNTPs or other reversible terminators are added together with the labeled nucleotide analogues to maintain fidelity of the polymerase reaction, and a further chase step with, for example, unlabeled 3′-O-azidomethyl dNTPs, would typically follow the addition of the labeled nucleotides.

### Example 5: Single-Color Sequencing by Synthesis Using an Orthogonal Set or ddNTP Analogues, with SS Linked Dye or Anchor, or TCO-Carbamate Liked Dye or Anchor, Taking Advantage of a Diels-Alder Pyridazine Elimination Reaction

A novel “click to release” chemistry has been reported (Rossin et al 2016, 2018; Versteegen et al 2018) in which a TCO carbamate bond linkage to a tag can be cleaved by tetrazine, releasing the tag and carbon dioxide. These authors developed the approach for triggered drug release of an antibody drug conjugate.

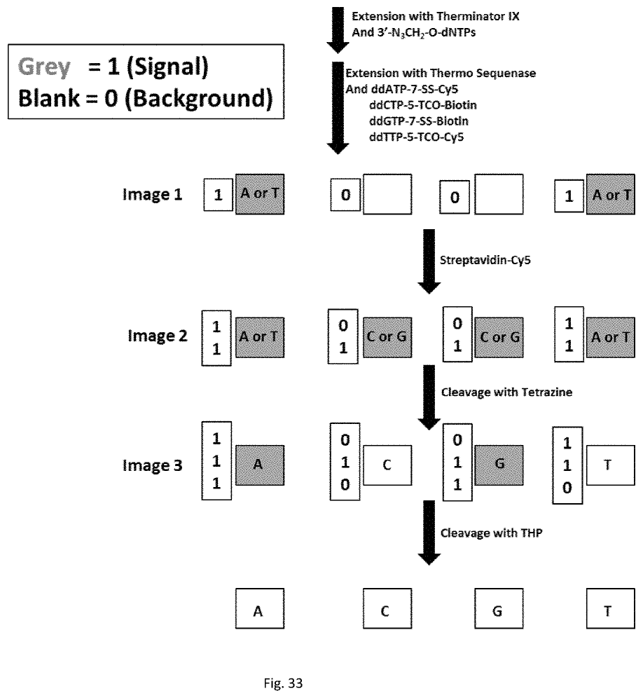

It is herein disclosed that this concept can be unexpectedly adapted to create a completely novel approach that eliminates the use of Azo linkers (or can be an alternative to other established linkers such as allyl or 2-nitrobenzyl) for performing single-color SBS. This approach is described and exemplified in a hybrid approach involving dideoxynucleotides as described in this section and exemplified in FIG. 33.

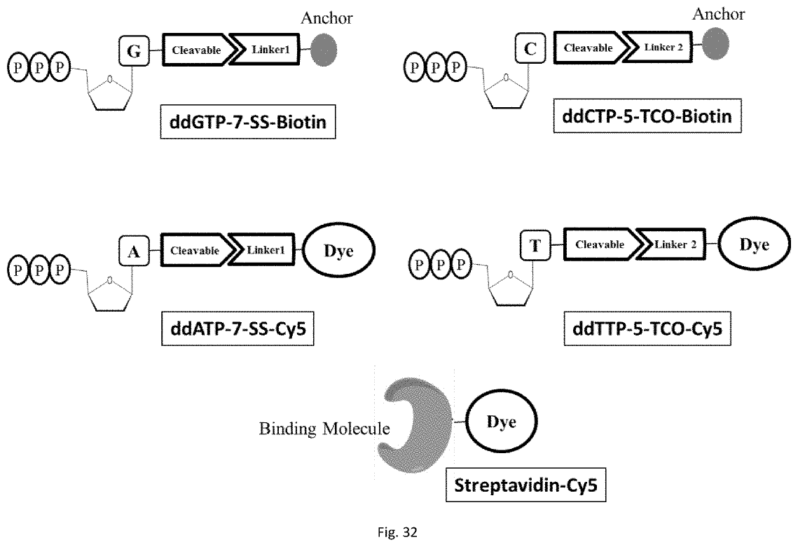

Four different nucleotides are designed with the general structures illustrated in FIG. 32. In this example, the first two are shown as ddNTP analogues in which either Cy5 (shown for ddA) or biotin (shown for ddG) are attached to the base by an SS bond. The other two are ddNTP analogues in which Cy5 (shown for ddT) or biotin (shown for ddC) are attached to the base via a TCO-carbamate linker. As usual, the majority of template-loop-primers (or primers in other template-bound primer arrangements) on the surface are extended with reversible terminators such as 3′-O-azidomethyl dNTPs, after which extension is carried out with the above four ddNTP analogues. After this step, imaging will reveal Cy5 fluorescence for incorporation by either the ddA or ddT analogues. Labeling with streptavidin-Cy5 will attach the dye to the biotin on the ddG and ddC analogues, resulting in cumulative fluorescence for all four nucleotides. Next, tetrazine is added to click to the TCO moiety triggering elimination of carbon dioxide and release of the dyes from the ddC and ddT analogues.

Finally, the disulfide bonds are cleaved to remove all the dye molecules of the ddNTP analogues and restore the 3′-OH group on primers extended with the reversible terminators, in readiness for the next cycle of SBS. In this case, considering the imaging after the extension, labeling and cleavage reactions, where 1 is a positive Cy5 signal and 0 indicates background Cy5 signal, incorporation of A is encoded by 111, C by 010, T by 110 and G by 011; considering only the first and third of these imaging steps, the following encoding results: 11 for A, 00 for C, 10 for T and 01 for G. Example ddNTP analogues that can be used in this general SBS method of invention are presented in FIG. 34. A detailed embodiment of this general SBS scheme is shown in FIG. 35. Alternatives to the SS cleavable group in linker 1, alternative dyes and alternative anchors can also be used.

The same design can be used also with NRTs in a standard SBS design or with virtual terminators. In these cases, a chase step with, for example, unlabeled 3′-O-azidomethyl dNTPs, would typically follow the addition of the labeled nucleotides.

### Example 6: Single Color Sequencing by Synthesis Using a Set or ddNTP Analogues, One with Dye Only, One with Dye and Anchor 1, One with Anchor 1 Only, and One with Both Anchors 1 and 2, all with SS Linkers, Taking Advantage of a Dye Quencher

Carlson et al (2018) described the use of a BHQ-Tetrazine compound to carry out the click to release reaction described in the previous method. It is herein disclosed that even without the elimination reaction, BHQ-Tetrazine could unexpectedly be used as a convenient and effective binding molecule for bringing a quencher in close enough proximity to a dye on a nucleotide analogue via its ability to bind to TCO, making possible another single-color fluorescent SBS approach.

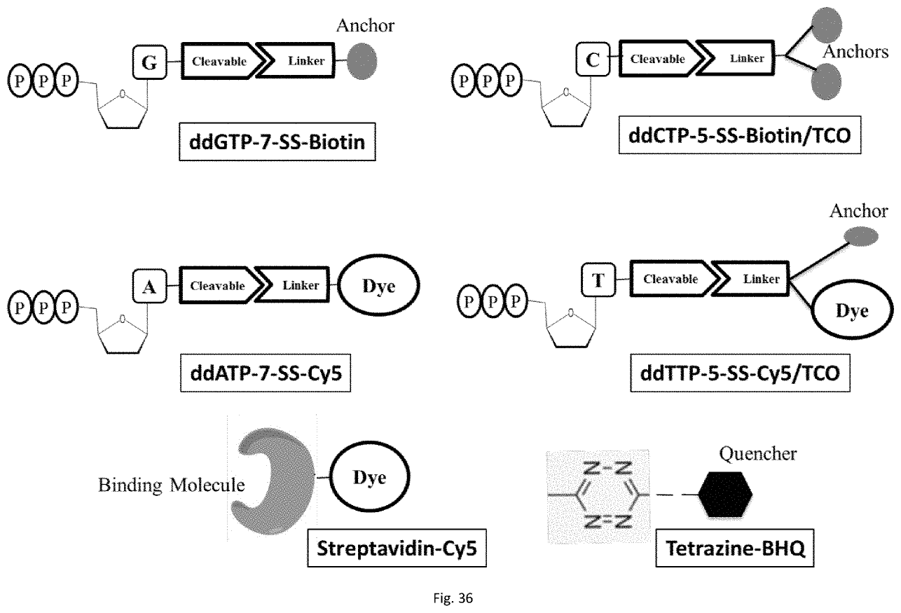

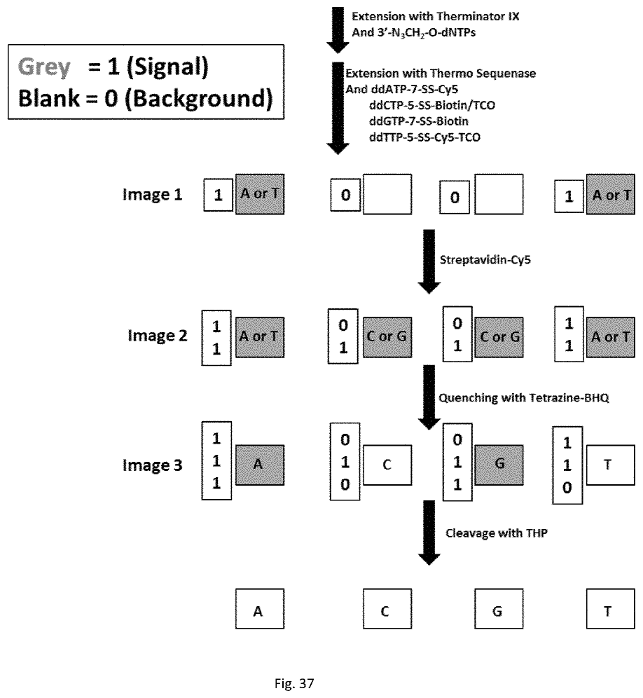

This general SBS approach, provided by the invention, is first described herein in the context of a combined ddNTP/NRT approach, an example of which is shown in a general scheme in FIG. 37. Four different nucleotides are designed with the general structures illustrated in FIG. 36. The first two shown in this example are ddNTP analogues in which either Cy5 (shown for ddA) or biotin (shown for ddG) are attached to the base by an SS linker. The third is a novel ddNTP analogue (shown for ddT) in which Cy5 is attached to the base via an SS linker and in which TCO is attached to the Cy5.

Finally, the fourth ddNTP analogue (shown for ddC) has both biotin and TCO attached to the base via an SS bond, the latter via a short branched chain.

In the example, the majority of template-loop-primers on the surface are extended with reversible terminators such as 3′-O-azidomethyl dNTPs, after which extension is carried out with the above four ddNTP analogues. After this step, imaging will reveal Cy5 fluorescence for incorporation by either the ddA or ddT analogues.

Labeling with streptavidin-Cy5 will attach the dye to the biotin on the ddG and ddC analogues, resulting in cumulative fluorescence for all four nucleotides. Next incubation is carried out with a tetrazine-BHQ quencher, which will attach to the TCO group on the ddT and ddC analogues, quenching the Cy5 fluorescence in these molecules, while not affecting the fluorescence of Cy5 on the ddA and ddG analogues. Finally, the disulfide bonds are cleaved to remove all the dye molecules of the ddNTP analogues and restore the 3′-OH group on primers extended with the reversible terminators, in readiness for the next cycle of SBS.

Using the typical encoding scheme where 1 indicates a positive Cy5 signal and 0 a background signal, with imaging after the extension, labeling and quenching steps, incorporation of A will be encoded by 111, C by 010, G by 011, and T by 110; with consideration of just the first and third of these imaging steps, the encoding will be 11 for A, 00 for C, 01 for G, and 10 for T.

Dye-quencher combinations other than Cy5 and BHQ can be used in this SBS approach. Exemplary nucleotides useful in this SBS approach are presented in FIG. 38. A detailed embodiment of this SBS approach is presented in FIG. 39. Other dyes and replacements for Anchor 1 and the SS cleavable group could be used. Moreover, Anchor 2 is not limited to TCO. Any anchor can be used for binding the quencher.

Indeed, as shown in Examples 11 and 12, tetrazine is used as the anchor and TCO-BHQ3 is the anchor binding molecule-quencher.

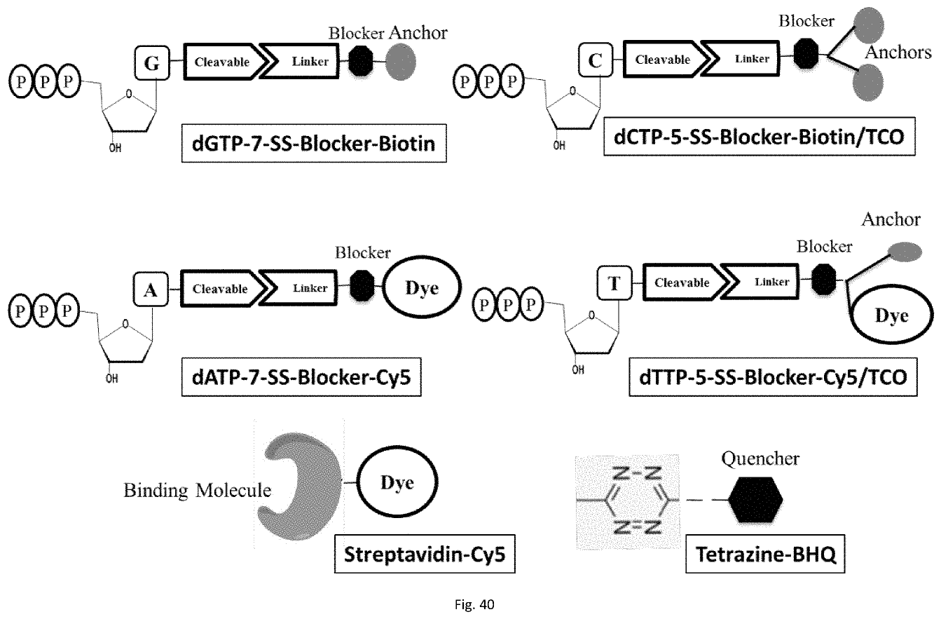

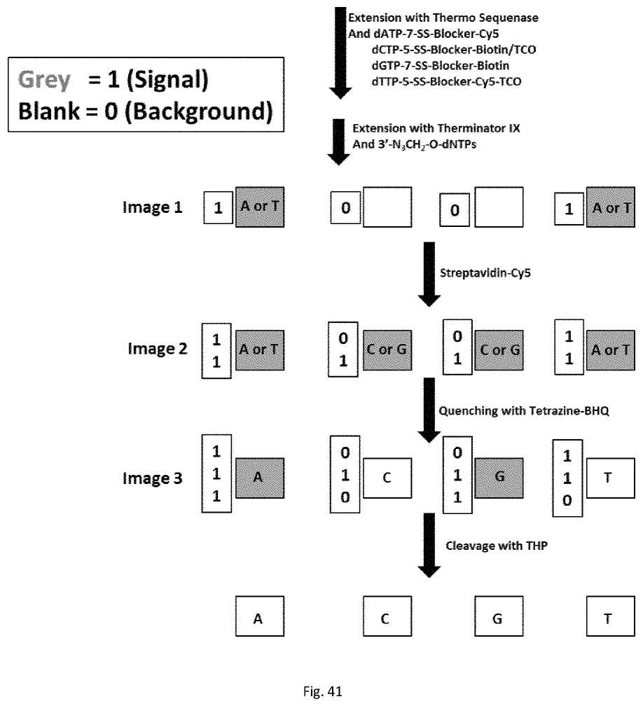

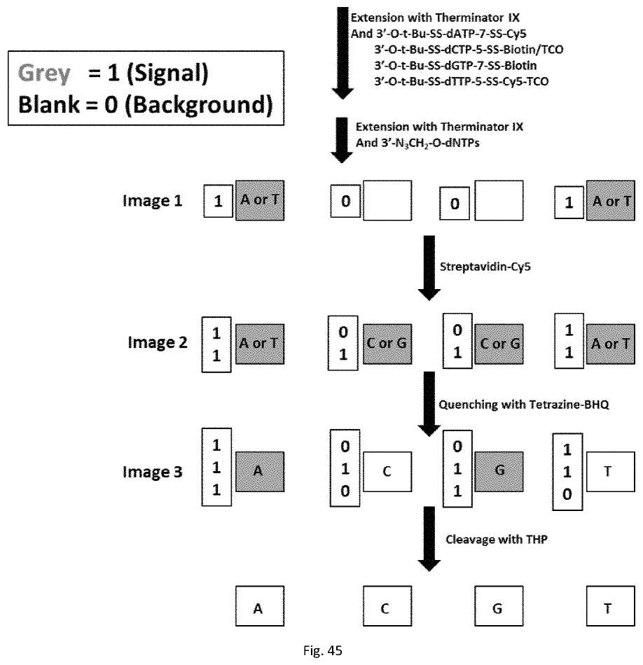

The invention also provides analogous SBS approaches using reversible terminators in place of the dideoxynucleotide analogues, for example: (1) approaches using virtual terminator analogues in which a cleavable blocking group is attached to the base as exemplified in FIGS. 40-43; and (2) approaches using 3′-O-t-butyl-dNTP analogues as exemplified in FIGS. 44-47. The protocols are essentially identical to the protocol with ddNTP analogues except that non-fluorescent reversible terminators are only needed during a chase step.

### Example 7: Single Color Sequencing by Synthesis Using a Set of ddNTP Analogues, on with Cy5, Om with pH-Responsive Fluor HCyC-646, Om with Cy5 and TCO Anchor, and One with HCyC-646 and TCO Anchor, all Attached to Base Via SS Linkers, Using a Dye Quencher Attached to Tetrazine

Some of the SBS approaches provided by the invention and exemplified above, were described with the use of a fluorescent dye, HCyC-646, that exhibits fluorescence at pH 5-6, but not at a higher pH (Example 2), as well as the use of a quencher, BHQ (Example 6). In this SBS approach, these concepts are combined to generate another novel single-color SBS approach.

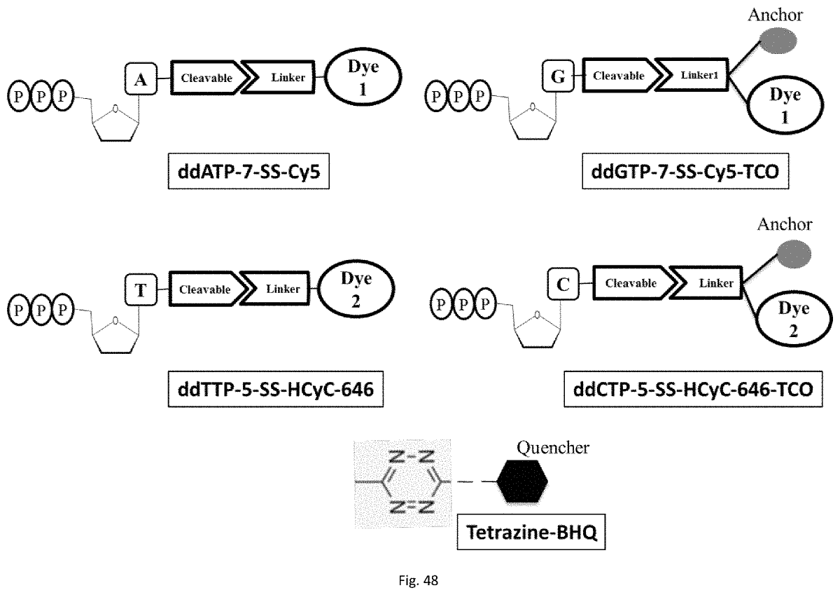

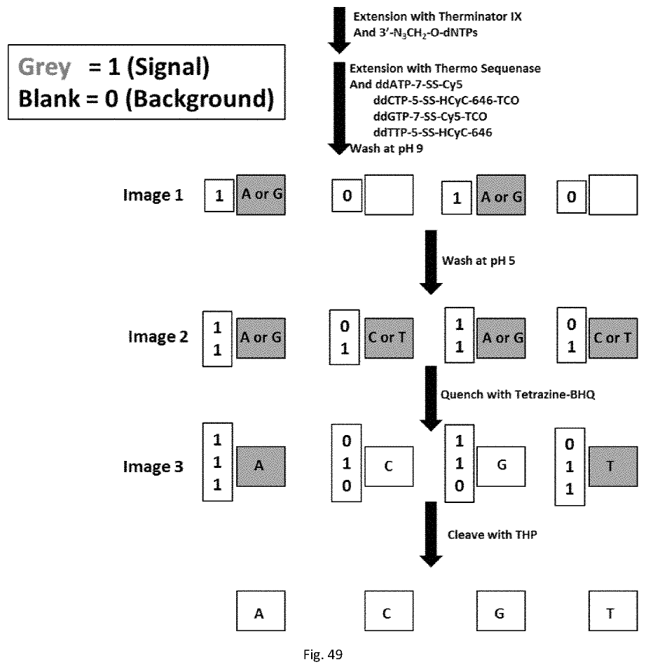

For the purpose of illustration, this approach is described using a combined ddNTP/NRT approach as exemplified in the general scheme in FIG. 49. Four different nucleotides are designed with the general structures illustrated in FIG. 48. The first two are ddNTP analogues in which either Cy5 (shown for ddA) or HCyC-646 (shown for ddT) are attached to the base by an SS linker. The other two also have Cy5 (shown for ddG) or HCyC-646 (shown for ddC) attached to the base via an SS linker, but have a TCO anchor attached to the dye. In this exemplary scheme, the majority of template-loop-primers (or other template-bound primer arrangements) on the surface are extended with reversible terminators such as 3′-O-azidomethyl dNTPs, after which extension is carried out with the above four ddNTP analogues. After a wash carried out at pH 9 where HCyC-646 does not fluoresce at around 670 nm, imaging will reveal Cy5 fluorescence for incorporation by either the ddA or ddG analogues. A subsequent wash at pH 5 will result in fluorescence of the HCyC-646 on ddT and ddC, resulting in cumulative fluorescence for all four nucleotides. Next incubation is carried out with a tetrazine-BHQ quencher, which will attach to the TCO group on the ddG and ddC analogues, quenching the Cy5 in these molecules; following a wash at pH 5, only the ddA and ddT nucleotide analogues will display fluorescence. Finally, the disulfide bonds are cleaved to remove all the dye molecules of the ddNTP analogues and restore the 3′-OH group on primers extended with the reversible terminators, in readiness for the next cycle of SBS.

Using the encoding scheme where 1 indicates a positive Cy5 signal and 0 a background signal, with imaging after the extension, labeling and quenching steps, A will be encoded by 111, C by 010, G by 110, and T by 011; with consideration of just the first and third of these imaging steps, the encoding will be 11 for A, 00 for C, 10 for G, and 01 for T. Examples of ddNTP analogues useful in this SBS approach are presented in FIG. 50. A detailed exemplary scheme of this SBS approach is presented in FIG. 51. Dyes other than Cy5 and HCyC-646, a cleavable group other than SS, and an anchor other than TCO may be used.

In a variant of this approach, the pH 9 wash and the first pH 5 wash are reversed. In this case, imaging after the pH 5 wash will indicate incorporation by any of the four nucleotide analogues.

Imaging after the pH 9 wash will result in loss of fluorescence of HCyC-646 on ddC and ddT analogues, with remaining fluorescence due to Cy5 on ddA and ddG analogues. All other steps and the ultimate determination of which nucleotide analogue was incorporated are the same as described above in this example. In this variant, the encoding based on the three imaging steps will be 111 for incorporation of A, 100 for C, 110 for G and 101 for T, with technically only the last two imaging steps sufficient (11 for A, 00 for C, 10 for G and 01 for T).

The TCO anchor can be replaced with alternate anchors, so long as the quencher is attached to the appropriate anchor binding molecule.

For instance, if the anchor is biotin, streptavidin-BHQ can be used, or if the anchor is an azide, dibenzocyclooctyne-BHQ can be used.

This general SBS approach can also use NRTs or virtual terminators. In these cases, a chase step with, for example, unlabeled 3′-O-azidomethyl dNTPs, would typically follow the addition of the labeled nucleotides.

### Example 8: Single Color Sequencing by Synthesis Using an Orthogonal Set of ddNTP Analogues, Two with Cy5 (One with SS Linker and One with SS-TCO Linker) and Two with Biotin (One with SS Linker and One with SS-TCO-Linker)

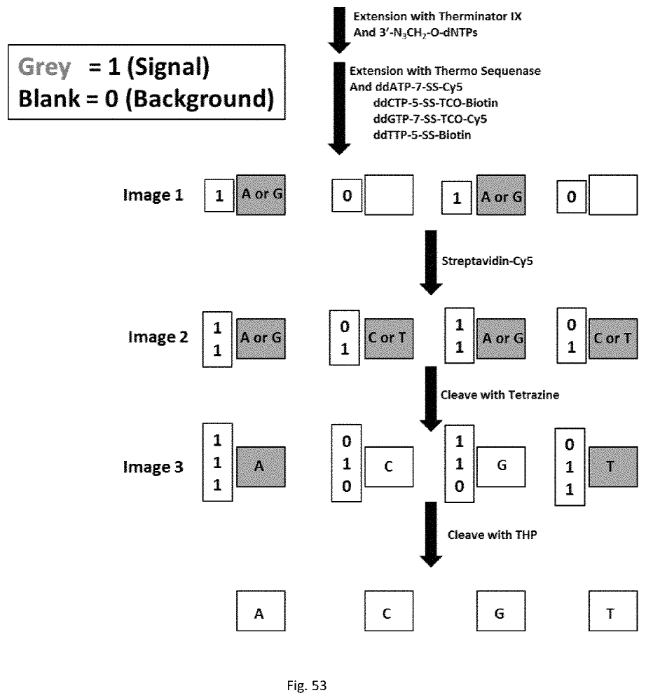

Example 5 above introduced the novel use of the TCO carbamate linker and the click-to-cleave concept. Although the TCO linker should be effectively cleaved by tetrazine, in case the cleavage is not complete, in this example, an SS group is situated within the linker proximal to the TCO group (i.e., closer to the base). In this way, a final cleavage with THP should remove all remaining dye. This concept is shown herein in the context of a combined ddNTP/NRT approach exemplified in the general scheme in FIG. 53.

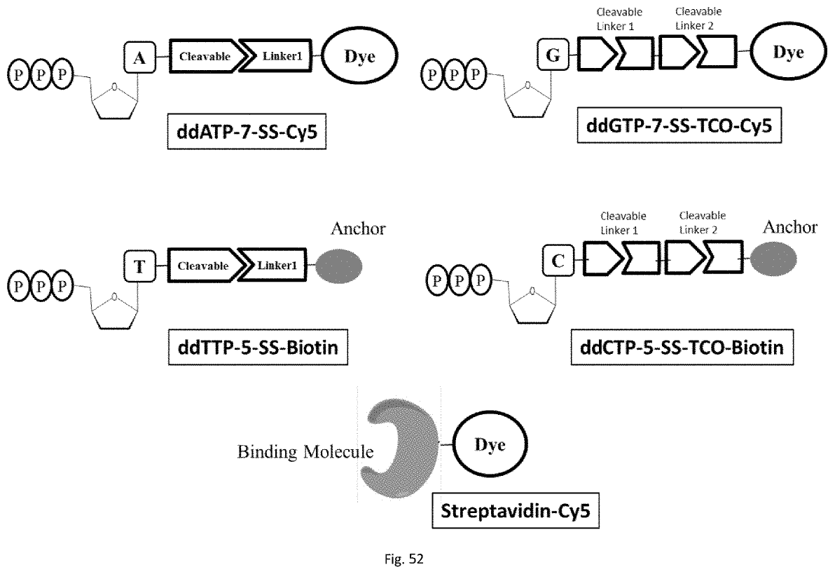

Four different nucleotides are designed with the general structures illustrated in FIG. 52. The first two are ddNTP analogues in which either Cy5 (shown for ddA) or Biotin (shown for ddT) are attached to the base by an SS linker. The other two also have Cy5 (shown for G) or Biotin (shown for C) attached to the base, but in this case via a linker containing both a proximal SS group and a more distal TCO group. In this exemplary scheme, the majority of template-loop-primers (or primers in other template-bound primer arrangements) on the surface are extended with reversible terminators such as 3′-O-azidomethyl dNTPs, after which extension is carried out with the above four ddNTP analogues. After a wash, imaging will reveal Cy5 fluorescence for incorporation by either the ddA or ddG analogues. A subsequent labeling with streptavidin-Cy5 will attach Cy5 to the ddT and ddC analogues, resulting in cumulative fluorescence for all four nucleotides. Next incubation is carried out with a tetrazine compound, which will react with the TCO group on the ddG and ddC analogues, resulting in removal of the Cy5; following a wash step, imaging will only result in fluorescence for the ddA and ddT nucleotide analogues. Finally, the disulfide bonds are cleaved to remove all the dye molecules from the ddNTP analogues and restore the 3′-OH group on primers extended with the reversible terminators, in readiness for the next cycle of SBS. Using the typical encoding scheme where 1 indicates a positive Cy5 signal and 0 a background signal, with imaging after the extension, labeling and quenching steps, A will be encoded by 111, C by 010, G by 110, and T by 011; with consideration of just the first and third of these imaging steps, the encoding will be 11 for A, 00 for C, 10 for G, and 01 for T.

Examples of ddNTP analogues useful in this SBS approach provided by the invention are presented in FIG. 54. A detailed embodiment of this SBS approach is presented in FIG. 55. A dye other than Cy5, a cleavable group other than SS, and an anchor other than biotin may be used. Moreover, other cleavable groups could replace the TCO, provided they are cleaved efficiently and specifically.

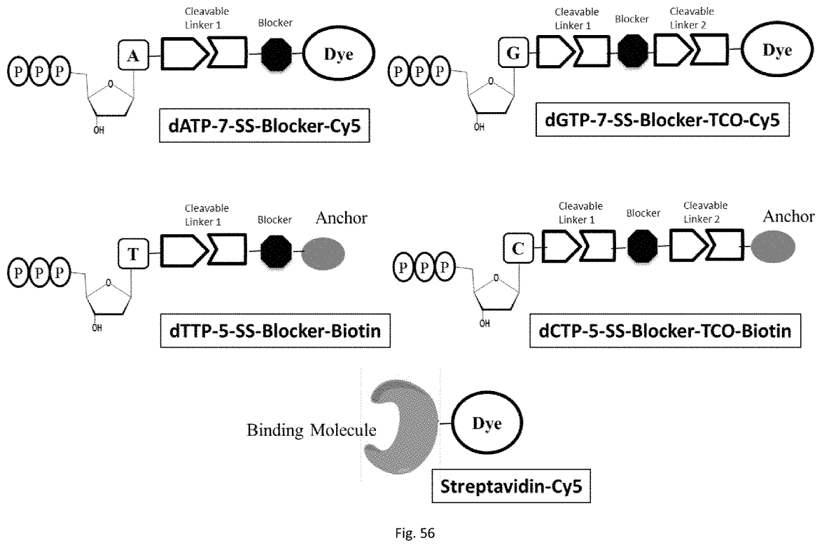

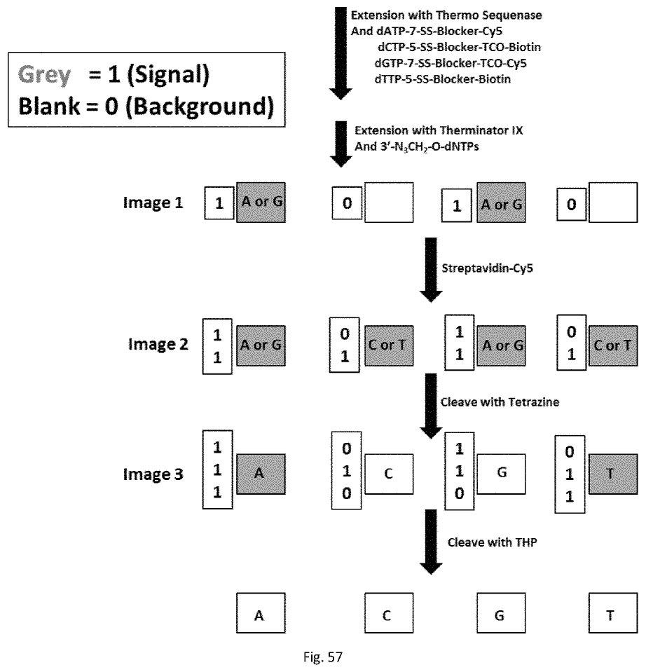

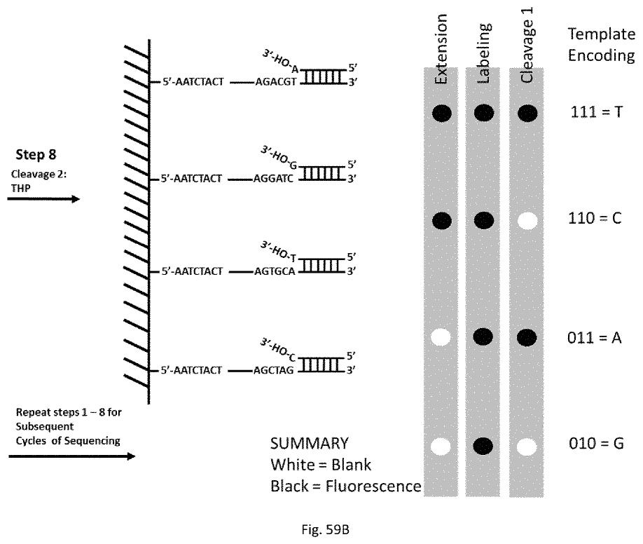

Analogous SBS approaches provided by the invention are disclosed herein. Approaches using virtual terminators analogues in place of the dideoxynucleotide analogues are exemplified in FIGS. 56-59.

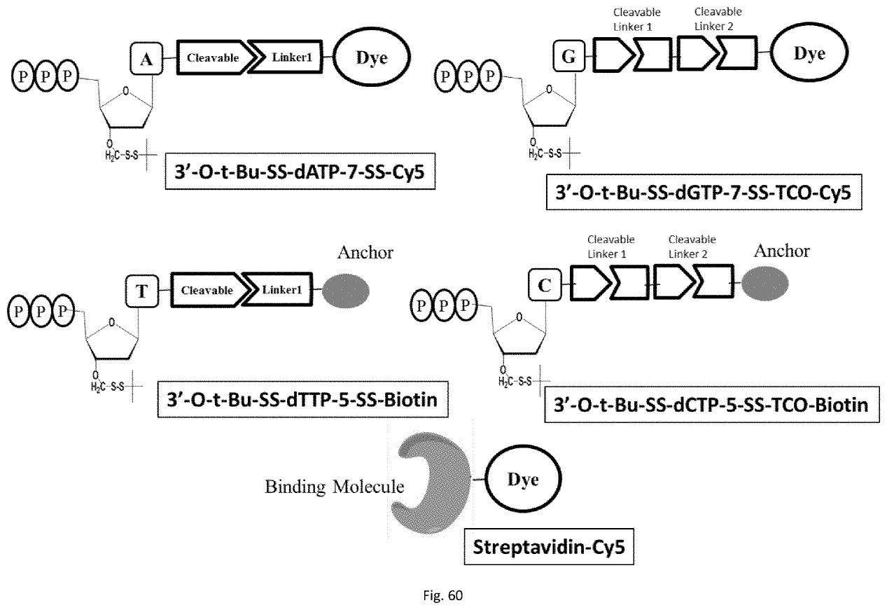

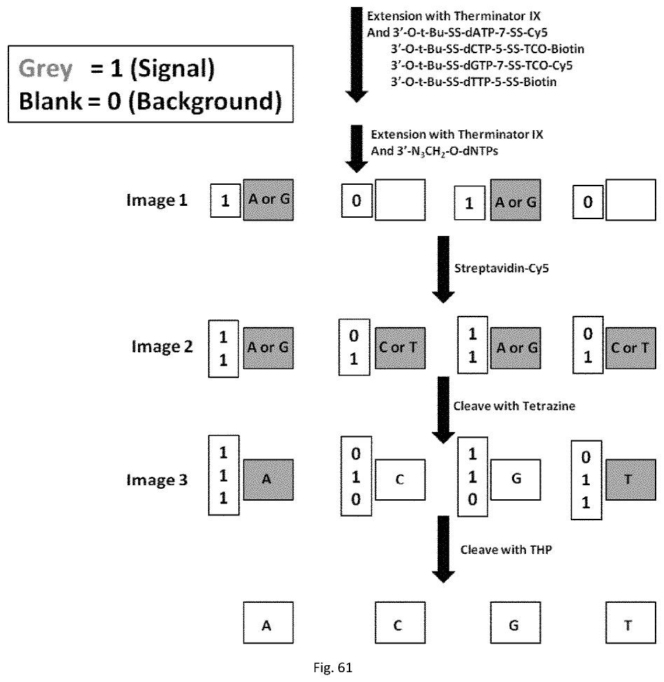

Approaches using 3′-O-t-butyl-dNTP analogues are exemplified in FIGS. 60-63. The protocols are essentially identical to the protocol with ddNTP analogues except that non-fluorescent reversible terminators are only needed during a chase step.

### Example 9: Single Color Sequencing by Synthesis Using in Orthogonal Set or ddNTP Analogues, Two with Cy5 (One with SS Linker and One with SS-TCO Carbmate Linker) and Two with HCyC-646 (One with SS Linker and One with SS-TCO 20 Carbmate Linker)

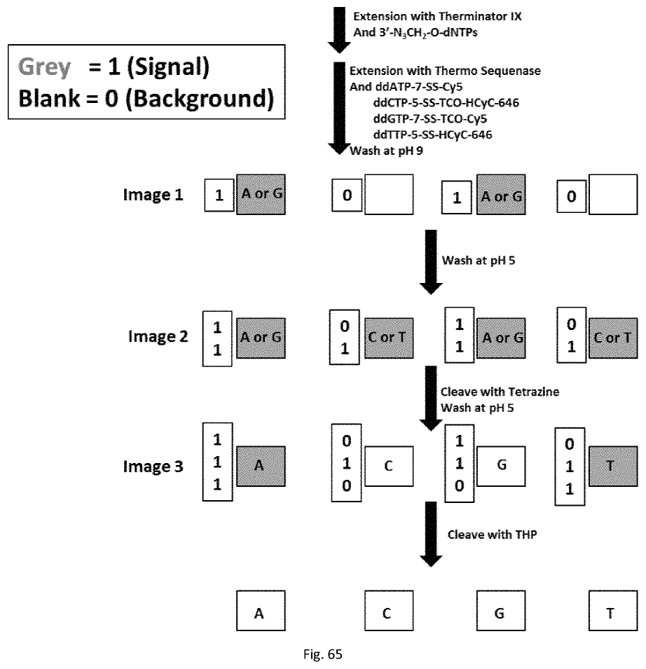

This SBS approach provided by the invention combines the features of above schemes using HCyC-646, a dye with pH responsive fluorescence, as well as those taking advantage of the novel click-to-release TCO linker. As in the previous example, a linker containing both an SS and TCO linker is used for two of the ddNTP analogues. An approach using this concept using the combined ddNTP/NRT format is shown herein in the exemplary scheme in FIG. 65.

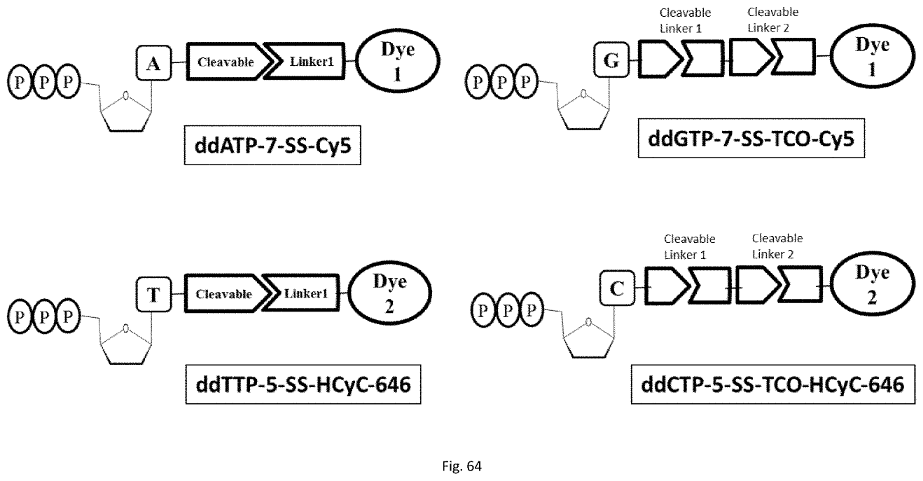

Four different nucleotides are designed with the general structures illustrated in FIG. 64. The first two are ddNTP analogues in which either Cy5 (shown for ddA) or HCyC-646 (shown for ddT) are attached to the base by an SS linker. The other two also have Cy5 (shown for ddG) or HCyC-646 (shown for C) attached to the base, but in this case via a linker containing both a proximal SS group (closer to the base) and a more distal TCO group (closer to the dye). In this exemplary scheme, the majority of template-loop-primers (or other template-bound primer arrangements) on the surface are extended with reversible terminators such as 3′-O-azidomethyl dNTPs, after which extension is carried out with the above four ddNTP analogues. After a wash at pH 9, imaging will reveal Cy5 fluorescence for incorporation by either the ddA or ddG analogues. A subsequent imaging after a pH 5 wash will allow fluorescence of HCyC-646 on ddT and ddC, resulting in cumulative fluorescence for all four nucleotides. Next incubation is carried out with a tetrazine compound, which will react with the TCO group on the ddG and ddC analogues, resulting in removal of the Cy5; following a wash step at pH 5, imaging will only result in fluorescence for the ddA and ddT nucleotide analogues. Finally, the disulfide bonds are cleaved to remove all the dye molecules of the ddNTP analogues and restore the 3′-OH group on primers extended with the reversible terminators, in readiness for the next cycle of SBS.

Using the typical encoding scheme where 1 indicates a positive Cy5 signal and 0 a background signal, with imaging after the extension, labeling and quenching steps, A will be encoded by 111, C by 010, G by 110, and T by 011; with consideration of just the first and third of these imaging steps, the encoding will be 11 for A, 00 for C, 10 for G, and 01 for T. Examples of ddNTP analogues useful in this SBS approach are presented in FIG. 66. A detailed example of this novel SBS approach is presented in FIG. 67. A dye other than Cy5, a cleavable group other than SS, and an anchor other than biotin may be used. Conditionally fluorescent dyes other than HCyC-646 may also be used.

In a variant of this approach, the pH 9 wash and the first pH 5 wash are reversed. In this case, imaging after the pH 5 wash will indicate incorporation by any of the four nucleotide analogues.

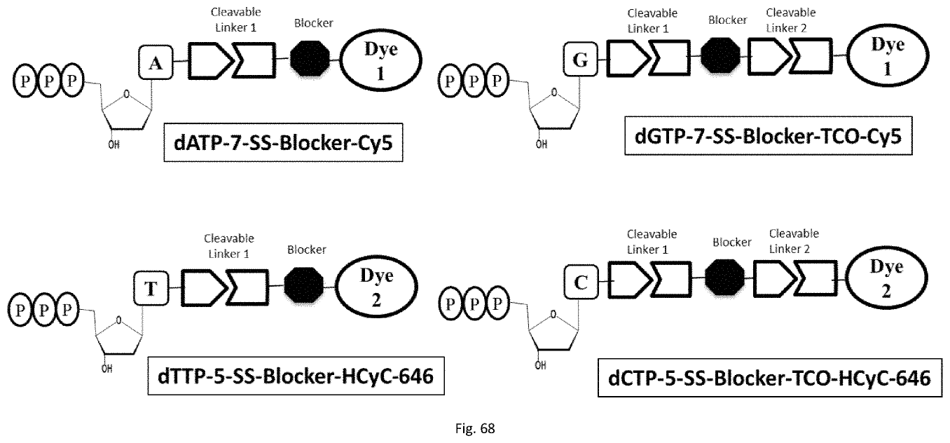

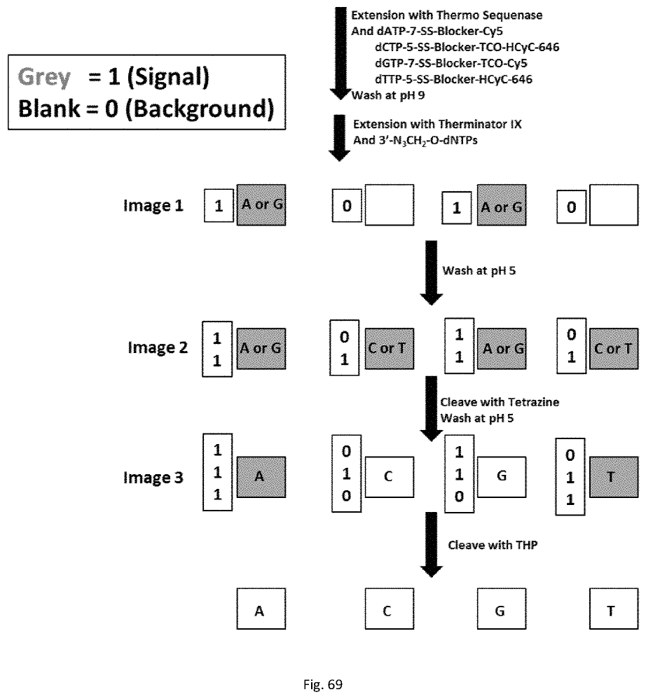

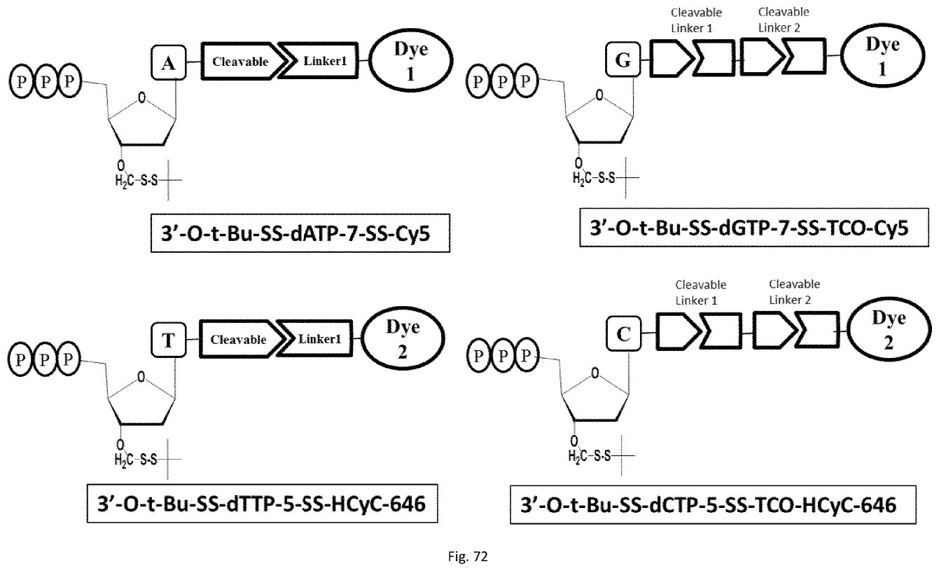

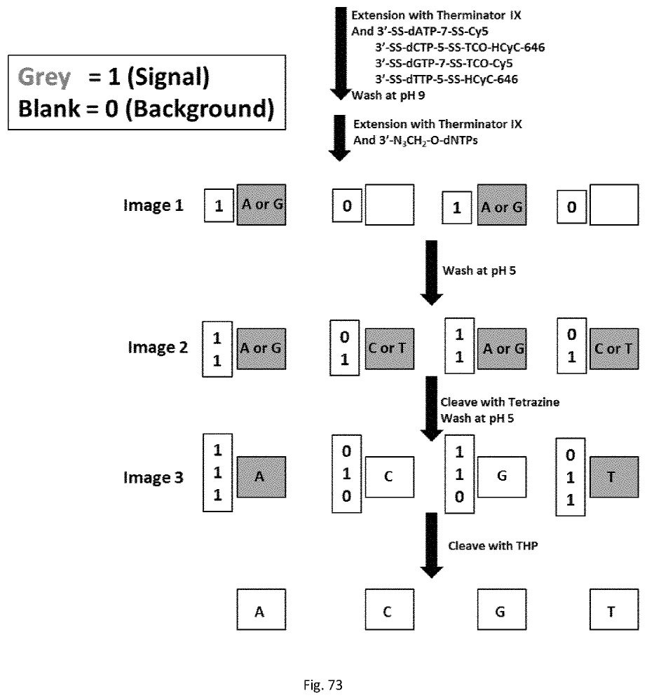

Imaging after the pH 9 wash will result in loss of fluorescence of HCyC-646 on ddC and ddT analogues, with remaining fluorescence due to Cy5 on ddA and ddG analogues. All other steps and the ultimate determination of which nucleotide analogue was incorporated are the same as described above in this example. In this variant, the encoding based on the three imaging steps is 111 for A, 100 for C, 110 for G and 101 for T. Imaging after only the second and third imaging steps is sufficient, in which case the encoding will be 11 for A, 00 for C, 10 for G and 01 for T. Herein are disclosed equivalent structures and schemes for this example using reversible terminators instead of the ddNTP hybrid approach: either (1) virtual terminators, bearing blocking groups between the base and the label (FIGS. 68-71), or (2) 3′-blocked nucleotide reversible terminators (FIGS. 72-75).

### Example 9A: Single Color Sequencing by Synthesis Using an Orthogonal Set or ddNTP Analogues, Two with Cy5 (One with SS Linker and One with Alternate Cleavable Linker) and Two with HCyC-646 (One with SS Linker and One with Alternate Cleavable Liner)

In this version of Example 9, the only difference is that instead of the TCO carbamate linker on two of the nucleotide analogues requiring the tetrazine click-to-cleave reaction, cleavable groups such as Azo, 2-Nitrobenzyl or Allyl are used. The other two nucleotide analogues still have SS linkers. The nucleotides with the alternate cleavable group may in addition have an SS group between the base and this second cleavable group. The SBS scheme and digital encoding is otherwise identical to that shown in Example 9, where instead of using tetrazine in the click-to-cleave reaction, the cleavage is carried out with sodium dithionite for Azo, 340 nm light for 2-Nitrobenzyl, or Pd(0) for Allyl.

### Example 10: Single Color Sequencing by Synthesis Using a Set or ddNTP Analogues, Two with Cy5 and Two with HCyC-646, all with SS Linker, with Two Extension Steps

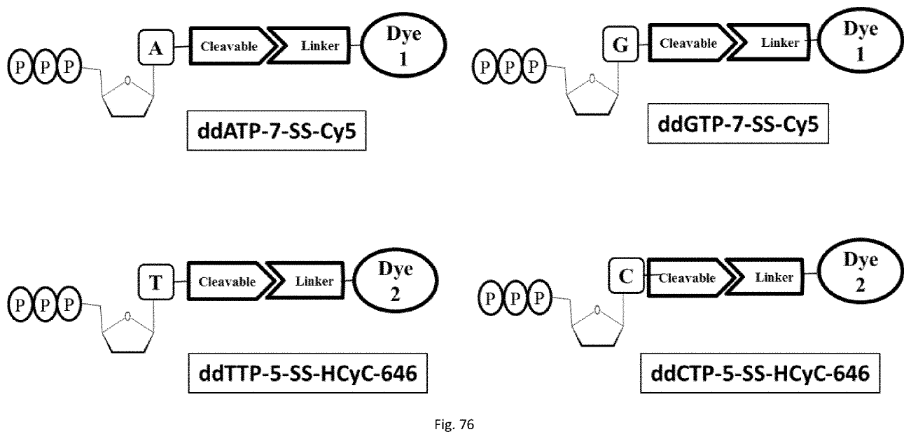

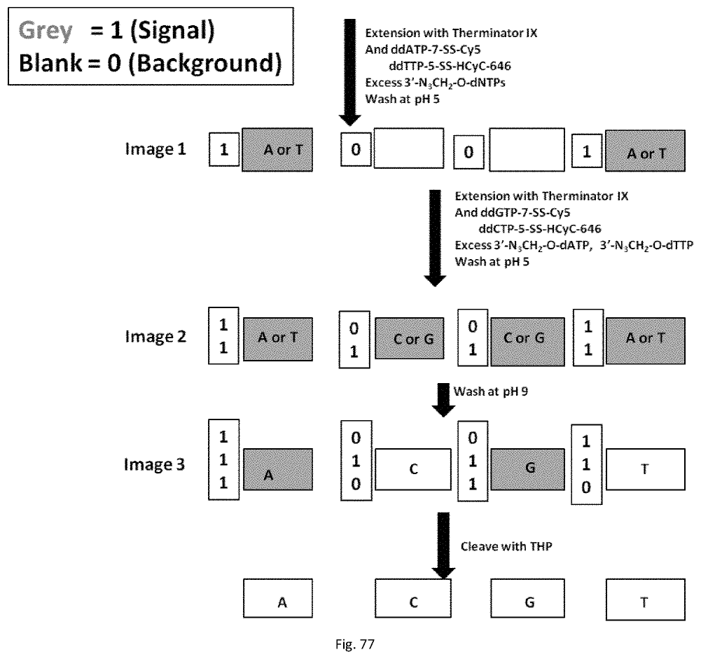

This example avoids the use of labeling or quenching steps, advantageously only requiring washes at different pH's and routine THP cleavage at the end. To achieve this, two of the nucleotide analogues are added at a time. For the purpose of illustration, this novel SBS approach is shown in a combined ddNTP/NRT format as exemplified in FIG. 77. Four different nucleotides are designed with the general structures illustrated in FIG. 76. All are ddNTP analogues in which either Cy5 (shown for ddA and ddG) or HCyC-646 (shown for ddT and ddC) are attached to the base by the readily cleavable SS linker. The first extension is carried out with the ddA and ddT analogues in the presence of sufficient 3′-O-azidomethyl dNTPs to result in the majority (>95%) of template-loop-primers (or primers in other template-bound primer arrangements) on the surface being extended with the reversible terminators, with sufficient incorporation of ddATP or ddTTP analogues to reveal fluorescence.

After a wash at pH 5, imaging will reveal Cy5 and HCyC-646 fluorescence indicating incorporation by the ddA or ddT analogues respectively. Next the ddCTP and ddGTP analogues are added with excess 3′-O-azidomethyl dA and dT to ensure high fidelity incorporation. Imaging after a pH 5 wash will reveal Cy5 or HCyC-646 fluorescence, indicating incorporation of ddC or ddG. Switching to pH 9 will reduce HCyC-646 fluorescence to near background levels.

Thus, loss of fluorescence signals will indicate incorporation by ddT if it was previously determined that either ddT or ddA was incorporated, and will indicate incorporation by ddC if it was previously determined that either ddC or ddG was incorporated.

Finally, the disulfide bonds are cleaved to remove all the dye molecules from the ddNTP analogues and restore the 3′-OH group on primers extended with the reversible terminators, in readiness for the next cycle of SBS. Using the encoding scheme where 1 indicates a positive Cy5 or HCyC-646 fluorescence signal and 0 a background signal, with imaging after the extension steps and the pH switch steps, A will be encoded by 111, C by 010, G by 011, and T by 110.

Examples of ddNTP analogues useful in this SBS approach are presented in FIG. 78. A detailed example of this SBS approach provided by the invention is presented in exemplary FIG. 79. A dye other than Cy5, a cleavable group other than SS, and a conditionally fluorescent dye other than HCyC-646 may also be used.

In variants of this approach, the pH 5 washes and the pH 9 wash after either or both extension steps can be reversed. Depending on the order of the pH 5 and pH 9 washes, different encoding will be obtained for A, C, G and T, but all will be equally simple to decode, though an extra wash will be needed if the first wash is done at pH 9, and a 4-step encoding will be required. For instance, if after the first extension, imaging at pH 9 after the first extension, imaging after a switch to pH 5, imaging at pH 9 after the second extension, and imaging after another switch to pH 5, will result in encoding of 1111 for incorporation by A, 0011 for incorporation by G, 0101 for incorporation by T, and 0001 for incorporation by C. The same design can be used also with NRTs in a standard SBS design or with virtual terminators. In these cases, an optimal amount of unlabeled 3′-O-azidomethyl dNTPs or other reversible terminators are added together with the labeled nucleotide analogues to maintain fidelity of the polymerase reaction, and a further chase step with, for example, unlabeled 3′-O-azidomethyl dNTPs, would typically follow the addition of the labeled nucleotides.

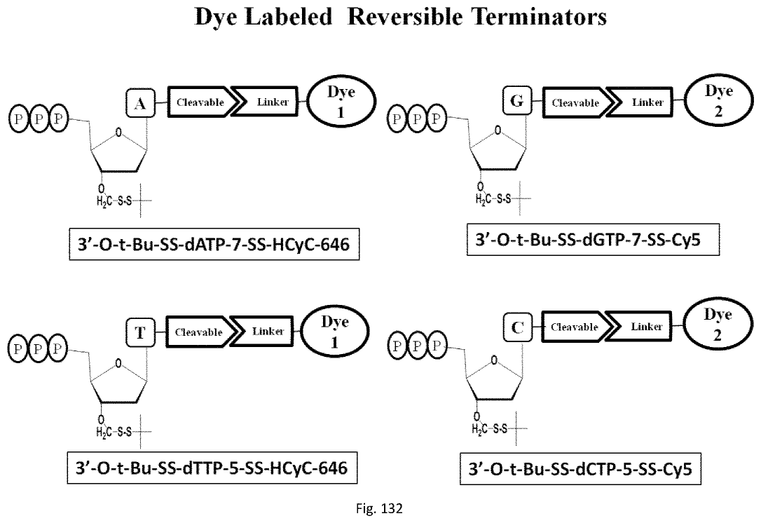

As an example, the approach with cleavable fluorescent 3′-t-Butyl-SS-dNTPs (reversible terminators) in place of the cleavable fluorescent ddNTPs is presented. A generalized set of dye labeled cleavable reversible terminators in FIG. 132, a simplified presentation of the scheme in FIG. 133, example structures of reversible terminators used in this variation in FIG. 134, and a detailed example of this SBS approach in exemplary FIG. 135 is included. Finally, using this approach, 20 cycles of accurate sequencing by synthesis is demonstrated in FIG. 136.

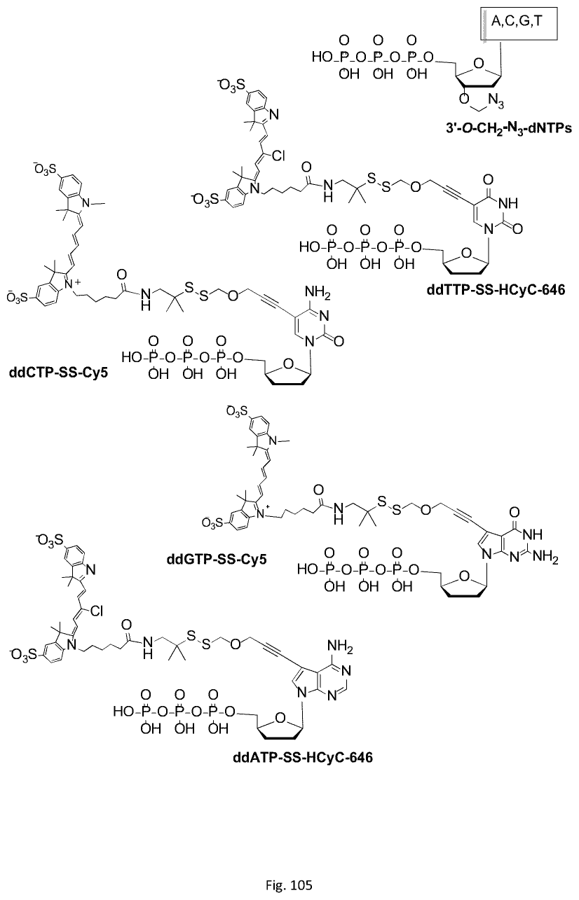

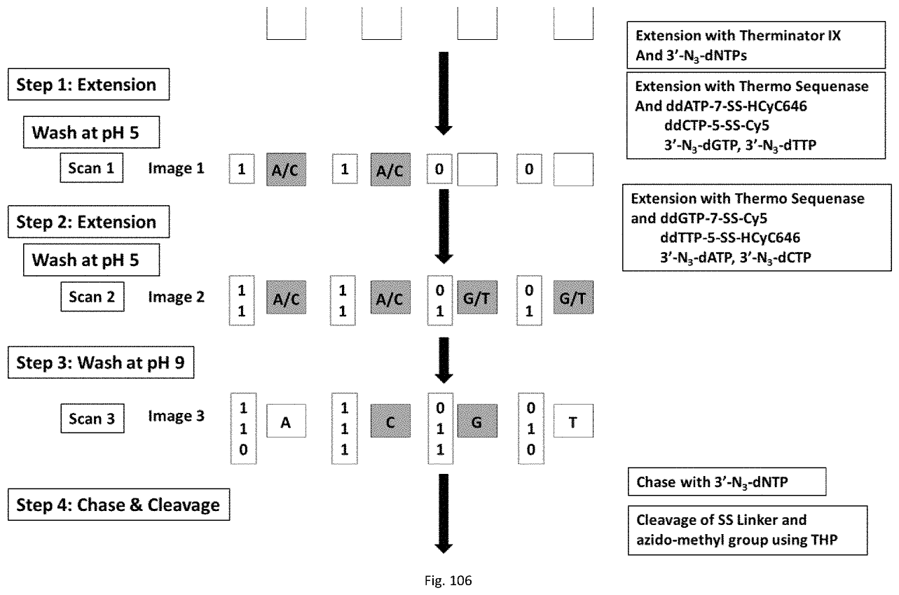

Example 10 Experimental Results: Demonstrations of the ability to synthesize the pH responsive dye HCyC-646 and attach it to ddNTPs, as well the ability of these modified ddNTPs to be incorporated by DNA polymerase, and evidence that they can be used in a sequencing by synthesis protocol on a surface are presented here: Two HCyC-646-labeled ddNTPs (ddATP-SS-HCyC-646 and ddTTP-SS-HCyC-646) were synthesized for use in an SBS scheme including ddGTP-SS-Cy5 and ddCTP-SS-Cy5, along with 3′-O-azidomethyl dNTPs. [This is essentially the same as in FIGS. 77 and 79, but note that the nucleotide analogues used here are not identical to the nucleotide analogues shown in FIGS. 76 and 78.] The structures of these nucleotide analogues are presented in FIG. 105 and the generalized SBS scheme in which they can be used is presented in FIG. 106.

Briefly, following incorporation of the four 3′-O-azidomethyl dNTPs into the majority of primer strands (˜95%) in the presence of Therminator IX, two of the nucleotides, ddATP-SS-HCyC-646 and ddCTP-SS-Cy5, along with 3′-O-azidomethyl dTTP and 3′-O-azidomethyl dGTP are added in the presence of Thermo Sequenase. After a wash at pH 5, imaging will result in fluorescent signals being obtained for incorporation of either the ddATP-SS-HCyC-646 or the ddCTP-SS-Cy5.

Next, the remaining labeled nucleotides, ddTTP-SS-HCyC-646 and ddGTP-SS-Cy5, along with 3′-O-azidomethyl dATP and 3′-O-azidomethyl dCTP, are added in the presence of Thermo Sequenase. After a wash at pH 5, imaging will result in fluorescent signals being obtained for incorporation of either the ddTTP-SS-HCyC-646 or the ddGTP-SS-Cy5. A subsequent wash step is performed at pH 9, which will eliminate the fluorescence for the two ddNTP analogues labeled with HCyC-646.

Thus, if it was previously determined that either A or C was incorporated, loss of signal indicates it was A and remaining signal is indicative of C. Similarly, if it was previously determined that either T or G was incorporated, loss of signal indicates it was T and remaining signal is indicative of G. Finally, cleavage of the SS bonds in the linker will remove the dyes and at the same time restore the 3′-OH group in the majority of primer strands that were extended with one of the 3′-O-azidomethyl dNTPs.

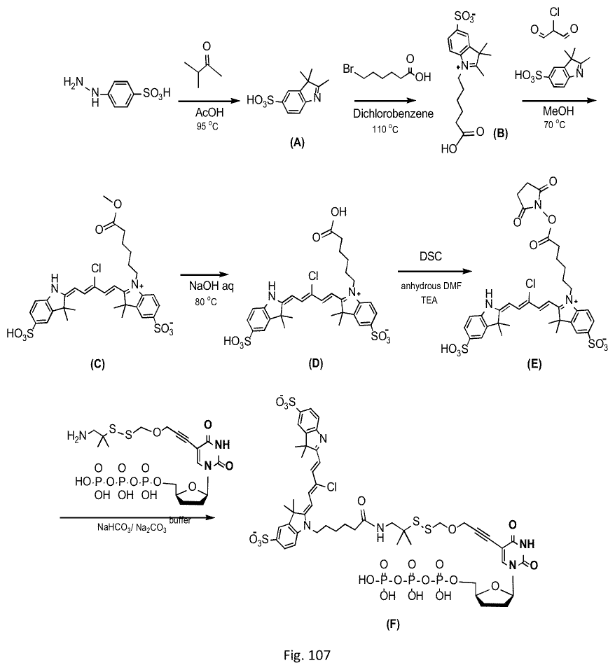

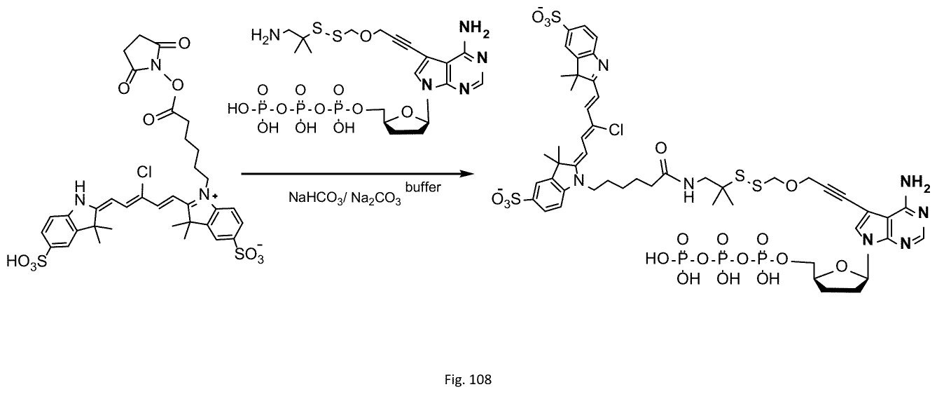

The detailed synthetic scheme for synthesis of HCyC-646 (FIG. 107) and its conjugation to ddTTP (FIG. 107) or to ddATP (FIG. 108) is as follows: The synthesis of pH sensitive HCyC-646 dye was carried out essentially the same way as reported (Hilderbrand et al 2008) with some modifications as described here (FIG. 107). The starting indoles, 2, 3, 3-trimethyindoleninium-5-sulfonate (A) and 1-(e-carboxypentyl)-2,3,3-trimethyindoleninium-5-sulfonate (B) were prepared according to published procedures (Mujumdar et al 1993).

a) HCyC-646 Methyl Eater (C): To a solution of 1-(e-carboxypentyl)-2,3,3-trimethyindoleninium-5-sulfonate (B, 884 mg, 2.5 mmol) in methanol (15 ml) was added chloromalonaldehyde (266 mg, 2.5 mmol) and the solution was heated at 70° C. in a thick-walled glass pressure reactor for 30 min giving a greenish-yellow solution. After cooling, 2,3,3-trimethyindoleninium-5-sulfonate (A, 598 mg, 2.5 mmol) was added and the pressure reactor was re-sealed and heated at 70° C. for 5 h giving a dark blue solution. After cooling, the solution was concentrated and purified on a reverse-phase C18 column (50 g RPC-C18 packed in a glass column) with increasing concentration of acetonitrile in water (0, 5-15%). The fractions eluted with 5-15% acetonitrile in water were combined and concentrated in vacuo to give a brownish solid. MALDI TOF MS: calculated for C₃₂H₃₈ClN₂O₈S₂, 677, found 679.

b) HCyC-646 acid, D: HCyC-646 Methyl Ester (C) obtained above was hydrolyzed with 1M NaOH (7 ml) at 80° C. for 1.5 h. After cooling, the solution was acidified with 10% aq. HCl and the solution was concentrated in vacuo. The blue solution was purified on a reverse-phase C18 column (50 g RPC-C18 packed in a glass column) with increasing concentration of acetonitrile in water (0, 5-20%). The dark blue solution eluted last was collected and concentrated. This was further subjected to cation exchange chromatography (Dowex 50W X8 H⁺ resin) to give pure D. MALDI TOF MS: calculated for C₃₁H₃₆ClN₂O₈S₂, 663.15, found 663.5

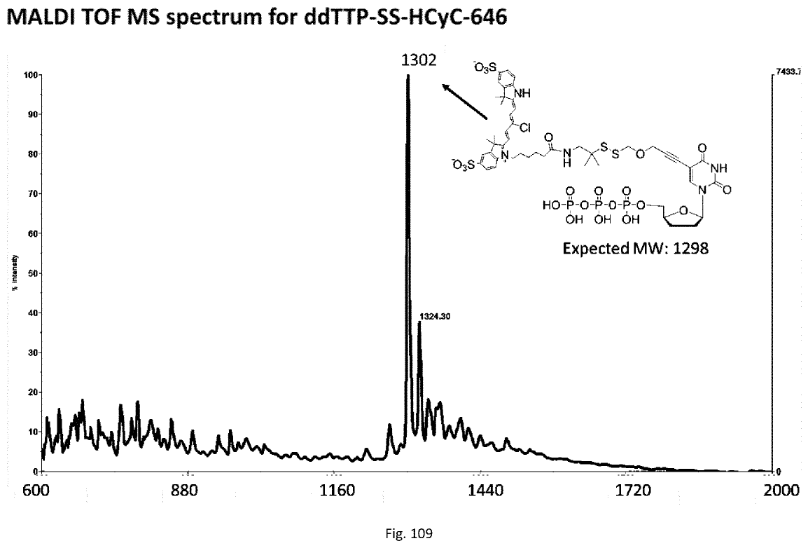

c) HCyC-646 NHS and conjugation with 5-Amino-SS-ddTTP (FIGS. 107 and 108): The dried HCyC-646 acid (D, 11 mg, 16.5 μmol, 1 eq) was dissolved in 200 μL anhydrous DMF and 5.5 mg of pre-dried N,N-discuccinimidyl carbonate (DSC) (5.5 mg, 21.6 μmol, 1.3 eq) and 16 μL of triethylamine (11.7 mg, 116.2 μmol, 7 eq) were added to the solution. The reaction mixture was stirred at 30° C. for 40 min. The formation of HCyC-646-NHS ester (E) was monitored by thin layer chromatography using dichloromethane and methanol (4:1). Then, ddTTP-SS-NH₂ (Ju et al 2018) (1.3 mg, 1.9 μmol) dissolved in 250 μL of NaHCO₃/NaCO₃ buffer (pH 8.5) was added to the HCyC-646-NHS ester reaction mixture (FIG. 107). The reaction was stirred overnight at 30° C. with exclusion of light. Purification was performed using ion exchange chromatography followed by reverse-phase HPLC (HFIP buffer and MeOH). The compound, ddTTP-SS-HCyC-646 (F), was characterized by MALDI TOF MS (calc'd, 1298 Da, found: 1302 Da). The obtained mass spectrum is shown in FIG. 109).

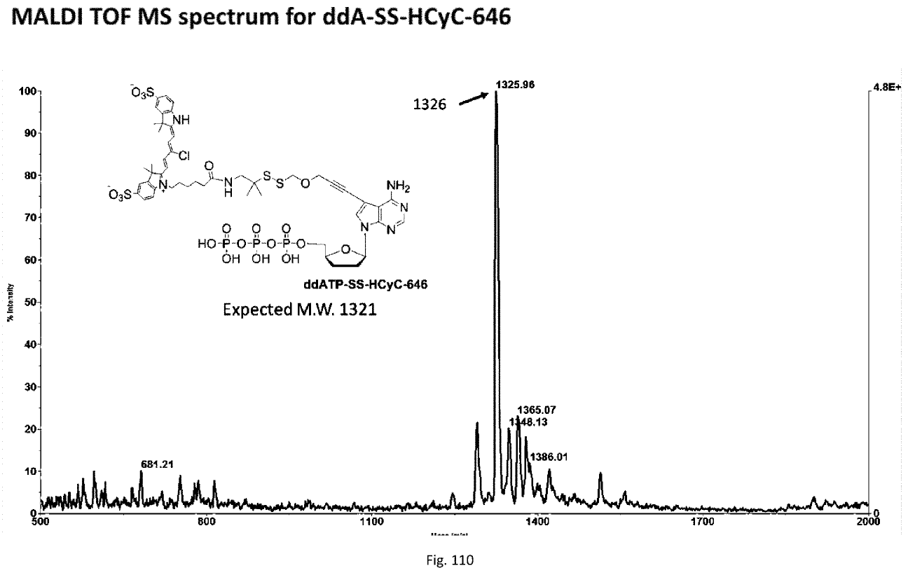

Similarly, HCyC-646-NHS ester (E) dissolved in anhydrous DMF was reacted with ddATP-SS-NH₂ (1.48 mg, 2.15 μmol) in 250 μL of NaHCO₃/NaCO₃ buffer (pH 8.5) (FIG. 108). The reaction was stirred overnight at 30° C. with exclusion of light. Purification was performed using ion exchange chromatography followed by reverse-phase HPLC purification (HFIP buffer and MeOH). The compound, ddATP—SS-HCyC-646, was characterized by MALDI TOF MS (calc'd, 1326 Da, found: 1321 Da). The obtained mass spectrum is shown in FIG. 110.

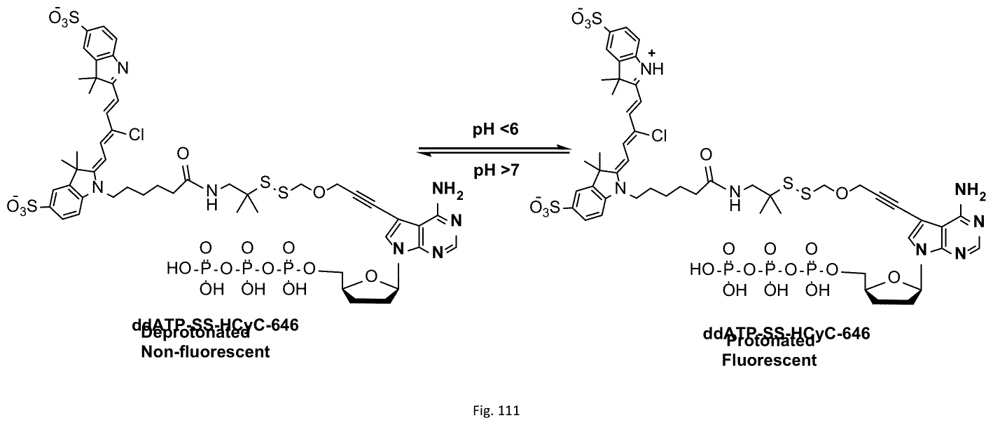

The synthesis of other types of nucleotides with cleavable linker conjugated with HCyC-646 can essentially be carried out as outlined above. The dye, HCyC-646, in its non-protonated form is non-fluorescent (pH >7) and becomes fluorescent when it is protonated (pH<6). The pKa value for HCyC-646 is 6.2. The structures of the non-protonated and protonated dye are shown in FIG. 111.

A solution test for extension and THP cleavage of the ddTTP-SS-HCyC-646 was next performed.

Extension Reaction with ddTTP-SS-HCyC-646:

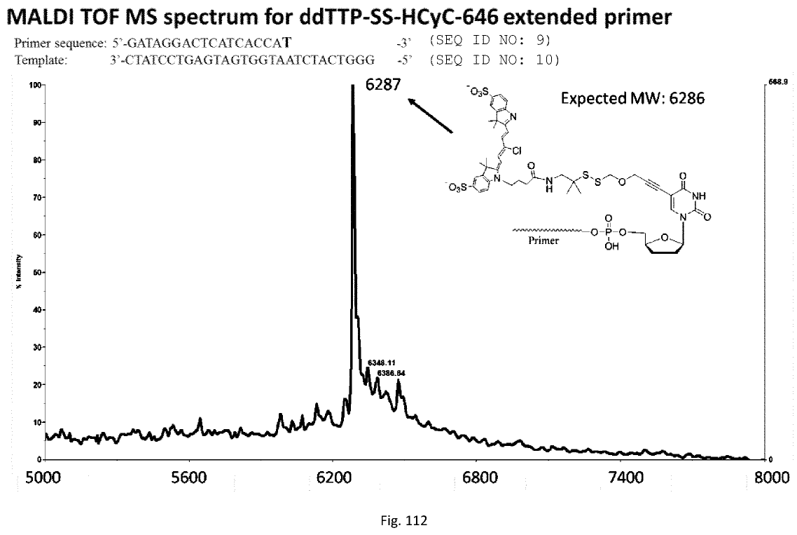

A single base DNA extension reaction (SBE) was performed with ddTTP—SS-HCyC-646, using primer E8TT (5′-GATAGGACTCATCACCA-3′ [SEQ ID NO: 5]) and a synthetic DNA template (5′-ACGGAGAACGAAGAGAAAAGGATAGGACTCATCACCATTAGATGACCCTGCCTTGCCTTGTCGAACTC CACGCACAAACACGGACAGGACCCTCTCTGGCCGCGTGTCTCCTTC-3′ [SEQ ID NO: 6]) based on a portion of exon 8 of the human p53 gene as follows. 2 μL of 10× concentrated buffer, 1 μL of primer E8TT (MW: 5163, 100 μM), 3 μL of Exon 8 template (20 μM), 1.5 μL of nucleotide (ddTTP-SS-HCyC-646, 167 μM), 1 μL of mutant DNA Polymerase T9 (10 U/l) and 11.5 μL of dH₂O were combined in a total volume of 20 μL. The reaction consisted of 38 cycles at 65° C. for 30 seconds and 45° C. for 30 seconds. Single base extension was confirmed by MALDI-TOF MS (FIG. 112) as indicated by the major peak at 6287 Da (calc'd, 6286 Da) in the spectrum. No primer peak (5163 Da) was observed indicating that essentially all the primer was extended and the incorporation rate was 100%.

Detailed Method for Immobilization of DNA on Slides:

The 5′-amino-modified self-priming template DNA was dissolved in 50 mM sodium phosphate buffer, pH 9.0 at a concentration of 30 μM and spotted on NHS ester-derivatized CodeLink slides (Surmodics Inc., MN) using a SpotArray 72 microarray printing robot (PerkinElmer, MA). The slides were incubated overnight in a humid chamber containing a solution of saturated sodium chloride at 37° C. to immobilize the DNA. Unreacted NHS ester groups were quenched by incubating the slides in a solution of 50 mM 3-amino-1-propanol in 100 mM tris-HCl buffer, pH 9.0 for 2 hours at ambient temperature. The slides were briefly rinsed in boiling water, air-dried under compressed air and stored desiccated in a dark container until use. Four different templates were spotted in separate rectangular areas on the slides. These primer-loop-templates (self-priming templates) were designed such that the first incorporated nucleotide analogue in an SBS reaction will be T, G, C and A, respectively; the second incorporated nucleotide analogue will be A, A, A and T respectively; and the third incorporated nucleotide analogue will be G, G, T and T, respectively.

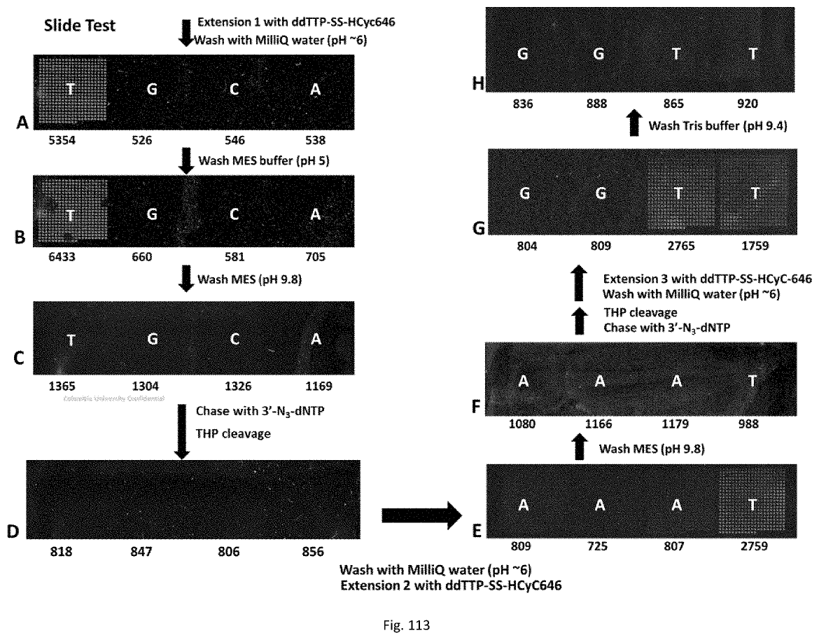

Testing of ddTTP-SS-HCyC-646 on a Glass Slide: To test the utility of nucleotide analogues conjugated with HCyC-646 for SBS reactions, the following series of reaction, washing and imaging steps were carried out on the above slides with the results presented in FIG. 113. All extension reactions were carried out by placing 60 μl of the relevant solution within a chamber covering all four spotted areas of the slide. Washing was performed by dipping the slides in a buffer solution for various periods of time. Imaging for HCyC-646 was conducted after drying the slides in a four-color fluorescent scanner using the 633 nm laser and an emission window centering around 670 nm.

Step 1. Extension with ddTTP-SS-HCyC-646: 60 μL solution consisting of 2 μL 2 μM ddT-SS-HCyC-646, 6 μL 1 U/ul Therminator IX, 6 μL 10× Thermo Pol Buffer, 46 μL water at 65° C. for 10 min.

Step 2. Wash with Milli-Q water (pH ˜6) and perform first scan.

Step 3. Dip in 0.1M MES (2-[N-morpholino]ethanesulfonic acid hydrate buffer, pH 5 for 3 min and perform second scan.

Step 4. Dip in 50 mM MES (sodium salt), pH 9.8 for 3 min and perform third scan.

Step 5. Wash with 0.1% Tween-20 in PBS, then Milli-Q water.

Step 6. Chase: 60 μL solution consisting of 5 μL each 1 μM 3′-O—N₃-dATP, 3′-O—N₃-dCTP, 3′-O—N₃-dTTP and 3′-O—N₃-dGTP, 6 μL 1 U/μL Therminator IX, 6 μL 10× Thermo Pol buffer, 8 μL water) at 65° C. for 10 min.

Step 7. Wash with 0.1% Tween-20 in PBS, then Milli-Q water.

Step 8. THP cleavage: 60 μL solution consisting of 6 μL 10×THP, 6 μL 200 mM NaCl, 12 μL 100 mM sodium borate, 36 μL water) at 65° C. for 10 min and perform fourth scan.

Step 9. Repeated steps 1-8 for second and third cycles of SBS (scans are only shown after extension and pH change steps in these cycles in FIG. 113).

As seen in FIG. 113, imaging after the first extension step (Panel A) results in a positive fluorescence signal only over the left rectangular area of the slide indicating successful and specific incorporation of the ddTTP-SS-HCyC-646 nucleotide. Imaging after the pH 5 wash (Panel B) does not show any further increase in the fluorescence signal in the left spotted area of the slide. Imaging after the pH 9.8 wash (Panel C) results in loss of the fluorescence signal, as expected for the HCyC-646 dye. Finally, imaging after the chase and THP cleavage (Panel D) again shows a background fluorescence signal in all areas of the slide. In the second cycle, imaging after extending again with ddTTP-SS-HCyC-646 and washing with water (pH 6) (Panel E) reveals a positive signal only in the right rectangular area of the slide as expected for T incorporation in the second cycle. Imaging after the pH 9.8 wash (Panel F) shows the expected loss of fluorescence of the HCyC-646 dye. In the third cycle, imaging after extending again with ddTTP-SS-HCyC-646 and washing with water (pH 6) (Panel G) reveals a positive signal in the two right-most spotted areas of the slide, as expected for T incorporation in the third cycle. Finally, imaging after a pH 9.4 tris buffer wash (Panel H) results in loss of fluorescence, as expected for HCyC-646.

Continuous Sequencing by Synthesis on a Slide Using the One-Color SBS Method of Example 10 with ddATP-7-SS-HCyC-646, ddTTP-5-SS-HCyC-646, ddCTP-5-SS-Cy5, ddGTP-7-SS-Cy5 and Four 3′-O-Azidomothyl dNTPs.

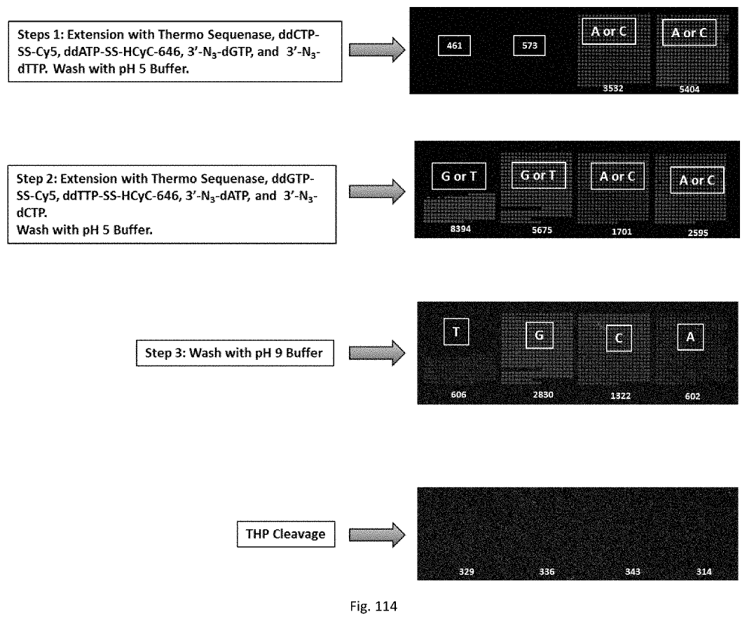

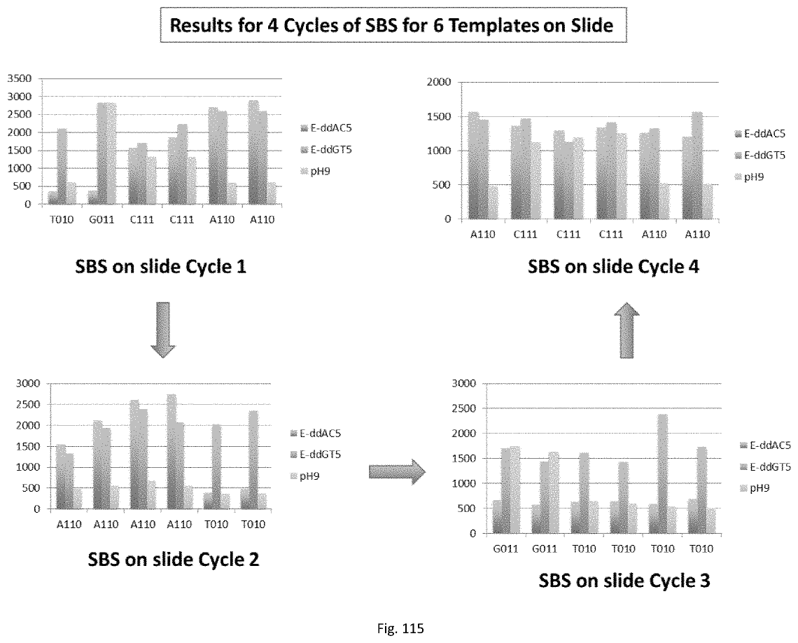

Using the protocol illustrated in FIG. 106, continuous sequencing by synthesis results were obtained as demonstrated in FIG. 114 and FIG. 115. The following steps were carried out in each cycle of SBS:

Step 1. Slides prepared as described above were incubated with 60 μl of a solution consisting of 1 μl ddATP-SS-HCyC-646 (1 μM), 4 μl ddCTP-SS-Cy5 (0.1 μM), 4 μl 3′-O-azidomethyl-dGTP (0.1 μM), 4 μl 3′-O-azidomethyl-dTTP (0.1 μM), 6 μl Thermo Sequenase (1 unit/μl), 6 μl 10× Thermo Sequenase Buffer, 35 μl water for 10 min at 65° C. They were washed in 10 mM Tris-HCl buffer, pH 5, a pH at which both Cy5 and HCyC-646 exhibit fluorescence, and imaged as described above to detect either Cy5 or HCyC-646 fluorescence.

Step 2. Slides were incubated with 60 μl of a solution consisting of 1 μl ddTTP-SS-HCyC-646 (1 μM), 4 μl ddGTP-SS-Cy5 (0.1 μM), 4 μl 3′-O-azidomethyl-dCTP (0.1 μM), 4 μl 3′-O-azidomethyl-dATP (0.1 PM), 6 μl Thermo Sequenase (1 unit/μl), 6 μl 10× Thermo Sequenase Buffer, 35 μl water for 10 min at 65° C. They were washed in 10 mM Tris-HCl buffer, pH 5, a pH at which both Cy5 and HCyC-646 exhibit fluorescence, and re-imaged.

Step 3. Slides were washed with 10 mM Tris buffer, pH 9, a pH at which Cy5 but not HCyC-646 exhibits fluorescence, and re-imaged.

Step 4. Slides were incubated with a 60 μL solution consisting of 6 μL 10×THP, 6 μL 200 mM NaCl, 12 μL 100 mM sodium borate, 36 μL water) at 65° C. for 10 min, and re-imaged.

The results for a typical cycle are illustrated in FIG. 114. Imaging after Step 1 is shown in the top panel. The two right-most areas of the slide show fluorescence, indicating incorporation of either A or C, since only the ddATP and ddCTP analogues were added and both have dyes (HCyC-646 and Cy5, respectively) that fluoresce at pH 5.

Imaging after Step 2 is shown in the next panel. Now fluorescence appears in the two left-most areas of the slide, indicating incorporation of either T or G, since the ddTTP and ddGTP analogues were added at this step, and again both contain dyes (HCyC-646 and Cy5, respectively) that fluoresce at pH 5. Imaging after step 3 indicates loss of fluorescence in the left and right areas of the slide. Because HCyC-646 is not expected to exhibit fluorescence at pH 9 while Cy5 will exhibit fluorescence at this pH, this reveals which base was incorporated in each of the four areas of the slide, T, G, C and A from left to right. Imaging after step 4 shows just background fluorescence due to cleavage of the dyes by THP.

FIG. 115 shows the results for 4 continuous cycles of 1-color SBS, where the bar graphs at top left, bottom left, bottom right and top right indicate the results for Cycles 1, 2, 3 and 4, respectively.

In each bar graph, each group of 3 bars indicates a template spotted in a different area of the slide, where the six groups of three bars represent Template 1, Template 2, Template 3, Template 3, Template 4 and Template 4 from left to right (two of the templates were spotted in duplicate areas). In each bar graph, the imaging results (in arbitrary units on the y-axis) are shown after Step 1 (left bar in each group of 3), after Step 2 (middle bar in each group of 3) and after Step 3 (right bar in each group of 3). The encoding under each group of 3 bars indicates the incorporated nucleotide (010 for T, 011 for G, 111 for C and 110 for A). Thus, Template 1 shows incorporation of T in Cycle 1, A in Cycle 2, G in Cycle 3 and A in Cycle 4. Correct incorporation results are obtained for each template in each of the four continuous SBS cycles.

Continuous Sequencing by Synthesis on a Slide Using the Once-Color SBS Method of Example 10 with 3′-tBu-SS-dATP-7-SS-HCyC-646, 3′-tBu-SS-dCTP-5-SS-Cy5, 3′-tBu-SS-dGTP-7-SS-Cy5, 3′-tBu-SS-dTTP-5-SS-HCyC-646 and the Four 3′-O-Azidomethyl dNTPs.

Using a slight variant of the protocol illustrated in FIG. 133, continuous sequencing by synthesis results were obtained for 20 bases as demonstrated in FIG. 136.

The following procedure was implemented:

Step 1. Glass slides prepared as described above were incubated with 40 μl of a solution consisting of 1 μl 0.5 μM 3′-tBu-SS-dATP-7-SS-HCyC-646, 1 μl 0.5 μM 3′-tBu-SS-dCTP-5-SS-Cy5, 4 units of Therminator IX, 4 μl 10× ThermoPol buffer, 4 μl 20 mM MnCl₂ for 10 min at 65° C.

Step 2. Slides were washed with 20 μM citric acid buffer, pH 5, and imaged.

Step 3. Slides were incubated with a 40 μl solution consisting of 1 μl 0.5 μM 3′-tBu-SS-dGTP-7-SS-Cy5, 1 μl 0.5 μM 3′-tBu-SS-dTTP-5-SS-HCyC-646, 4 units of Therminator IX, 4 μl 10× ThermoPol buffer, 4 μl 20 mM MnCl₂ for 10 min at 65° C.

Step 4. Slides were washed with 20 μM citric acid buffer, pH 5, and re-imaged.

Step 5. Slides were washed with 20 μM Tris buffer, pH 9, and re-imaged.

Step 6. Slides were incubated with 60 μl of a chase solution consisting 5 μl each of 1 μM 3′-O—N₃-dATP, 3′-O—N₃-dCTP, 3′-O—N₃-dGTP and 3′-O—N₃-dTTP, 6 μl 1 U/μl Therminator IX, 6 μl 10× ThermoPol buffer, 8 μl water for 10 min at 65° C. followed by a wash with 0.1% Tween-20 in PBS and a Milli-Q water rinse.

Step 7. Slides were incubated with a 60 μl solution consisting of 6 μl 10×THP, 6 μl 200 mM NaCl, 12 μl 100 mM sodium borate, 36 μl water at 65° C. for 5 min, and re-imaged.

The concentrations of fluorescent nucleotide analogues were increased in each cycle, ranging from 125 nM at the start to 750 nM by the 20^{th} cycle.

### Example 11: Single Color Sequencing by Synthesis Using a Set of ddNTP Analogues, Two with Cy5 and Two with HCyC-646, all with SS Linker, with One Extension and One Quenching Step

In this example, a quenching step is required, but advantageously all four ddNTP analogues can be added concurrently and no separate labeling step is needed. This SBS approach again takes advantage of a conditionally fluorescent dye, such as HCyC-646. As in Example 10, advantageously only a final SS cleavage step is needed.

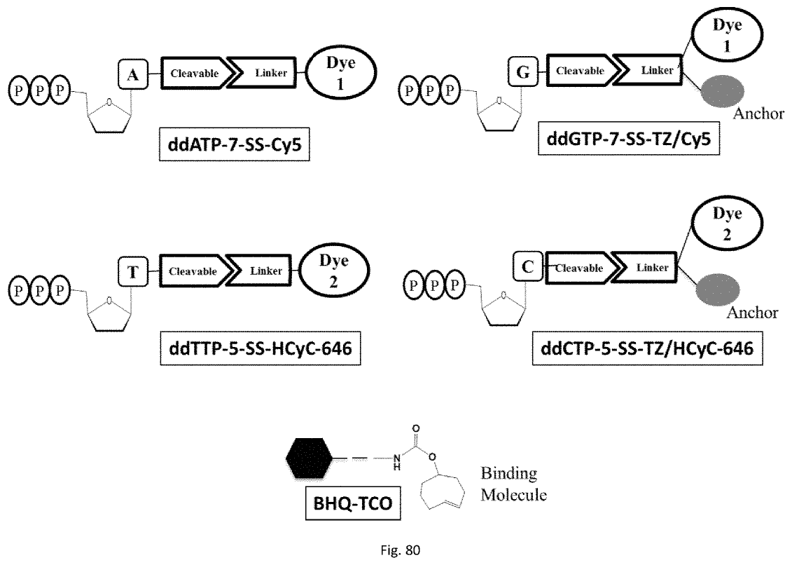

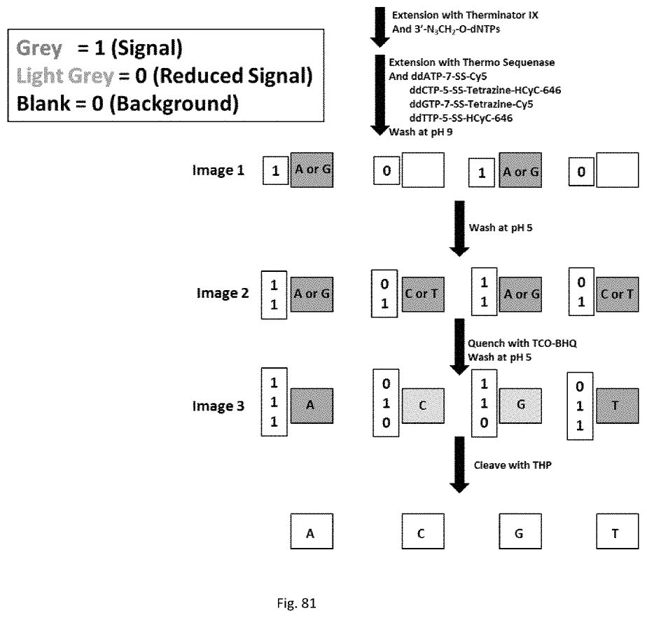

For the purpose of illustration, this novel SBS approach provided by the invention is described in a combined ddNTP/NRT format, as exemplified in the scheme shown in FIG. 81. Four different nucleotides are designed with the general structures illustrated in FIG. 80. Two have Cy5 (ddA and ddG) and two have HCyC-646 (ddT and ddC), however while all are attached to the base via an SS linker, one with Cy5 (ddG) and one with HCyC-646 (ddC) have a tetrazine anchor for binding of TCO-BHQ3, which will quench the dyes. In this exemplary scheme, the majority of template-loop-primers (or primers in other template-bound primer arrangements) on the surface are extended with reversible terminators such as 3′-O-azidomethyl dNTPs, after which extension is carried out with the above four ddNTP analogues. After a wash at pH 9, imaging will reveal Cy5 fluorescence for incorporation by either the ddA or ddG analogues. A subsequent imaging after a pH 5 wash will allow fluorescence of HCyC-646 on ddT and ddC, resulting in cumulative fluorescence for all four nucleotides. Binding of TCO-BHQ to the ddC and ddG and incubation at pH 5 will result in loss of fluorescence due to quenching of both of these nucleotide analogues, resulting in fluorescence remaining only on the ddA and ddT analogues. Finally, the disulfide bonds are cleaved to remove all the dye molecules from the ddNTP analogues and restore the 3′-OH group on primers extended with the reversible terminators, in readiness for the next cycle of SBS. Using the encoding scheme where 1 indicates a positive Cy5 signal and 0 a background signal, with imaging after the extension, labeling and quenching steps, A will be encoded by 111, C by 010, G by 110, and T by 011; with consideration of just the first and third of these imaging steps, the encoding will be 11 for A, 00 for C, 10 for G, and 01 for T.

Examples of ddNTP analogues useful in this novel SBS approach are presented in FIG. 82. A detailed example of this SBS approach is presented in exemplary FIG. 83. A dye other than Cy5, a conditionally fluorescent dye other than HCyC-646, a cleavable group other than SS, an anchor other than tetrazine, and a variety of alternative quenchers specific for the attached dyes may be used.

In a variant of this approach, the pH 9 wash and the first pH 5 wash are reversed. In this case, imaging after the pH 5 wash will indicate incorporation by any of the four nucleotide analogues.

Imaging after the pH 9 wash will result in loss of fluorescence of HCyC-646 on ddC and ddT analogues, with remaining fluorescence due to Cy5 on ddA and ddG analogues. All other steps and the ultimate determination of which nucleotide analogue was incorporated are the same as described above in this example. In this variant, encoding based on the three imaging steps will be 111 for A, 100 for C, 110 for G and 101 for T; if only the last two imaging steps are considered, the encoding will be 11 for A, 00 for C, 10 for G and 01 for T.

The tetrazine anchor can be replaced with alternate anchors, so long as the quencher is attached to the appropriate anchor binding molecule. For instance, if the anchor is biotin, streptavidin-BHQ can be used, or if the anchor is an azide, dibenzocyclooctyne-BHQ can be used.

The same design can be used also with NRTs in a standard SBS design or with virtual terminators. In these cases, a chase step with, for example, unlabeled 3′-O-azidomethyl dNTPs, would typically follow the addition of the labeled nucleotides.

### Example 12: Single Color Sequencing by Synthesis Using a Set of ddNTP Analogues, Two with Dyes and Two with Dyes and Anchors, all with SS Linker, Requiring Two Extensions, Om Labeling Nd Om Quenching Step

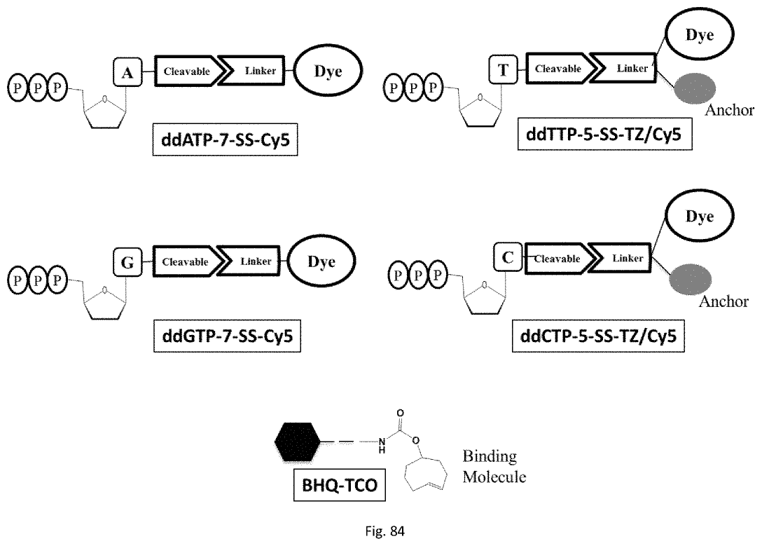

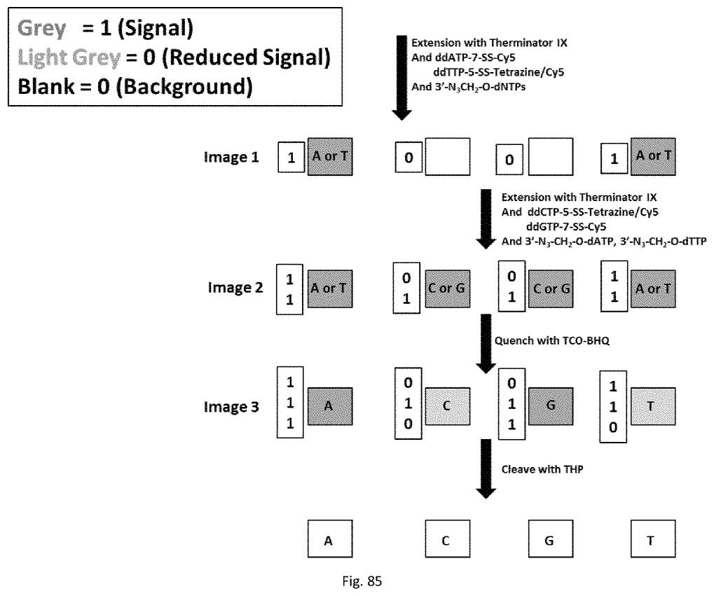

In this novel SBS approach provided by the invention, similar to the approach provided in Example 10, two of the ddNTP analogues are added at a time. However, in this approach a labeling and a quenching step, rather than a dye with conditional fluorescence, are used. For the purpose of illustration, this approach is described herein in a combined ddNTP/NRT format as exemplified in the scheme shown in FIG. 85. Four different nucleotides are designed with the general structures illustrated in FIG. 84. All are ddNTP analogues in which Cy5 is attached to the base by the readily cleavable SS linker. But for two of these (ddC and ddT), the anchor tetrazine is also attached via the same linker, designed for the attachment of a quencher. The first extension is carried out with the ddA and ddT analogues in the presence of sufficient 3′-O-azidomethyl dNTPs to result in the majority (>95%) of template-loop-primers (or other template-bound primer arrangements) on the surface being extended with the reversible terminators, with sufficient incorporation of ddATP or ddTTP analogues to reveal fluorescence. Next the ddCTP and ddGTP analogues are added with excess 3′-O-azidomethyl dA and dT to ensure high fidelity incorporation. Imaging after a wash will reveal Cy5 fluorescence, indicating incorporation of ddC or ddG. Incubation with TCO-BHQ3 will result in quenching of the Cy5 on the ddC and ddT analogues, resulting in full fluorescence only for the ddA and ddG analogues. Finally, the disulfide bonds are cleaved to remove all the dye molecules from the ddNTP analogues and restore the 3′-OH group on primers extended with the reversible terminators, in readiness for the next cycle of SBS. Using the encoding scheme where 1 indicates a positive Cy5 signal and 0 a background signal, with imaging after the extension, labeling and quenching steps, A will be encoded by 111, C by 010, G by 011, and T by 110; with consideration of just the first and third of these imaging steps, the encoding will be 11 for A, 00 for C, 01 for G, and 10 for T.

Examples of ddNTP analogues useful in this novel SBS approach are presented in FIG. 86. A more detailed example of this SBS approach is presented in exemplary FIG. 87. Dyes other than Cy5, cleavable groups other than SS, and anchors other than tetrazine could be used with this scheme.

The same design can be used also with NRTs in a standard SBS design or with virtual terminators. In these cases, an optimal amount of unlabeled 3′-O-azidomethyl dNTPs or other reversible terminators are added together with the labeled nucleotide analogues to maintain fidelity of the polymerase reaction, and a further chase step with, for example, unlabeled 3′-O-azidomethyl dNTPs, would typically follow the addition of the labeled nucleotides.

### Example 13: Single Molecule Energy Transfer DNA Sequencing by Synthesis, Using Virtual Terminator Nucleotide Analogues, Containing an Energy Transfer Donor Dye and Either of Two Anchors Attached to the Base by a Dithio Linker, Requiring Two Extension and Two Labeling Steps for Attachment of Either a pH-Responsive or a pH Unresponsive Energy Transfer Acceptor Dye

The use of the pH-responsive dye HCyC-646 was described in some of the above examples. Here, this dye along with pH-unresponsive dyes such as Cy5 and Cy3 are used to accomplish single molecule energy transfer-based DNA sequencing by synthesis. The approach is described here for virtual terminators (nucleotides with a cleavable blocking group that greatly reduces incorporation efficiency, attached to the base, as well as 3′-blocked nucleotide reversible terminators). Since this is a single molecule approach, ddNTPs cannot be used in this scheme or the similar scheme in Example 14.

This approach takes advantage of the key properties of energy transfer (ET). The donor dye is excited at a wavelength where the acceptor dye has negligible absorbance; however, if these dyes are placed in proximity, ET from donor to acceptor results in emission of the acceptor dye. This large spectral separation (“Stokes shift”) eliminates non-specific fluorescence due to the presence of unbound acceptor dye-containing molecules. Because of the wide spectral gap between the absorption of the donor dye and the emission of the acceptor dye, wider band pass filters can be used to collect more photons for increased sensitivity, at the same time avoiding undesirable portions of the intervening spectrum so as to reduce background fluorescence, allowing higher signal-to-noise ratios and more accurate base calling, leading to single molecule SBS. Anchors are utilized to bring donor and acceptor dyes within 10 nm for efficient energy transfer.

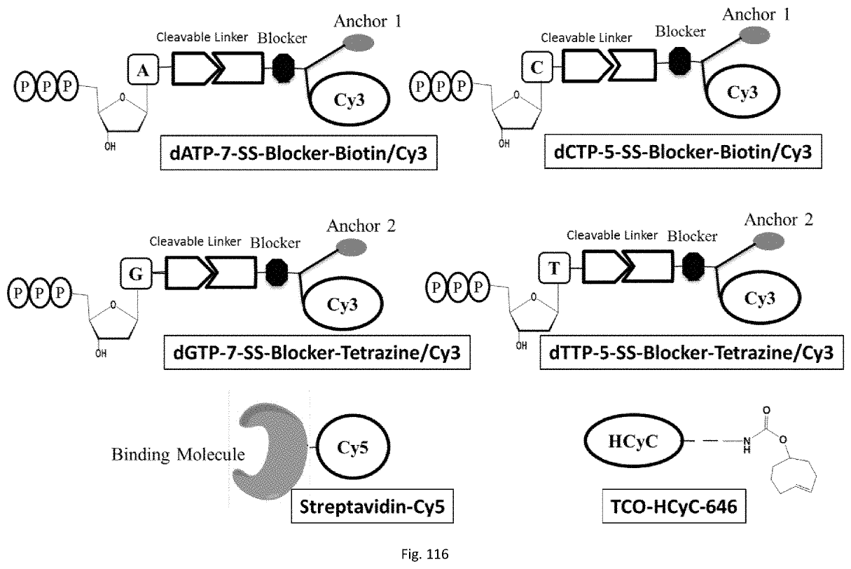

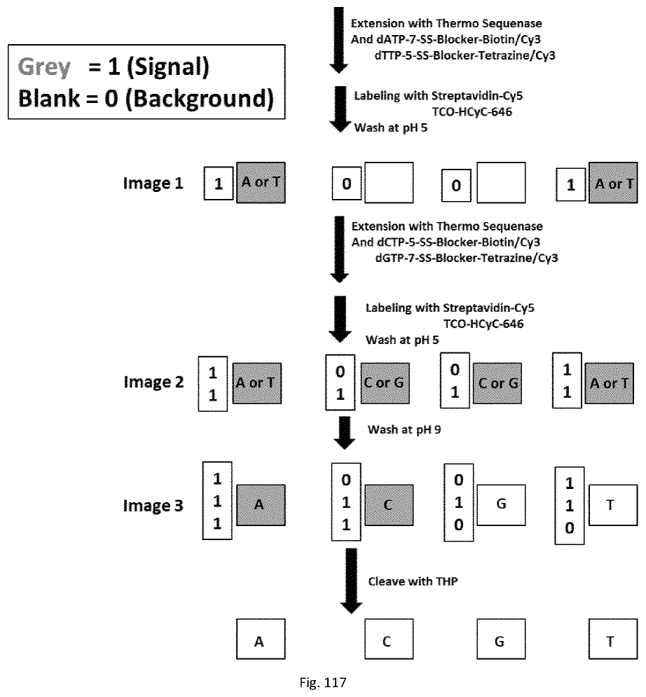

The donor dye Cy3 and either of two anchors, biotin or tetrazine in the example presented (FIG. 116), are attached to the base distal to the blocker in the case of the virtual terminators. Two of the nucleotides have biotin and Cy3, and the other two nucleotides have TCO and Cy3. Following the logic of exemplary FIG. 117, extension is performed with two of the virtual terminators (A and T), both of which have Cy3, A having biotin and T having tetrazine. A labeling step is performed with streptavidin-Cy5 and TCO-HCyC-646 added at the same time. Then the labeling buffer is replaced with a pH 5 buffer to allow fluorescence emission from both Cy5 and HCyC-646 after excitation of the donor dye Cy3. The above steps are repeated with the remaining two virtual terminators (C and G), which like the first pair, both have Cy3, but C has biotin and G has TCO. Another labeling step with streptavidin-Cy5 and TCO-HCyC-646 is performed followed by another pH 5 buffer wash and imaging step utilizing energy transfer. A final buffer wash at pH 9 and a third imaging step will clarify which of the four virtual terminators was incorporated, since at this pH Cy5 (on A and C) will have a positive fluorescence signal but HCyC-646 (on T and G) will display only background fluorescence.

Thus, if it was previously determined that either A or T was incorporated and the fluorescence remains, it indicates A incorporation; if the fluorescence is lost, it indicates T was incorporated. Similarly, if it was previously determined that either C or G was incorporated and the fluorescence remains, it indicates C was incorporated; if the fluorescence is lost, it indicates G was incorporated.

Since the emission profiles of both acceptor dyes are nearly identical, like the majority of approaches disclosed herein, this is essentially a one-color method.

Finally, cleavage with THP will be performed to cleave all the linkers, removing any dye and also restoring the 3′-OH group. If a positive signal is indicated by the integer 1 and a background signal by a 0, then based on imaging after the first extension and labeling steps and a low pH wash, after the second extension and labeling steps and a low pH wash and after the subsequent high pH wash, incorporation of A will be encoded by 111, C by 011, G by 010 and T by 110.

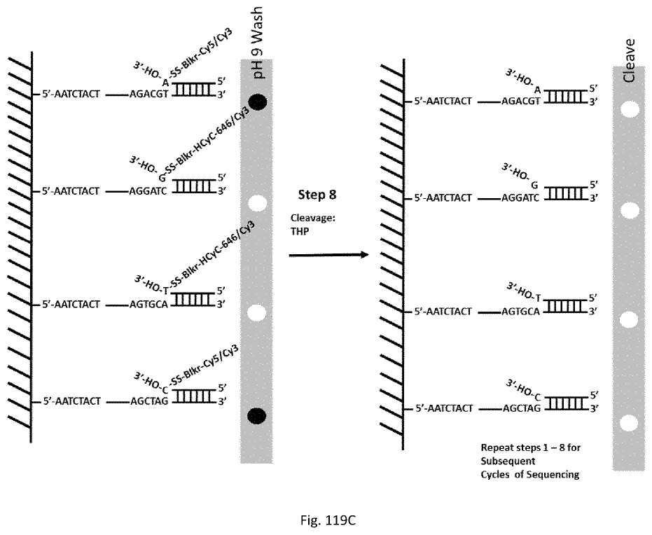

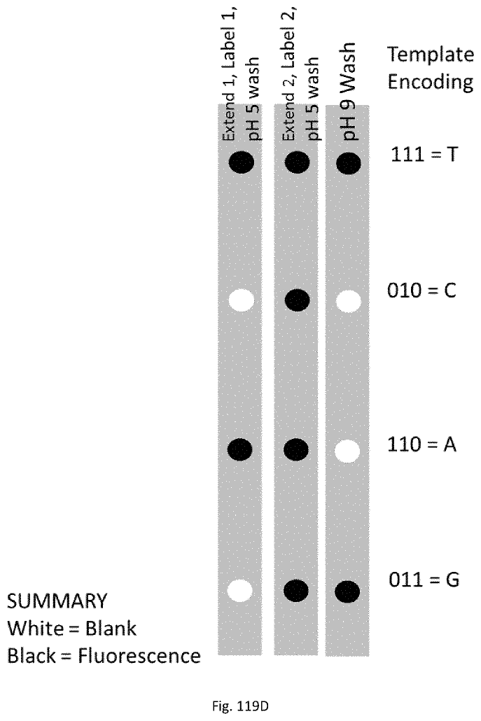

The middle imaging step is not essential, as the first and third imaging steps will be sufficient to distinguish incorporation of A (11), C (01), G (00) and T (10). Structures of the four nucleotide analogues are presented in FIG. 118, and a detailed embodiment of the general SBS scheme provided by the invention is presented in FIG. 119.

A variant of this scheme in which the order of the second pH 5 incubation and the pH 9 incubation is reversed is also included herein. In a similar encoding scheme the incorporation of A would be represented as 111, C as 011, G as 001 and T as 101 with all 3 imaging steps, and if using only the first two imaging steps, 11 for A, 01 for C, 00 for G and 10 for T.

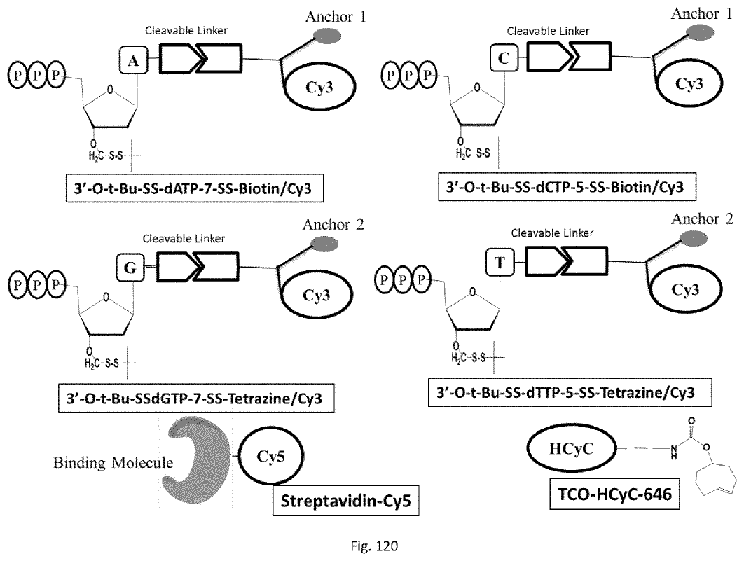

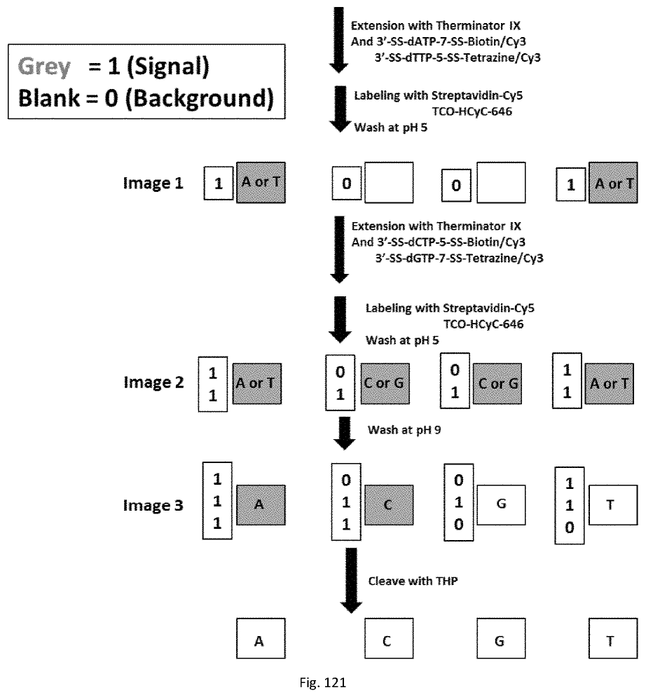

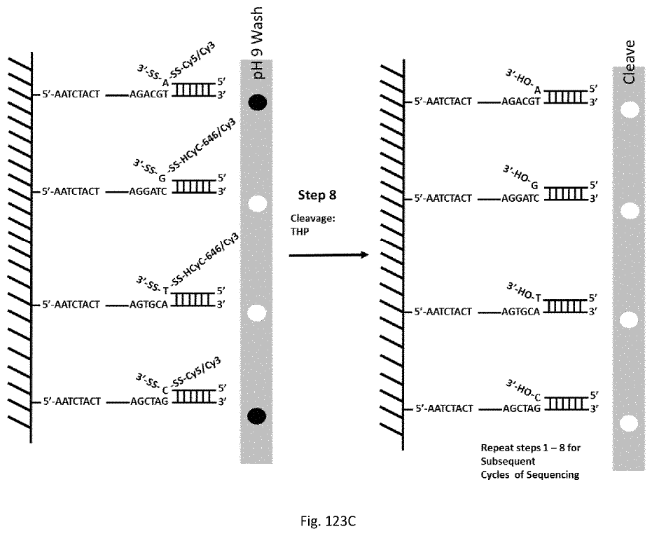

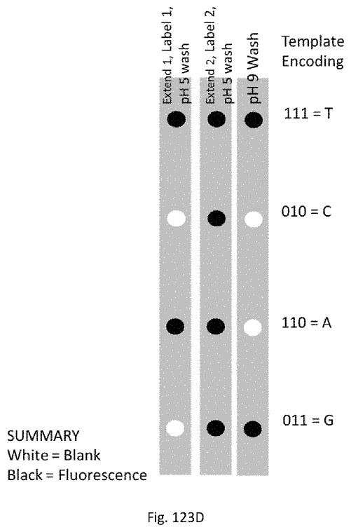

Another exemplary embodiment of this SBS approach using 3′-blocked reversible terminators is disclosed in FIGS. 120-123.

In another embodiment of this SBS approach, the nucleotides can be synthesized with Cy3 and either Cy5 or HCyC-646 already attached at an appropriate distance from the Cy3 for efficient energy transfer; in this case the labeling step is not needed.

In yet another embodiment of this SBS approach, clusters of the donor dyes and the anchors, at optimal distances apart, can result in an even higher signal and better sensitivity for single molecule SBS.

### Example 14: Single Molecule Energy Transfer DNA Sequencing by Synthesis, Using an Orthogonal Set of Virtual Terminator Nucleotide Analogues, Containing an Energy Transfer Donor Dye and Either of Two Anchors Attached to the Base by Either a Dithio or Azo Linker, Requiring One Extension and One Labeling Step for Attachment of Either a pH-Responsive or a pH Unresponsive Energy Transfer Acceptor Dye, and Two Cleavage Steps

This approach is similar to that of Example 13, again involving the use of energy transfer and pH-responsive and pH-unresponsive dyes as energy transfer acceptors. However, in this scheme, each of the nucleotide analogues has a different combination of linker and anchor, in addition to the Cy3 donor dye which is present on all four; this allows all four virtual terminator analogues to be added at the same time.

This approach takes advantage of the key properties of energy transfer. The donor dye is excited at a wavelength where the acceptor dye has negligible absorbance; however, if these dyes are placed in proximity, ET from donor to acceptor results in emission of the acceptor dye. This large spectral separation (“Stokes shift”) eliminates non-specific fluorescence due to the presence of unbound acceptor dye-containing molecules. Because of the wide spectral gap between the absorption of the donor dye and the emission of the acceptor dye, wider band pass filters can be used to collect more photons for increased sensitivity, at the same time avoiding undesirable portions of the intervening spectrum so as to reduce background fluorescence, allowing higher signal-to-noise ratios and more accurate base calling, leading to single molecule SBS. We utilize anchors to bring donor and acceptor dyes within 10 nm for efficient energy transfer.

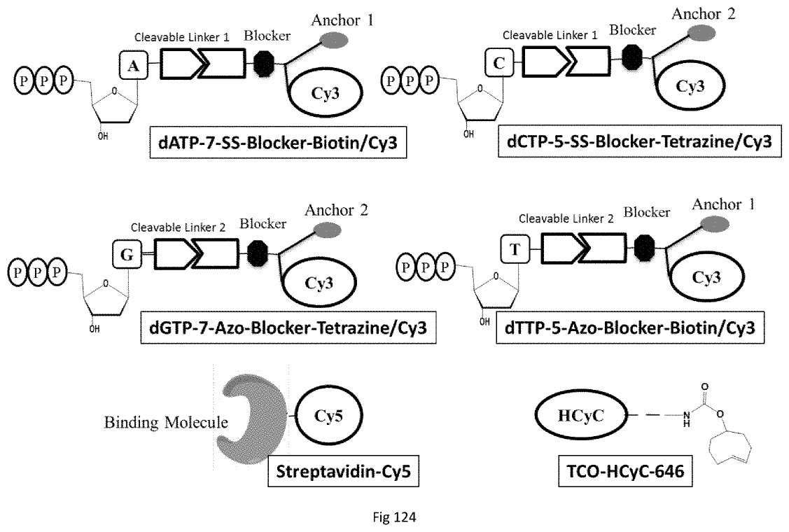

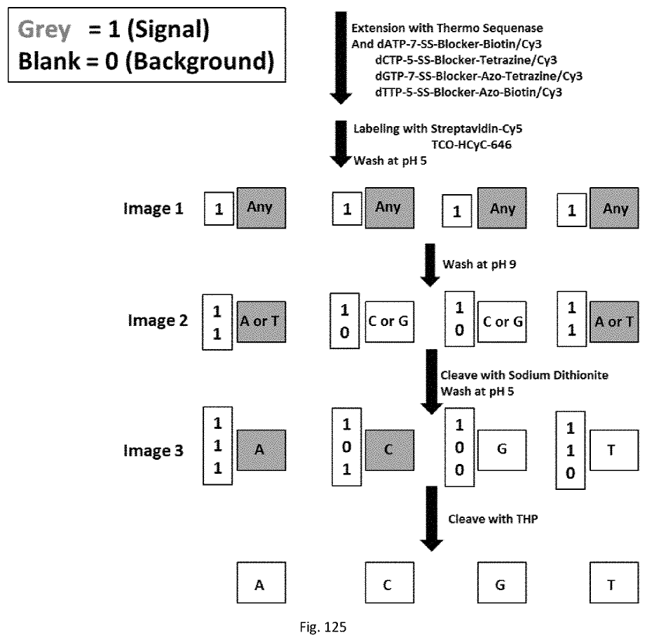

The donor dye Cy3 and either of two anchors, biotin or tetrazine in the example presented (FIG. 124), are attached to the base distal to the blocker in the case of the virtual terminators. In the example shown, one of the nucleotides (A) has biotin and Cy3 attached via an SS linker, the second nucleotide (C) has tetrazine and Cy3 attached via an SS linker, the third (G) has tetrazine and Cy3 attached via an Azo linker, and the fourth (T) has biotin and Cy3 attached via an Azo linker. Following the logic of exemplary FIG. 125, extension is performed with the four virtual terminators at the same time. A labeling step is performed with streptavidin-Cy5 and TCO-HCyC-646 added at the same time. Then the buffer is replaced with a pH 5 buffer to allow fluorescence emission from both Cy5 and HCyC-646 after excitation of the donor dye Cy3. At this imaging step, a positive fluorescence signal will be obtained regardless of which nucleotide was incorporated, as Cy5 and HCyC-646 both fluoresce well at this pH. A buffer switch to pH 9 and a second imaging will result in only background fluorescence from the HCyC-646 on the C and G nucleotide analogues. Thus, at this step, a positive fluorescence signal will indicate incorporation by A or T, and background fluorescence will indicate incorporation by C or G. Next, cleavage of the Azo linkers by sodium dithionite and imaging at pH 5 will clarify which of the four virtual terminators was incorporated.

Thus, if it was previously determined that either A or T was incorporated and the fluorescence remains, it indicates A incorporation; if the fluorescence is lost, it indicates T was incorporated. Similarly, if it was previously determined that either C or G was incorporated and the fluorescence remains, it indicates C was incorporated; if the fluorescence is lost, it indicates G was incorporated.

Since the emission profiles of both acceptor dyes are nearly identical, like the majority of approaches disclosed herein, this is essentially a one-color method.

Finally, cleavage with THP will be performed to cleave the SS linkers, removing any remaining dyes and also restoring the 3′-OH group. If a positive signal is indicated by the integer 1 and a background signal by a 0, then based on imaging after the extension and labeling step and a low pH wash, after the high pH wash and after the Azo cleavage and low pH wash, incorporation of A will be encoded by 111, C by 101, G by 100 and T by 110.

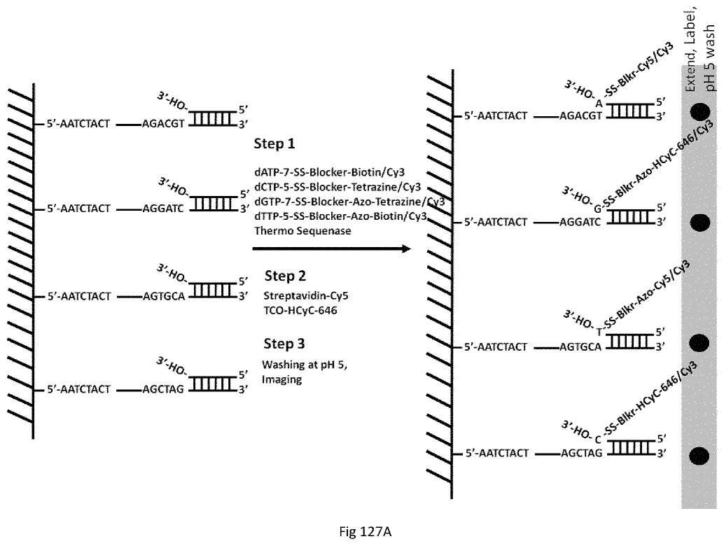

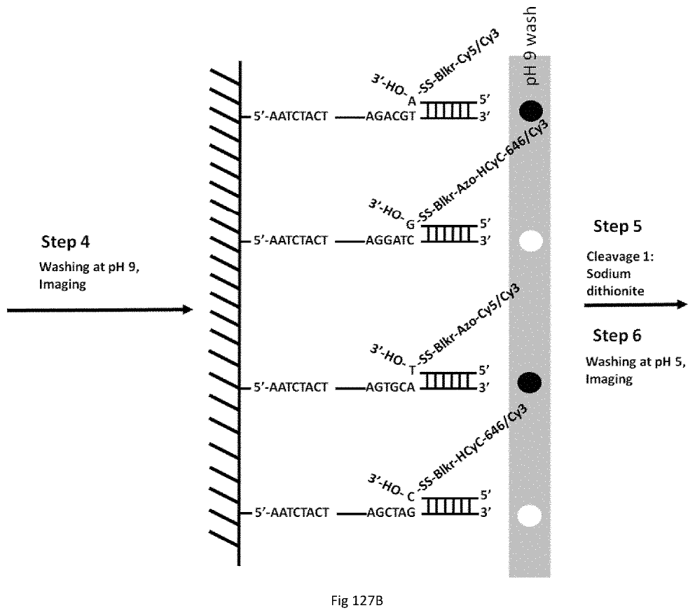

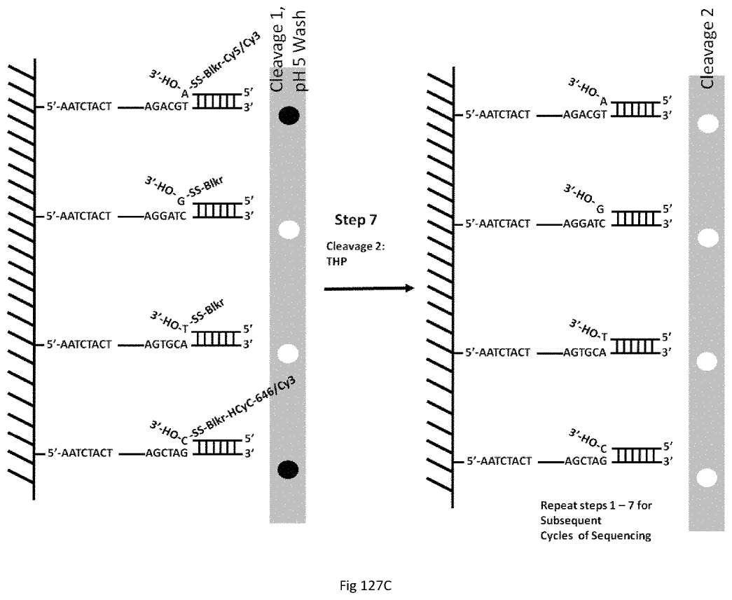

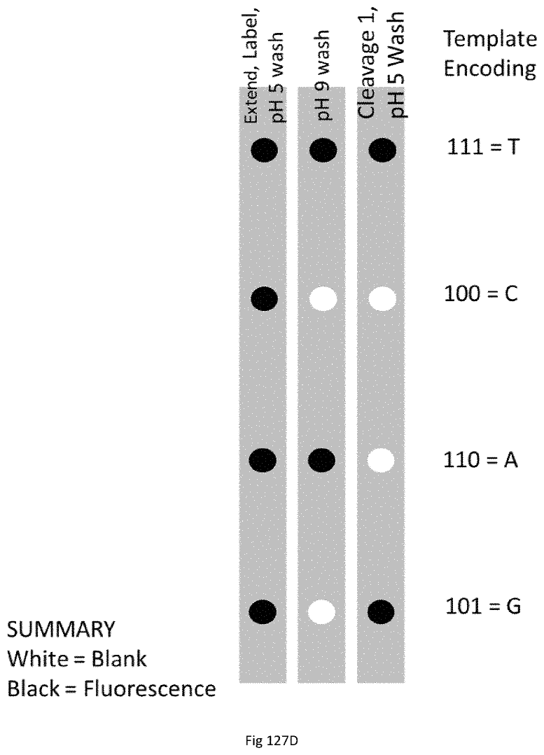

The first imaging step is not essential, as the second and third imaging steps will be sufficient to distinguish incorporation of A (11), C (01), G (00) and T (10). Structures of the four nucleotide analogues are presented in FIG. 126, and a detailed embodiment of the general SBS scheme provided by the invention is presented in FIG. 127.

A variant of this scheme in which the order of the first pH 5 incubation and the pH 9 incubation is reversed is also included herein. In a similar encoding scheme the incorporation of A would be represented as 111, C as 011, G as 010 and T as 110 with all 3 imaging steps, and if using only the first and third imaging steps, 11 for A, 01 for C, 00 for G and 10 for T.

In these figures, the SS group is proximal to the blocker (closer to the base) and the Azo group is distal to the blocker (closer to the dye and anchor), and SS groups are present even in the Azo linker containing molecules. In this configuration, while the dyes on the Azo linker-containing nucleotides are removed specifically by sodium dithionite, the blockers on all the nucleotides are removed in the final THP treatment step.

Any pair of dyes having essentially identical spectral properties to each other can be used, so long as one of them fluoresces conditionally, e.g., at a particular pH. Though biotin and tetrazine are used in this example, other pairs of anchors and anchor binding molecules can be used. For instance, if the anchor is phenyldiboronic acid, salicyclhydroxamic acid-Cy5 or salicyclhydroxamic acid-Cy5 can be used, or if the anchor is an azide, dibenzocyclooctyne-HCyC-646 or dibenzocyclooctyne-Cy5 can be used. Though the cleavable linker in the example contains an SS group, alternative cleavable groups may be present in the linker.

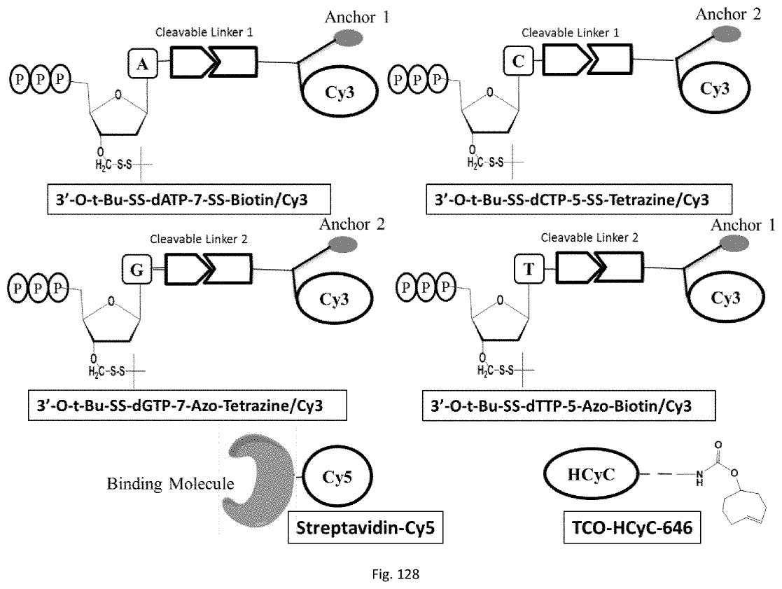

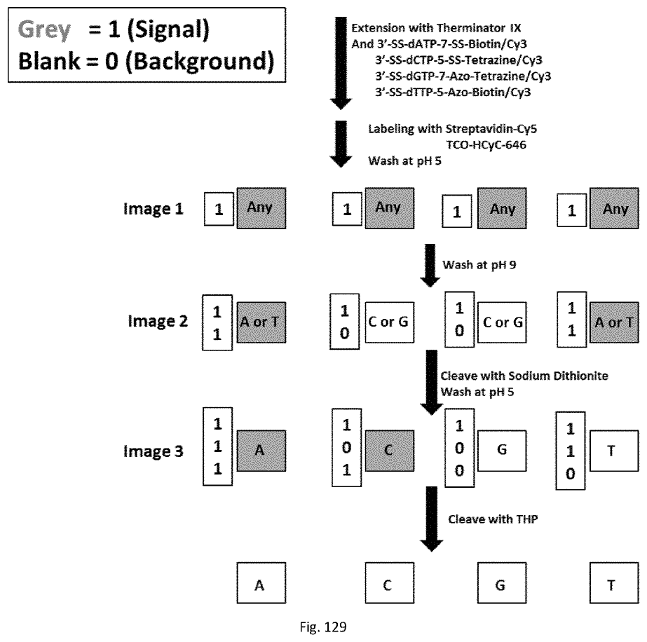

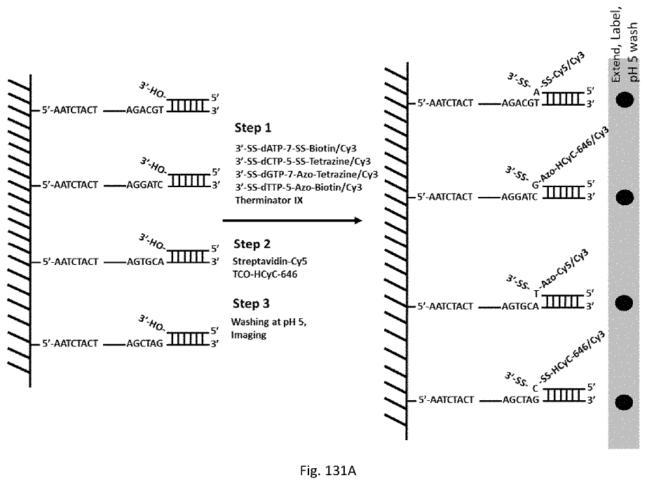

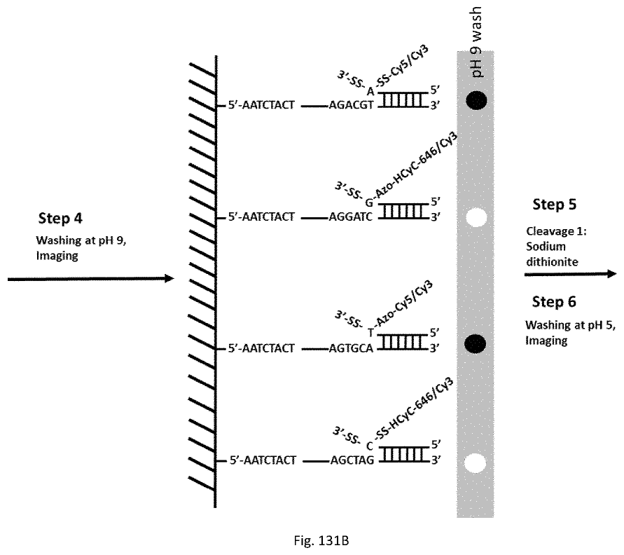

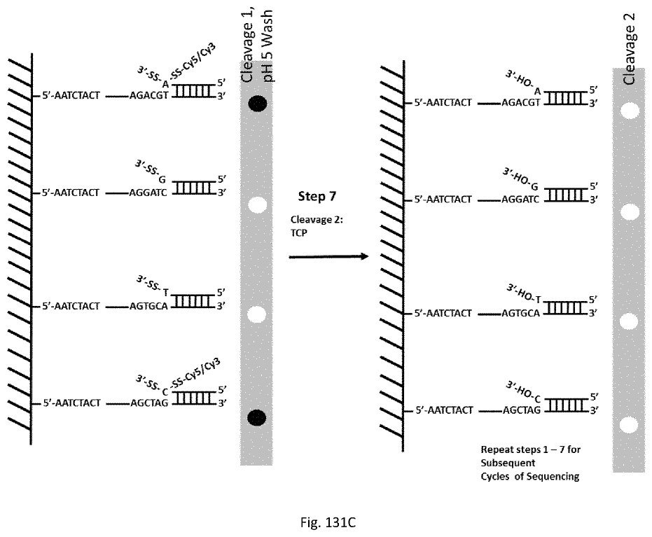

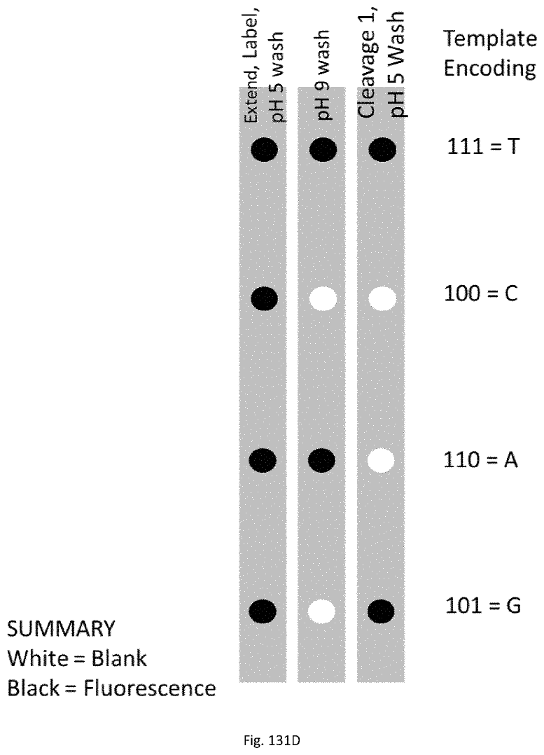

Another exemplary embodiment of this SBS approach using 3′-blocked reversible terminators is disclosed in FIGS. 128-131.

In another embodiment of this SBS approach, the nucleotides can be synthesized with Cy3 and either Cy5 or HCyC-646 already attached at an appropriate distance from the Cy3 for efficient energy transfer; in this case the labeling step is not needed.

In yet another embodiment of this SBS approach, clusters of the donor dyes and the anchors, at optimal distances apart, can result in an even higher signal and better sensitivity for single molecule SBS.

Nucleotide Analogues Synthesis

Methods for synthesizing some of the novel nucleotide analogues presented herein can be found in FIGS. 88-103 and the following descriptions.

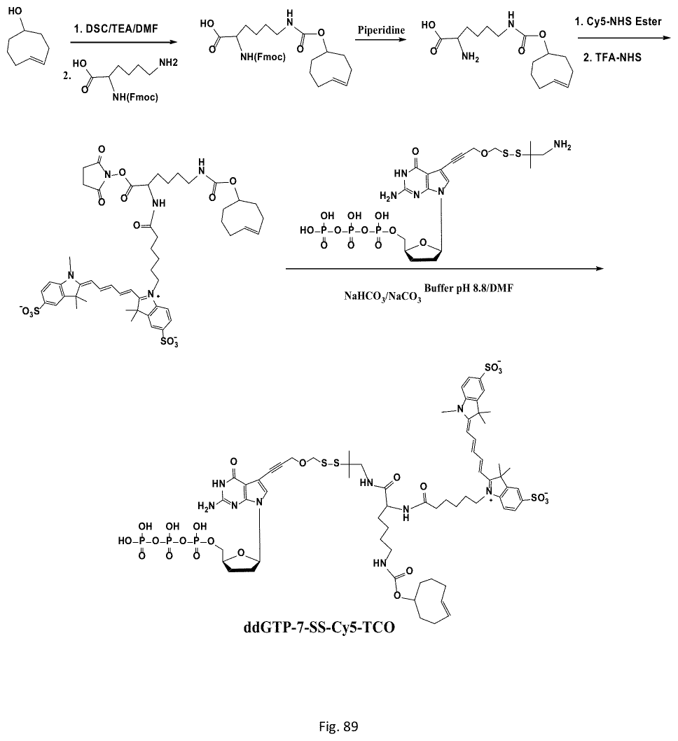

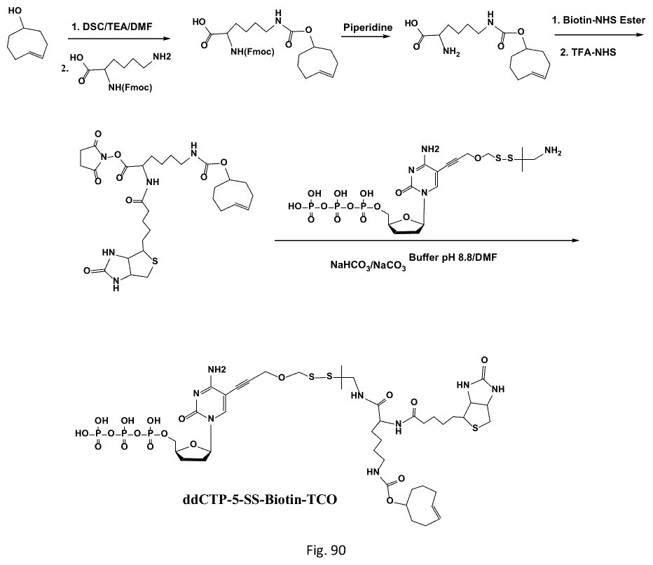

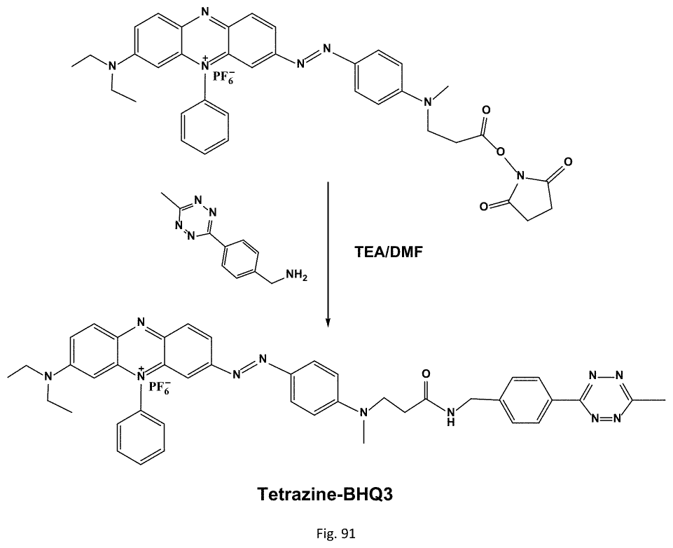

Synthesis of ddNTP-SS-Label(dye or anchor)-TCO (FIG. 88, FIG. 89 and FIG. 90) starts from 5-Hydroxyl-TCO, which is coupled to the 6-NH₂ of α-amino protected lysine via a carbamate bond by treatment with DSC and TEA. After removal of the a-amino protection group Fmoc with piperidine, either Dye (e.g., Cy5) NHS ester or Anchor (e.g., biotin) NHS ester is used to label TCO derivatized lysine, giving Dye (e.g., Cy5) or Anchor (e.g., biotin) labeled TCO-lysine which is further treated with TFA NHS yielding Dye or Anchor labeled TCO-lysine-NHS ester. Dye or Anchor labeled TCO-lysine-NHS ester can be used to couple with Amino-SS(DTM) linker and further converted to Dye or Anchor labeled TCO-SS(DTM) linker-NHS ester, which can be coupled to amino group derivatized ddNTPs (ddNTP-NH₂) yielding ddNTP-SS-Label (Dye or Anchor) -TCO (FIG. 88). Dye or Anchor labeled TCO-lysine-NHS ester can also be used to couple with amino-SS(DTM) linker derivatized ddNTPs (ddNTP-SS-NH₂) yielding ddNTP-SS-Label (Dye or Anchor)-TCO (FIG. 89, FIG. 90).

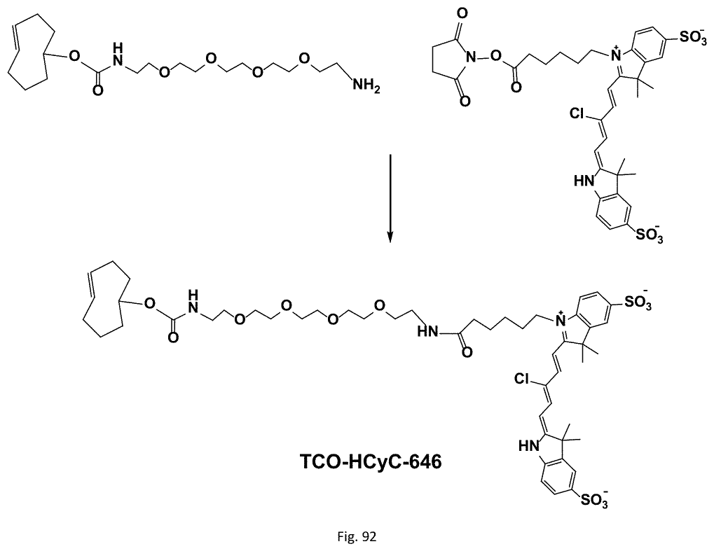

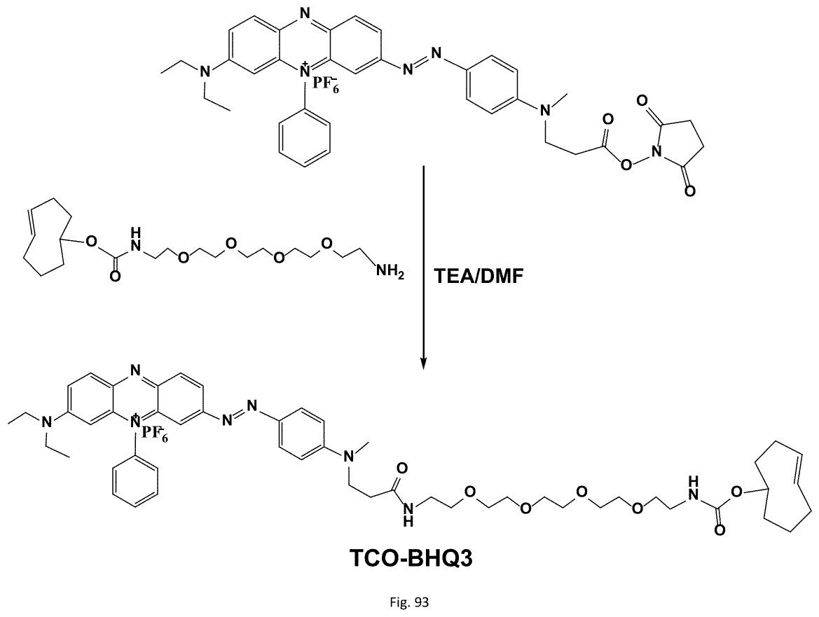

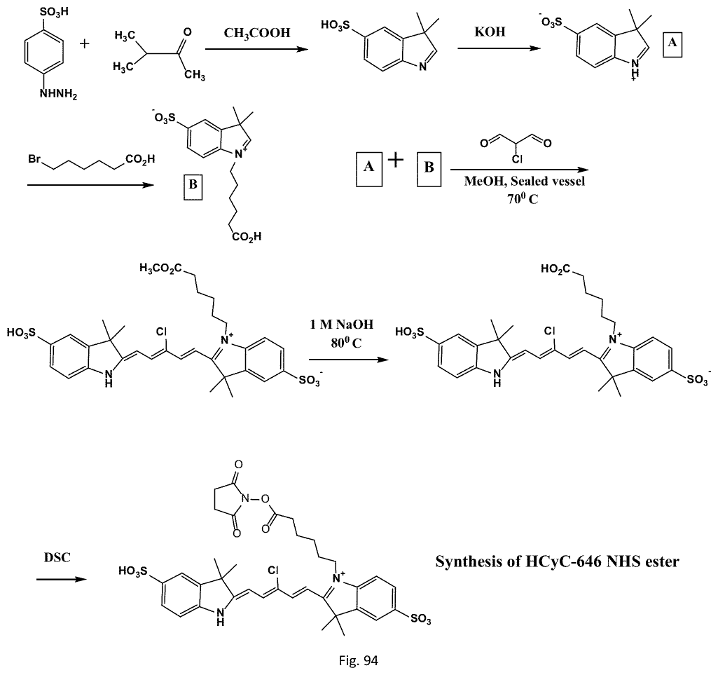

Tetrazine BHQ3 conjugate can be synthesized by coupling commercially available BHQ3 NHS ester and tetrazine-NH₂ (FIG. 91). BHQ3 NHS ester and HCyC646 NHS ester can be coupled with commercially available TCO-PEG-NH₂, yielding TCO-HCyC646 and TCO-BHQ3 conjugates (FIG. 92, FIG. 93). Synthesis of HCyc-646 NHS ester is shown in FIG. 94.

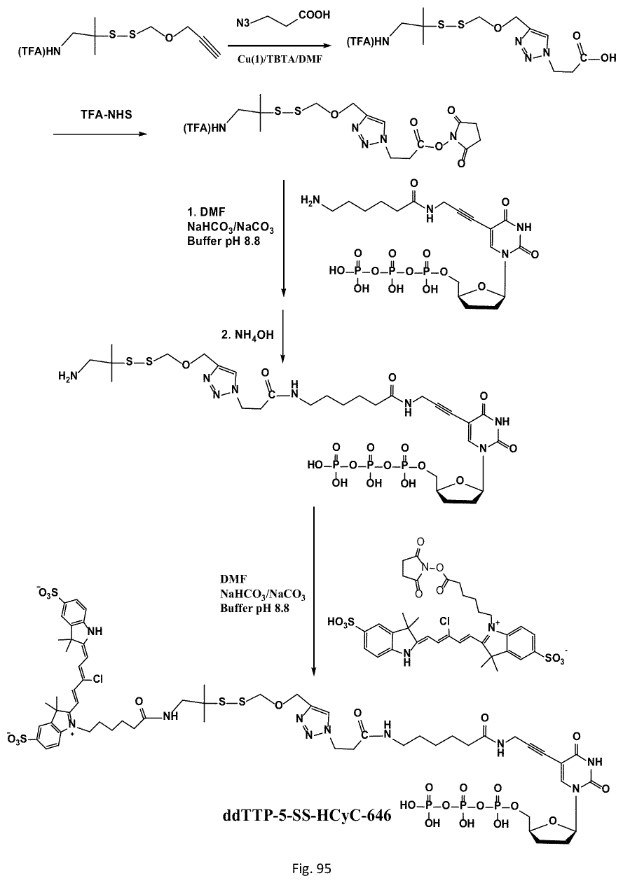

Synthesis of amino-SS(DTM) linker derivatized ddNTPs (ddNTP-SS-NH₂) start with propargyl alcohol derivatized SS(DTM) linker, which is first coupled with azido propionic acid, then converted to a TFA protected amino-SS(DTM) linker NHS ester. The resulting SS linker NHS ester can be coupled with amino group derivatized ddNTPs (ddNTP-NH₂) yielding amino-SS(DTM) linker derivatized ddNTPs (ddNTP-SS-NH₂). Coupling of HCyC-646 NHS ester and ddNTP-SS-NH₂ afford ddNTP-SS-HCyc-646 (FIG. 95).

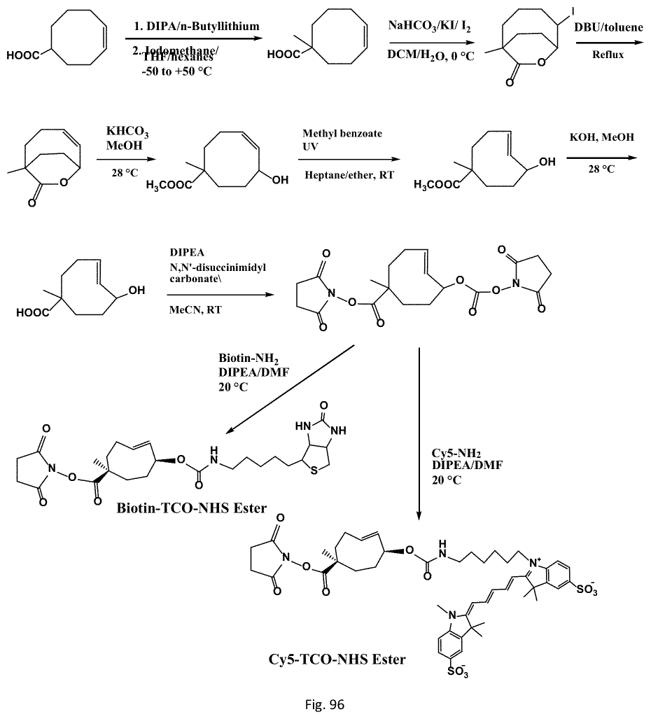

Synthesis of NHS-trans-Cyclooctene-Label (Dye or Anchor) (FIG. 96): Starting from carboxylic acid cyclooctene, following the reported method (Rossin 2016), a methyl group is introduced near the carboxylic acid cyclooctene, to prevent epimerization during lactone hydrolysis and to enable regioselective conjugation. Iodolactonization and subsequent hydrogen iodide elimination result in enelactone. Hydrolysis gives the desired ring-opened cyclooctene with the hydroxyl positioned cis relative to the methyl ester. UV irradiation affords a mixture of the two possible TCO isomers with the hydroxyl respectively in the axial and equatorial position and the methyl ester respectively in the equatorial and axial positions. After hydrolysis of the methyl ester, straightforward separation affords the expected isomer (HOOC-TCO-OH). DSC activation results in the bis-NHS derivative of TCO, and subsequent reaction with 1 equiv Dye-NH₂ (Cy5) or Anchor-NH₂ (Biotin) affords the desired selective reaction with the NHS-carbonate vs the sterically hindered NHS-ester, leading to axial NHS-TCO-Dye or NHS-TCO-Anchor (FIG. 96).

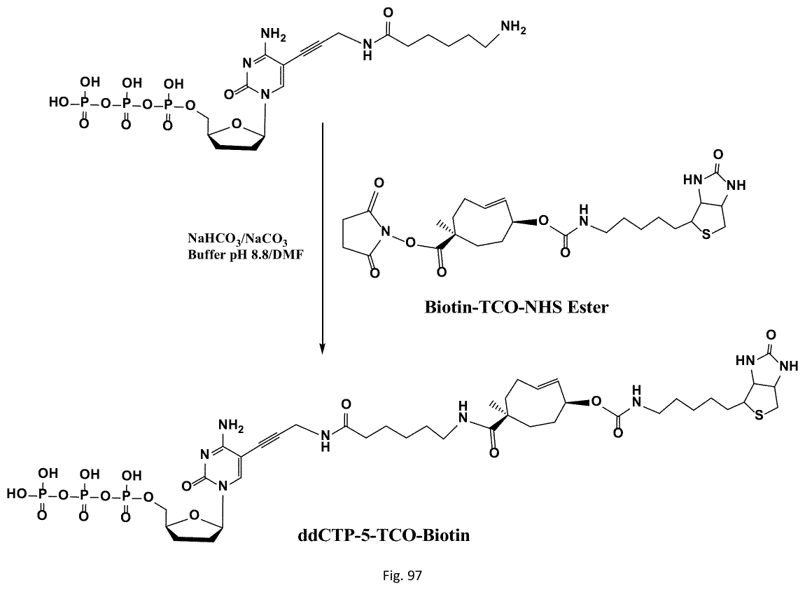

NHS-TCO-Dye or NHS-TCO-Anchor are used to couple with ddNTP-NH₂ in DMF/0.1M Na₂CO₃/NaHCO₃ buffer (pH. 8.8) affording ddNTP-TCO-Dye(or Anchor) (FIG. 97).

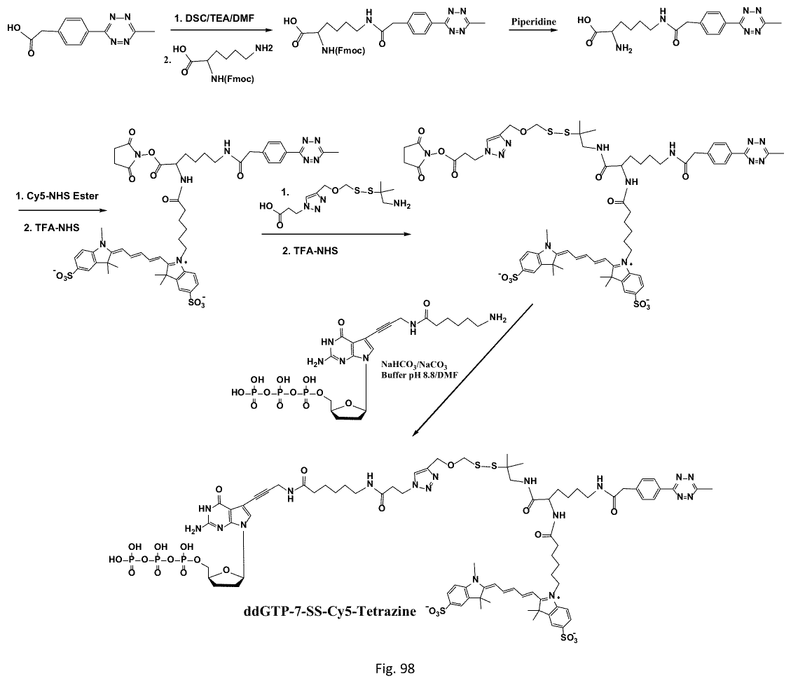

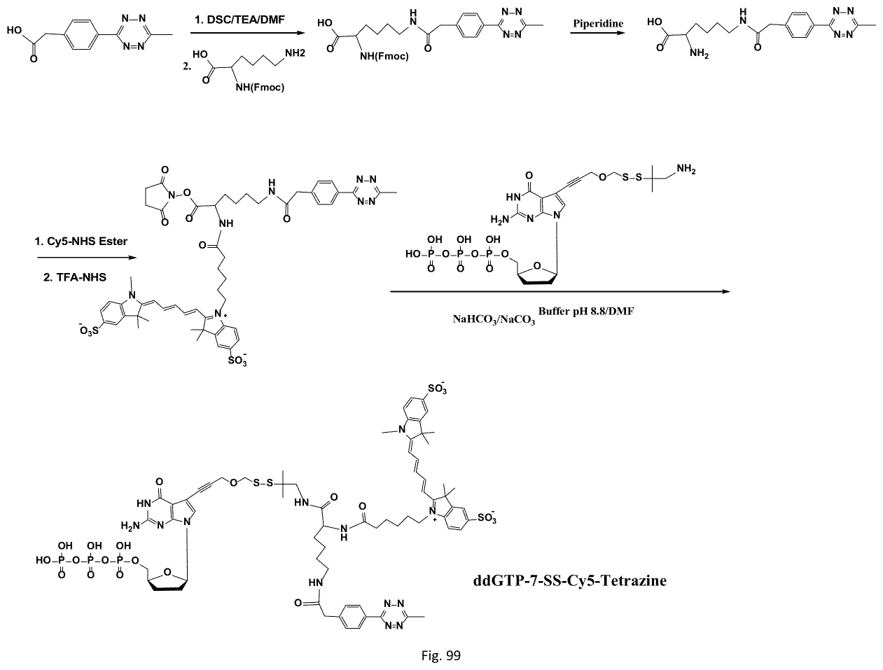

Synthesis of ddNTP-SS-Label(Dye or Anchor)-Tetrazine (FIG. 98, FIG. 99) starts from carboxlic acid derivatized Tetrazine, which is coupled to the 6-NH₂ of a-amino protected lysine via an amide bond by treatment with DSC and TEA. After removal of a-amino protection group Fmoc with piperidine, either Dye (e.g., Cy5) NHS ester or Anchor (e.g., biotin) NHS ester are used to label Tetrazine derivatized lysine, giving Dye (e.g., Cy5) or Anchor (e.g., biotin) labeled TCO-lysine which is further treated with TFA NHS yielding Dye or Anchor labeled Tetrazine-lysine-NHS ester. Dye or Anchor labeled Tetrazine-lysine-NHS ester can be used to couple with Amino-SS(DTM) linker and further converted to Dye or Anchor labeled Tetrazine-SS(DTM) linker-NHS ester, which can be coupled to amino group derivatized ddNTPs (ddNTP-NH₂) yielding ddNTP-SS-Label(Dye or Anchor)-Tetrazine (FIG. 98). Dye or Anchor labeled Tetrazine-lysine-NHS ester can also be used to couple with amino-SS(DTM) linker derivatized ddNTPs (ddNTP-SS-NH₂) yielding ddNTP-SS-Label(Dye or Anchor)-Tetrazine (FIG. 99).

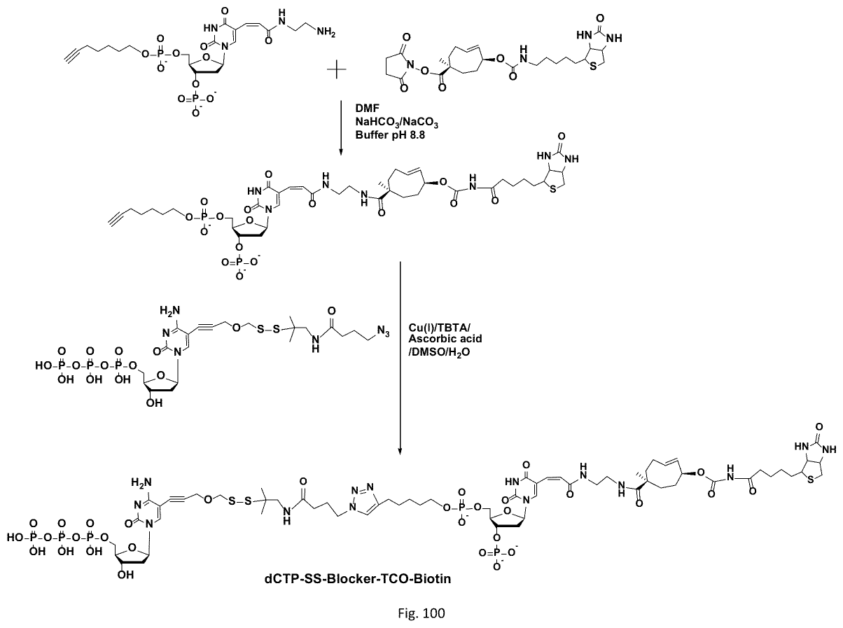

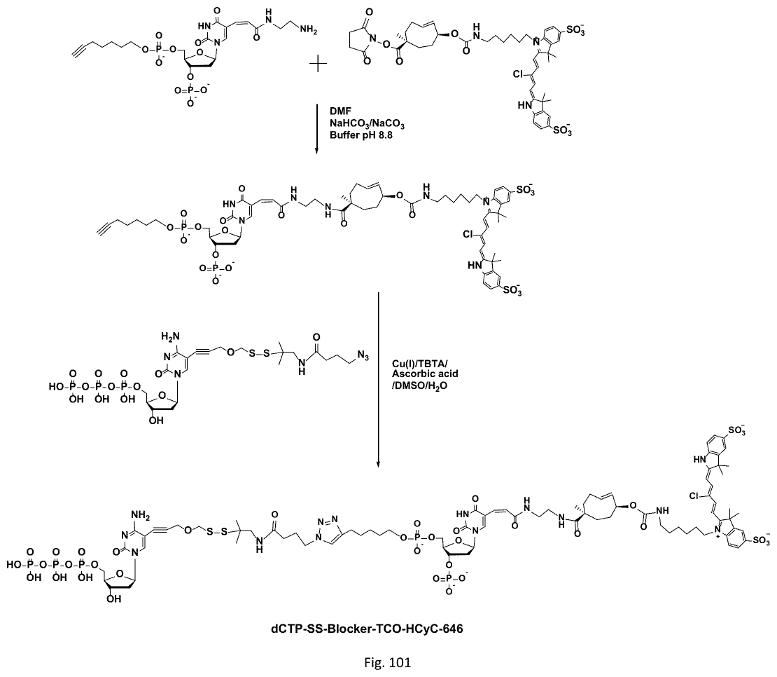

NHS-TCO-Dye or NHS-TCO-Anchor are used to couple with both amino group and terminal alkyne derivatized 3′,5′-diphosphate nucleotides (as incorporation blocking moieties in virtual terminators) affording alkyne-diphosphate-TCO-Dye(or Anchor), which is further coupled with azido and SS(DTM) derivatized dNTPs (dNTP-SS-N₃) via a Cu(I) catalyzed click reaction, yielding dNTP-SS-Blocker-TCO-Anchor(biotin as example) and dNTP-SS-Blocker-TCO-Dye(HCyC-646 as example) (FIG. 100 and FIG. 101 as examples).

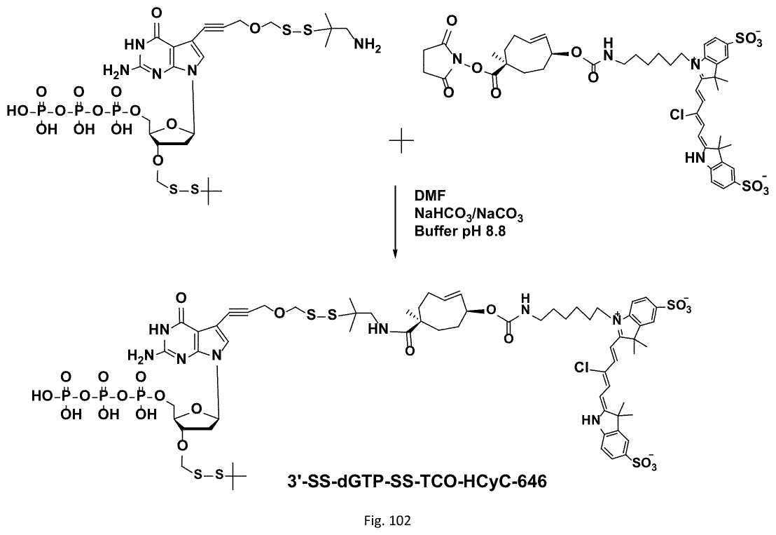

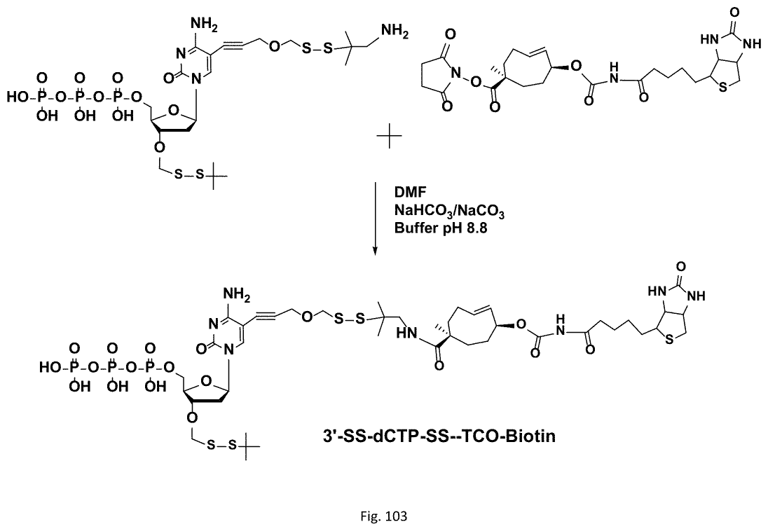

NHS-TCO-Dye or NHS-TCO-Anchor are used to couple with 3′-O-SS(DTM)-dNTP-SS-NH₂ in DMF/0.1M Na₂CO₃/NaHCO₃ buffer (pH. 8.8) affording 3′-O-SS(DTM)-dNTP-SS-TCO-Dye(or Anchor) (FIG. 102, FIG. 103 as examples).

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

### REFERENCES

Bentley D R et al (2008) Accurate whole human genome sequencing using reversible terminator chemistry. Nature. 456: 53-59.
Bowers, J., Mitchell, J., Beer, E., Buzby, P. R., Causey, M., Efcavitch, J. W., Jarosz, M., Krzymanska-Olejnik, E., Kung, L., Lipson, D., Lowman, G. M., Marappan, S., McInerney, P., Platt, A., Roy, A., Siddiqi, S. M., Steinmann, K., Thompson, J. F. (2009) Virtual terminator nucleotides for next-generation DNA sequencing. Nature Meth 6:593-595.
Braslavsky I, Hebert B, Kartalov E, Quake SR (2003) Sequence information can be obtained from single DNA molecules. Proc Natl Acad Sci USA 100:3960-3964.
Duthie R S, Kalve I M, Samols S B, Hamilton S, Livshin I, Khot M, Nampali S, Kumar S, Fuller C W (2002) Novel cyanine dye-labeled dideoxynucleoside triphosphates for DNA sequencing. Bioconj Chem 13:699-706.
Eid J et al (2009) Real-time DNA sequencing from single polymerase molecules. Science. 323: 133-138.
Fuller C, Kumar S, et al (2016) Real-time single molecule electronic DNA sequencing by synthesis using tagged-nucleotides on Genia chip. Proc Natl Acad Sci USA 113:5233-5238.
Guo J, Xu N, Li Z, Zhang S, Wu J, Ki, D H, Marma M S, Meng Q, Cao H, Li X, Shi S, Yu L, Kalachikov S, Russo J J, Turro N J, Ju J (2008) Four-color DNA sequencing with 3′-O-modified nucleotide reversible terminators and chemically cleavable fluorescent dideoxynucleotides. Proc Natl Acad Sci USA. 105: 914-950.
Harris T D et al (2008) Single-molecule DNA sequencing of a viral genome. Science. 320: 106-109.
Hilderbrand SA, Kelly K A, Niedre M, Weissleder R (2008) Near infrared fluorescence-based bacteriophage particles for ratiometric pH imaging. Bioconjug Chem. 19: 1635-1639.
Hobbs F W Jr, Trainor GL (1992) U.S. Pat. No. 5,151,507 A. Alkynylamino-nucleotides. Hung S-C, Ju J, Mathies RA, Glazer AN (1996) Cyanine dyes with high absorption cross section as donor chromophores in energy transfer primers. Anal Biochem 243:15-27.
Hyman ED (1988) A new method of sequencing DNA. Anal Biochem. 174: 423-436.
Ju J (2007) DNA sequencing by nanopore using modified nucleotides. U.S. Pat. No. 8,889,348 B2, US 20090298072 A1.
Ju J et al (2006) Four-color DNA sequencing by synthesis using cleavable fluorescent nucleotide reversible terminators. Proc Natl Acad Sci USA. 103: 19635-19640.
Ju J, Chen X, Li X, Li Z, Hsieh M-K, Chien M, Shi S, Ren J, Guo C, Kumar S, Russo JJ, Tao C, Jockusch S, Kalachikov S (2017) WO 2017/205336 A1. Nucleotide derivatives and methods of use thereof.
Ju J, Cho Y, Kumar S, Kalachikov S, Tao C, Chien M, Russo JJ (2016) WO 2016/154215 A1. Polymer tagged nucleotides for single molecule electronic SNP assay.
Ju J, Li X, Kumar S, Chen X, Russo J, Chien M, Jockusch S, Tao C, Wang X, Kalachikov S, Morozova I, Shi S, (2019) PCT/US2019/022326. Nucleotide Analogues and Use Thereof for Nucleic Acid Sequencing and Analysis.
Ju J, Glazer AN, Mathies RA (1996) Cassette labeling for facile construction of energy transfer fluorescent primers. Nucleic Acids Res 24:1144-1148.
Ju J, Kim DH, Guo J, Meng Q, Li Z, Cao H (2016) US 2016/0024574 A1. DNA sequencing with non-fluorescent nucleotide reversible terminators and cleavable label modified nucleotide terminators.
Ju J, Kumar S, Li Z, Tao C, Chien M, Russo JJ, Kalachikov S, Shepard K, Rosenstein JK (2013) DNA sequencing by synthesis using modified nucleotides and nanopore detection. EP 2652153 A2 (WO 2012083249 A2) Ju J, Kumar S, Russo JJ, Jockusch S, Li Z, Li X, Kalachikov S, Morozova I (2019) Fluorescence energy transfer-based single molecule/ensemble DNA sequencing by synthesis. US 2019/0153527 A1.
Ju J, Li Z, Tong A, Russo JJ (2003) Combinatorial fluorescence energy transfer tags and their applications for multiplex genetic analyses. U.S. Pat. No. 6,627,748 B1.
Ju J, Li X, Chen X, Kumar S, Russo J, Chien M (2018) 3′-O-Modified nucleotide analogues with different cleavable linkers for attaching fluorescent labels to the base for DNA sequencing by synthesis. WO 2018/183538 A1 (US 2018/024895).
Ju J, Li X, Chen X, Li Z, Kumar S, Shi S, Guo C, Ren J, Hsieh M-K, Chien M, Tao C, Erturk E, Kalachikov S, Russo JJ (2017) WO 2017/058953 A1 (US 2018/0274024). Design and synthesis of novel disulfide linker based nucleotides as reversible terminators for DNA sequencing by synthesis.
Ju J, Wu J, Li Z (2017) U.S. Pat. No. 9,624,539. DNA sequencing by synthesis using Raman and Infrared spectroscopy detection.
Ju J, et al (2003) Massive parallel method for decoding DNA and RNA. U.S. Pat. No. 6,664,079.
Kumar S, Sood A (2006) Labeled Nucleoside Polyphosphates. U.S. Pat. No. 7,041,812 Kumar S, Tao C, Chien M, Hellner B, Balijepalli A, Robertson JWF, Li Z, Russo JJ, Reiner JE, Kasianowicz J, Ju J (2012) PEG-Labeled Nucleotides and Nanopore Detection for Single Molecule DNA Sequencing by Synthesis. Scientific Reports 2, 684.
Li Z et al (2003) A photocleavable fluorescent nucleotide for DNA sequencing and analysis. Proc Natl Acad Sci USA. 100: 414-419. Margulies M et al (2005) Genome sequencing in microfabricated high-density picolitre reactors. Nature. 437: 376-380.
Mitra RD, Shendure J, Olejnik J, EdytaKrzymanska 0, Church GM (2003) Fluore scent in situ sequencing on polymerase colonies. Anal Biochem 320:55-65.
Mujumdar RB, Ernst L A, Mujumdar SR, Lewis CJ, Waggoner AS (1993) Cyanine dye labeling reagents: Sulfoindocyanine succinimidyl esters. Bioconj Chem 4:105-111.
Ronaghi M et al (1998) A sequencing method based on real-time pyrophosphate. Science. 281: 363-365.
Rossin R et al (2016) Triggered drug release from an antibody-drug conjugate using fast “click-to-release” chemistry in mice. Bioconjug Chem. 27: 1697-1706.
Rossin R et al (2018) Chemically triggered drug release from an antibody-drug conjugate leads to potent antitumour activity in mice. Nature Commun. 9: 1484.
Rothberg J M et al (2011) An integrated semiconductor device enabling non-optical genome sequencing. Nature. 475:348-352.
Ruparel H et al (2005) Design and synthesis of a 3′-O-allyl photocleavable fluorescent nucleotide as a reversible terminator for DNA sequencing by synthesis. Proc Natl Acad Sci USA. 102: 5932-5937.
Seo, T S, Bai X, Kim DH, Meng Q, Shi S, Ruparel H, Li Z, Turro N J, Ju J (2005) Four-color DNA sequencing by synthesis on a chip using photocleavable fluorescent nucleotides. Proc Natl Acad Sci USA 102:5926-5931.
Turcatti G, Romieu A, Fedurco M, Tairi A-P (2008) A new class of reversible fluorescent nucleotides: synthesis and optimization as reversible terminators for DNA sequencing by synthesis. Nucleic Acids Res 36:e25.
Versteegen R M et al (2018) Click-to-release from trans-cyclooctenes: mechanistic insights and expansion of scope from established carbamate to remarkable ether cleavage. Ang Chem. 57: 10494-10499.
Wu J et al (2007) 3′-O-modified nucleotides as reversible terminators for pyrosequencing. Proc Natl Acad Sci USA. 104: 16462-16467.
Yang YY, Grammel M, Raghavan AS, Charron G, Hang HC (2010) Comparative analysis of cleavable azobenzene-based affinity tags for biorthogonal chemical proteomics. Chem Biol 17:1212-1222.

1-138. (canceled)

139. A nucleotide analogue having the structure: 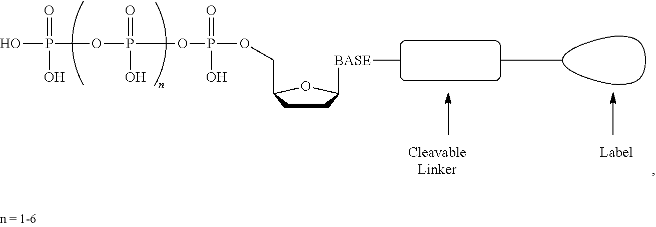
wherein:
BASE comprises adenine, guanine, cytosine, thymine, uracil, hypoxanthine or analog thereof;
Cleavable Linker comprises DTM, Azo, 2-Nitrobenzyl, Allyl, Azidomethyl, or TCO Derivative, and is attached to the base via 5 position of pyrimidines (C, U) or 7 position of deazapurines (A, G, I); and
Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye; or 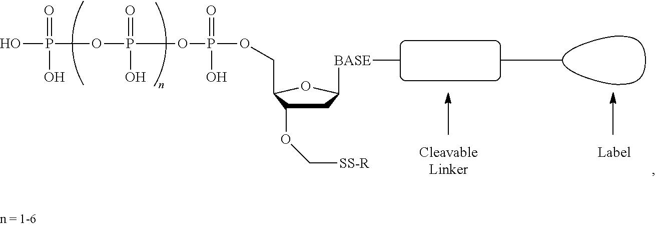
wherein:
BASE comprises adenine, guanine, cytosine, thymine, uracil, hypoxanthine or analog thereof;
R comprises methyl, ethyl, propyl, t-butyl, aryl, alkyl aryl;
Cleavable Linker comprises DTM, Azo, 2-Nitrobenzyl, Allyl, Azidomethyl or TCO Derivative; and
Label comprises a fluorescent dye, a pH responsive fluorescent dye, a cluster of a fluorescent dye, a cluster of a pH responsive fluorescent dye, an anchor for attachment of a fluorescent dye, a cluster of an anchor for attachment of fluorescent dyes, or an anchor and dye; or 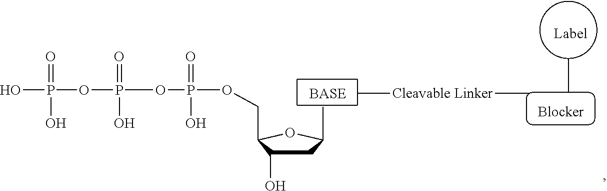
wherein:
BASE comprises adenine, guanine, cytosine, thymine, uracil, hypoxanthine or analog thereof;
Cleavable Linker comprises DTM, Azo, 2-Nitrobenzyl, Allyl, Azidomethyl, or TCO Derivative, or more than one of these cleavable linkers, including the special case where one cleavable linker is present between the base and the blocker and a second different cleavable linker is present between the blocker and the label;
Blocker is a nucleotide or oligonucleotide comprising 2-50 monomer units of abasic sugars or modified nucleosides or a combination thereof; and blocker is connected to the 5-position of pyrimidines (C, U) and 7-position of deazapurines (A, G, I) via a cleavable linker;
wherein a Blocker is a moiety that, after incorporation, prevents further incorporation of additional nucleotides or nucleotide analogues into a primer strand; and
Label comprises a fluorescent dye, a pH responsive fluorescent dye, a cluster of a fluorescent dye, a cluster of a pH responsive fluorescent dye, an anchor for attachment of a fluorescent dye, a cluster of an anchor for attachment of fluorescent dyes, or an anchor and dye, wherein the label is attached to the blocker; or 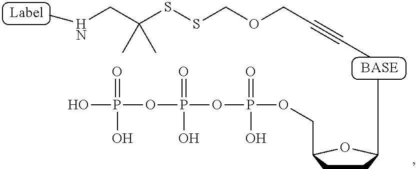
wherein:
BASE comprises adenine, guanine, cytosine, thymine, uracil, hypoxanthine or analog thereof; and
Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye; or 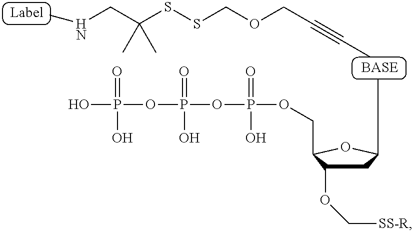
wherein:
BASE comprises adenine, guanine, cytosine, thymine, uracil, hypoxanthine or analog thereof;
Label comprises a fluorescent dye, a pH responsive fluorescent dye, a cluster of fluorescent dyes, a cluster of pH responsive fluorescent dyes, an anchor for dye attachment, an anchor cluster for dye attachment, or an anchor and dye; and
R comprises methyl, ethyl, propyl, t-butyl, aryl, alkyl aryl.

140. The nucleotide analogue of claim 139, wherein Label is selected from fluoresceins, rhodamines, cyanines, ATTO and Dyomics dyes; or wherein Label comprises Cy3, Cy5, or HCyC-646.

141. The nucleotide analogue of claim 139, wherein Label is an energy transfer dye or a cluster of energy transfer dyes with a donor and acceptor dye, wherein the donor dye may be a quantum dot.

142. The nucleotide analogue of claim 139, wherein Label is an energy transfer dye, and the donor dye is Fluorescein, CyA, Cy3 and the acceptor dye is Rhodamine 110, R6G, TAMRA, ROX, Cy5, HCyC-646, Alexa 647; or wherein Label is an anchor, and the anchor comprises Biotin, DBCO, TCO, or Tetrazine, and wherein the anchor binding molecule comprises Streptavidin, Azide, Tetrazine, or TCO and further comprises a single dye, a cluster of a single dyes, or energy transfer dyes.

143. The nucleotide analogue of claim 139, wherein the anchor binding molecule is labeled with an organic dye or a quantum dot.

144. The nucleotide analogue of claim 139, wherein Cleavable Linker is DTM based; or wherein Cleavable Linker is azo based.

145. A composition comprising four different types of the nucleotide analogue of claim 139, wherein a different dye is attached to each of the four different nucleotide analogues; or wherein two of the nucleotide analogues comprise a first dye and the remaining two nucleotide analogues comprise a second dye; or wherein one type of dye is attached to each of the four nucleotides via the same cleavable linker; or wherein one type of dye is attached to each of the four nucleotides via a different cleavable linker.

146. A nucleotide analogue having the structure: 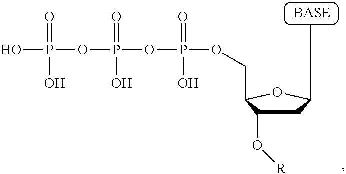
wherein:
BASE comprises adenine, guanine, cytosine, uracil, thymine, hypoxanthine or analogue thereof; and
R is a cleavable chemical group comprising alkyl DTM, Azo, 2-Nitrobenzyl, Allyl and Azidomethyl Derivatives; or 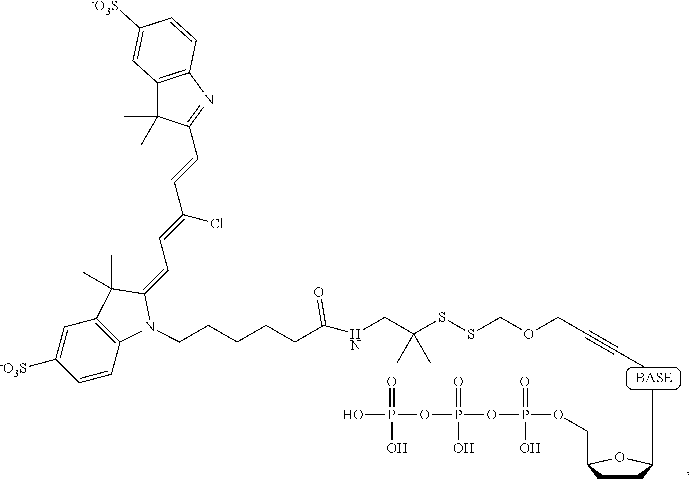
wherein BASE comprises adenine, guanine, cytosine, thymine, uracil, hypoxanthine or analog thereof; or 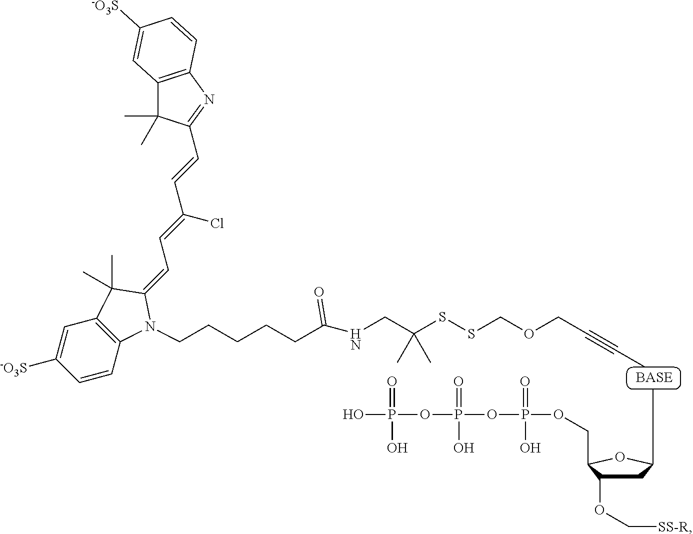
wherein:
BASE comprises adenine, guanine, cytosine, thymine, uracil, hypoxanthine or analog thereof; and
R comprises methyl, ethyl, propyl, t-butyl, aryl, alkyl aryl; or 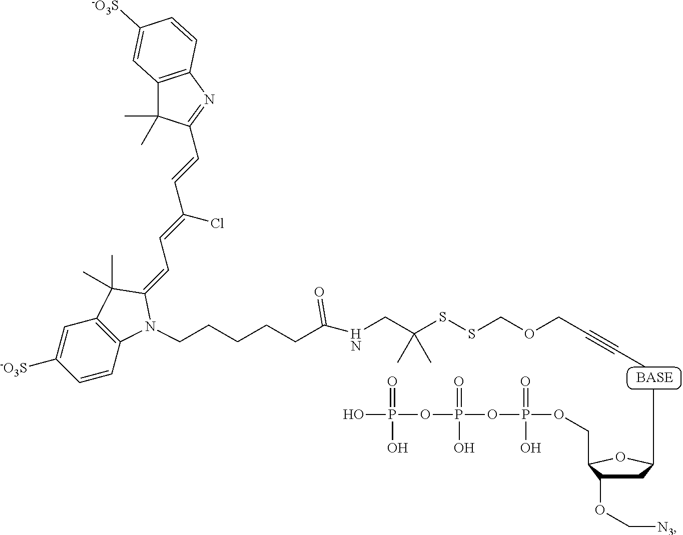
wherein BASE comprises adenine, guanine, cytosine, thymine, uracil, hypoxanthine or analog thereof.

147. A method of sequencing a nucleic acid comprising:
[A]
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) (A) fluorescently labeled dideoxynucleotide analogue comprising a base and a fluorescent label linked to the base via a cleavable linker, (B) pH-responsive fluorescently labeled dideoxynucleotide analogue comprising a base and a pH-responsive fluorescent label linked to the base via a cleavable linker, (C) two different anchor labeled dideoxynucleotide analogues, wherein each analogue comprises a different anchor attached to the base via a cleavable linker,
wherein the cleavable linkers are cleavable by an identical cleaving agent;
(ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (B) a pH-responsive fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, and (C) two different anchor labeled nucleotide analogues comprising a base and an anchor attached to the base via a cleavable linker and a blocking group at the 3′-OH position, wherein said blocking group prevents incorporation of a subsequent nucleotide analogue into the extended primer strand, wherein each analogue comprises a different anchor attached to the base via a cleavable linker,
wherein the cleavable linkers and 3′-O blocking groups are cleavable by an identical cleaving agent; or
(iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand, and (C) two different anchor labeled nucleotide analogues comprising a base, a blocking group linked to the base via a cleavable linker, and an anchor linked to the base distal to the blocking group, wherein said blocking group prevents or greatly reduces incorporation of a subsequent nucleotide analogue into the extended primer strand, wherein each analogue comprises a different anchor attached to the base via a cleavable linker,
wherein the cleavable linkers are cleavable by an identical cleaving agent;
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label has same or similar absorption and emission profile as the fluorescent label on the fluorescently labeled nucleotide analogue and identifying any fluorescence signal due to incorporation of a labeled nucleotide analogue from step (b);
e) contacting the incorporated nucleotide analogue from step (b) with (A) an anchor binding group that binds to the anchor of only one of the anchor labeled nucleotide analogues of step (b), wherein the anchor binding group comprises the same fluorescent label as the fluorescently labeled nucleotide analogue of step (b), and (B) an anchor binding group that binds only to the anchor of the remaining anchor labeled nucleotide analogue, wherein the anchor binding group comprises the same pH-responsive fluorescent label as the pH-responsive fluorescently labeled nucleotide analogue of step (b);
f) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of an anchor labeled nucleotide analogue from step (b);
g) washing the incorporated nucleotide analogue from step (b) at a pH at which the pH-responsive fluorescent label no longer has the same or similar absorption and emission profile as the fluorescent label on the fluorescently labeled nucleotide analogue and identifying any fluorescence signal due to incorporation of a labeled nucleotide analogue from step (b);
h) cleaving the cleavable linker from the incorporated nucleotide analogue, thereby removing any label, anchor, or blocking group from the incorporated nucleotide analogue of step (b);
i) cleaving the 3′-O blocking group from any incorporated nucleotide analogue from step (c); and
j) iteratively repeating steps (b) to (i) for each residue of the nucleic acid to be sequenced; or
[B]
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via a carbamyl TCO linker, (C) an anchor labeled dideoxynucleotide analogue comprising a base and an anchor attached to the base via the first cleavable linker, and (D) an anchor labeled dideoxynucleotide analogue comprising a base and an anchor attached to the base via a carbamyl TCO linker;
(ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via a carbamyl TCO linker, and a blocking group at the 3′-OH position, wherein said blocking group prevents incorporation of a subsequent nucleotide analogue into the extended primer strand, (C) an anchor labeled nucleotide analogue comprising a base and an anchor attached to the base via the first cleavable linker, and a blocking group at the 3′-OH position, wherein said blocking group prevents incorporation of a subsequent nucleotide analogue into the extended primer strand, and (D) an anchor labeled nucleotide analogue comprising a base and an anchor attached to the base via a carbamyl TCO linker, and a blocking group at the 3′-OH position, wherein said blocking group prevents incorporation of a subsequent nucleotide analogue into the extended primer strand, wherein the 3′-O blocking group and the first cleavable linker are cleavable by the same cleaving agent; or
(iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a carbamyl TCO linker, and a fluorescent label linked to the base distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a first cleavable linker, and an anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a carbamyl TCO linker, and an anchor linked to the base distal to the blocking group, wherein said blocking group prevents or greatly reduces incorporation of a subsequent nucleotide analogue into the extended primer strand,
wherein the fluorescent labels on each analogue are the same,
wherein the anchors on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the anchor of the provided anchor labeled nucleotide analogues of step (b), wherein said anchor binding group comprises a fluorescent label identical to the fluorescent label of the fluorescently labeled nucleotide analogues of step (b);
f) identifying any fluorescence signal due to the binding of the anchor binding group to the anchor of any incorporated anchor labeled nucleotide analogue of step (b);
g) contacting the incorporated nucleotide analogue with a tetrazine derivative to click to the TCO moiety of the carbamyl TCO linker to release any label or anchor linked by a carbamyl TCO linker and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
iteratively repeating steps (b) to (h) for each residue of the nucleic acid to be sequenced; or
[C]
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) (A) a fluorescently labeled dideoxynucleotide analogue comprising a base and a fluorescent label linked to the base via a cleavable linker, (B) a fluorescently labeled dideoxynucleotide analogue comprising a base and a fluorescent label and a first anchor linked to the base via a cleavable linker, (C) an anchor labeled dideoxynucleotide analogue comprising a base and the first anchor and a second anchor attached to the base via a cleavable linker, and (D) an anchor labeled dideoxynucleotide analogue comprising a base and the second anchor attached to the base via a cleavable linker, wherein the cleavable linkers are cleavable by an identical cleavage agent;
(ii) (A) a fluorescently labeled nucleotide analogue comprising a base and a fluorescent label linked to the base via a cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label and a first anchor linked to the base via a cleavable linker, and a blocking group at the 3′-OH position, wherein said blocking group prevents incorporation of a subsequent nucleotide analogue into the extended primer strand, (C) an anchor labeled nucleotide analogue comprising a base and the first anchor and a second anchor attached to the base via the cleavable linker, and a blocking group at the 3′-OH position, wherein said blocking group prevents incorporation of a subsequent nucleotide analogue into the extended primer strand, and (D) an anchor labeled nucleotide analogue comprising a base and the second anchor attached to the base via a cleavable linker, and a blocking group at the 3′-OH position, wherein said blocking group prevents incorporation of a subsequent nucleotide analogue into the extended primer strand, wherein the cleavable linkers and the 3′-O blocking group are cleavable by an identical cleavage agent; or
(iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a cleavable linker, and a fluorescent label and a first anchor linked to the base distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a cleavable linker, and the first anchor and a second anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a cleavable linker, and the second anchor linked to the base distal to the blocking group, wherein said blocking group prevents or greatly reduces incorporation of a subsequent nucleotide analogue into the extended primer strand, wherein the cleavable linkers are cleavable by an identical cleavage agent,
wherein the fluorescent labels on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue;
e) contacting the incorporated nucleotide analogue from step (b) with an anchor binding group that binds to the second anchor of the nucleotide analogues of step (b), wherein said anchor binding group comprises a fluorescent label identical to fluorescent label of the fluorescently labeled nucleotide analogues of step (b);
f) identifying any fluorescence signal due to the binding of the anchor binding group to the anchor of any incorporated nucleotide analogue of step (b);
g) contacting the incorporated nucleotide analogue with a second anchor binding group that binds to the first anchor of the nucleotide analogues of step (b) and comprises a moiety that quenches the fluorescent signal of any fluorescent label attached to the nucleotide analogue to which the anchor binding group attaches, and identifying any fluorescence signal due to incorporation of a fluorescently labeled nucleotide analogue;
h) contacting the incorporated nucleotide analogue with a cleaving agent that cleaves the cleavable linker and any 3′-O blocking group; and
iteratively repeating steps (b) to (h) for each residue of the nucleic acid to be sequenced; or
[D]
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via the first cleavable linker and a carbamyl TCO linker attached distal to the first cleavable linker, (C) an anchor labeled dideoxynucleotide analogue comprising a base and an anchor linked to the base via the first cleavable linker and a carbamyl TCO linker attached distal to the first cleavable linker, and (D) an anchor labeled dideoxynucleotide analogue comprising a base and an anchor attached to the base via the first cleavable linker;
(ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, (C) an anchor labeled nucleotide analogue comprising a base and an anchor attached to the base via the first cleavable linker, and a blocking group at the 3′-OH position, wherein said blocking group prevents incorporation of a subsequent nucleotide analogue into the extended primer strand, and (D) an anchor labeled nucleotide analogue comprising a base and an anchor linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, wherein the 3′-OH blocking group and the first cleavable linker are cleavable by the same agent; or
(iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via the first cleavable linker, and a fluorescent label linked to the base via a carbamyl TCO linker distal to the blocking group, wherein said blocking group prevents or greatly reduces incorporation of a subsequent nucleotide analogue into the extended primer strand, (C) an anchor labeled nucleotide analogue comprising a base, a blocking group linked to the base via a first cleavable linker, and an anchor linked to the base distal to the blocking group, wherein said blocking group prevents or greatly reduces incorporation of a subsequent nucleotide analogue into the extended primer strand, and (D) an anchor labeled nucleotide analogue comprising a base, a blocking group linked to the base via a the first cleavable linker, and an anchor linked to the base via a carbamyl TCO linker distal to the blocking group, wherein said blocking group prevents or greatly reduces incorporation of a subsequent nucleotide analogue into the extended primer strand,
wherein the fluorescent labels on each analogue are the same,
wherein the anchors on each analogue are the same;
c) extending unextended primers with a nucleotide analogue without any base modifications and comprising a 3′-O blocking group, wherein step (c) occurs before, simultaneously, or after step (b);
d) identifying any fluorescence signal due to incorporation of a fluorescently labeled nucleotide analogue in step (b);
e) contacting the incorporated nucleotide analogue from step (b) with an anchor binding group that binds to the anchor of the provided anchor labeled nucleotide analogues of step (b), wherein said anchor binding group comprises a fluorescent label identical to the fluorescent label of the fluorescently labeled nucleotide analogues of step (b);
f) identifying any fluorescence signal due to the binding of the anchor binding group to the anchor of any incorporated anchor labeled nucleotide analogue of step (b);
g) contacting the incorporated nucleotide analogue with a tetrazine derivative to click to the TCO moiety of the carbamyl TCO linker to release any label or anchor linked by a carbamyl TCO linker and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced; or
[E]
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) (A) a fluorescently labeled dideoxynucleotide analogue comprising a base and a fluorescent label linked to the base via a first cleavable linker, (B) a fluorescently labeled dideoxynucleotide analogue comprising a base and a fluorescent label linked to the base via the first cleavable linker and a carbamyl TCO linker attached distal to the first cleavable linker, (C) a pH-responsive fluorescently labeled dideoxynucleotide analogue comprising a base and a pH-responsive fluorescent label linked to the base via the first cleavable linker and a carbamyl TCO linker attached distal to the first cleavable linker, and (D) a pH-responsive fluorescently labeled dideoxynucleotide analogue comprising a base and a pH-responsive fluorescent label attached to the base via the first cleavable linker;
(ii) (A) a fluorescently labeled nucleotide analogue comprising a base and a fluorescent label linked to the base via a first cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, (C) a pH-responsive fluorescently labeled nucleotide analogue comprising a base and a pH-responsive fluorescent label attached to the base via the first cleavable linker, and a blocking group at the 3′-OH position, wherein said blocking group prevents incorporation of a subsequent nucleotide analogue into the extended primer strand, and (D) a pH-responsive fluorescently labeled nucleotide analogue comprising a base and a pH-responsive fluorescent label linked to the base via the first cleavable linker and a carbamyl TCO linker attached distal to the first cleavable linker, and a blocking group at the 3′-OH position, wherein said blocking group prevents incorporation of a subsequent nucleotide analogue into the extended primer strand, wherein the 3′-OH blocking group and the first cleavable linker are cleavable by the same agent; or
(iii) (A) a fluorescently labeled nucleotide analogue comprising a base, a blocking group linked to the base via a first cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via the first cleavable linker, and a fluorescent label linked to the base via a carbamyl TCO linker distal to the blocking group, wherein said blocking group prevents or greatly reduces incorporation of a subsequent nucleotide analogue into the extended primer strand, (C) a pH-responsive fluorescently labeled nucleotide analogue nucleotide analogue comprising a base, a blocking group linked to the base via a first cleavable linker, and a pH-responsive fluorescent label linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a the first cleavable linker, and a pH-responsive fluorescent label linked to the base via a carbamyl TCO linker distal to the blocking group, wherein said blocking group prevents or greatly reduces incorporation of a subsequent nucleotide analogue into the extended primer strand,
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label does not have the same or similar absorption and emission profile as the fluorescent label on the fluorescently labeled nucleotide analogues and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b), wherein steps (d) and (e) may be performed in the reverse order;
f) contacting the incorporated nucleotide analogue with tetrazine to click the TCO moiety of the carbamyl TCO linker to release any label linked by a carbamyl TCO linker;
g) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b);
h) contacting the incorporated nucleotide analogue with a cleaving agent that cleaves the first cleavable linker and any 3′-O blocking group; and
i) iteratively repeating steps (b) to (h) for each residue of the nucleic acid to be sequenced; or
[F]
a) providing a plurality of nucleic acid templates each hybridized to a primer and a nucleic acid polymerase, wherein each template has the same sequence as the nucleic acid to be sequenced;
b) contacting the nucleic acid templates with four different labeled nucleotide analogues and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) (A) a fluorescently labeled dideoxynucleotide analogue comprising a base and a fluorescent label linked to the base via a cleavable linker, (B) a fluorescently labeled dideoxynucleotide analogue comprising a base and a fluorescent label and an anchor linked to the base via a cleavable linker, (C) a pH-responsive fluorescently labeled dideoxynucleotide analogue comprising a base and a pH-responsive fluorescent label linked to the base via a cleavable linker, and (D) a pH-responsive fluorescently labeled dideoxynucleotide analogue comprising a base and pH-responsive fluorescent label and anchor attached to the base via a cleavable linker, wherein the cleavable linkers are cleavable by an identical cleaving agent;
(ii) (A) a fluorescently labeled nucleotide analogue comprising a base and a fluorescent label linked to the base via a cleavable linker, and a blocking group at the 3′-OH position, wherein said blocking group prevents incorporation of a subsequent nucleotide analogue into the extended primer strand, (B) a fluorescently labeled nucleotide analogue comprising a base and a fluorescent label and an anchor linked to the base via a cleavable linker, and a blocking group at the 3′-OH position, wherein said blocking group prevents incorporation of a subsequent nucleotide analogue into the extended primer strand, (C) a pH-responsive fluorescently labeled nucleotide analogue comprising a base and pH-responsive fluorescent label linked to the base via a cleavable linker, and a blocking group at the 3′-OH position, wherein said blocking group prevents incorporation of a subsequent nucleotide analogue into the extended primer strand, and (D) a pH-responsive fluorescently labeled nucleotide analogue comprising a base and a pH-responsive fluorescent label and anchor attached to the base via a cleavable linker, and a blocking group at the 3′-OH position, wherein said blocking group prevents incorporation of a subsequent nucleotide analogue into the extended primer strand, wherein the cleavable linkers and the 3′-O blocking group are cleavable by an identical cleaving agent; or
(iii) (A) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, (C) a pH-responsive fluorescently labeled nucleotide analogue comprising a base, a blocking group linked to the base via a cleavable linker, a pH-responsive fluorescent label linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a cleavable linker, and a pH-responsive fluorescent label and an anchor linked to the base distal to the blocking group, wherein said blocking group prevents or greatly reduces incorporation of a subsequent nucleotide analogue into the extended primer strand, wherein the cleavable linkers are cleavable by an identical cleavage agent,
c) extending unextended primers with a nucleotide analogue without any base modifications and comprising a 3′-O blocking group, wherein step (c) occurs before, simultaneously, or after step (b);
d) washing away any unincorporated nucleotide analogues at a pH at which the pH-responsive fluorescent label does not have the same or similar absorption and emission profile as the fluorescent label on the fluorescently labeled nucleotide analogues and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogues and identifying any fluorescence signal due to incorporation of a labeled nucleotide analogue from step (b), wherein steps (d) and (e) may be performed in the reverse order;
f) contacting the incorporated nucleotide analogue from step (b) with an anchor binding group that binds to the anchor of the nucleotide analogues of step (b), wherein said anchor binding group comprises a moiety that quenches the fluorescent label of the fluorescently labeled nucleotide analogues of step (b);
g) washing away any unbound anchor binding group comprising a quenching moiety at a pH at which the pH-responsive fluorescent label has same or similar absorption and emission profile as the fluorescent label on the fluorescently labeled nucleotide analogues and identifying any fluorescence signal due to incorporation of a labeled nucleotide analogue from step (b);
h) contacting the incorporated nucleotide analogue with a cleaving agent that cleaves the cleavable linker and any 3′-O blocking group; and
i) iteratively repeating steps (b) to (h) for each residue of the nucleic acid to be sequenced; or
[G]
a) providing a plurality of nucleic acid templates each hybridized to a primer, wherein each template has the same sequence as the nucleic acid to be sequenced, and providing a nucleic acid polymerase;
b) contacting the nucleic acid templates with four different labeled nucleotide analogues (A, C, T, G) and under conditions permitting the nucleic acid polymerase to extend the primers with one of the labeled nucleotide analogues if the nucleotide analogue is complementary to a nucleotide residue which is immediately 5′ to the nucleotide residue of the nucleic acid template hybridized to the 3′ terminal nucleotide residue of the primer, wherein the four different labeled nucleotide analogues are either:
(i) (A) a fluorescently labeled dideoxynucleotide analogue comprising a base and a fluorescent label linked to the base via a cleavable linker, (B) a pH-responsive fluorescently labeled dideoxynucleotide analogue comprising a base and a pH-responsive fluorescent label linked to the base via a cleavable linker, (C) a fluorescently labeled dideoxynucleotide analogue comprising a base and a fluorescent label and anchor linked to the base via a cleavable linker, and (D) a pH-responsive fluorescently labeled dideoxynucleotide analogue comprising a base and a pH-responsive fluorescent label and identical anchor linked to the base via a cleavable linker,
wherein the cleavable linkers are cleavable by an identical cleaving agent;
(ii) (A) a fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, (B) a pH-responsive fluorescently labeled nucleotide analogue comprising a base and a fluorescent label linked to the base via a cleavable linker and a blocking group at the 3′-OH position, wherein said blocking group prevents incorporation of a subsequent nucleotide analogue into the extended primer strand, (C) a fluorescently labeled nucleotide analogue comprising a base and a fluorescent label and anchor linked to the base via a cleavable linker and a blocking group at the 3′-OH position, wherein said blocking group prevents incorporation of a subsequent nucleotide analogue into the extended primer strand, and (D) a pH-responsive fluorescently labeled nucleotide analogue comprising a base and a fluorescent label and identical anchor linked to the base via a cleavable linker and a blocking group at the 3′-OH position, wherein said blocking group prevents incorporation of a subsequent nucleotide analogue into the extended primer strand,
wherein the cleavable linkers and 3′-O blocking groups are cleavable by an identical cleaving agent; or
(iii) a fluorescently labeled nucleotide analogue comprising a base, a blocking group linked to the base via a cleavable linker, and a fluorescent label linked to the base distal to the blocking group, wherein said blocking group prevents or greatly reduces incorporation of a subsequent nucleotide analogue into the extended primer strand, (B) a pH-responsive fluorescently labeled nucleotide analogue comprising a base, a blocking group linked to the base via a cleavable linker, and a pH-responsive fluorescent label linked to the base distal to the blocking group, wherein said blocking group prevents or greatly reduces incorporation of a subsequent nucleotide analogue into the extended primer strand, (C) a fluorescently labeled nucleotide analogue comprising a base, a blocking group linked to the base via a cleavable linker, and a fluorescent label and anchor linked to the base distal to the blocking group, wherein said blocking group prevents or greatly reduces incorporation of a subsequent nucleotide analogue into the extended primer strand, and (D) a pH-responsive fluorescently labeled nucleotide analogue comprising a base, a blocking group linked to the base via a cleavable linker, and a pH-responsive fluorescent label and identical anchor linked to the base distal to the blocking group, wherein said blocking group prevents or greatly reduces incorporation of a subsequent nucleotide analogue into the extended primer strand,
wherein the cleavable linkers are cleavable by an identical cleaving agent;
c) extending unextended primers with a nucleotide analogue without any base modifications and comprising a 3′-O blocking group, wherein step (c) occurs before, simultaneously or after step (b);
d) washing away any unincorporated nucleotide analogues at a pH at which the pH-responsive fluorescent label does not have the same or similar absorption and emission profile as the fluorescent label on the fluorescently labeled nucleotide analogue and identifying any fluorescence signal due to incorporation of a labeled nucleotide analogue from step (b);
e) washing away any unincorporated nucleotide analogues at a pH at which the pH-responsive fluorescent label has the same or similar absorption and emission profile as the fluorescent label on the fluorescently labeled nucleotide analogue and identifying any fluorescence signal due to incorporation of an anchor labeled nucleotide analogue from step (b);
f) contacting the incorporated nucleotide analogue with an anchor binding group that binds to the anchor of the nucleotide analogues of step (b) and comprises a moiety that quenches the fluorescent signal of any fluorescent label attached to the nucleotide analogue to which the anchor binding group attaches, and identifying any fluorescence signal due to incorporation of a fluorescently labeled nucleotide analogue;
g) contacting the incorporated nucleotide analogue with a cleaving agent that cleaves the cleavable linker and any 3′-O blocking group; and
h) iteratively repeating steps (b) to (g) for each residue of the nucleic acid to be sequenced,
thereby obtaining the sequence of the nucleic acid.

148. The method of claim 147, wherein the four different labeled nucleotide analogues are the nucleotide analogues of step (b) (I) and are selected from the nucleotide analogues of claim 139.

149. The method of claim 147, wherein the four different labeled nucleotide analogues are the nucleotide analogues of step (b) (II) and are selected from the nucleotide analogues of claim 1.

150. The method of claim 147, wherein the four different labeled nucleotide analogues are the nucleotide analogues of step (b) (III) and are selected from the nucleotide analogues of claim 1.

151. The method of claim 147, wherein the label with pH-responsive fluorescence is HCyC-646 and the label with pH-unresponsive fluorescence is Cy5.

152. The method of claim 147, wherein step (c) occurs before step (b); or wherein step (c) occurs after step (b).

153. The method of claim 147, wherein the nucleotide analogue(s) without any base modifications and comprising a 3′-O blocking group in step (c) is a nucleotide analogue selected from claim 146.

154. The method of claim 147, wherein the nucleotide analogues added in step (c) are incorporated into primers of greater than 90% the nucleic acid templates.

155. The method of claim 147, wherein the nucleotide analogues added in step (c) are incorporated into primers of greater than 95% the nucleic acid templates.

156. The method of claim 147, wherein if there is an anchor present, the anchor comprises biotin, TCO, tetrazine, or DBCO, and the corresponding anchor binding molecule comprises streptavidin, azide, tetrazine and TCO.

157. The method of claim 147, wherein the fluorescent dye comprises organic dyes comprising xanthine, cyanine and ATTO dyes, quantum dots and clusters of organic dyes and quantum dots.

158. A kit comprising the appropriate nucleotides, labeling reagents, quenching reagents and other buffer components necessary to carry out the sequencing by synthesis reactions in claim 147.
